(19) United States
(12) Patent Application Publication
(10) Pub. No.: US 2025/0382293 A1
(43) Pub. Date: Dec. 18, 2025

(54) COMPOUNDS AND METHODS FOR YAP/TEAD MODULATION AND INDICATIONS THEREFOR

(71) Applicant: OPNA BIO SA, Epalinges (CH)

(72) Inventor: Somenath CHOWDHURY, South San Francisco, CA (US)

(21) Appl. No.: 19/278,896

(22) Filed: Jul. 24, 2025

Related U.S. Application Data
(63) Continuation of application No. PCT/US24/13073, filed on Jan. 26, 2024.
(60) Provisional application No. 63/441,627, filed on Jan. 27, 2023.

(51) Int. Cl. C07D 471/04; A61K 31/4375; A61K 31/4545; A61K 31/4725; A61K 31/519; A61K 45/06; C07D 217/06; C07D 217/08; C07D 401/04; C07D 401/06; C07D 401/12; C07D 409/06; C07D 413/06; C07D 417/06

(52) U.S. Cl. CPC C07D 471/04; A61K 31/4375; A61K 31/4545; A61K 31/4725; A61K 31/519; A61K 45/06; C07D 217/06; C07D 217/08; C07D 401/04; C07D 401/06; C07D 401/12; C07D 409/06; C07D 413/06; C07D 417/06

(57) ABSTRACT

Disclosed are compounds of Formula (I):

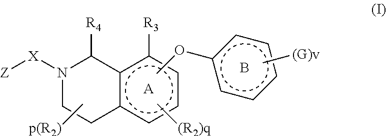

or a pharmaceutically acceptable salt, a tautomer, a stereoisomer, or a deuterated analog thereof, wherein ring A, ring B, G, R₂, R₃, R₄, p, q, v, X, and Z are as described in any of the embodiments described in this disclosure; compositions thereof; and uses thereof.

### RELATED APPLICATIONS

This application is continuation of International Application No. PCT/US2024/013073, which designated the United States and was filed on Jan. 26, 2024, published in English, which claims the benefit of priority of U.S. Provisional Application No. 63/441,627 filed Jan. 27, 2023. The entire contents of the above-referenced applications are incorporated by reference herein.

### FIELD

The present disclosure relates to organic compounds useful for therapy in mammals, and in particular for modulating the interaction of YAP and TEAD for treatment of various diseases associated with the Hippo signaling pathway.

### BACKGROUND

YAP and TEAD are two proteins involved in the Hippo signaling pathway, which modulates tissue homeostasis, cell proliferation, tumoral transformation and apoptosis. This pathway involves a series of kinases leading to the phosphorylation of two transcriptional co-activators, YAP and TAZ. YAP/TAZ do not comprise a DNA binding domain, but they bind to TEAD transcription factor family (TEAD-1, TEAD-2, TEAD-3 and TEAD-4) to mediate target gene expression such as connective tissue growth factor (CTGF), cysteine-rich angiogenic inducer 61 (CYR61) and others to promote cell growth, proliferation, migration, and survival. (Gandhi T. K. Boopathy et al., Role of Hippo Pathway-YAP/TAZ Signaling in Angiogenesis, Front Cell Dev Biol. 2019; 7:49). When upstream kinases are inactive, YAP and TAZ are not phosphorylated and translocate to the nucleus, binding to TEAD. Deregulation of the Hippo pathway is involved in a broad variety of tumors, including breast, therefore, its targeting represents an approach for the treatment of cancers that harbor functional alterations of this pathway (Dominguez-Berrocal et al., New Therapeutic Approach for Targeting Hippo Signalling Pathway. Sci Rep 9, 4771(2019). As an example, one of the small molecules used to target this signalling pathway is Verteporfin, which associates to YAP and inhibits binding to TEAD.

Compounds that modulate, and more specifically, inhibit the interaction between YAP and TEAD (i.e., YAP/TEAD inhibitors), and thereby reduce the expression of YAP/TEAD target genes and display anti-proliferative effects in cancer cell lines controlled by the Hippo signaling pathway represent a new class of potential therapeutics capable of modulating tumor growth and other diseases. As there are no YAP/TEAD inhibitors that are currently approved for the treatment or prevention of diseases in humans, there is an unmet need for new compounds that are capable of modulating YAP/TEAD.

### SUMMARY

One embodiment of the disclosure relates to novel compounds, as described in any of the embodiments herein, or a pharmaceutically acceptable salt, a tautomer, a stereoisomer or a deuterated analog thereof, wherein these novel compounds can modulate YAP/TEAD.

Another embodiment of this disclosure relates to a compound of Formula (I):

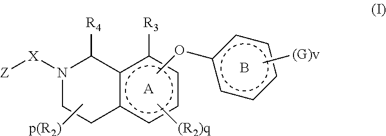

or a pharmaceutically acceptable salt, a tautomer, a stereoisomer, or a deuterated analog thereof, wherein ring A, ring B, R², R³, R⁴, G, p, q, v, X, and Z are as described in any of the embodiments (including any of the subembodiments thereof) in this disclosure.

Other embodiments and sub-embodiments of Formula (I) are further described herein in this disclosure.

Another embodiment of the disclosure relates to a pharmaceutical composition comprising a compound according to Formula (I) or any embodiment and sub-embodiment of Formula (I) described herein in this disclosure, or a pharmaceutically acceptable salt, a tautomer, a stereoisomer or a deuterated analog of any of these compounds, and a pharmaceutically acceptable carrier or excipient.

Another embodiment of the disclosure relates to a pharmaceutical composition comprising a compound according to Formula (I), or any embodiment of Formula (I) described herein in this disclosure, or a pharmaceutically acceptable salt, a tautomer, a stereoisomer or a deuterated analog of any of these compounds, and another therapeutic agent.

Another embodiment of this disclosure relates to a method for treating a subject with a disease or condition mediated, at least in part, by YAP/TEAD, said method comprising administering to the subject an effective amount of a compound according to Formula (I), or any embodiment of Formula (I) described in this disclosure, or a pharmaceutically acceptable salt, a tautomer, a stereoisomer or a deuterated analog of any of these compounds, or a pharmaceutical composition of any of the compounds as described in this disclosure.

Also provided herein is the use of a compound according to Formula (I), or any embodiment of Formula (I) described in this disclosure, or a pharmaceutically acceptable salt, a tautomer, a stereoisomer or a deuterated analog of any of these compounds, or a pharmaceutical composition of any of the compounds as described in this disclosure, for the treatment of a disease or condition mediated by YAP/TEAD.

Additional embodiments are described are further described in the Detailed Description of this disclosure.

### DETAILED DESCRIPTION

### I. Definitions

As used herein the following definitions apply unless clearly indicated otherwise:

It is noted here that as used herein and the appended claims, the singular forms “a,” ” ““an,” and “the” include plural reference unless the context clearly dictates otherwise.

Unless a point of attachment indicates otherwise, the chemical moieties listed in the definitions of the variables of Formula (I) of this disclosure, and all the embodiments thereof, are to be read from left to right, wherein the right hand side is directly attached to the parent structure as defined. However, if a point of attachment (e.g., a dash “-”) is shown on the left hand side of the chemical moiety (e.g., —C₁-C₆alkyl-N(R⁶)₂), then the left hand side of this chemical moiety is attached directly to the parent moiety as defined.

It is assumed that when considering generic descriptions of compounds described herein for the purpose of constructing a compound, such construction results in the creation of a stable structure. That is, one of ordinary skill in the art would recognize that, theoretically, some constructs would not normally be considered as stable compounds (that is, sterically practical and/or synthetically feasible).

“Alkyl,” by itself, or as part of another substituent, means, unless otherwise stated, a straight or branched chain hydrocarbon, having the number of carbon atoms designated (i.e., C₁-C₆ means one to six carbons). Representative alkyl groups include straight and branched chain alkyl groups having 1, 2, 3, 4, 5, 6, 7, 8, 9, 10, 11 or 12 carbon atoms. Further representative alkyl groups include straight and branched chain alkyl groups having 1, 2, 3, 4, 5, 6, 7 or 8 carbon atoms. Examples of alkyl groups include methyl, ethyl, n-propyl, isopropyl, n-butyl, t-butyl, isobutyl, sec-butyl, n-pentyl, n-hexyl, n-heptyl, n-octyl, and the like. For each of the definitions herein (e.g., alkyl, alkoxy, arylalkyl, cycloalkylalkyl, heterocycloalkylalkyl, heteroarylalkyl, etc.), when a prefix is not included to indicate the number of carbon atoms in an alkyl portion, the alkyl moiety or portion thereof will have 12 or fewer main chain carbon atoms or 8 or fewer main chain carbon atoms or 6 or fewer main chain carbon atoms. For example, C₁-C₆alkyl refers to a straight or branched hydrocarbon having 1, 2, 3, 4, 5 or 6 carbon atoms and includes, but is not limited to, —CH₃, C₂alkyl, C₃alkyl, C₄alkyl, C₅alkyl, C₆alkyl, C₁-C₂alkyl, Czalkyl, C₃alkyl, C₁-C₃alkyl, C₁-C₄alkyl, C₁-C₅alkyl, C₁-C₆alkyl, C₂-C₃alkyl, C₂-C₄alkyl, C₂. C₅alkyl, C₂-C₆alkyl, C₃-C₄alkyl, C₃-C₅alkyl, C₃-C₆alkyl, C₄-C₅alkyl, C₄-C₆alkyl, C₅-C₆ alkyl and C₆alkyl. While it is understood that substitutions are attached at any available atom to produce a stable compound, when optionally substituted alkyl is an R group of a moiety such as —OR (e.g. alkoxy), —SR (e.g. thioalkyl), —NHR (e.g. alkylamino), —C(O) NHR, and the like, substitution of the alkyl R group is such that substitution of the alkyl carbon bound to any O, S, or N of the moiety (except where N is a heteroaryl ring atom) excludes substituents that would result in any O, S, or N of the substituent (except where N is a heteroaryl ring atom) being bound to the alkyl carbon bound to any O, S, or N of the moiety.

“Alkylene” by itself or as part of another substituent means a linear or branched saturated divalent hydrocarbon moiety derived from an alkane having the number of carbon atoms indicated in the prefix. For example, (i.e., C₁-C₆ means one to six carbons; C₁-C₆alkylene is meant to include methylene, ethylene, propylene, 2-methylpropylene, pentylene, hexylene and the like). C₁₄ alkylene includes methylene —CH₂—, ethylene —CH₂CH₂—, propylene —CH₂CH₂CH₂—, and isopropylene —CH(CH₃)CH₂—, —CH₂CH(CH₃)—, —CH₂—(CH₂)₂CH₂—, —CH₂—CH(CH₃)CH₂—, —CH₂—C(CH₃)₂—CH₂—CH₂CH(CH₃)—. Typically, an alkyl (or alkylene) group will have from 1 to 24 carbon atoms, with those groups having 10 or fewer, 8 or fewer, or 6 or fewer carbon atoms. When a prefix is not included to indicate the number of carbon atoms in an alkylene portion, the alkylene moiety or portion thereof will have 12 or fewer main chain carbon atoms or 8 or fewer main chain carbon atoms, 6 or fewer main chain carbon atoms, or 4 or fewer main chain carbon atoms, or 3 or fewer main chain carbon atoms, or 2 or fewer main chain carbon atoms, or 1 carbon atom.

“Alkenyl” refers to a linear monovalent hydrocarbon radical or a branched monovalent hydrocarbon radical having the number of carbon atoms indicated in the prefix and containing at least one double bond. For example, C₂-C₆ alkenyl is meant to include ethenyl, propenyl, and the like.

“Alkoxy” or “alkoxyl” refers to a —O-alkyl group, where alkyl is as defined herein. By way of example, “C₁-C₆alkoxy” refers to a —O—C₁-C₆alkyl group, where alkyl is as defined herein. While it is understood that substitutions on alkoxy are attached at any available atom to produce a stable compound, substitution of alkoxy is such that O, S, or N (except where N is a heteroaryl ring atom), are not bound to the alkyl carbon bound to the alkoxy O. Further, where alkoxy is described as a substituent of another moiety, the alkoxy oxygen is not bound to a carbon atom that is bound to an O, S, or N of the other moiety (except where N is a heteroaryl ring atom), or to an alkene or alkyne carbon of the other moiety.

“Amino” or “amine” denotes the group NH2.

“Aryl” by itself, or as part of another substituent, unless otherwise stated, refers to a monocyclic, bicyclic or polycyclic polyunsaturated aromatic hydrocarbon radical containing 6 to 14 ring carbon atoms, which can be a single ring or multiple rings (up to three rings) which are fused together or linked covalently. Aryl, however, does not encompass or overlap in any way with heteroaryl defined below. If one or more aryl rings are fused with a heteroaryl ring, the resulting ring system is heteroaryl. Non-limiting examples of unsubstituted aryl groups include phenyl, 1-naphthyl and 2-naphthyl. The term “arylene” refers to a divalent aryl, wherein the aryl is as defined herein.

“Arylalkyl” or “aralkyl” refers to (alkylene)-aryl, where the alkylene group is as defined herein and has the indicated number of carbon atoms, or if unspecified having six or fewer main chain carbon atoms or four or fewer main chain carbon atoms; and aryl is as defined herein. Examples of arylalkyl include benzyl, phenethyl, 1-methylbenzyl, and the like.

“Cycloalkyl” or “Carbocycle” or “Carbocyclic” by itself, or as part of another substituent, unless otherwise stated, refers to saturated or partially unsaturated, nonaromatic monocyclic ring, bridged rings, sprio rings, fused rings (e.g., bicyclic or tricyclic carbon ring systems), or cubane, having the number of carbon atoms indicated in the prefix or if unspecified having 3-6, also 4-6, and also 5-6 ring members per ring, such as cyclopropyl, cyclopentyl, cyclohexyl, where one or two ring carbon atoms may optionally be replaced by a carbonyl. Further, the term cycloalkyl is intended to encompass ring systems fused to an aromatic ring (e.g., of an aryl or heteroaryl), regardless of the point of attachment to the remainder of the molecule. Cycloalkyl refers to hydrocarbon rings having the indicated number of ring atoms (e.g., C₃-6 cycloalkyl and 3-6 membered cycloalkyl both mean three to six ring carbon atoms). The term “cycloalkenyl” refers to a cycloalkyl having at least one unit of unsaturation. A substituent of a cycloalkyl or cycloalkenyl may be at the point of attachment of the cycloalkyl or cycloalkenyl group, forming a quaternary center.

“Cycloalkylalkyl” refers to an-(alkylene)-cycloalkyl group where alkylene as defined herein has the indicated number of carbon atoms or if unspecified having six or fewer carbon atoms; and cycloalkyl is as defined herein has the indicated number of carbon atoms or if unspecified having 3-10, also 3-8, and also 3-6, ring members per ring. By way of example, 4-6 membered cycloalkyl-C₁-C₆alkyl refers to a cycloalkyl with 4-6 carbon atoms attached to an alkylene chain with 1-6 carbon atoms, wherein the alkylene chain is attached to the parent moiety. Other exemplary cycloalkylalkyl includes, e.g., cyclopropylmethylene, cyclobutylethylene, cyclobutylmethylene, and the like.

“Halogen” or “halo” refers to all halogens, that is, chloro (C₁), fluoro (F), bromo (Br), or iodo (I).

The term “haloalkyl” refers to an alkyl substituted by one to seven halogen atoms. Haloalkyl includes monohaloalkyl or polyhaloalkyl. For example, the term “C₁-Cohaloalkyl” is meant to include trifluoromethyl, difluoromethyl, 2,2,2-trifluoroethyl, 4-chlorobutyl, 3-bromopropyl, and the like.

The term “haloalkoxy” refers to an alkoxy substituted by one to seven halogen atoms. Haloalkoxy includes monohaloalkoxy or polyhaloalkoxy. For example, the term “C₁-C₆haloalkoxy” is meant to include trifluoromethoxy, difluoromethoxy, 2,2,2-trifluoroethoxy, 4-chlorobutoxy, 3-bromopropoxy, and the like.

“Heteroatom” is meant to include oxygen (O), nitrogen (N), and sulfur(S).

“Heteroaryl” refers to a monocyclic or bicyclic aromatic ring radical containing 5-9 ring atoms (also referred to in this disclosure as a 5-9 membered heteroaryl, including monocyclic aromatic ring radicals containing 5 or 6 ring atoms (also referred to in this disclosure as a 5-6 membered heteroaryl), containing one or more, 14, 13, or 12, heteroatoms independently selected from the group consisting of O, S, and N. Any aromatic ring or ring system containing at least one heteroatom is a heteroaryl regardless of the point of attachment (i.e., through any one of the fused rings). Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable compound is produced. Examples of heteroaryl groups include, but are not limited to, pyridyl, pyridazinyl, pyrazinyl, indolizinyl, benzo[b] thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, indolyl, triazinyl, quinoxalinyl, cinnolinyl, phthalazinyl, benzotriazinyl, benzimidazolyl, benzopyrazolyl, benzotriazolyl, benzisoxazolyl, isobenzofuryl, isoindolyl, indolizinyl, benzotriazinyl, thienopyridyl, thienopyrimidinyl, pyrazolopyrimidinyl, imidazopyridines, benzothiaxolyl, benzothienyl, quinolyl, isoquinolyl, indazolyl, pteridinyl and thiadiazolyl. “Nitrogen containing heteroaryl” refers to heteroaryl wherein at least one of the ring heteroatoms is N.

“Heteroarylalkyl” refers to (alkylene)-heteroaryl, where the alkylene group is as defined herein and has the indicated number of carbon atoms, or if unspecified having six or fewer main chain carbon atoms or four or fewer main chain carbon atoms; and heteroaryl is as defined herein.

“Heterocycloalkyl” refers to a saturated or partially unsaturated non-aromatic cycloalkyl group that contains from one to five heteroatoms selected from N, O, S (including S(O) and S(O)₂), or P (including phosphine oxide) wherein the nitrogen, sulfur, and phosphorous atoms are optionally oxidized, and the nitrogen atom(s) are optionally quarternized, the remaining ring atoms being C, where one or two C atoms may optionally be present as a carbonyl. Further, the term heterocycloalkyl is intended to encompass any ring or ring system containing at least one heteroatom that is not a heteroaryl, regardless of the point of attachment to the remainder of the molecule. Heterocycloalkyl groups include those having a ring with a formally charge-separated aromatic resonance structure, for example, N-methylpyridonyl. The heterocycloalkyl may be substituted with one or two oxo groups, and can include sulfone and sulfoxide derivatives. The heterocycloalkyl may be a monocyclic, a fused bicyclic or a fused polycyclic ring system of 3 to 12, 4 to 10, 5 to 10, or 5 to 6 ring atoms in which one to five ring atoms are heteroatoms selected from —N═, —N—, —O—, —S—, —S(O)—, or —S(O)₂— and further wherein one or two ring atoms are optionally replaced by a —C(O)— group. As an example, a 4-6 membered heterocycloalkyl is a heterocycloalkyl with 4-6 ring members having at least one heteroatom. The heterocycloalkyl can also be a heterocyclic alkyl ring fused with a cycloalkyl. Non limiting examples of heterocycloalkyl groups include pyrrolidinyl, piperidinyl, morpholinyl, pyridonyl, and the like. A heterocycloalkyl group can be attached to the remainder of the molecule through a ring carbon or a heteroatom. “Heterocycloalkenyl” refers to a heterocycloalkyl having at least one unit of unsaturation. A substituent of a heterocycloalkyl or heterocycloalkenyl may be at the point of attachment of the heterocycloalkyl or heterocycloalkenyl group, forming a quaternary center.

“Heterocycloalkylalkyl” refers to (alkylene)-heterocycloalkyl, where the alkylene group is as defined herein and has the indicated number of carbon atoms, or if unspecified having six or fewer main chain carbon atoms or four or fewer main chain carbon atoms; and heterocycloalkyl is as defined herein.

“Hydroxyl” or “hydroxy” refers to the group OH. The term “hydroxyalkyl” or “hydroxyalkylene” refers to an alkyl group or alkylene group, respectively as defined herein, substituted with 1-5 hydroxy groups.

“Optional substituents” or “optionally substituted” as used throughout the disclosure means that the substitution on a compound may or may not occur, and that the description includes instances where the substitution occurs and instances in which the substitution does not.

For example, the phrase “optionally substituted with 1-3 Tl groups” means that the Tl group may but need not be present. It is assumed in this disclosure that optional substitution on a compound occurs in a way that would result in a stable compound.

As used herein in connection with compounds of the disclosure, the term “synthesizing” and like terms means chemical synthesis from one or more precursor materials.

As used herein, the term “composition” refers to a formulation suitable for administration to an intended animal subject for therapeutic purposes that contains at least one pharmaceutically active compound and at least one pharmaceutically acceptable carrier or excipient.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

“Pharmaceutically acceptable salt” refers to a salt which is acceptable for administration to a patient, such as a mammal (e.g., salts having acceptable mammalian safety for a given dosage regime). Contemplated pharmaceutically acceptable salt forms include, without limitation, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug. Such salts can be derived from pharmaceutically acceptable inorganic or organic bases and from pharmaceutically acceptable inorganic or organic acids, depending on the particular substituents found on the compounds described herein.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. In another example, a salt can be prepared by reacting the free base and acid in an organic solvent.

When compounds of the present disclosure contain relatively acidic functionalities, base addition salts can be obtained by contacting the neutral form of such compounds with a sufficient amount of the desired base (i.e. a primary, secondary, tertiary, quaternary, or cyclic amine; an alkali metal hydroxide; alkaline earth metal hydroxide; or the like), either neat or in a suitable inert solvent. The desired acid can be, for example, a pyranosidyl acid (such as glucuronic acid or galacturonic acid), an alpha-hydroxy acid (such as citric acid or tartaric acid), an amino acid (such as aspartic acid or glutamic acid), an aromatic acid (such as benzoic acid or cinnamic acid), a sulfonic acid (such as p-toluenesulfonic acid or ethanesulfonic acid), or the like. In some embodiments, salts can be derived from pharmaceutically acceptable acids such as acetic, trifluoroacetic, propionic, ascorbic, benzenesulfonic, benzoic, camphorsulfonic, citric, ethanesulfonic, fumaric, glycolic, gluconic, glucoronic, glutamic, hippuric, hydrobromic, hydrochloric, isethionic, lactic, lactobionic, maleic, malic, malonic, mandelic, oxalic, methanesulfonic, mucic, naphthalenesulfonic, nicotinic, nitric, pamoic, pantothenic, phosphoric, succinic, sulfuric, sulfamic, hydroiodic, carbonic, tartaric, p-toluenesulfonic, pyruvic, aspartic, benzoic, cinnamic, anthranilic, mesylic, salicylic, p-hydroxybenzoic, phenylacetic, embonic (pamoic), ethanesulfonic, benzenesulfonic, 2-hydroxyethanesulfonic, sulfanilic, stearic, cyclohexylsulfamic, cyclohexylaminosulfonic, quinic, algenic, hydroxybutyric, galactaric and galacturonic acid and the like.

Also included are salts of amino acids such as arginate and the like, and salts of organic acids like glucuronic or galactunoric acids and the like (see, for example, Berge, S. M. et al., “Pharmaceutical Salts,” J. Pharmaceutical Science, 1977, 66:1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

The pharmaceutically acceptable salt of the different compounds may be present as a complex. Examples of complexes include 8-chlorotheophylline complex (analogous to, e.g., dimenhydrinate:diphenhydramine 8-chlorotheophylline (1:1) complex; Dramamine) and various cyclodextrin inclusion complexes.

The term “deuterated” as used herein alone or as part of a group, means substituted deuterium atoms. The term “deuterated analog” as used herein alone or as part of a group, means substituted deuterium atoms in place of hydrogen. The deuterated analog of the disclosure may be a fully or partially deuterium substituted derivative. In some embodiments, the deuterium substituted derivative of the disclosure holds a fully or partially deuterium substituted alkyl, aryl or heteroaryl group.

The disclosure also embraces isotopically-labeled compounds of the present disclosure which are identical to those recited herein, but for the fact that one or more atoms are replaced by an atom having an atomic mass or mass number different from the atomic mass or mass number usually found in nature. All isotopic variations of the compounds of the present disclosure, whether radioactive or not, are intended to be encompassed within the scope of the present disclosure. Examples of isotopes that can be incorporated into compounds of the disclosure include isotopes of hydrogen, carbon, nitrogen, oxygen, phosphorous, fluorine, and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and ¹²⁵¹. Unless otherwise stated, when a position is designated specifically as “H” or “hydrogen,” the position is understood to have hydrogen at its natural abundance isotopic composition or its isotopes, such as deuterium (D) or tritium (³H). Certain isotopically-labeled compounds of the present disclosure (e.g., those labeled with ³H and ¹⁴C) are useful in compound and/or substrate tissue distribution assays. Tritiated (i.e., ³H) and carbon-¹⁴ (i.e., ¹⁴° C.) and fluorine-18 (¹⁸F) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present disclosure can generally be prepared by following procedures analogous to those described in the Schemes and in the Examples herein below, by substituting an isotopically labeled reagent for a non-isotopically labeled reagent.

“Prodrugs” means any compound which releases an active parent drug according to Formula (I) in vivo when such prodrug is administered to a subject. Prodrugs of a compound of Formula (I) are prepared by modifying functional groups present in the compound of Formula (I) in such a way, either in routine manipulation or in vivo, that the modifications may be cleaved in vivo to release the parent compound. Prodrugs may proceed from prodrug form to active form in a single step or may have one or more intermediate forms which may themselves have activity or may be inactive. Some prodrugs are activated enzymatically to yield the active compound, or a compound which, upon further chemical reaction, yields the active compound. Prodrugs include compounds of Formula (I) wherein a hydroxy, amino, carboxyl or sulfhydryl group in a compound of Formula (I) is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), amides, guanidines, carbamates (e.g., N,N-dimethylaminocarbonyl) of hydroxy functional groups in compounds of Formula (I), and the like. Other examples of prodrugs include, without limitation, carbonates, ureides, solvates, or hydrates of the active compound. Preparation, selection, and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

As described in The Practice of Medicinal Chemistry, Ch. 31-32 (Ed. Wermuth, Academic Press, San Diego, CA, 2001), prodrugs can be conceptually divided into two non-exclusive categories, bioprecursor prodrugs and carrier prodrugs. Generally, bioprecursor prodrugs are compounds that are inactive or have low activity compared to the corresponding active drug compound, that contain one or more protective groups and are converted to an active form by metabolism or solvolysis. Both the active drug form and any released metabolic products should have acceptably low toxicity. Typically, the formation of active drug compound involves a metabolic process or reaction that is one of the following types:

(1) Oxidative reactions: Oxidative reactions are exemplified without limitation to reactions such as oxidation of alcohol, carbonyl, and acid functionalities, hydroxylation of aliphatic carbons, hydroxylation of alicyclic carbon atoms, oxidation of aromatic carbon atoms, oxidation of carbon-carbon double bonds, oxidation of nitrogen-containing functional groups, oxidation of silicon, phosphorus, arsenic, and sulfur, oxidative N-dealkylation, oxidative O- and S-dealkylation, oxidative deamination, as well as other oxidative reactions.

(2) Reductive reactions: Reductive reactions are exemplified without limitation to reactions such as reduction of carbonyl functionalities, reduction of alcohol functionalities and carbon-carbon double bonds, reduction of nitrogen-containing functional groups, and other reduction reactions.

(3) Reactions without change in the oxidation state: Reactions without change in the state of oxidation are exemplified without limitation to reactions such as hydrolysis of esters and ethers, hydrolytic cleavage of carbon-nitrogen single bonds, hydrolytic cleavage of non-aromatic heterocycles, hydration and dehydration at multiple bonds, new atomic linkages resulting from dehydration reactions, hydrolytic dehalogenation, removal of hydrogen halide molecule, and other such reactions.

Carrier prodrugs are drug compounds that contain a transport moiety, e.g., that improves uptake and/or localized delivery to a site(s) of action. Desirably for such a carrier prodrug, the linkage between the drug moiety and the transport moiety is a covalent bond, the prodrug is inactive or less active than the drug compound, the prodrug and any release transport moiety are acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. (See, e.g., Cheng et al., U.S. Patent Publ. No. 2004/0077595, incorporated herein by reference.) Such carrier prodrugs are often advantageous for orally administered drugs. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g. stability, water solubility, suppression of an undesirable organoleptic or physiochemical property). For example, lipophilicity can be increased by esterification of hydroxyl groups with lipophilic carboxylic acids, or of carboxylic acid groups with alcohols, e.g., aliphatic alcohols.

The term “carrier” is also meant to include microspheres, liposomes, micelles, nanoparticles (naturally-equipped nanocarriers, for example, exosomes), and the like. It is known that exosomes can be highly effective drug carriers, and there are various ways in which drugs can be loaded into exosomes, including those techniques described in J Control Release. 2015 Dec. 10; 219:396-405, the contents of which are incorporated by reference in its entirety.

Metabolites, e.g., active metabolites, overlap with prodrugs as described above, e.g., bioprecursor prodrugs. Thus, such metabolites are pharmacologically active compounds or compounds that further metabolize to pharmacologically active compounds that are derivatives resulting from metabolic process in the body of a subject. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques known in the art. See, e.g., Bertolini et al., 1997, J. Med. Chem., 40:2011-2016; Shan et al., 1997, J Pharm Sci 86(7): 756-757; Bagshawe, 1995, Drug Dev. Res., 34:220-230.

“Tautomer” means compounds produced by the phenomenon wherein a proton of one atom of a molecule shifts to another atom. See, Jerry March, Advanced Organic Chemistry: Reactions, Mechanisms and Structures, Fourth Edition, John Wiley & Sons, pages 69-74(1992). The tautomers also refer to one of two or more structural isomers that exist in equilibrium and are readily converted from one isomeric form to another. Examples of include keto-enol tautomers, such as acetone/propen-2-ol, imine-enamine tautomers and the like, ring-chain tautomers, such as glucose/2,3,4,5,6-pentahydroxy-hexanal and the like, the tautomeric forms of heteroaryl groups containing a —N═C(H)—NH— ring atom arrangement, such as pyrazoles, imidazoles, benzimidazoles, triazoles, and tetrazoles. Where the compound contains, for example, a keto or oxime group or an aromatic moiety, tautomeric isomerism (‘tautomerism’) can occur. The compounds described herein may have one or more tautomers and therefore include various isomers. A person of ordinary skill in the art would recognize that other tautomeric ring atom arrangements are possible. All such isomeric forms of these compounds are expressly included in the present disclosure.

“Isomers” mean compounds that have identical molecular Formulae but differ in the nature or sequence of bonding of their atoms or in the arrangement of their atoms in space. Isomers that differ in the arrangement of their atoms in space are termed “stereoisomers.” “Stereoisomer” and “stereoisomers” refer to compounds that exist in different stereoisomeric forms, for example, if they possess one or more asymmetric centers or a double bond with asymmetric substitution and, therefore, can be produced as individual stereoisomers or as mixtures. Stereoisomers include enantiomers and diastereomers. Stereoisomers that are not mirror images of one another are termed “diastereomers” and those that are non-superimposable mirror images of each other are termed “enantiomers.” When a compound has an asymmetric center, for example, an atom such as carbon bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.” As another example, stereoisomers include geometric isomers, such as cis- or trans-orientation of substituents on adjacent carbons of a double bond. Unless otherwise indicated, the description is intended to include individual stereoisomers as well as mixtures. The methods for the determination of stereochemistry and the separation of stereoisomers are well-known in the art (see discussion in Chapter 4 of ADVANCED ORGANIC CHEMISTRY, 6th edition J. March, John Wiley and Sons, New York, 2007) differ in the chirality of one or more stereocenters.

“Hydrate” refers to a complex formed by combination of water molecules with molecules or ions of the solute. “Solvate” refers to a complex formed by combination of solvent molecules with molecules or ions of the solute. The solvent can be an organic compound, an inorganic compound, or a mixture of both. Solvate is meant to include hydrate. Some examples of solvents include, but are not limited to, methanol, N,N-dimethylformamide, tetrahydrofuran, dimethylsulfoxide, and water. In general, the solvated forms are equivalent to unsolvated forms and are encompassed within the scope of the present disclosure.

In the context of the use, testing, or screening of compounds that are or may be modulators, the term “contacting” means that the compound(s) are caused to be in sufficient proximity to a particular molecule, complex, cell, tissue, organism, or other specified material that potential binding interactions and/or chemical reaction between the compound and other specified material can occur.

By “assaying” is meant the creation of experimental conditions and the gathering of data regarding a particular result of the exposure to specific experimental conditions. For example, enzymes can be assayed based on their ability to act upon a detectable substrate. A compound can be assayed based on its ability to bind to a particular target molecule or molecules.

As used herein, the terms “ligand” and “modulator” are used equivalently to refer to a compound that changes (i.e., increases or decreases) the activity of a target biomolecule, e.g., an enzyme such as those described herein. Generally a ligand or modulator will be a small molecule, where “small molecule refers to a compound with a molecular weight of 1500 Daltons or less, 1000 Daltons or less, 800 Daltons or less, or 600 Daltons or less. Thus, an “improved ligand” is one that possesses better pharmacological and/or pharmacokinetic properties than a reference compound, where “better” can be defined by one skilled in the relevant art for a particular biological system or therapeutic use.

The term “binds” in connection with the interaction between a target and a potential binding compound indicates that the potential binding compound associates with the target to a statistically significant degree as compared to association with proteins generally (i.e., non-specific binding). Thus, the term “binding compound” refers to a compound that has a statistically significant association with a target molecule. In some embodiments, a binding compound interacts with a specified target with a dissociation constant (K_{D}) of 10 mM or less, 1,000 μM or less, 100 μM or less, 10 μM or less, 1 μM or less, 1,000 nM or less, 100 nM or less, 10 nM or less, or 1 nM or less. In the context of compounds binding to a target, the terms “greater affinity” and “selective” indicates that the compound binds more tightly than a reference compound, or than the same compound in a reference condition, i.e., with a lower dissociation constant. In some embodiments, the greater affinity is at least 2, 3, 4, 5, 8, 10, 50, 100, 200, 400, 500, 1000, or 10,000-fold greater affinity.

The terms “modulate,” “modulation,” and the like refer to the ability of a compound to increase or decrease the function and/or expression of a target, such as the interaction between YAP and TEAD, where such function may include transcription regulatory activity and/or binding. Modulation may occur in vitro or in vivo. Modulation, as described herein, includes the inhibition, antagonism, partial antagonism, activation, agonism or partial agonism of a function or characteristic associated with YAP/TEAD, either directly or indirectly, and/or the upregulation or downregulation of the expression YAP/TEAD, either directly or indirectly. In another embodiment, the modulation is direct. Inhibitors or antagonists are compounds that, e.g., bind to, partially or totally block stimulation, decrease, prevent, inhibit, delay activation, inactivate, desensitize, or downregulate signal transduction. Activators or agonists are compounds that, e.g., bind to, stimulate, increase, open, activate, facilitate, enhance activation, activate, sensitize or upregulate signal transduction.

As used herein, the terms “treat,” “treating,” “therapy,” “therapies,” and like terms refer to the administration of material, e.g., any one or more compound(s) as described herein in an amount effective to inhibit YAP/TEAD. In other embodiments, the terms “treat,” “treating,” “therapy,” “therapies,” and like terms refer to the administration of material, e.g., any one or more compound(s) as described herein is an amount effective to prevent, alleviate, or ameliorate one or more symptoms of a disease or condition, i.e., indication, and/or to prolong the survival of the subject being treated.

The terms “prevent,” “preventing,” “prevention” and grammatical variations thereof as used herein, refers to a method of partially or completely delaying or precluding the onset or recurrence of a disease, disorder or condition and/or one or more of its attendant symptoms or barring a subject from acquiring or reacquiring a disorder or condition or reducing a subject's risk of acquiring or requiring a disorder or condition or one or more of its attendant symptoms.

As used herein, the term “subject,” “animal subject,” and the like refers to a living organism including, but not limited to, human and non-human vertebrates, e.g. any mammal, such as a human, other primates, sports animals and animals of commercial interest such as cattle, horses, ovines, or porcines, rodents, or pets such as dogs and cats.

“Unit dosage form” refers to a composition intended for a single administration to treat a subject suffering from a disease or medical condition. Each unit dosage form typically comprises each of the active ingredients of this disclosure plus pharmaceutically acceptable excipients. Examples of unit dosage forms are individual tablets, individual capsules, bulk powders, liquid solutions, ointments, creams, eye drops, suppositories, emulsions or suspensions. Treatment of the disease or condition may require periodic administration of unit dosage forms, for example: one unit dosage form two or more times a day, one with each meal, one every four hours or other interval, or only one per day. The expression “oral unit dosage form” indicates a unit dosage form designed to be taken orally.

The term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

In the present context, the term “therapeutically effective” or “effective amount” indicates that a compound or material or amount of the compound or material when administered is sufficient or effective to prevent, alleviate, or ameliorate one or more symptoms of a disease, disorder or medical condition being treated, and/or to prolong the survival of the subject being treated. The therapeutically effective amount will vary depending on the compound, the disease, disorder or condition and its severity and the age, weight, etc., of the mammal to be treated. In general, satisfactory results in subjects are indicated to be obtained at a daily dosage of from about 0.1 to about 10 g/kg subject body weight. In some embodiments, a daily dose ranges from about 0.10 to 10.0 mg/kg of body weight, from about 1.0 to 3.0 mg/kg of body weight, from about 3 to 10 mg/kg of body weight, from about 3 to 150 mg/kg of body weight, from about 3 to 100 mg/kg of body weight, from about 10 to 100 mg/kg of body weight, from about 10 to 150 mg/kg of body weight, or from about 150 to 1000 mg/kg of body weight. The dosage can be conveniently administered, e.g., in divided doses up to four times a day or in sustained-release form.

As used herein, the term “YAP/TEAD mediated disease or condition” (which is also meant to mean “YAP or TEAD mediated disease or condition” or “YAP and/or TEAD mediated disease or condition”) refers to a disease or condition in which the biological function of YAP/TEAD affect the development and/or course of the disease or condition, and/or in which modulation of the interaction of YAP/TEAD (such as YAP/TEAD mediated transcription) alters the development, course, and/or symptoms. A of YAP/TEAD mediated disease or condition includes a disease or condition for which the disruption YAP/TEAD interactions (for example, by TEAD inhibition), and/or inhibition of YAP/TEAD mediated transcripton provides a therapeutic benefit, e.g, wherein treatment with YAP/TEAD inhibitors, including compounds described herein, provides a therapeutic benefit to the subject suffering from or at risk of the disease or condition. A YAP/TEAD mediated disease or condition is intended to include a cancer that harbors loss of function mutations in YAP/TEAD, or a cancer where there is activation of YAP/TEAD. A YAP/TEAD mediated disease or condition is also intended to include various human carcinomas, including those of colon, lung, pancreas, and ovary, as well as diseases or conditions associated with tumor neovascularization, and invasiveness.

Also in the context of compounds binding to a biomolecular target, the term “greater specificity” indicates that a compound binds to a specified target to a greater extent than to another biomolecule or biomolecules that may be present under relevant binding conditions, where binding to such other biomolecules produces a different biological activity than binding to the specified target. Typically, the specificity is with reference to a limited set of other biomolecules, e.g., in the case of YAP or TEAD. In particular embodiments, the greater specificity is at least 2, 3, 4, 5, 8, 10, 50, 100, 200, 400, 500, or 1000-fold greater specificity.

As used herein in connection with binding compounds or ligands, the term “specific for YAP/TEAD,” and terms of like import mean that a particular compound binds to YAP or TEAD to a statistically greater extent than to other epigenetic targets that may be present in a particular sample. Also, where biological activity other than binding is indicated, the term “specific for YAP or TEAD” indicates that a particular compound has greater biological effect associated with binding YAP or TEAD than to other enzymes, e.g., enzyme activity inhibition. In some embodiments, compounds described herein are specific for TEAD inhibition.

The term “first line cancer therapy” refers to therapy administered to a subject as an initial regimen to reduce the number of cancer cells. First line therapy is also referred to as induction therapy, primary therapy and primary treatment. First-line therapy can be an administered combination with one or more agents. A summary of currently accepted approaches to first line treatment for certain disease can be found in the NCI guidelines for such diseases.

The term “second line cancer therapy” refers to a cancer treatment that is administered to a subject who does not respond to first line therapy, that is, often first line therapy is administered or who has a recurrence of cancer after being in remission. In certain embodiments, second line therapy that may be administered includes a repeat of the initial successful cancer therapy, which may be any of the treatments described under “first line cancer therapy.” A summary of the currently accepted approaches to second line treatment for certain diseases is described in the NCI guidelines for such diseases.

The term “refractory” refers to wherein a subject fails to respond or is otherwise resistant to cancer therapy or treatment. The cancer therapy may be first-line, second-line or any subsequently administered treatment. In certain embodiments, refractory refers to a condition where a subject fails to achieve complete remission after two induction attempts. A subject may be refractory due to a cancer cell's intrinsic resistance to a particular therapy, or the subject may be refractory due to an acquired resistance that develops during the course of a particular therapy.

As used herein, any variable followed by either a subscript or a superscript interger, for example Rₓ or R^{x} where x is an integer, are intended to be interchangeable and synonymous.

In addition, abbreviations as used herein have respective meanings as follows:

| | |
|---|---|
| δ | Chemical Shift |
| ° C. | Degree Celsius |
| Ac | Acetyl |
| BOC | Tert-butoxycarbonyl |
| Cbz | Carbobenzyloxy |
| DCC | N,N′-Dicyclohexylcarbodiimide |
| DCM | Dichloromethane |
| DIPEA/DIEA | N,N-Diisopropylethylamine |
| DMF | Dimethylformamide |
| DMSO | Dimethylsulfoxide |
| EDC | 1-Ethyl-3-(3-dimethylaminopropyl)carbodiimide |
| EtOAc | Ethyl Acetate |
| ESI | Electrospray ionization |
| Fmoc | 9-Fluorenylmethyloxycarbonyl |
| ¹H NMR | Proton Nuclear Magnetic Resonance Spectroscopy |
| HATU | 1-[Bis(dimethylamino)methylene]-1H-1,2,3- |
| | triazolo[4,5-b]pyridinium 3-oxide hexafluorophosphate |
| HBTU | 3-[Bis(dimethylamino)methyliumyl]-3H-benzotriazol- |
| | 1-oxide hexafluorophosphate |
| HPLC | High Performance Liquid Chromatography |
| hr(s) | Hour(s) |
| IC₅₀ | Half minimal (50%) inhibitory concentration |
| LCMS/LC-MS | Liquid Chromatography Mass Spectrometry |
| [M + H+]+ | Mass peak plus hydrogen |
| or (MH)+ | |
| [M − H−]− | Mass peak minus hydrogen |
| or (MH)− | |
| m/z | Mass-to-charge ratio |
| Me | Methyl |
| MeCN | Acetonitrile |
| MeOH | Methanol |
| min(s) | Minute(s) |
| MS | Mass spectrometry |
| NMP | N-Methyl-2-pyrrolidone |
| Pd(dppf)Cl₂ | [1,1′-Bis(diphenylphos- |
| | phino)ferrocene]dichloropalladium(II) |
| Ph | Phenyl |
| Pinacol | 2,3-Dimethylbutane-2,3-diol |
| RP | Reverse phase |
| RT | Room temperature |
| TEA | Triethylamine |
| TFA | Trifluoroacetic Acid |
| THF | Tetrahydrofuran |
| Triflate | Trifluoromethanesulfonate |

### II. Compounds

Embodiment 1 of this disclosure relates to a compound having Formula (I):

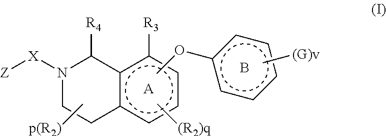

or a pharmaceutically acceptable salt, a tautomer, a stereoisomer, or a deuterated analog thereof, wherein:
ring A is phenyl or a 5- or 6-membered heteroaryl comprising 1-4 heteroatoms selected from N, O and S;
ring B is phenyl or a 5- or 6-membered heteroaryl comprising 1-4 heteroatoms selected from N, O and S,
each G is independently selected from halogen, OH, CN, alkyl optionally substituted with one or more R⁵, and alkoxy optionally substituted with one or more R⁵;
each R² is independently H, halogen, —C(O)O-alkyl, or C₁-C₃alkyl optionally substituted with 1-3 halogens, provided that not more than one R² is —C(O)O-alkyl;
R³ is H; halogen; alkenyl optionally substituted with cycloalkyl or heterocycloalkyl; heterocycloalkyl optionally substituted with —C(O)-alkyl; heterocycloalkenyl optionally substituted with C(O)-alkyl; heterocycloalkylalkyl optionally substituted with C(O)-alkyl; or heteroaryl optionally substituted with haloalkyl, cycloalkyl, or cycloalkylalkyl;
R⁴ is H; alkyl; cycloalkyl optionally substituted with haloalkyl or —C(O)-alkenyl; heterocycloalkyl optionally substituted with —C(O)-alkyl, —C(O)-alkenyl, or —C(O)-cycloalkyl; or heterocycloalkylalkyl optionally substituted with —C(O)-alkyl, —C(O)—CH₂—OH, or heteroaryl;
R⁵ is halogen or OH;
X is —(CH₂)ₘ—S(O)₂—, —(CH₂)ₙ—C(O)—, or —C(O)O—, wherein the right-hand side indicates the point of attachment to Z;
Z is —NR⁶R⁷, C₁-C₆alkyl optionally substituted with 1-4 R⁸, —C(CH₃)═CH₂, —CH₂—CH═C═O, cycloalkyl optionally substituted with 1-4 R⁹, heterocycloalkyl optionally substituted with 1-4 R¹⁰, aryl optionally substituted with 1-4 R¹⁰, or heteroaryl optionally substituted with 1-4 R¹⁰;
R⁶ is hydrogen, —S(O)₂—C₁-C₃alkyl, C₁-C₆alkyl, cycloalkyl, heterocycloalkyl, aryl, or heteroaryl, wherein each C₁-C₆alkyl, cycloalkyl, heterocycloalkyl, aryl, or heteroaryl is optionally substituted with 1-3 R¹¹;
R⁷ is hydrogen or C₁-C₆alkyl; or
R⁶ and R⁷, together with the nitrogen to which they are attached, form a heterocycloalkyl;
R⁸ is —NR⁶R⁷, CN, hydroxy, alkoxy, haloalkoxy, —S—C₁-C₃alkyl, —S(O)—C₁-C₃alkyl, —S(O)₂—C₁-C₃alkyl, —S(O)₂—C₁-C₃haloalkyl, —S(O)₂—NH₂, —S(O)(NH)-C₁-C₃alkyl, —C(O)—NH2, —C(O)OH, —C(O)O-C₁-C₃alkyl, —C(O)C₁-C₃haloalkyl, —N(H)—C(O)—NR⁶R⁷, —N(H)—C(O)—C₁-C₃alkyl optionally substituted with —NR¹²R¹³ or CN, —N(H)—C(O)—C₂-C₆alkenyl, —N(H)—C(O)—C₁-C₃haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₅haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰; or
two R⁸, together with the carbon atom to which they are attached, join together to form a cycloalkyl;
each R⁹ is independently CN, —S(O)₂—C₁-C₃alkyl, or —N(C₁-3alkyl)₂;
each R¹⁰ is independently hydroxy, halogen, CN, C₁-C₆alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkenyl, —S(O)₂-C₁-C₃alkyl, —S(O)₂—C₂-C₆alkenyl, or —S(O)₂—NH₂;
each R¹¹ is independently hydroxy, CN, alkoxy, —S(O)₂—C₁-C₃alkyl, or cycloalkyl;
each R¹² and R¹³ are independently hydrogen, C₁-C₆alkyl, or C₃-C₆cycloalkyl;
v is 0, 1, 2, 3, or 4;
p is 0, 1, 2, 3, or 4;
q is 0, 1, or 2;
n is 0, 1, or 2; and
m is 0, 1, 2, or 3;
provided that ring A and/or ring B is a 5- or 6-membered heteroaryl comprising 1-4 heteroatoms selected from N, O and S.

Embodiment 1a of this disclosure relates to a compound having Formula (I):

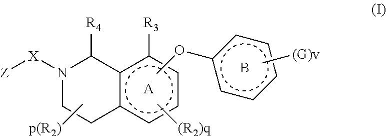

or a pharmaceutically acceptable salt, a tautomer, a stereoisomer, or a deuterated analog thereof, wherein:
ring A is phenyl or a 5- or 6-membered heteroaryl comprising 1-4 heteroatoms selected from N, O and S;
ring B is phenyl or a 5- or 6-membered heteroaryl comprising 1-4 heteroatoms selected from N, O and S,
each G is independently selected from halogen, OH, CN, alkyl optionally substituted with one or more R⁵, and alkoxy optionally substituted with one or more R⁵;
each R² is independently H, halogen, —C(O)O-alkyl, or C₁-C₃alkyl optionally substituted with 1-3 halogens, provided that not more than one R² is —C(O)O-alkyl;
R³ is H; halogen; alkenyl optionally substituted with cycloalkyl or heterocycloalkyl; heterocycloalkyl optionally substituted with —C(O)-alkyl; heterocycloalkenyl optionally substituted with C(O)-alkyl; heterocycloalkylalkyl optionally substituted with C(O)-alkyl; or heteroaryl optionally substituted with haloalkyl, cycloalkyl, or cycloalkylalkyl;
R⁴ is H; alkyl; cycloalkyl optionally substituted with haloalkyl or —C(O)-alkenyl; heterocycloalkyl optionally substituted with —C(O)-alkyl, —C(O)-alkenyl, or —C(O)-cycloalkyl; or heterocycloalkylalkyl optionally substituted with —C(O)-alkyl, —C(O)—CH₂—OH, or heteroaryl;
R⁵ is halogen or OH;
X is a bond, —(CH₂)ₘ—S(O)₂—, —(CH₂)ₙ—C(O)—, or —C(O)O—, wherein the right-hand side indicates the point of attachment to Z;
Z is —NR⁶R⁷, C₁-C₆ alkyl optionally substituted with 1-4 R⁸, C₂-C₆ alkenyl; —C(CH₃)═CH₂, —CH₂—CH═C═O, cycloalkyl optionally substituted with 1-4 R⁹, heterocycloalkyl optionally substituted with 1-4 R¹⁰, aryl optionally substituted with 1-4 R¹⁰, or heteroaryl optionally substituted with 1-4 R¹⁰;
R⁶ is hydrogen, —S(O)₂—C₁-C₃alkyl, C₁-C₆alkyl, cycloalkyl, heterocycloalkyl, aryl, or heteroaryl, wherein each C₁-C₆alkyl, cycloalkyl, heterocycloalkyl, aryl, or heteroaryl is optionally substituted with 1-3 R¹¹;
R⁷ is hydrogen or C₁-C₆alkyl; or
R⁶ and R⁷, together with the nitrogen to which they are attached, form a heterocycloalkyl;
R⁸ is halogen, —NR⁶R⁷, CN, hydroxy, alkoxy, haloalkyl, haloalkoxy, —S—C₁-C₃alkyl, —S(O)—C₁-C₃alkyl, —S(O)₂—C₁-C₃alkyl, —S(O)₂—C₁-C₃haloalkyl, —S(O)₂—NH₂, —S(O)₂ (NH)-C₁-C₃alkyl, —C(O)—NH₂, —C(O)OH, —C(O)O—C₁-C₃alkyl, —C(O)C₁-C₃haloalkyl, —N(H)—C(O)—NR⁶R⁷, —N(H)—C(O)—C₁-C₃alkyl optionally substituted with —NR¹²R¹³ or CN, —N(H)—C(O)—C₂-C₆alkenyl, —N(H)—C(O)—C₁-C₃haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₃haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰; or
two R⁸, together with the carbon atom to which they are attached, join together to form a cycloalkyl;
each R⁹ is independently CN, —S(O)₂—C₁-C₃alkyl, or —N(C₁-3alkyl)₂;
each R¹⁰ is independently hydroxy, halogen, CN, C₁-C₆ alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkyl, —NH—C(O)-alkenyl, N(H)—S(O)₂—C₁-C₃alkyl, —S(O)₂—C₁-C₃alkyl, —S(O)₂—C₁-C₃haloalkyl, —S(O)₂—C₂-C₆alkenyl, —S(O)₂—NH₂, or —S(O)₂—N(H)—C₁-C₃alkyl;
each R¹¹ is independently hydroxy, CN, alkoxy, —S(O)₂—C₁-C₃alkyl, or cycloalkyl;
each R¹² and R¹³ are independently hydrogen, C₁-C₆alkyl, or C₃-C₆cycloalkyl;
v is 0, 1, 2, 3, or 4;
p is 0, 1, 2, 3, or 4;
q is 0, 1, or 2;
n is 0, 1, or 2; and
m is 0, 1, 2, or 3;
   provided that if X is —(CH₂)ₘ—S(O)₂—, —(CH₂)ₙ—C(O)—, or —C(O)O—, then ring A and/or ring B is a 5- or 6-membered heteroaryl comprising 1-4 heteroatoms selected from N, O and S when —O— is attached to Ring A at the 5 or 7 position.

Embodiment 2 of this disclosure relates to a compound having Formula (Ia) or (Ib):

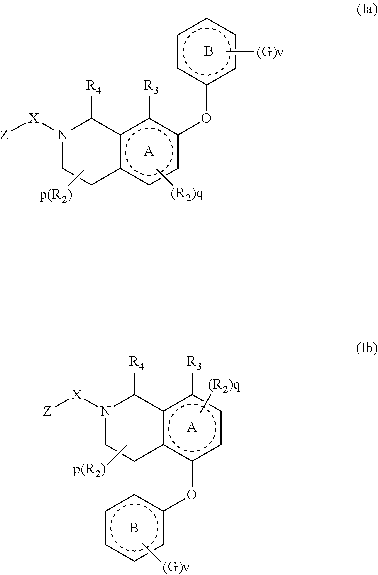

or a pharmaceutically acceptable salt, a tautomer, a stereoisomer, or a deuterated analog thereof, wherein:
ring A is phenyl or a 5- or 6-membered heteroaryl comprising 1-4 heteroatoms selected from N, O and S,
ring B is phenyl or a 5- or 6-membered heteroaryl comprising 1-4 heteroatoms selected from N, O and S,
   each G is independently selected from halogen, OH, CN, alkyl optionally substituted with one or more R⁵, and alkoxy optionally substituted with one or more R⁵;
each R² is independently H, halogen, —C(O)O-alkyl, or C₁-C₃alkyl optionally substituted with 1-3 halogens, provided that not more than one R² is —C(O)O-alkyl;
R³ is H; halogen; alkenyl optionally substituted with cycloalkyl or heterocycloalkyl; heterocycloalkyl optionally substituted with —C(O)-alkyl; heterocycloalkenyl optionally substituted with C(O)-alkyl; heterocycloalkylalkyl optionally substituted with C(O)-alkyl; or heteroaryl optionally substituted with haloalkyl, cycloalkyl, or cycloalkylalkyl;
R⁴ is H; alkyl; cycloalkyl optionally substituted with haloalkyl or —C(O)-alkenyl; heterocycloalkyl optionally substituted with —C(O)-alkyl, —C(O)-alkenyl, or —C(O)-cycloalkyl; or heterocycloalkylalkyl optionally substituted with —C(O)-alkyl, —C(O)—CH₂—OH, or heteroaryl;
R⁵ is halogen or OH;
X is —(CH₂)ₘ—S(O)₂—, —(CH₂)ₙ—C(O)—, or —C(O)O—, wherein the right-hand side indicates the point of attachment to Z;
Z is —NR⁶R⁷, C₁-C₆alkyl optionally substituted with 1-4 R⁸, —C(CH₃)═CH₂, —CH₂—CH═C═O, cycloalkyl optionally substituted with 1-4 R⁹, heterocycloalkyl optionally substituted with 1-4 R¹⁰, aryl optionally substituted with 1-4 R¹⁰, or heteroaryl optionally substituted with 1-4 R¹⁰;
R⁶ is hydrogen, C₁-C₆alkyl, cycloalkyl, heterocycloalkyl, aryl, or heteroaryl, wherein each C₁-C₆alkyl, cycloalkyl, heterocycloalkyl, aryl, or heteroaryl is optionally substituted with 1-3 R¹¹;
R⁷ is hydrogen or C₁-C₆alkyl; or
R⁶ and R⁷, together with the nitrogen to which they are attached, form a heterocycloalkyl;
R⁸ is —NR⁶R⁷, CN, hydroxy, alkoxy, haloalkoxy, —S—C₁-C₃alkyl, —S(O)—C₁-C₃alkyl, —S(O)₂—C₁-C₃alkyl, —S(O)₂—C₁-C₃haloalkyl, —S(O)₂—NH₂, —S(O)(NH)-C₁-C₃alkyl, —C(O)—NH₂, —C(O)OH, —C(O)O—C₁-C₃alkyl, —C(O)C₁-C₃haloalkyl, —N(H)—C(O)—NR⁶R⁷, —N(H)—C(O)—C₁-C₃alkyl optionally substituted with —NR¹²R¹³ or CN, —N(H)—C(O)—C₂-C₆alkenyl, —N(H)—C(O)—C₁-C₃haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₅haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰; or
two R⁸, together with the carbon atom to which they are attached, join together to form a cycloalkyl;
each R⁹ is independently CN, —S(O)₂—C₁-C₃alkyl, or —N(C₁-3alkyl)₂;
each R¹⁰ is independently hydroxy, halogen, CN, C₁-C₆alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkenyl, —S(O)₂—C₁-C₃alkyl, —S(O)₂—C₂-C₆alkenyl, or —S(O)₂—NH₂;
R¹¹ is CN;
each R¹² and R¹³ are independently hydrogen, C₁-C₆alkyl, or C₃-C₆cycloalkyl;
v is 0, 1, 2, 3, or 4;
p is 0, 1, 2, 3, or 4;
q is 0, 1, or 2;
n is 0, 1, or 2; and
m is 0, 1, 2, or 3;
provided that ring A and/or ring B is a 5- or 6-membered heteroaryl comprising 1-4 heteroatoms selected from N, O and S.

Embodiment 2a of this disclosure relates to a compound having Formula (Ia), (Ib), or (Ic):

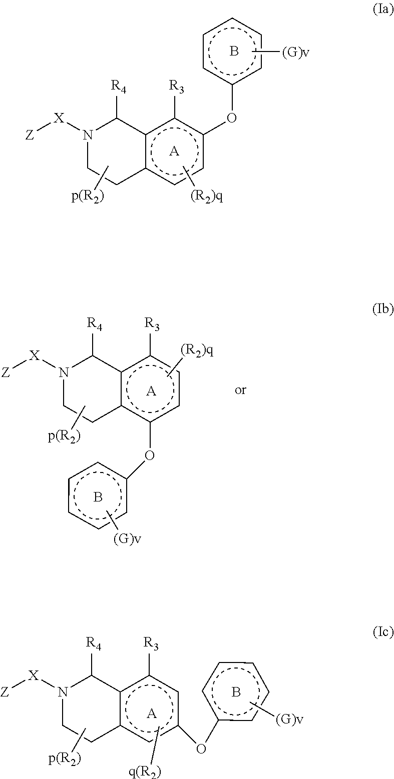

or a pharmaceutically acceptable salt, a tautomer, a stereoisomer, or a deuterated analog thereof, wherein:
ring A is phenyl or a 5- or 6-membered heteroaryl comprising 1-4 heteroatoms selected from N, O and S;
ring B is phenyl or a 5- or 6-membered heteroaryl comprising 1-4 heteroatoms selected from N, O and S,
each G is independently selected from halogen, OH, CN, alkyl optionally substituted with one or more R⁵, and alkoxy optionally substituted with one or more R⁵;
each R² is independently H, halogen, —C(O)O-alkyl, or C₁-C₃alkyl optionally substituted with 1-3 halogens, provided that not more than one R² is —C(O)O-alkyl;
R³ is H; halogen; alkenyl optionally substituted with cycloalkyl or heterocycloalkyl; heterocycloalkyl optionally substituted with —C(O)-alkyl; heterocycloalkenyl optionally substituted with C(O)-alkyl; heterocycloalkylalkyl optionally substituted with C(O)-alkyl; or heteroaryl optionally substituted with haloalkyl, cycloalkyl, or cycloalkylalkyl;
R⁴ is H; alkyl; cycloalkyl optionally substituted with haloalkyl or —C(O)-alkenyl; heterocycloalkyl optionally substituted with —C(O)-alkyl, —C(O)-alkenyl, or —C(O)-cycloalkyl; or heterocycloalkylalkyl optionally substituted with —C(O)-alkyl, —C(O)—CH₂—OH, or heteroaryl;
R⁵ is halogen or OH;
X is a bond, —(CH₂)ₘ—S(O)₂—, —(CH₂)ₙ—C(O)—, or —C(O)O—, wherein the right-hand side indicates the point of attachment to Z;
Z is —NR⁶R⁷, C₁-C₆ alkyl optionally substituted with 1-4 R⁸, C₂-C₆ alkenyl; —C (CH₃)═CH₂, —CH₂—CH═C═O, cycloalkyl optionally substituted with 1-4 R⁹, heterocycloalkyl optionally substituted with 1-4 R¹⁰, aryl optionally substituted with 1-4 R¹⁰, or heteroaryl optionally substituted with 1-4 R¹⁰;
R⁶ is hydrogen, —S(O)₂—C₁-C₃alkyl, C₁-C₆alkyl, cycloalkyl, heterocycloalkyl, aryl, or heteroaryl, wherein each C₁-C₆alkyl, cycloalkyl, heterocycloalkyl, aryl, or heteroaryl is optionally substituted with 1-3 R¹¹;
R⁷ is hydrogen or C₁-C₆alkyl; or
R⁶ and R⁷, together with the nitrogen to which they are attached, form a heterocycloalkyl;
R⁸ is halogen, —NR⁶R⁷, CN, hydroxy, alkoxy, haloalkyl, haloalkoxy, —S—C₁-C₃alkyl, —S(O)—C₁-C₃alkyl, —S(O)₂—C₁-C₃alkyl, —S(O)₂—C₁-C₃haloalkyl, —S(O)₂—NH₂, —S(O)₂ (NH)—C₁-C₃alkyl, —C(O)—NH₂, —C(O)OH, —C(O)O—C₁-C₃alkyl, —C(O)C₁-C₃haloalkyl, —N(H)—C(O)—NR⁶R⁷, —N(H)—C(O)—C₁-C₃alkyl optionally substituted with —NR¹²R¹³ or CN, —N(H)—C(O)—C₂-C₆alkenyl, —N(H)—C(O)—C₁-C₃haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₃haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰; or
two R⁸, together with the carbon atom to which they are attached, join together to form a cycloalkyl;
each R⁹ is independently CN, —S(O)₂—C₁-C₃alkyl, or —N(C₁-3alkyl)₂;
each R¹⁰ is independently hydroxy, halogen, CN, C₁-C₆ alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkyl, —NH—C(O)-alkenyl, N(H)—S(O)₂—C₁-C₃alkyl, —S(O)₂—C₁-C₃alkyl, —S(O)₂—C₁-C₃haloalkyl, —S(O)₂—C₂-C₆alkenyl, —S(O)₂—NH₂, or —S(O)₂—N(H)—C₁-C₃alkyl;
each R¹¹ is independently hydroxy, CN, alkoxy, —S(O)₂—C₁-C₃alkyl, or cycloalkyl;
each R¹² and R¹³ are independently hydrogen, C₁-C₆alkyl, or C₃-C₆cycloalkyl;
v is 0, 1, 2, 3, or 4;
p is 0, 1, 2, 3, or 4;
q is 0, 1, or 2;
n is 0, 1, or 2; and
m is 0, 1, 2, or 3;
   provided that if X is —(CH₂)ₘ—S(O)₂—, —(CH₂)ₙ—C(O)—, or —C(O)O—, then ring A and/or ring B is a 5- or 6-membered heteroaryl comprising 1-4 heteroatoms selected from N, O and S when —O— is attached to Ring A at the 5 or 7 position.

In an embodiment, X is a bond. In an embodiment, when X is a bond, Z is heterocycloalkyl optionally substituted with 1-4 R¹⁰.

In an embodiment, X is —(CH₂)ₘ—S(O)₂—, —(CH₂)ₙ—C(O)—, or —C(O)O—, wherein the right-hand side indicates the point of attachment to Z.

Embodiment 3 of this disclosure relates to the compound according to Embodiment 1 or Embodiment 2, wherein:
ring A is phenyl or a 5- or 6-membered heteroaryl comprising 1-4 heteroatoms selected from N, O and S,
ring B is phenyl or a 5- or 6-membered heteroaryl comprising 1-4 heteroatoms selected from N, O and S,
each G is independently selected from halogen, OH, CN, alkyl optionally substituted with one or more R⁵, and alkoxy optionally substituted with one or more R⁵;
each R² is independently H, halogen, —C(O)O-alkyl, or C₁-C₃alkyl optionally substituted with 1-3 halogens, provided that not more than one R² is —C(O)O-alkyl;
R³ is H; halogen; alkenyl optionally substituted with cycloalkyl or heterocycloalkyl; heterocycloalkyl optionally substituted with —C(O)-alkyl; heterocycloalkenyl optionally substituted with C(O)-alkyl; heterocycloalkylalkyl optionally substituted with C(O)-alkyl; or heteroaryl optionally substituted with haloalkyl, cycloalkyl, or cycloalkylalkyl;
R⁴ is H; alkyl; cycloalkyl optionally substituted with haloalkyl or —C(O)-alkenyl; heterocycloalkyl optionally substituted with —C(O)-alkyl, —C(O)-alkenyl, or —C(O)-cycloalkyl; or heterocycloalkylalkyl optionally substituted with —C(O)-alkyl, —C(O)—CH₂—OH, or heteroaryl;
R⁵ is halogen or OH;
X is —(CH₂)ₘ—S(O)₂—, —(CH₂)ₙ—C(O)—, or —C(O)O—, wherein the right-hand side indicates the point of attachment to Z;
Z is —NR⁶R⁷, C₁-C₆alkyl optionally substituted with 1-4 R⁸, —CH₂—CH═C—O, aryl optionally substituted with 1-4 R¹⁰, or heteroaryl optionally substituted with 1-4 R¹⁰;
R⁶ is hydrogen, C₁-C₆alkyl, cycloalkyl, heterocycloalkyl, aryl, or heteroaryl, wherein each C₁-C₆alkyl, cycloalkyl, heterocycloalkyl, aryl, or heteroaryl is optionally substituted with 1-3 R¹¹.
R⁷ is hydrogen or C₁-C₆alkyl; or
R⁶ and R⁷, together with the nitrogen to which they are attached, form a heterocycloalkyl;
R⁸ is —NR⁶R⁷, CN, hydroxy, alkoxy, haloalkoxy, —S—C₁-C₃alkyl, —S(O)—C₁-C₃alkyl, —S(O)₂—C₁-C₃alkyl, —S(O)₂—C₁-C₃haloalkyl, —S(O)₂—NH₂, —S(O)(NH)-C₁-C₃alkyl, —C(O)—NH₂, —C(O)OH, —C(O)O—C₁-C₃alkyl, —C(O)C₁-C₃haloalkyl, —N(H)—C(O)—NR⁶R⁷, —N(H)—C(O)—C₁-C₃alkyl optionally substituted with —NR¹²R¹³ or CN, —N(H)—C(O)—C₂-C₆alkenyl, —N(H)—C(O)—C₁-C₃haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₃haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰; or
two R⁸, together with the carbon atom to which they are attached, join together to form a cycloalkyl;
each R¹⁰ is independently hydroxy, halogen, CN, C₁-C₆alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkenyl, —S(O)₂—C₁-C₃alkyl, —S(O)₂—C₂-C₆alkenyl, or —S(O)₂—NH₂;
R¹¹ is CN;
each R¹² and R¹³ are independently hydrogen, C₁-C₆alkyl, or C₃-C₆cycloalkyl;
v is 0, 1, 2, 3, or 4;
p is 0, 1, 2, 3, or 4;
q is 0, 1, or 2;
n is 0, 1, or 2; and
m is 0, 1, 2, or 3;
provided that ring A and/or ring B is a 5- or 6-membered heteroaryl comprising 1-4 heteroatoms selected from N, O and S.

Embodiment 3a of this disclosure relates to the compound according to Embodiment 1a or Embodiment 2a, wherein:
ring A is phenyl or a 5- or 6-membered heteroaryl comprising 1-4 heteroatoms selected from N, O and S,
ring B is phenyl or a 5- or 6-membered heteroaryl comprising 1-4 heteroatoms selected from N, O and S,
each G is independently selected from halogen, OH, CN, alkyl optionally substituted with one or more R⁵, and alkoxy optionally substituted with one or more R⁵;
each R² is independently H, halogen, —C(O)O-alkyl, or C₁-C₃alkyl optionally substituted with 1-3 halogens, provided that not more than one R² is —C(O)O-alkyl;
R³ is H; halogen; alkenyl optionally substituted with cycloalkyl or heterocycloalkyl; heterocycloalkyl optionally substituted with —C(O)-alkyl; heterocycloalkenyl optionally substituted with C(O)-alkyl; heterocycloalkylalkyl optionally substituted with C(O)-alkyl; or heteroaryl optionally substituted with haloalkyl, cycloalkyl, or cycloalkylalkyl;
R⁴ is H; alkyl; cycloalkyl optionally substituted with haloalkyl or —C(O)-alkenyl; heterocycloalkyl optionally substituted with —C(O)-alkyl, —C(O)-alkenyl, or —C(O)-cycloalkyl; or heterocycloalkylalkyl optionally substituted with —C(O)-alkyl, —C(O)—CH₂—OH, or heteroaryl;
R⁵ is halogen or OH;
X is —(CH₂)ₘ—S(O)₂—, —(CH₂)ₙ—C(O)—, or —C(O)O—, wherein the right-hand side indicates the point of attachment to Z;
Z is —NR⁶R⁷, C₂-C₆alkenyl, C₁-C₆alkyl optionally substituted with 1-4 R⁸, —CH₂—CH═C═O, aryl optionally substituted with 1-4 R¹⁰, or heteroaryl optionally substituted with 1-4 R¹⁰;
R⁶ is hydrogen, C₁-C₆alkyl, cycloalkyl, heterocycloalkyl, aryl, or heteroaryl, wherein each C₁-C₆alkyl, cycloalkyl, heterocycloalkyl, aryl, or heteroaryl is optionally substituted with 1-3 R¹¹;
R⁷ is hydrogen or C₁-C₆alkyl; or
R⁶ and R⁷, together with the nitrogen to which they are attached, form a heterocycloalkyl;
R⁸ is —NR⁶R⁷, CN, halogen, hydroxy, alkoxy, haloalkyl, haloalkoxy, —S—C₁-C₃alkyl, —S(O)—C₁-C₃alkyl, —S(O)₂—C₁-C₃alkyl, —S(O)₂—C₁-C₃haloalkyl, —S(O)₂—NH₂, —S(O)₂ (NH)-C₁-C₃alkyl, —C(O)—NH₂, —C(O)OH, —C(O)O—C₁-C₃alkyl, —C(O)C₁-C₃haloalkyl, —N(H)—C(O)—NR⁶R⁷, —N(H)—C(O)—C₁-C₃alkyl optionally substituted with —NR¹²R¹³ or CN, —N(H)—C(O)—C₂-C₆alkenyl, —N(H)—C(O)—C₁-C₃haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₃haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰; or
two R⁸, together with the carbon atom to which they are attached, join together to form a cycloalkyl;
each R¹⁰ is independently hydroxy, halogen, CN, C₁-C₆alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkyl, —NH—C(O)-alkenyl, N(H)—S(O)₂—C₁-C₃alkyl, —S(O)₂—C₁-C₃alkyl, —S(O)₂—C₁-C₃haloalkyl, —S(O)₂—C₂-C₆alkenyl, —S(O)₂—NH₂, or —S(O)₂—N(H)—C₁-C₃alkyl;
R¹¹ is CN;
each R¹² and R¹³ are independently hydrogen, C₁-C₆alkyl, or C₃-C₆cycloalkyl;
v is 0, 1, 2, 3, or 4;
p is 0, 1, 2, 3, or 4;
q is 0, 1, or 2;
n is 0, 1, or 2; and
m is 0, 1, 2, or 3;
   provided that if X is —(CH₂)ₘ—S(O)₂—, —(CH₂)ₙ—C(O)—, or —C(O)O—, then ring A and/or ring B is a 5- or 6-membered heteroaryl comprising 1-4 heteroatoms selected from N, O and S when —O— is attached to Ring A at the 5 or 7 position.

Embodiment 3b of this disclosure relates to the compound according to Embodiment 1a or Embodiment 2a, wherein:
ring A is phenyl or a 5- or 6-membered heteroaryl comprising 1-4 heteroatoms selected from N, O and S,
ring B is phenyl or a 5- or 6-membered heteroaryl comprising 1-4 heteroatoms selected from N, O and S,
each G is independently selected from halogen, OH, CN, alkyl optionally substituted with one or more R⁵, and alkoxy optionally substituted with one or more R⁵;
each R² is independently H, halogen, —C(O)O-alkyl, or C₁-C₃alkyl optionally substituted with 1-3 halogens, provided that not more than one R² is —C(O)O-alkyl;
R³ is H; halogen; alkenyl optionally substituted with cycloalkyl or heterocycloalkyl; heterocycloalkyl optionally substituted with —C(O)-alkyl; heterocycloalkenyl optionally substituted with C(O)-alkyl; heterocycloalkylalkyl optionally substituted with C(O)-alkyl; or heteroaryl optionally substituted with haloalkyl, cycloalkyl, or cycloalkylalkyl;
R⁴ is H; alkyl; cycloalkyl optionally substituted with haloalkyl or —C(O)-alkenyl; heterocycloalkyl optionally substituted with —C(O)-alkyl, —C(O)-alkenyl, or —C(O)-cycloalkyl; or heterocycloalkylalkyl optionally substituted with —C(O)-alkyl, —C(O)—CH₂—OH, or heteroaryl;
R⁵ is halogen or OH;
X is bond;
Z is heterocycloalkyl optionally substituted with 1-4 R¹⁰;
R⁶ is hydrogen, C₁-C₆alkyl, cycloalkyl, heterocycloalkyl, aryl, or heteroaryl, wherein each C₁-C₆alkyl, cycloalkyl, heterocycloalkyl, aryl, or heteroaryl is optionally substituted with 1-3 R¹¹;
R⁷ is hydrogen or C₁-C₆alkyl; or
R⁶ and R⁷, together with the nitrogen to which they are attached, form a heterocycloalkyl;
R⁸ is —NR⁶R⁷, CN, halogen, hydroxy, alkoxy, haloalkyl, haloalkoxy, —S—C₁-C₃alkyl, —S(O)—C₁-C₃alkyl, —S(O)₂—C₁-C₃alkyl, —S(O)₂—C₁-C₃haloalkyl, —S(O)₂—NH₂, —S(O)₂ (NH)—C₁-C₃alkyl, —C(O)—NH₂, —C(O)OH, —C(O)O—C₁-C₃alkyl, —C(O)C₁-C₃haloalkyl, —N(H)—C(O)—NR⁶R⁷, —N(H)—C(O)—C₁-C₃alkyl optionally substituted with —NR¹²R¹³ or CN, —N(H)—C(O)—C₂-C₆alkenyl, —N(H)—C(O)—C₁-C₃haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₃haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰; or
two R⁸, together with the carbon atom to which they are attached, join together to form a cycloalkyl;
each R¹⁰ is independently hydroxy, halogen, CN, C₁-C₆alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkyl, —NH—C(O)-alkenyl, N(H)—S(O)₂—C₁-C₃alkyl, —S(O)₂—C₁-C₃alkyl, —S(O)₂—C₁-C₃haloalkyl, —S(O)₂—C₂-Calkenyl, —S(O)₂—NH₂, or —S(O)₂—N(H)—C₁-C₃alkyl;
R¹¹ is CN;
each R¹² and R¹³ are independently hydrogen, C₁-C₆alkyl, or C₃-C₆cycloalkyl;
v is 0, 1, 2, 3, or 4;
p is 0, 1, 2, 3, or 4;
q is 0, 1, or 2;
n is 0, 1, or 2; and
m is 0, 1, 2, or 3;
provided that if X is —(CH₂)ₘ—S(O)₂—, —(CH₂)ₙ—C(O)—, or —C(O)O—, then ring A and/or ring B is a 5- or 6-membered heteroaryl comprising 1-4 heteroatoms selected from N, O and S when —O— is attached to Ring A at the 5 or 7 position.

Embodiment 4 of this disclosure relates to the compound according to Embodiment 1 or Embodiment 2, wherein:
ring A is phenyl or a 5- or 6-membered heteroaryl comprising 1-4 heteroatoms selected from N, O and S,
ring B is phenyl or a 5- or 6-membered heteroaryl comprising 1-4 heteroatoms selected from N, O and S,
v is 0, 1, 2 or 3;
each G is independently selected from halogen, CN, and C₁-C₃alkyl optionally substituted with 1-3 R⁵;
each R² is H, halogen, or CH₃;
R⁵ is halogen or OH;
X is —(CH₂)ₘ—S(O)₂— or —(CH₂)ₙ—C(O)—; and
Z is —NR⁶R⁷, C₁-C₆alkyl optionally substituted with 1-3 R⁸, cycloalkyl optionally substituted with 1-3 R⁹, heterocycloalkyl optionally substituted with 1-3 R¹⁰, aryl optionally substituted with 1-3 R¹⁰, or heteroaryl optionally substituted with 1-3 R¹⁰.

Embodiment 4a of this disclosure relates to the compound according to Embodiment 1a or Embodiment 2a, wherein:
ring A is phenyl or a 5- or 6-membered heteroaryl comprising 1-4 heteroatoms selected from N, O and S,
ring B is phenyl or a 5- or 6-membered heteroaryl comprising 1-4 heteroatoms selected from N, O and S,
v is 0, 1, 2 or 3;
each G is independently selected from halogen, CN, and C₁-C₃alkyl optionally substituted with 1-3 R⁵;
each R² is H, halogen, or CH₃; R⁵ is halogen or OH;
X is —(CH₂)ₘ—S(O)₂— or —(CH₂)ₙ—C(O)—; and
Z is —NR⁶R⁷, C₂-Calkenyl, C₁-C₆alkyl optionally substituted with 1-3 R⁸, cycloalkyl optionally substituted with 1-3 R⁹, heterocycloalkyl optionally substituted with 1-3 R¹⁰, aryl optionally substituted with 1-3 R¹⁰, or heteroaryl optionally substituted with 1-3 R¹⁰;
provided that ring A and/or ring B is a 5- or 6-membered heteroaryl comprising 1-4 heteroatoms selected from N, O and S when —O— is attached to Ring A at the 5 or 7 position.

Embodiment 4b of this disclosure relates to the compound according to Embodiment 1a or Embodiment 2a, wherein:
ring A is phenyl or a 5- or 6-membered heteroaryl comprising 1-4 heteroatoms selected from N, O and S,
ring B is phenyl or a 5- or 6-membered heteroaryl comprising 1-4 heteroatoms selected from N, O and S,
v is 0, 1, 2 or 3;
each G is independently selected from halogen, CN, and C₁-C₃alkyl optionally substituted with 1-3 R⁵;
each R² is H, halogen, or CH₃; R⁵ is halogen or OH; and
X is a bond and Z is heterocycloalkyl optionally substituted with 1-4 R¹⁰.

Embodiment 5 of this disclosure relates to the compound according to Embodiment 4, wherein:
ring A is phenyl or a 5- or 6-membered heteroaryl comprising 1-4 heteroatoms selected from N, O and S,
ring B is phenyl or a 5- or 6-membered heteroaryl comprising 1-4 heteroatoms selected from N, O and S,
v is 0, 1, or 2;
each G is independently selected from C₁, F, CN, and C₁-C₃alkyl substituted with 1-3 R⁵;
each R² is H, C₁, F, or CH₃; and
R⁵ is halogen.

Embodiment 5a of this disclosure relates to the compound according to Embodiment 4a or Embodiment 4b, wherein:
ring A is phenyl or a 5- or 6-membered heteroaryl comprising 1-4 heteroatoms selected from N, O and S,
ring B is phenyl or a 5- or 6-membered heteroaryl comprising 1-4 heteroatoms selected from N, O and S,
v is 0, 1, or 2;
each G is independently selected from C₁, F, CN, and C₁-C₃alkyl substituted with 1-3 R⁵;
each R² is H, C₁, F, or CH₃; and
R⁵ is halogen.

Embodiment 6 of this disclosure relates to the compound of Embodiment 5, wherein R⁵ is Cl or F.

Embodiment 6a of this disclosure relates to the compound of Embodiment 5a, wherein R⁵ is Cl or F.

Embodiment 7 of this disclosure relates to the compound of one of Embodiment 1 or Embodiment 2 or Embodiment 3, having one of the following formulae:

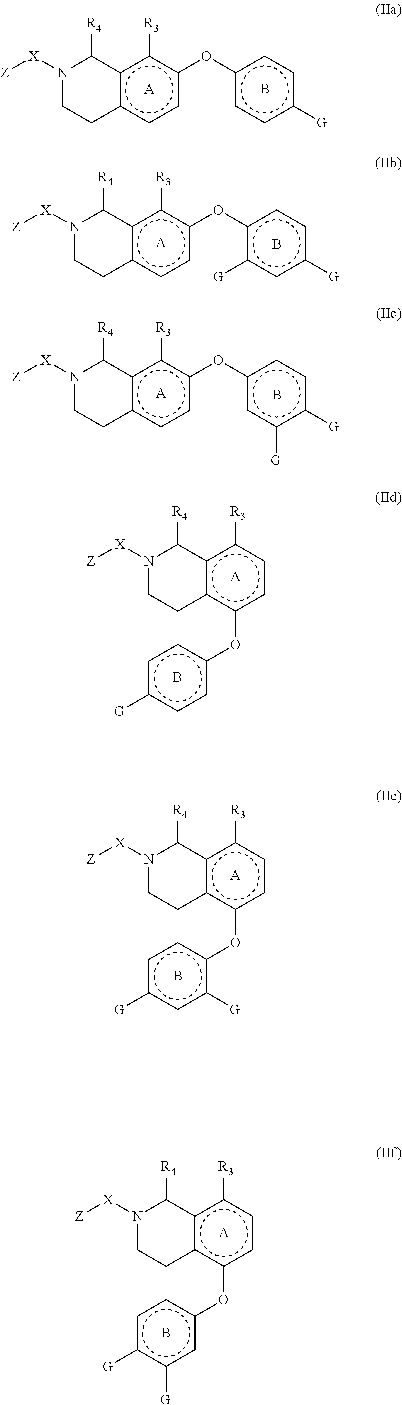

or a pharmaceutically acceptable salt, a tautomer, a stereoisomer, or a deuterated analog of any of formulae (IIa), (IIb), (IIc), (IId), (IIe), or (IIf); wherein:
each G is independently selected from C₁, F, CN, and C₁-C₃alkyl substituted with 1-3 R⁵; and
R⁵ is halogen.

Embodiment 7a of this disclosure relates to the compound of one of Embodiment 1a or Embodiment 2a or Embodiment 3a, having one of the following formulae:

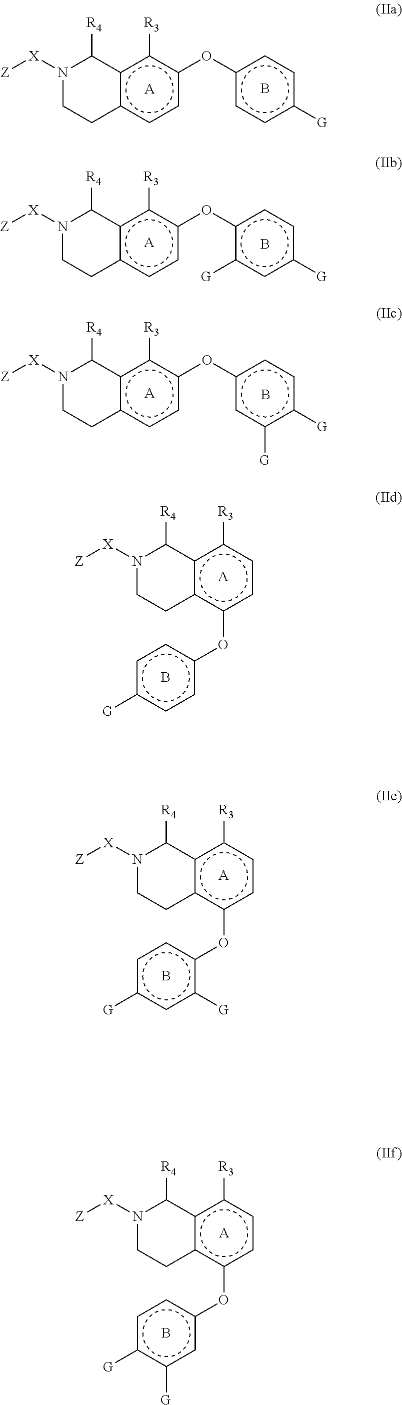

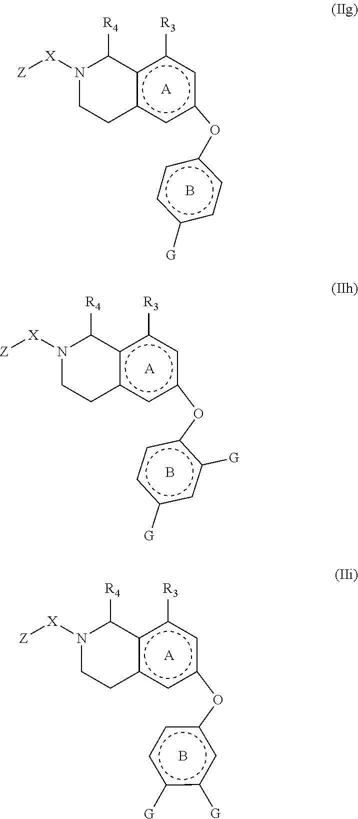

or a pharmaceutically acceptable salt, a tautomer, a stereoisomer, or a deuterated analog of any of formulae (IIa), (IIb), (IIc), (IId), (Ile), (IIf), (IIg), (IIh), or (IIi); wherein:
each G is independently selected from C₁, F, CN, and C₁-C₃alkyl substituted with 1-3 R⁵;
R⁵ is halogen.

Embodiment 8 of this disclosure relates to the compound according to Embodiment 7, and having one of the following formulae:

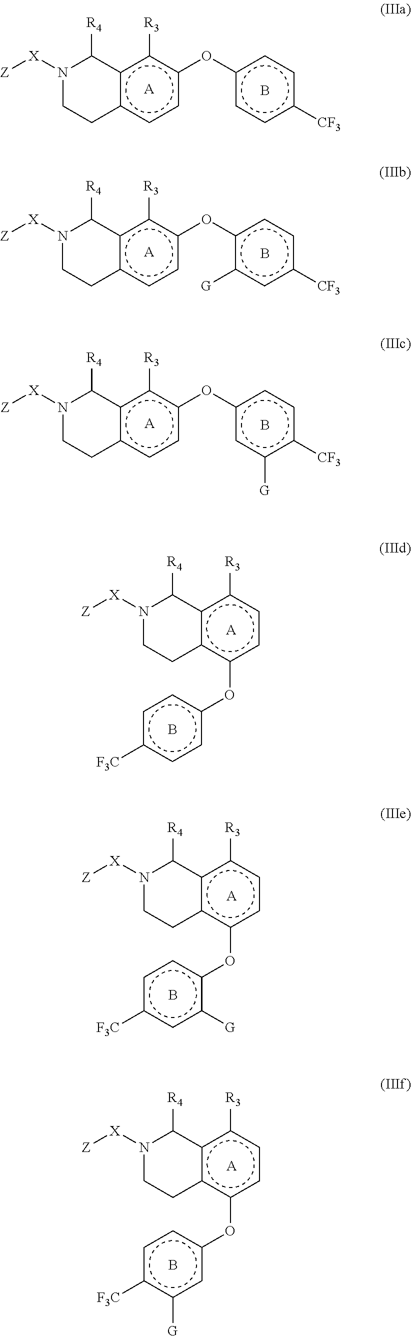

or a pharmaceutically acceptable salt, a tautomer, a stereoisomer, or a deuterated analog of any of formulae (IIIa), (IIIb), (IIIc), (IIId), (IIIe) or (IIIf); wherein:
G is Cl, F, or CN.

Embodiment 8a of this disclosure relates to the compound according to Embodiment 7a, having one of the following formulae:

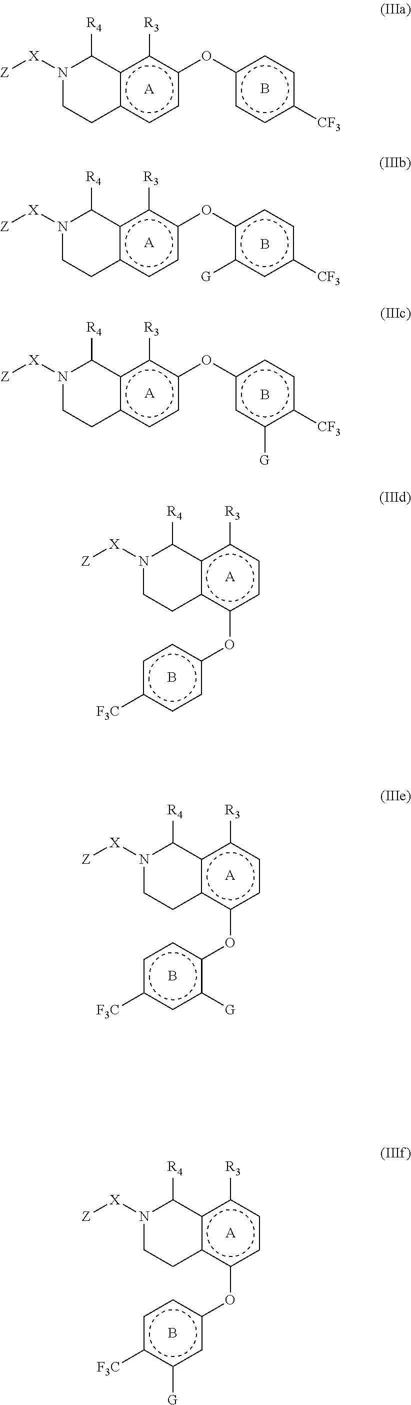

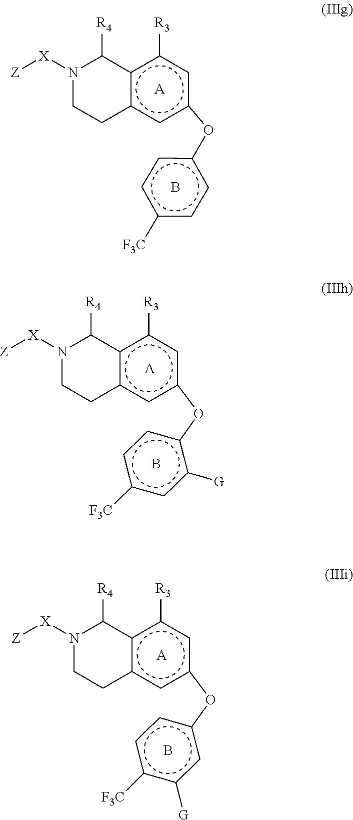

or a pharmaceutically acceptable salt, a tautomer, a stereoisomer, or a deuterated analog of any of formulae (IIIa), (IIIb), (IIIc), (IIId), (IIIe), (IIIf), (IIIg), (IIIh), or (IIIi); wherein:
G is C₁, F, or CN.

Embodiment 9 of this disclosure relates to the compound of one of Embodiment 7 or Embodiment 8, wherein R³ is H; halogen; C₂-C₄alkenyl optionally substituted with cyclopropyl or heterocycloalkyl; heterocycloalkyl optionally substituted with C(O)—CH₃; heterocycloalkenyl optionally substituted with C(O)—CH₃; heterocycloalkylalkyl optionally substituted with C(O)—CH₃; or 5-6 membered heteroaryl optionally substituted with haloalkyl, cyclopropyl, or cyclopropyl-CH₂—.

Embodiment 9a of this disclosure relates to the compound of one of Embodiment 7a or Embodiment 8a, wherein R³ is H; halogen; C₂-C₄alkenyl optionally substituted with cyclopropyl or heterocycloalkyl; heterocycloalkyl optionally substituted with C(O)—CH₃; heterocycloalkenyl optionally substituted with C(O)—CH₃; heterocycloalkylalkyl optionally substituted with C(O)—CH₃; or 5-6 membered heteroaryl optionally substituted with haloalkyl, cyclopropyl, or cyclopropyl-CH₂—.

Embodiment 10 of this disclosure relates to the compound of one of Embodiments 7-9, wherein R⁴ is H; C₁-C₃alkyl; cycloalkyl optionally substituted with haloalkyl or —C(O)-alkenyl; heterocycloalkyl optionally substituted with —C(O)—CH₃, —C(O)—CH═CH₂, or —C(O)-cyclopropyl; heterocycloalkylalkyl optionally substituted with —C(O)—CH₃; —C(O)—CH₂—OH; or 5-6 membered heteroaryl.

Embodiment 10a of this disclosure relates to the compound of one of Embodiment 7a, Embodiment 8a, or Embodiment 9a, wherein R⁴ is H; C₁-C₃alkyl; cycloalkyl optionally substituted with haloalkyl or —C(O)-alkenyl; heterocycloalkyl optionally substituted with —C(O)—CH₃, —C(O)—CH—CH₂, or —C(O)-cyclopropyl; heterocycloalkylalkyl optionally substituted with —C(O)—CH₃; —C(O)—CH₂—OH; or 5-6 membered heteroaryl.

Embodiment 11 of this disclosure relates to the compound of Embodiment 1 or Embodiment 2, having one of the following formulae:

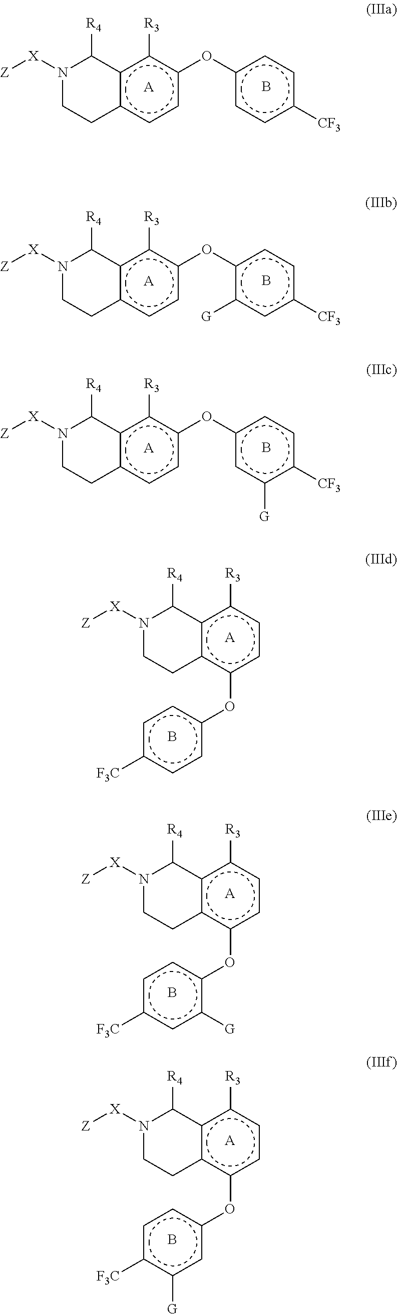

or a pharmaceutically acceptable salt, a tautomer, a stereoisomer, or a deuterated analog of any of formulae (IIIa), (IIIb), (IIIc), (IIId), (IIIe) or (IIIf); wherein:
G is C₁, F, or CN;
R³ is H; halogen; C₂-C₄alkenyl optionally substituted with cyclopropyl or heterocycloalkyl; heterocycloalkyl optionally substituted with C(O)—CH₃; heterocycloalkenyl optionally substituted with C(O)—CH₃; heterocycloalkylalkyl optionally substituted with C(O)—CH₃; or 5-6 membered heteroaryl optionally substituted with haloalkyl, cyclopropyl, or cyclopropyl-CH₂—;
R⁴ is H; C₁-C₃alkyl; cycloalkyl optionally substituted with haloalkyl or —C(O)-alkenyl; heterocycloalkyl optionally substituted with —C(O)—CH₃—C(O)—CH═CH₂, or —C(O)-cyclopropyl; heterocycloalkylalkyl optionally substituted with —C(O)—CH₃; —C(O)—CH₂—OH; or 5-6 membered heteroaryl;
X is —(CH₂)ₙ—C(O)—;
Z is —NR⁶R⁷, C₁-C₆alkyl optionally substituted with 1-4 R⁸, C₃-C₆cycloalkyl optionally substituted with 1-4 R⁹, 5-10 membered heterocycloalkyl optionally substituted with 1-4 R¹⁰, C₆-C₁₂aryl optionally substituted with 1-4 R¹⁰, or 5-10 membered heteroaryl optionally substituted with 1-4 R¹⁰; and
n is 0, 1, or 2.

Embodiment 11a of this disclosure relates to the compound of Embodiment 1a or Embodiment 2a, having one of the following formulae:

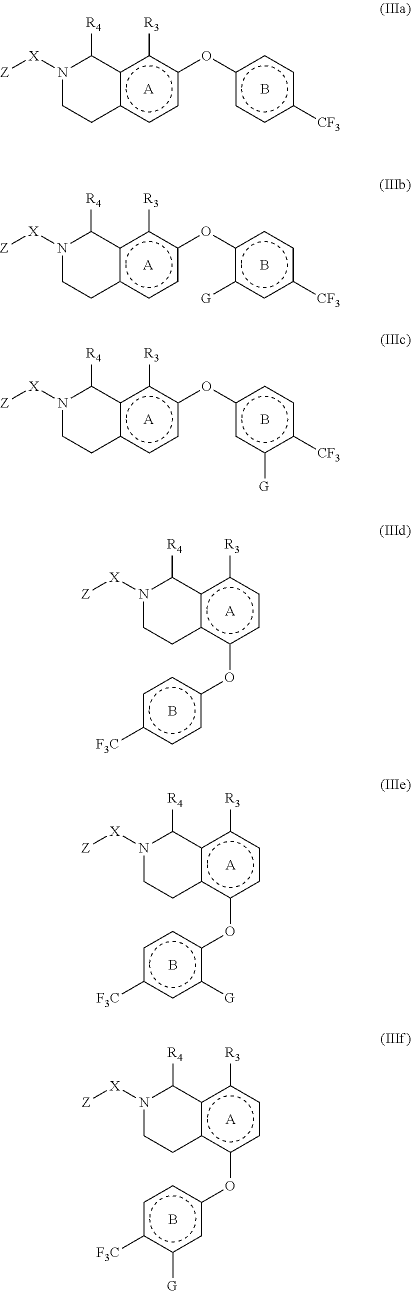

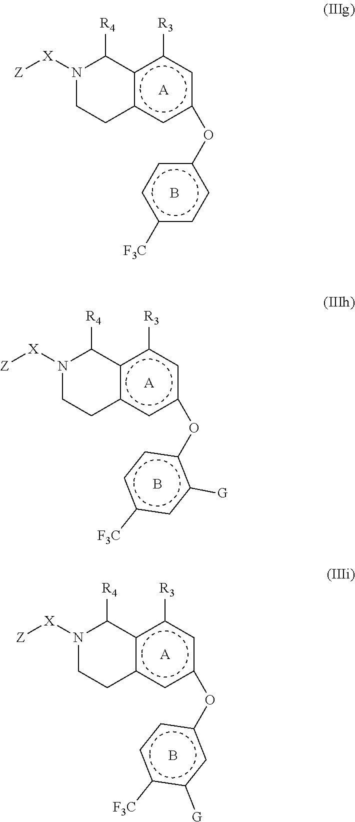

or a pharmaceutically acceptable salt, a tautomer, a stereoisomer, or a deuterated analog of any of formulae (IIIa), (IIIb), (IIIc), (IIId), (IIIe), (IIIf), (IIIg), (IIIh), or (IIIi); wherein:
G is C₁, F, or CN;
R³ is H; halogen; C₂-C₄alkenyl optionally substituted with cyclopropyl or heterocycloalkyl; heterocycloalkyl optionally substituted with C(O)—CH₃; heterocycloalkenyl optionally substituted with C(O)—CH₃; heterocycloalkylalkyl optionally substituted with C(O)—CH₃; or 5-6 membered heteroaryl optionally substituted with haloalkyl, cyclopropyl, or cyclopropyl-CH₂—;
R⁴ is H; C₁-C₃alkyl; cycloalkyl optionally substituted with haloalkyl or —C(O)-alkenyl; heterocycloalkyl optionally substituted with —C(O)—CH₃—C(O)—CH═CH₂, or —C(O)-cyclopropyl; heterocycloalkylalkyl optionally substituted with —C(O)—CH₃; —C(O)—CH₂—OH; or 5-6 membered heteroaryl;
X is —(CH₂)ₙ—C(O)—;
Z is —NR⁶R⁷, C₂-C₆alkenyl, C₁-C₆alkyl optionally substituted with 1-4 R⁸, C₃-C₆cycloalkyl optionally substituted with 1-4 R⁹, 5-10 membered heterocycloalkyl optionally substituted with 1-4 R¹⁰, C₆-C₁₂aryl optionally substituted with 1-4 R¹⁰, or 5-10 membered heteroaryl optionally substituted with 1-4 R¹⁰; and
n is 0, 1, or 2.

Embodiment 12 of this disclosure relates to the compound of Embodiment 11, wherein:
Z is —NR⁶R⁷, C₁-C₄alkyl optionally substituted with 1-3 R⁸, C₃-C₆cycloalkyl optionally substituted with 1-3 R⁹, 5-7 membered heterocycloalkyl optionally substituted with 1-3 R¹⁰, C₆-C₁₀aryl optionally substituted with 1-3 R¹⁰, or 5-7 membered heteroaryl optionally substituted with 1-3 R¹⁰; and
n is 0 or 1. Preferably Z is substituted with 1-3 R⁸. Preferably Z is substituted with 1 R⁸. Preferably Z is substituted with 2 R⁸. Preferably Z is substituted with 3 R⁸. Preferably Z is substituted with 1-3 R⁹. Preferably Z is substituted with 1 R⁹. Preferably Z is substituted with 2 R⁹. Preferably Z is substituted with 3 R⁹. Preferably Z is substituted with 1-3 R¹⁰. Preferably Z is substituted with 1 R¹⁰. Preferably Z is substituted with 2 R¹⁰. Preferably Z is substituted with 3 R¹⁰.

Embodiment 12a of this disclosure relates to the compound of Embodiment 11a, wherein:
Z is —NR⁶R⁷, C₂-C₄alkenyl, C₁-C₄alkyl optionally substituted with 1-3 R⁸, C₃-C₆cycloalkyl optionally substituted with 1-3 R⁹, 5-7 membered heterocycloalkyl optionally substituted with 1-3 R¹⁰, C₆-C₁₀aryl optionally substituted with 1-3 R¹⁰, or 5-7 membered heteroaryl optionally substituted with 1-3 R¹⁰; and
n is 0 or 1. Preferably Z is substituted with 1-3 R⁸. Preferably Z is substituted with 1 R⁸. Preferably Z is substituted with 2 R⁸. Preferably Z is substituted with 3 R⁸. Preferably Z is substituted with 1-3 R⁹. Preferably Z is substituted with 1 R⁹. Preferably Z is substituted with 2 R⁹. Preferably Z is substituted with 3 R⁹. Preferably Z is substituted with 1-3 R¹⁰. Preferably Z is substituted with 1 R¹⁰. Preferably Z is substituted with 2 R¹⁰. Preferably Z is substituted with 3 R¹⁰.

Embodiment 13 of this disclosure relates to the compound of Embodiment 11, wherein:
Z is —NR⁶R⁷, C₂-C₄alkyl optionally substituted with 1-3 R⁸, C₆-C₁₂aryl optionally substituted with 1-3 R¹⁰, or 5-10 membered heteroaryl optionally substituted with 1-3 R¹⁰; and
n is 0 or 1.

Embodiment 13a of this disclosure relates to the compound of Embodiment 11a, wherein:
Z is —NR⁶R⁷, C₂-C₄alkenyl, C₂-C₄alkyl optionally substituted with 1-3 R⁸, C₆-C₁₂aryl optionally substituted with 1-3 R¹⁰, or 5-10 membered heteroaryl optionally substituted with 1-3 R¹⁰; and
n is 0 or 1.

Embodiment 14 of this disclosure relates to the compound of Embodiment 11, wherein:
Z is —NR⁶R⁷; and
n is 0 or 1.

Embodiment 14a of this disclosure relates to the compound of Embodiment 11a, wherein:
Z is —NR⁶R⁷; and
n is 0 or 1.

Embodiment 15 of this disclosure relates to the compound of Embodiment 11, wherein: Z is C₆-C₁₂aryl optionally substituted with 1-3 R¹⁰; and
n is 0 or 1. Preferably Z is substituted with 1-3 R¹⁰. Preferably Z is substituted with 1 R¹⁰. Preferably Z is substituted with 2 R¹⁰. Preferably Z is substituted with 3 R¹⁰.

Embodiment 15a of this disclosure relates to the compound of Embodiment 11a, wherein:
Z is C₆-C₁₂aryl optionally substituted with 1-3 R¹⁰; and
n is 0 or 1. Preferably Z is substituted with 1-3 R¹⁰. Preferably Z is substituted with 1 R¹⁰. Preferably Z is substituted with 2 R¹⁰. Preferably Z is substituted with 3 R¹⁰.

Embodiment 16 of this disclosure relates to the compound of Embodiment 11, wherein:
Z is 5-10 membered heteroaryl optionally substituted with 1-3 R¹⁰; and
n is 0 or 1. Preferably Z is substituted with 1-3 R¹⁰. Preferably Z is substituted with 1 R¹⁰. Preferably Z is substituted with 2 R¹⁰. Preferably Z is substituted with 3 R¹⁰.

Embodiment 16a of this disclosure relates to the compound of Embodiment 11a, wherein:
Z is 5-10 membered heteroaryl optionally substituted with 1-3 R¹⁰; and
n is 0 or 1. Preferably Z is substituted with 1-3 R¹⁰. Preferably Z is substituted with 1 R¹⁰. Preferably Z is substituted with 2 R¹⁰. Preferably Z is substituted with 3 R¹⁰.

Embodiment 17 of this disclosure relates to the compound of Embodiments 1-16, wherein X—Z is selected from

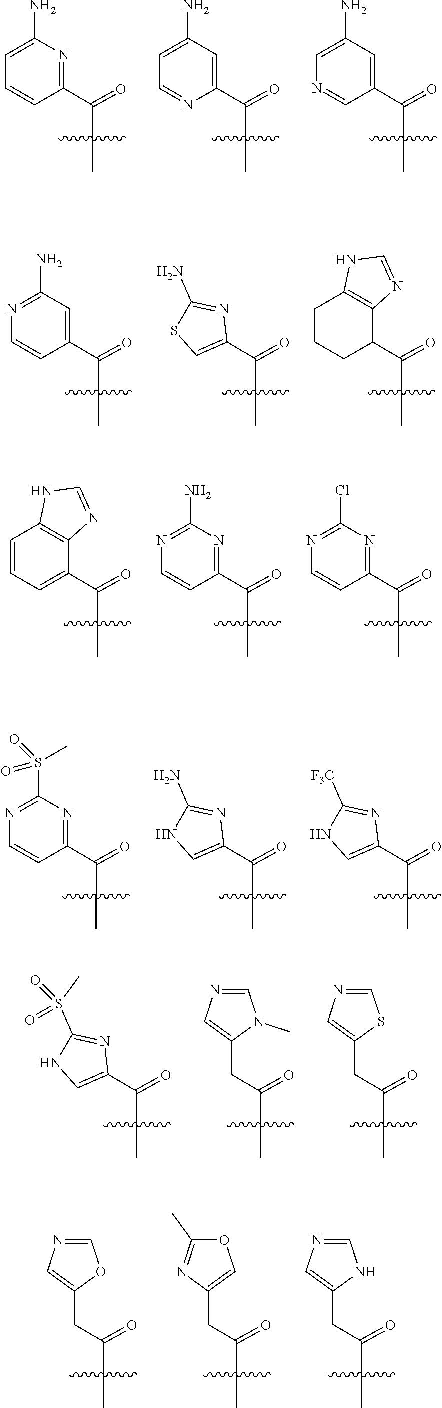

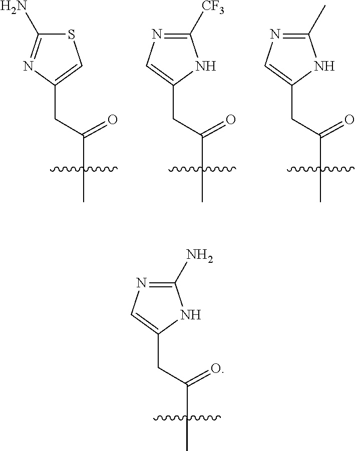

Embodiment 17a of this disclosure relates to the compound of Embodiment 1a, Embodiment 2a, Embodiment 3a, Embodiment 4a, Embodiment 4b, Embodiment 5a, Embodiment 6a, Embodiment 7a, Embodiment 8a, Embodiment 9a, Embodiment 10a, Embodiment 11a, Embodiment 12a, Embodiment 13a, Embodiment 14a, Embodiment 15a, 5 or Embodiment 16a, wherein X—Z is selected from

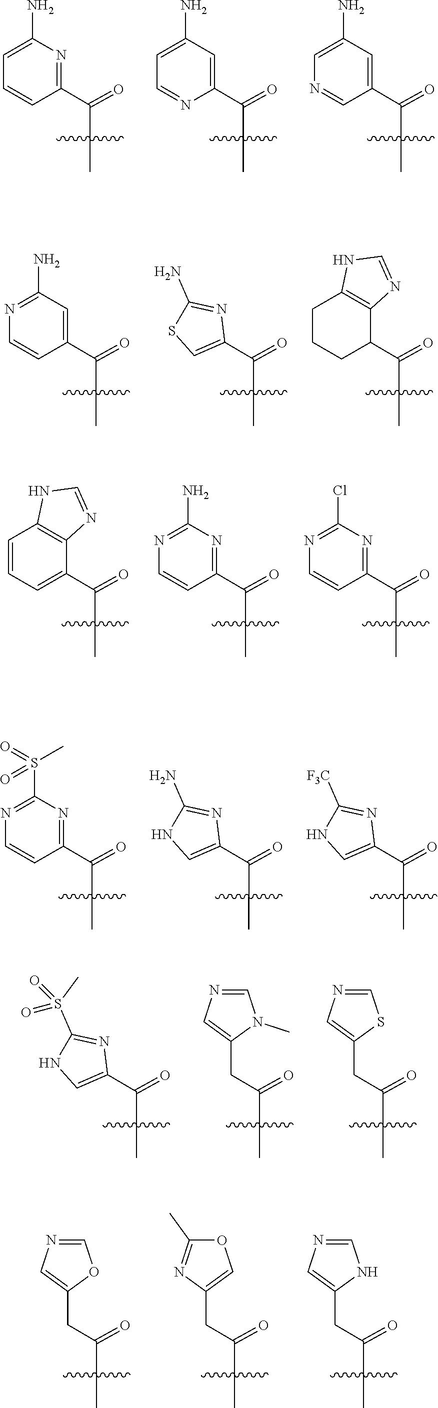

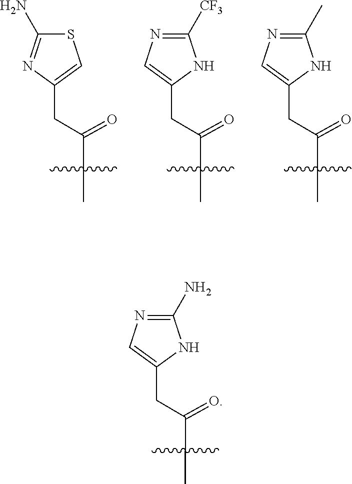

Embodiment 18 of this disclosure relates to the compound of Embodiment 11, wherein:
Z is C₂-C₄alkyl optionally substituted with 1-3 R⁸; and
n is 0 or 1. Preferably Z is substituted with 1-3 R⁸. Preferably Z is substituted with 1 R⁸. Preferably Z is substituted with 2 R⁸. Preferably Z is substituted with 3 R⁸.

Embodiment 18a of this disclosure relates to the compound of Embodiment 11a, wherein:
Z is C₂-C₄alkenyl or C₂-C₄alkyl optionally substituted with 1-3 R⁸; and
n is 0 or 1.

Preferably Z is C₂-C₄alkenyl. Preferably, Z is C₂ alkenyl.

Preferably Z is C₂-C₄alkyl optionally substituted with 1-3 R⁸. Preferably Z is substituted with 1-3 R⁸. Preferably Z is substituted with 1 R⁸. Preferably Z is substituted with 2 R⁸. Preferably Z is substituted with 3 R⁸.

Embodiment 19 of this disclosure relates to the compound of Embodiment 18, wherein Z is —(CH₂)₂-S(O)₂—CH₃ or —CH₂—N(H)—C(O)—NH₂. Preferably Z is —(CH₂)₂-S(O)₂—CH₃.

Embodiment 19a of this disclosure relates to the compound of Embodiment 18a, wherein Z is —(CH₂)₂-S(O)₂—CH₃ or —CH₂—N(H)—C(O)—NH₂. Preferably Z is —(CH₂)₂-S(O)₂—CH₃.

Embodiment 20 of this disclosure relates to the compound of one of Embodiments 1-19, wherein R³ is H and R⁴ is H.

Embodiment 20a of this disclosure relates to the compound of one of Embodiments 1a-19a, wherein R³ is H and R⁴ is H.

Embodiment 21 of this disclosure relates to the compound of one of Embodiments 1-20, wherein n is 1.

Embodiment 21a of this disclosure relates to the compound of one of Embodiments 1a-20a, wherein n is 1.

Embodiment 22 of this disclosure relates to the compound of one of Embodiments 1-20, wherein n is 0.

Embodiment 22a of this disclosure relates to the compound of one of Embodiments 1a-20a, wherein n is 0.

25 Embodiment 23 of this disclosure relates to the compound of one of Embodiments 1-22, wherein ring A is phenyl and ring B is 6 membered heteroaryl. Preferably the 6 membered heteroaryl is pyridine, pyridazine, pyrimidine, or pyrazine. Preferably the 6 membered heteroaryl is pyridine, or pyrimidine. Ring A and/or ring B can be further substituted as described herein.

Embodiment 23a of this disclosure relates to the compound of one of Embodiments 1a-22a, wherein ring A is phenyl and ring B is 6 membered heteroaryl. Preferably the 6 membered heteroaryl is pyridine, pyridazine, pyrimidine, or pyrazine. Preferably the 6 membered heteroaryl is pyridine, or pyrimidine. Ring A and/or ring B can be further substituted as described herein.

Embodiment 23b of this disclosure relates to the compound of one of Embodiments 1a-22a, wherein ring A is phenyl and ring B is phenyl.

Embodiment 24 of this disclosure relates to the compound of one of Embodiments 1-22, wherein ring A is a 6 membered heteroaryl and ring B is phenyl. Preferably the 6 membered heteroaryl is pyridine, pyridazine, pyrimidine, or pyrazine. Preferably the 6 membered heteroaryl is pyridine, or pyrimidine. Ring A and/or ring B can be further substituted as described herein.

Embodiment 24a of this disclosure relates to the compound of one of Embodiments 1a-22a, wherein ring A is a 6 membered heteroaryl and ring B is phenyl. Preferably the 6 membered heteroaryl is pyridine, pyridazine, pyrimidine, or pyrazine. Preferably the 6 membered heteroaryl is pyridine, or pyrimidine. Ring A and/or ring B can be further substituted as described herein.

Embodiment 25 of this disclosure relates to the compound of one of Embodiments 1-22, wherein ring A is a 6 membered heteroaryl and ring B is a 6 membered heteroaryl. Preferably the 6 membered heteroaryl at each instance is independently selected from pyridine, pyridazine, pyrimidine, or pyrazine. Preferably the 6 membered heteroaryl at each instance is independently selected from pyridine, or pyrimidine. Ring A and/or ring B can be further substituted as described herein.

Embodiment 25a of this disclosure relates to the compound of one of Embodiments 1a-22a, wherein ring A is a 6 membered heteroaryl and ring B is a 6 membered heteroaryl. Preferably the 6 membered heteroaryl at each instance is independently selected from pyridine, pyridazine, pyrimidine, or pyrazine. Preferably the 6 membered heteroaryl at each instance is independently selected from pyridine, or pyrimidine. Ring A and/or ring B can be further substituted as described herein.

Embodiment 26 of this disclosure relates to the compound of one of Embodiments 1-22, wherein ring A is phenyl and ring B is 5 membered heteroaryl. Preferably the 5 membered heteroaryl is pyrrole, furan, thiophene, thiazole, isothiazole, imidazole, triazole, tetrazole, pyrazole, oxazole, or isoxazole. Ring A and/or ring B can be further substituted as described herein.

Embodiment 26a of this disclosure relates to the compound of one of Embodiments 1a-22a, wherein ring A is phenyl and ring B is 5 membered heteroaryl. Preferably the 5 membered heteroaryl is pyrrole, furan, thiophene, thiazole, isothiazole, imidazole, triazole, tetrazole, pyrazole, oxazole, or isoxazole. Ring A and/or ring B can be further substituted as described herein.

Embodiment 27 of this disclosure relates to the compound of one of Embodiments 1-22, wherein ring A is a 5 membered heteroaryl and ring B is phenyl. Preferably the 5 membered heteroaryl is pyrrole, furan, thiophene, thiazole, isothiazole, imidazole, triazole, tetrazole, pyrazole, oxazole, or isoxazole. Ring A and/or ring B can be further substituted as described herein.

Embodiment 27a of this disclosure relates to the compound of one of Embodiments 1a-22a, wherein ring A is a 5 membered heteroaryl and ring B is phenyl. Preferably the 5 membered heteroaryl is pyrrole, furan, thiophene, thiazole, isothiazole, imidazole, triazole, tetrazole, pyrazole, oxazole, or isoxazole. Ring A and/or ring B can be further substituted as described herein.

Embodiment 28 of this disclosure relates to the compound of one of Embodiments 1-22, wherein ring A is a 5 membered heteroaryl and ring B is a 5 membered heteroaryl.

Preferably the 5 membered heteroaryl at each instance is independently selected from pyrrole, furan, thiophene, thiazole, isothiazole, imidazole, triazole, tetrazole, pyrazole, oxazole, or isoxazole. Ring A and/or ring B can be further substituted as described herein.

Embodiment 28a of this disclosure relates to the compound of one of Embodiments 1a-22a, wherein ring A is a 5 membered heteroaryl and ring B is a 5 membered heteroaryl. Preferably the 5 membered heteroaryl at each instance is independently selected from pyrrole, furan, thiophene, thiazole, isothiazole, imidazole, triazole, tetrazole, pyrazole, oxazole, or isoxazole. Ring A and/or ring B can be further substituted as described herein.

Embodiment 29 of this disclosure relates to the compound of one of Embodiments 1-22, wherein ring A is a 5 membered heteroaryl and ring B is a 6 membered heteroaryl. Preferably the 6 membered heteroaryl is selected from pyridine, pyridazine, pyrimidine, or pyrazine. Preferably the 6 membered heteroaryl is selected from pyridine, or pyrimidine. Preferably the 5 membered heteroaryl is selected from pyrrole, furan, thiophene, thiazole, isothiazole, imidazole, triazole, tetrazole, pyrazole, oxazole, or isoxazole. Ring A and/or ring B can be further substituted as described herein.

Embodiment 29a of this disclosure relates to the compound of one of Embodiments 1a-22a, wherein ring A is a 5 membered heteroaryl and ring B is a 6 membered heteroaryl. Preferably the 6 membered heteroaryl is selected from pyridine, pyridazine, pyrimidine, or pyrazine. Preferably the 6 membered heteroaryl is selected from pyridine, or pyrimidine. Preferably the 5 membered heteroaryl is selected from pyrrole, furan, thiophene, thiazole, isothiazole, imidazole, triazole, tetrazole, pyrazole, oxazole, or isoxazole. Ring A and/or ring B can be further substituted as described herein.

Embodiment 30 of this disclosure relates to the compound of one of Embodiments 1-22, wherein ring A is a 6 membered heteroaryl and ring B is a 5 membered heteroaryl. Preferably the 6 membered heteroaryl is selected from pyridine, pyridazine, pyrimidine, or pyrazine. Preferably the 6 membered heteroaryl is selected from pyridine, or pyrimidine. Preferably the 5 membered heteroaryl is selected from pyrrole, furan, thiophene, thiazole, isothiazole, imidazole, triazole, tetrazole, pyrazole, oxazole, or isoxazole. Ring A and/or ring B can be further substituted as described herein.

Embodiment 30a of this disclosure relates to the compound of one of Embodiments 1a-22a, wherein ring A is a 6 membered heteroaryl and ring B is a 5 membered heteroaryl. Preferably the 6 membered heteroaryl is selected from pyridine, pyridazine, pyrimidine, or pyrazine. Preferably the 6 membered heteroaryl is selected from pyridine, or pyrimidine. Preferably the 5 membered heteroaryl is selected from pyrrole, furan, thiophene, thiazole, isothiazole, imidazole, triazole, tetrazole, pyrazole, oxazole, or isoxazole. Ring A and/or ring B can be further substituted as described herein.

Embodiment 31 of this disclosure relates to a compound having one of the following formulae:

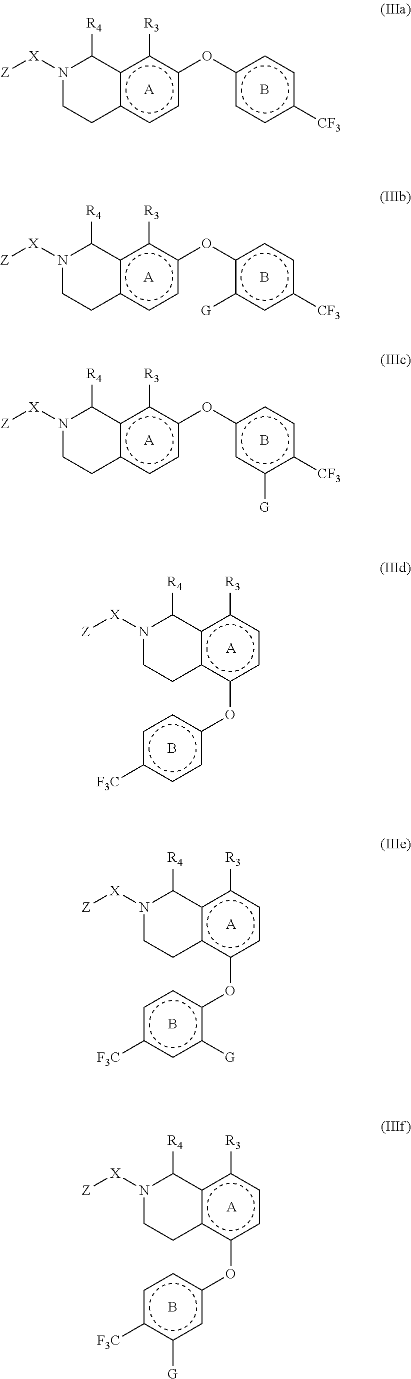

or a pharmaceutically acceptable salt, a tautomer, a stereoisomer, or a deuterated analog of any of formulae (IIIa), (IIIb), (IIIc), (IIId), (IIIe) or (IIIf); wherein:
G is C₁, F, or CN;
R³ is H; halogen; C₂-C₄alkenyl optionally substituted with cyclopropyl or heterocycloalkyl; heterocycloalkyl optionally substituted with C(O)—CH₃; heterocycloalkenyl optionally substituted with C(O)—CH₃; heterocycloalkylalkyl optionally substituted with C(O)—CH₃; or 5-6 membered heteroaryl optionally substituted with haloalkyl, cyclopropyl, or cyclopropyl-CH₂—;
R⁴ is H; C₁-C₃alkyl; cycloalkyl optionally substituted with haloalkyl or —C(O)-alkenyl; heterocycloalkyl optionally substituted with —C(O)—CH₃, —C(O)—CH═CH₂, or —C(O)-cyclopropyl; heterocycloalkylalkyl optionally substituted with —C(O)—CH₃; —C(O)—CH₂—OH; or 5-6 membered heteroaryl;
X is —(CH₂)ₙ—C(O)—;
Z is —NR⁶R⁷, C₂-C₄alkyl optionally substituted with 1-3 R⁸, C₆-C₁₂aryl optionally substituted with 1-3 R¹⁰, or 5-10 membered heteroaryl optionally substituted with 1-3 R¹⁰;
R⁶ is hydrogen, C₁-Calkyl, cycloalkyl, heterocycloalkyl, aryl, or heteroaryl, wherein each C₁-Calkyl cycloalkyl, heterocycloalkyl, aryl, or heteroaryl is optionally substituted with 1-3 R¹¹;
R⁷ is hydrogen or C₁-C₆alkyl; or
R⁸ is —NR⁶R⁷, CN, hydroxy, alkoxy, haloalkoxy, —S—C₁-C₃alkyl, —S(O)—C₁-C₃alkyl, —S(O)₂—C₁-C₃alkyl, —S(O)₂—C₁-C₃haloalkyl, —S(O)₂—NH₂, —S(O)(NH)-C₁-C₃alkyl, —C(O)—NH₂, —C(O)OH, —C(O)O—C₁-C₃alkyl, —C(O)C₁-C₃haloalkyl, —N(H)—C(O)—NR⁶R⁷, —N(H)—C(O)—C₁-C₃alkyl optionally substituted with —NR¹²R¹³ or CN, —N(H)—C(O)—C₂-C₆alkenyl, —N(H)—C(O)—C₁-C₃haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₃haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰; or
two R⁸, together with the carbon atom to which they are attached, join together to form a cycloalkyl;
each R¹⁰ is independently hydroxy, halogen, CN, C₁-C₆alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkenyl, —S(O)₂—C₁-C₃alkyl, —S(O)₂—C₂-C₆alkenyl, or —S(O)₂—NH₂; R¹¹ is CN;
each R¹² and R¹³ are independently hydrogen, C₁-C₆alkyl, or C₃-C₆cycloalkyl; and
n is 0 or 1;
provided that ring A and/or ring B is a 5- or 6-membered heteroaryl comprising 1-4 heteroatoms selected from N, O and S.

Embodiment 31a of this disclosure relates to a compound having one of the following formulae:

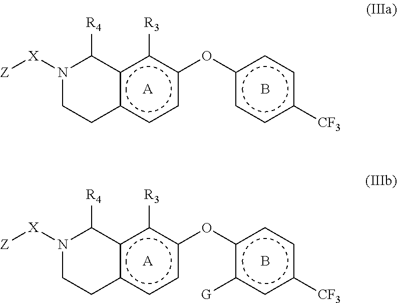

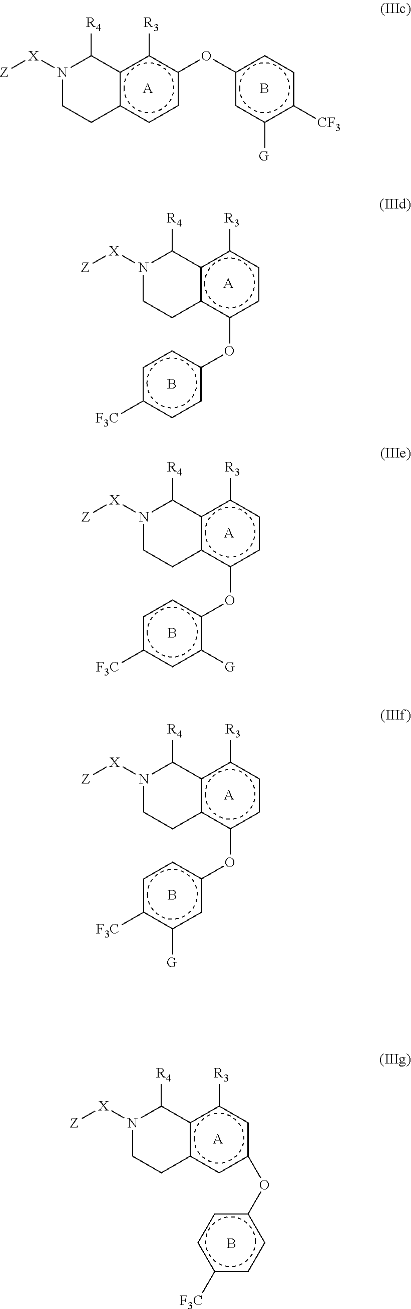

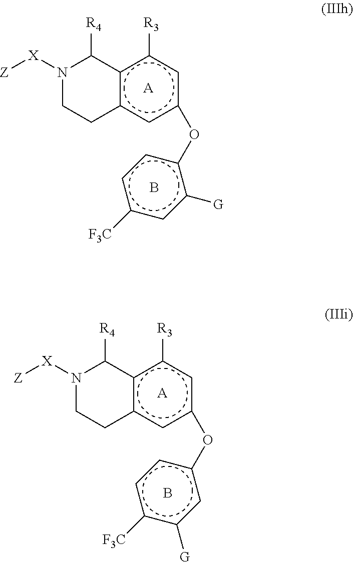

or a pharmaceutically acceptable salt, a tautomer, a stereoisomer, or a deuterated analog of any of formulae (IIIa), (IIIb), (IIIc), (IIId), (IIIe), (IIIf), (IIIg), (IIIh), or (IIIi); wherein:
G is C₁, F, or CN;
R³ is H; halogen; C₂-C₄alkenyl optionally substituted with cyclopropyl or heterocycloalkyl; heterocycloalkyl optionally substituted with C(O)—CH₃; heterocycloalkenyl optionally substituted with C(O)—CH₃; heterocycloalkylalkyl optionally substituted with C(O)—CH₃; or 5-6 membered heteroaryl optionally substituted with haloalkyl, cyclopropyl, or cyclopropyl-CH₂—;
R⁴ is H; C₁-C₃alkyl; cycloalkyl optionally substituted with haloalkyl or —C(O)-alkenyl; heterocycloalkyl optionally substituted with —C(O)—CH₃, —C(O)—CH═CH₂, or —C(O)-cyclopropyl; heterocycloalkylalkyl optionally substituted with —C(O)—CH₃; —C(O)—CH₂—OH; or 5-6 membered heteroaryl;
X is —(CH₂)ₙ—C(O)—;
Z is —NR⁶R⁷, C₂-C₄alkenyl, C₂-C₄alkyl optionally substituted with 1-3 R⁸, C₆-C₁₂aryl optionally substituted with 1-3 R¹⁰, or 5-10 membered heteroaryl optionally substituted with 1-3 R¹⁰;
R⁶ is hydrogen, C₁-C₆alkyl, cycloalkyl, heterocycloalkyl, aryl, or heteroaryl, wherein each C₁-C₆alkyl cycloalkyl, heterocycloalkyl, aryl, or heteroaryl is optionally substituted with 1-3 R¹¹;
R⁷ is hydrogen or C₁-C₆alkyl; or
R⁸ is halogen, —NR⁶R⁷, CN, hydroxy, alkoxy, haloalkyl, haloalkoxy, —S—C₁-C₃alkyl, —S(O)—C₁-C₃alkyl, —S(O)₂—C₁-C₃alkyl, —S(O)₂—C₁-C₃haloalkyl, —S(O)₂—NH₂, —S(O)₂ (NH)-C₁-C₃alkyl, —C(O)—NH₂, —C(O)OH, —C(O)O—C₁-C₃alkyl, —C(O)C₁-C₃haloalkyl, —N(H)—C(O)—NR⁶R⁷, —N(H)—C(O)—C₁-C₃alkyl optionally substituted with —NR¹²R¹³ or CN, —N(H)—C(O)—C₂-C₆alkenyl, —N(H)—C(O)—C₁-C₃haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₃haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰; or
two R⁸, together with the carbon atom to which they are attached, join together to form a cycloalkyl;
each R¹⁰ is independently hydroxy, halogen, CN, C₁-C₆alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkyl, —NH—C(O)-alkenyl, —NH—S(O)₂—C₁-C₃alkyl, —S(O)₂—C₁-C₃haloalkyl, —S(O)₂—C₁-C₃alkyl, —S(O)₂—C₂-Calkenyl, —S(O)₂—NH₂ or —S(O)₂—NH—C₁-C₃alkyl;
R¹¹ is CN;
each R¹² and R¹³ are independently hydrogen, C₁-C₆alkyl, or C₃-C₆cycloalkyl; and
n is 0 or 1;
   provided that ring A and/or ring B is a 5- or 6-membered heteroaryl comprising 1-4 heteroatoms selected from N, O and S when —O— is attached to Ring A at the 5 or 7 position.

Embodiment 32 of this disclosure relates to the compound of Embodiment 31, wherein Z is C₆-C₁₂aryl optionally substituted with 1-3 R¹⁰; and n is 0 or 1. Preferably Z is substituted with 1-3 R¹⁰. Preferably Z is substituted with 1 R¹⁰. Preferably Z is substituted with 2 R¹⁰. Preferably Z is substituted with 3 R¹⁰.

Embodiment 32a of this disclosure relates to the compound of Embodiment 31a, wherein Z is C₆-C₁₂aryl optionally substituted with 1-3 R¹⁰; and n is 0 or 1. Preferably Z is substituted with 1-3 R¹⁰. Preferably Z is substituted with 1 R¹⁰. Preferably Z is substituted with 2 R¹⁰. Preferably Z is substituted with 3 R¹⁰.

Embodiment 33 of this disclosure relates to the compound of Embodiment 31, wherein Z is 5-10 membered heteroaryl optionally substituted with 1-3 R¹⁰; and n is 0 or 1. Preferably Z is substituted with 1-3 R¹⁰. Preferably Z is substituted with 1 R¹⁰. Preferably Z is substituted with 2 R¹⁰. Preferably Z is substituted with 3 R¹⁰.

Embodiment 33a of this disclosure relates to the compound of Embodiment 31a, wherein Z is 5-10 membered heteroaryl optionally substituted with 1-3 R¹⁰; and n is 0 or 1. Preferably Z is substituted with 1-3 R¹⁰. Preferably Z is substituted with 1 R¹⁰. Preferably Z is substituted with 2 R¹⁰. Preferably Z is substituted with 3 R¹⁰.

Embodiment 34 of this disclosure relates to the compound of Embodiments 31-33, wherein X—Z is selected from

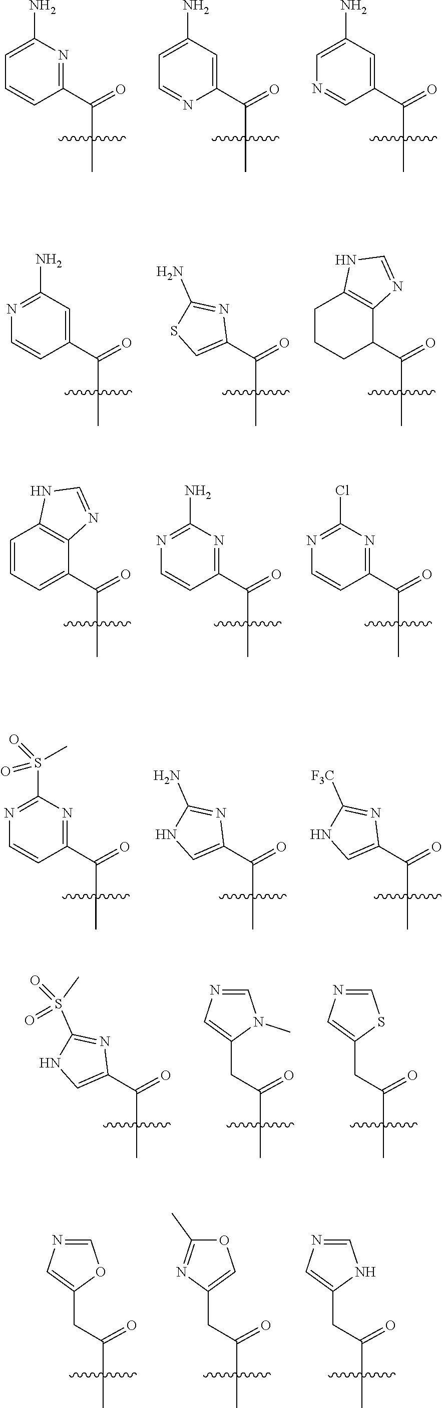

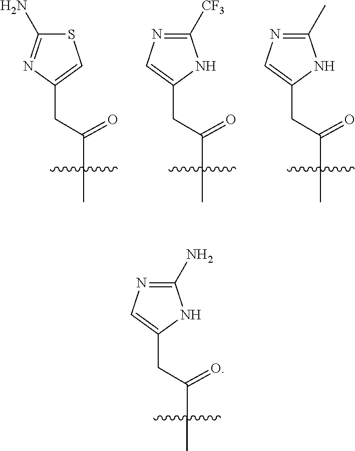

Embodiment 34a of this disclosure relates to the compound of Embodiment 31a, Embodiment 32a, or Embodiment 33a, wherein X—Z is selected from

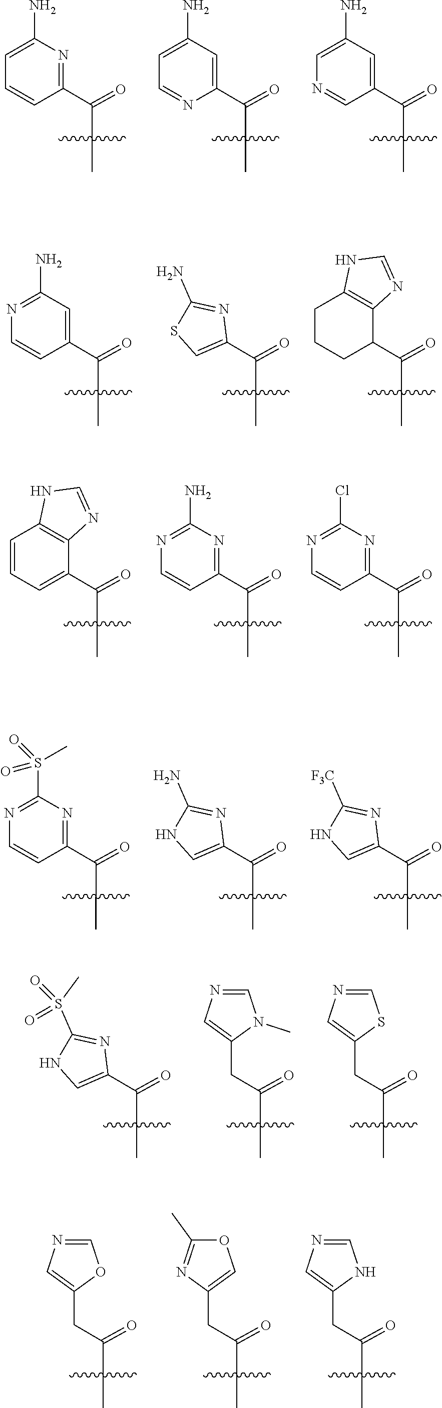

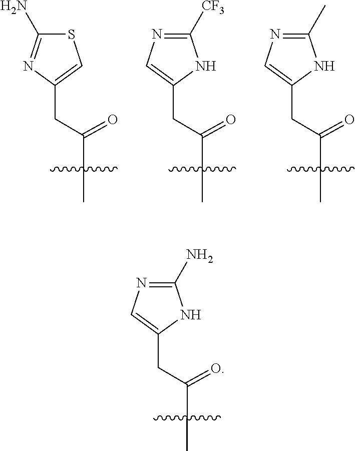

Embodiment 35 of this disclosure relates to the compound of Embodiment 31, wherein Z is C₂-C₄alkyl optionally substituted with 1-3 R⁸; and n is 0 or 1. Preferably Z is substituted with 1-3 R⁸. Preferably Z is substituted with 1 R⁸. Preferably Z is substituted with 2 R⁸. Preferably Z is substituted with 3 R⁸.

Embodiment 35a of this disclosure relates to the compound of Embodiment 31a, wherein Z is C₂-C₄alkenyl or C₂-C₄alkyl optionally substituted with 1-3 R⁸; and n is 0 or 1.

Preferably Z is C₂-C₄alkenyl.

Preferably Z is C₂-C₄alkyl optionally substituted with 1-3 R⁸. Preferably Z is substituted with 1-3 R⁸. Preferably Z is substituted with 1 R⁸. Preferably Z is substituted with 2 R⁸. Preferably Z is substituted with 3 R⁸.

Embodiment 36 of this disclosure relates to the compound of Embodiment 35, wherein Z is —(CH₂)₂—S(O)₂—CH₃ or —CH₂—N(H)—C(O)—NH₂. Preferably Z is —(CH₂)₂—S(O)₂—CH₃.

Embodiment 36a of this disclosure relates to the compound of Embodiment 35a, wherein Z is —(CH₂)₂—S(O)₂—CH₃ or —CH₂—N(H)—C(O)—NH₂. Preferably Z is —(CH₂)₂—S(O)₂—CH₃.

Embodiment 37 of this disclosure relates to the compound of one of Embodiments 31-36, wherein R³ is H and R⁴ is H.

Embodiment 37a of this disclosure relates to the compound of one of Embodiments 31a-36a, wherein R³ is H and R⁴ is H.

Embodiment 38 of this disclosure relates to the compound of one of Embodiments 31-37, wherein n is 1.

Embodiment 38a of this disclosure relates to the compound of one of Embodiments 31a-37a, wherein n is 1.

Embodiment 39 of this disclosure relates to the compound of one of Embodiments 31-37, wherein n is 0.

Embodiment 39a of this disclosure relates to the compound of one of Embodiments 31a-37a, wherein n is 0.

Embodiment 40 of this disclosure relates to the compound of one of Embodiments 31-39, wherein ring A is phenyl and ring B is 6 membered heteroaryl. Preferably the 6 membered heteroaryl is pyridine, pyridazine, pyrimidine, or pyrazine. Preferably the 6 membered heteroaryl is pyridine or pyrimidine. Ring A and/or ring B can be further substituted as described herein.

Embodiment 40a of this disclosure relates to the compound of one of Embodiments 31a-39a, wherein ring A is phenyl and ring B is 6 membered heteroaryl. Preferably the 6 membered heteroaryl is pyridine, pyridazine, pyrimidine, or pyrazine. Preferably the 6 membered heteroaryl is pyridine or pyrimidine. Ring A and/or ring B can be further substituted as described herein.

Embodiment 40b of this disclosure relates to the compound of one of Embodiments 31a-39a, wherein ring A is phenyl and ring B is phenyl.

Embodiment 41 of this disclosure relates to the compound of one of Embodiments 31-39, wherein ring A is a 6 membered heteroaryl and ring B is phenyl. Preferably the 6 membered heteroaryl is pyridine, pyridazine, pyrimidine, or pyrazine. Preferably the 6 membered heteroaryl is pyridine or pyrimidine. Ring A and/or ring B can be further substituted as described herein.

Embodiment 41a of this disclosure relates to the compound of one of Embodiments 31a-39a, wherein ring A is a 6 membered heteroaryl and ring B is phenyl. Preferably the 6 membered heteroaryl is pyridine, pyridazine, pyrimidine, or pyrazine. Preferably the 6 membered heteroaryl is pyridine or pyrimidine. Ring A and/or ring B can be further substituted as described herein.

Embodiment 42 of this disclosure relates to the compound of one of Embodiments 31-39, wherein ring A is a 6 membered heteroaryl and ring B is a 6 membered heteroaryl. Preferably the 6 membered heteroaryl at each instance is independently selected from pyridine, pyridazine, pyrimidine, or pyrazine. Preferably the 6 membered heteroaryl at each instance is independently selected from pyridine or pyrimidine. Ring A and/or ring B can be further substituted as described herein.

Embodiment 42a of this disclosure relates to the compound of one of Embodiments 31a-39a, wherein ring A is a 6 membered heteroaryl and ring B is a 6 membered heteroaryl. Preferably the 6 membered heteroaryl at each instance is independently selected from pyridine, pyridazine, pyrimidine, or pyrazine. Preferably the 6 membered heteroaryl at each instance is independently selected from pyridine or pyrimidine. Ring A and/or ring B can be further substituted as described herein.

Embodiment 43 of this disclosure relates to the compound of one of Embodiments 31-39, wherein ring A is phenyl and ring B is 5 membered heteroaryl. Preferably the 5 membered heteroaryl is pyrrole, furan, thiophene, thiazole, isothiazole, imidazole, triazole, tetrazole, pyrazole, oxazole, or isoxazole. Ring A and/or ring B can be further substituted as described herein.

Embodiment 43a of this disclosure relates to the compound of one of Embodiments 31a-39a, wherein ring A is phenyl and ring B is 5 membered heteroaryl. Preferably the 5 membered heteroaryl is pyrrole, furan, thiophene, thiazole, isothiazole, imidazole, triazole, tetrazole, pyrazole, oxazole, or isoxazole. Ring A and/or ring B can be further substituted as described herein.

Embodiment 44 of this disclosure relates to the compound of one of Embodiments 31-39, wherein ring A is a 5 membered heteroaryl and ring B is phenyl. Preferably the 5 membered heteroaryl is pyrrole, furan, thiophene, thiazole, isothiazole, imidazole, triazole, tetrazole, pyrazole, oxazole, or isoxazole. Ring A and/or ring B can be further substituted as described herein.

Embodiment 44a of this disclosure relates to the compound of one of Embodiments 31a-39a, wherein ring A is a 5 membered heteroaryl and ring B is phenyl. Preferably the 5 membered heteroaryl is pyrrole, furan, thiophene, thiazole, isothiazole, imidazole, triazole, tetrazole, pyrazole, oxazole, or isoxazole. Ring A and/or ring B can be further substituted as described herein.

Embodiment 45 of this disclosure relates to the compound of one of Embodiments 31-39, wherein ring A is a 5 membered heteroaryl and ring B is a 5 membered heteroaryl. Preferably the 5 membered heteroaryl at each instance is independently selected from pyrrole, furan, thiophene, thiazole, isothiazole, imidazole, triazole, tetrazole, pyrazole, oxazole, or isoxazole. Ring A and/or ring B can be further substituted as described herein.

Embodiment 45a of this disclosure relates to the compound of one of Embodiments 31a-39a, wherein ring A is a 5 membered heteroaryl and ring B is a 5 membered heteroaryl. Preferably the 5 membered heteroaryl at each instance is independently selected from pyrrole, furan, thiophene, thiazole, isothiazole, imidazole, triazole, tetrazole, pyrazole, oxazole, or isoxazole. Ring A and/or ring B can be further substituted as described herein.

Embodiment 46 of this disclosure relates to the compound of one of Embodiments 31-39, wherein ring A is a 5 membered heteroaryl and ring B is a 6 membered heteroaryl.

Preferably the 6 membered heteroaryl is selected from pyridine, pyridazine, pyrimidine, or pyrazine. Preferably the 6 membered heteroaryl is selected from pyridine, or pyrimidine. Preferably the 5 membered heteroaryl is selected from pyrrole, furan, thiophene, thiazole, isothiazole, imidazole, triazole, tetrazole, pyrazole, oxazole, or isoxazole. Ring A and/or ring B can be further substituted as described herein.

Embodiment 46a of this disclosure relates to the compound of one of Embodiments 31a-39a, wherein ring A is a 5 membered heteroaryl and ring B is a 6 membered heteroaryl. Preferably the 6 membered heteroaryl is selected from pyridine, pyridazine, pyrimidine, or pyrazine. Preferably the 6 membered heteroaryl is selected from pyridine, or pyrimidine. Preferably the 5 membered heteroaryl is selected from pyrrole, furan, thiophene, thiazole, isothiazole, imidazole, triazole, tetrazole, pyrazole, oxazole, or isoxazole. Ring A and/or ring B can be further substituted as described herein.

Embodiment 47 of this disclosure relates to the compound of one of Embodiments 31-39, wherein ring A is a 6 membered heteroaryl and ring B is a 5 membered heteroaryl. Preferably the 6 membered heteroaryl is selected from pyridine, pyridazine, pyrimidine, or pyrazine. Preferably the 6 membered heteroaryl is selected from pyridine, or pyrimidine. Preferably the 5 membered heteroaryl is selected from pyrrole, furan, thiophene, thiazole, isothiazole, imidazole, triazole, tetrazole, pyrazole, oxazole, or isoxazole. Ring A and/or ring B can be further substituted as described herein.

Embodiment 47a of this disclosure relates to the compound of one of Embodiments 31a-39a, wherein ring A is a 6 membered heteroaryl and ring B is a 5 membered heteroaryl. Preferably the 6 membered heteroaryl is selected from pyridine, pyridazine, pyrimidine, or pyrazine. Preferably the 6 membered heteroaryl is selected from pyridine, or pyrimidine. Preferably the 5 membered heteroaryl is selected from pyrrole, furan, thiophene, thiazole, isothiazole, imidazole, triazole, tetrazole, pyrazole, oxazole, or isoxazole. Ring A and/or ring B can be further substituted as described herein.

Embodiment 48 of this disclosure relates to the compound according to any of the preceding Embodiments, wherein the compound is a formic acid salt.

Embodiment 49 of this disclosure relates to a compound selected from Table 1, or a pharmaceutically acceptable salt thereof.

Embodiment 50 of this disclosure relates to the compound according to any of the preceding Embodiments, wherein the compound is a non-covalent inhibitor of TEAD.

Compounds contemplated herein are described with reference to both generic formulae and specific compounds. In addition, the compounds described herein may exist in a number of different forms or derivatives, all within the scope of the present disclosure. These include, for example, tautomers, stereoisomers, racemic mixtures, regioisomers, salts, prodrugs (e.g. carboxylic acid esters), and active metabolites.

It is understood that some compounds may exhibit tautomerism. In such cases, the formulae provided herein expressly depict only one of the possible tautomeric forms. It is therefore to be understood that the formulae provided herein are intended to represent any tautomeric form of the depicted compounds and are not to be limited merely to the specific tautomeric form depicted by the drawings of the formulae.

Likewise, some of the compounds according to the present disclosure may exist as stereoisomers as defined herein. All such single stereoisomers, racemates and mixtures thereof are intended to be within the scope of the present disclosure. Unless specified to the contrary, all such stereoisomeric forms are included within the formulae provided herein.

In some embodiments, a chiral compound of the present disclosure is in a form that contains at least 80% of a single isomer (60% enantiomeric excess (“e.e.”) or diastereomeric excess (“d.e.”)), or at least 85% (70% e.e. or d.e.), 90% (80% e.e. or d.e.), 95% (90% e.e. or d.e.), 97.5% (95% e.e. or d.e.), or 99% (98% e.e. or d.e.). As generally understood by those skilled in the art, an optically pure compound having one chiral center is one that consists essentially of one of the two possible enantiomers (i.e., is enantiomerically pure), and an optically pure compound having more than one chiral center is one that is both diastereomerically pure and enantiomerically pure. In some embodiments, the compound is present in optically pure form.

For compounds in which synthesis involves addition of a single group at a double bond, particularly a carbon-carbon double bond, the addition may occur at either of the double bond-linked atoms. For such compounds, the present disclosure includes both such regioisomers.

In addition to the present formulae and compounds described herein, the disclosure also includes prodrugs (generally pharmaceutically acceptable prodrugs), active metabolic derivatives (active metabolites), and their pharmaceutically acceptable salts.

Unless specified to the contrary, specification of a compound herein includes pharmaceutically acceptable salts of such compound.

In some embodiments, compounds of the disclosure are complexed with an acid or a base, including base addition salts such as ammonium, diethylamine, ethanolamine, ethylenediamine, diethanolamine, t-butylamine, piperazine, meglumine; acid addition salts, such as acetate, acetylsalicylate, besylate, camsylate, citrate, formate, fumarate, glutarate, hydrochlorate, maleate, mesylate, nitrate, oxalate, phosphate, succinate, sulfate, tartrate, thiocyanate and tosylate; and amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine. In some instances, the amorphous form of the complex is facilitated by additional processing, such as by spray-drying, mechanochemical methods such as roller compaction, or microwave irradiation of the parent compound mixed with the acid or base. Such methods may also include addition of ionic and/or non-ionic polymer systems, including, but not limited to, hydroxypropyl methyl cellulose acetate succinate (HPMCAS) and methacrylic acid copolymer (e.g. Eudragit® L100-55), that further stabilize the amorphous nature of the complex. Such amorphous complexes provide several advantages. For example, lowering of the melting temperature relative to the free base facilitates additional processing, such as hot melt extrusion, to further improve the biopharmaceutical properties of the compound. Also, the amorphous complex is readily friable, which provides improved compression for loading of the solid into capsule or tablet form.

### III. Formulations and Administration

Embodiment 51 of this disclosure relates to a pharmaceutical composition comprising a compound in any one of Embodiments 1-50, and a pharmaceutically acceptable carrier.

Embodiment 51a of this disclosure relates to a pharmaceutical composition comprising a compound in any one of Embodiments 1a-47a, Embodiment, 23b, or Embodiment 40b and a pharmaceutically acceptable carrier.

Embodiment 52 of this disclosure relates to the pharmaceutical composition of Embodiment 51, further comprising a second pharmaceutical agent.

Embodiment 52a of this disclosure relates to the pharmaceutical composition of Embodiment 51a, further comprising a second pharmaceutical agent.

Suitable dosage forms, in part, depend upon the use or the route of administration, for example, oral, transdermal, transmucosal, inhalant, or by injection (parenteral). Such dosage forms should allow the compound to reach target cells. Other factors are well known in the art, and include considerations such as toxicity and dosage forms that retard the compound or composition from exerting its effects. Techniques and formulations generally may be found in The Science and Practice of Pharmacy, 21st edition, Lippincott, Williams and Wilkins, Philadelphia, PA, 2005 (hereby incorporated by reference herein).

Compounds of the present disclosure (i.e. any of the compounds described in Embodiments 1-50, including any of the subembodiments thereof) can be formulated as pharmaceutically acceptable salts.

Compounds of the present disclosure (i.e. any of the compounds described in Embodiments 1a-47a, Embodiment, 23b, or Embodiment 40b, including any of the subembodiments thereof) can be formulated as pharmaceutically acceptable salts.

Carriers or excipients can be used to produce compositions. The carriers or excipients can be chosen to facilitate administration of the compound. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

The compounds can be administered by different routes including intravenous, intraperitoneal, subcutaneous, intramuscular, oral, transmucosal, rectal, transdermal, or inhalant. In some embodiments, the compounds can be administered by oral administration. For oral administration, for example, the compounds can be formulated into conventional oral dosage forms such as capsules, tablets, and liquid preparations such as syrups, elixirs, and concentrated drops.

For inhalants, compounds of the disclosure may be formulated as dry powder or a suitable solution, suspension, or aerosol. Powders and solutions may be formulated with suitable additives known in the art. For example, powders may include a suitable powder base such as lactose or starch, and solutions may comprise propylene glycol, sterile water, ethanol, sodium chloride and other additives, such as acid, alkali and buffer salts. Such solutions or suspensions may be administered by inhaling via spray, pump, atomizer, or nebulizer, and the like. The compounds of the disclosure may also be used in combination with other inhaled therapies, for example corticosteroids such as fluticasone propionate, beclomethasone dipropionate, triamcinolone acetonide, budesonide, and mometasone furoate; beta agonists such as albuterol, salmeterol, and formoterol; anticholinergic agents such as ipratropium bromide or tiotropium; vasodilators such as treprostinal and iloprost; enzymes such as DNAase; therapeutic proteins; immunoglobulin antibodies; an oligonucleotide, such as single or double stranded DNA or RNA, siRNA; antibiotics such as tobramycin; muscarinic receptor antagonists; leukotriene antagonists; cytokine antagonists; protease inhibitors; cromolyn sodium; nedocril sodium; and sodium cromoglycate.

Pharmaceutical preparations for oral use can be obtained, for example, by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP: povidone). If desired, disintegrating agents may be added, such as the cross-linked polyvinylpyrrolidone, agar, or alginic acid, or a salt thereof such as sodium alginate.

Dragee cores are provided with suitable coatings. For this purpose, concentrated sugar solutions may be used, which may optionally contain, for example, gum arabic, talc, poly-vinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Alternatively, injection (parenteral administration) may be used, e.g., intramuscular, intravenous, intraperitoneal, and/or subcutaneous. For injection, the compounds of the disclosure are formulated in sterile liquid solutions, such as in physiologically compatible buffers or solutions, such as saline solution, Hank's solution, or Ringer's solution. In addition, the compounds may be formulated in solid form and redissolved or suspended immediately prior to use. Lyophilized forms can also be produced.

Administration can also be by transmucosal, topical, transdermal, or inhalant means. For transmucosal, topical or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal).

The topical compositions of this disclosure are formulated as oils, creams, lotions, ointments, and the like by choice of appropriate carriers known in the art. Suitable carriers include vegetable or mineral oils, white petrolatum (white soft paraffin), branched chain fats or oils, animal fats and high molecular weight alcohol (greater than C₁₂). In another embodiment, the carriers are those in which the active ingredient is soluble. Emulsifiers, stabilizers, humectants and antioxidants may also be included as well as agents imparting color or fragrance, if desired. Creams for topical application are formulated from a mixture of mineral oil, self-emulsifying beeswax and water in which mixture the active ingredient, dissolved in a small amount solvent (e.g. an oil), is admixed. Additionally, administration by transdermal means may comprise a transdermal patch or dressing such as a bandage impregnated with an active ingredient and optionally one or more carriers or diluents known in the art. To be administered in the form of a transdermal delivery system, the dosage administration will, of course, be continuous rather than intermittent throughout the dosage regimen.

The amounts of various compounds to be administered can be determined by standard procedures taking into account factors such as the compound IC₅₀, the biological half-life of the compound, the age, size, and weight of the subject, and the indication being treated. The importance of these and other factors are well known to those of ordinary skill in the art. Generally, a dose will be between about 0.01 and 50 mg/kg, or 0.1 and 20 mg/kg of the subject being treated. Multiple doses may be used.

The compounds of the disclosure may also be used in combination with other therapies for treating the same disease. Such combination use includes administration of the compounds and one or more other therapeutics at different times, or co-administration of the compound and one or more other therapies. In some embodiments, dosage may be modified for one or more of the compounds of the disclosure or other therapeutics used in combination, e.g., reduction in the amount dosed relative to a compound or therapy used alone, by methods well known to those of ordinary skill in the art.

It is understood that use in combination includes use with other therapies, drugs, medical procedures etc., where the other therapy or procedure may be administered at different times (e.g. within a short time, such as within hours (e.g. 1, 2, 3, 4-24 hours), or within a longer time (e.g. 1-2 days, 2-4 days, 4-7 days, 1-4 weeks)) than a compound of the present disclosure, or at the same time as a compound of the disclosure. Use in combination also includes use with a therapy or medical procedure that is administered once or infrequently, such as surgery, along with a compound of the disclosure administered within a short time or longer time before or after the other therapy or procedure. In some embodiments, the present disclosure provides for delivery of compounds of the disclosure and one or more other drug therapeutics delivered by a different route of administration or by the same route of administration. The use in combination for any route of administration includes delivery of compounds of the disclosure and one or more other drug therapeutics delivered by the same route of administration together in any formulation, including formulations where the two compounds are chemically linked in such a way that they maintain their therapeutic activity when administered. In one aspect, the other drug therapy may be co-administered with one or more compounds of the disclosure. Use in combination by co-administration includes administration of co-formulations or formulations of chemically joined compounds, or administration of two or more compounds in separate formulations within a short time of each other (e.g. within an hour, 2 hours, 3 hours, up to 24 hours), administered by the same or different routes. Co-administration of separate formulations includes co-administration by delivery via one device, for example the same inhalant device, the same syringe, etc., or administration from separate devices within a short time of each other. Co-formulations of compounds of the disclosure and one or more additional drug therapies delivered by the same route includes preparation of the materials together such that they can be administered by one device, including the separate compounds combined in one formulation, or compounds that are modified such that they are chemically joined, yet still maintain their biological activity. Such chemically joined compounds may have a linkage that is substantially maintained in vivo, or the linkage may break down in vivo, separating the two active components.

### IV. Methods of Use

Disease indications and modulations YAP/TEAD
Exemplary Diseases Associated with YAP/TEAD

### Polycystic Kidney Disease

YAP and TAZ appear to serve functions in Polycystic kidney disease (PKD) progression. Increased YAP expression was also observed in human PKD patients. TAZ forms a complex with Polycystin-2 (PC2, the protein product of PKD1), thereby targeting it for ubiquitination and degradation. It was observed that TAZ knockout results in PKD, and also results in the down-regulation of other genes necessary for proper cilia development and function implicating YAP as a potential therapeutic target for PKD (Steven W Plouffe et al; Disease Implications of the Hippo/YAP Pathway; Trends Mol Med. 2015 April; 21(4): 212-222).

### Neurodegenerative Diseases

Hippo pathway components are involved in neurological diseases. For instance, studies reported that YAP/TAZ mediate gene transcription induced by AβPP, the precursor of Amyloid β which is believed to be a driver of Alzheimer's disease implicating YAP as a potential therapeutic target for Alzheimer's disease (Steven W Plouffe et al., 2015).

### Arrhythmogenic Cardiomyopathy and Holt-Oram Syndrome

The Hippo pathway plays a role in heart diseases. Arrhythmogenic right ventricular cardiomyopathy (ARVC) is characterized by thinning of the right ventricular walls, arrhythmias, and replacement of the myocardium with fibroadipocytes. It has been shown that YAP are phosphorylated in human ARVC hearts, and that overexpressing a constitutively active YAP mutant in cardiomyocytes results in adipogenesis, further supporting the role of the Hippo pathway in ARVC and implicating YAP as a potential therapeutic target for ARVC (Steven W Plouffe et al., 2015).

### Liver Cancer

YAP is frequently overexpressed in hepatocellular carcinoma (HCC) and is required to sustain increased cell proliferation and tumor growth. In addition, risk factors for HCC include hepatitis infection and exposure to xenobiotics, and these have also been implicated in activating YAP. For example, the Hepatitis B virus X protein (HBx) directly increases YAP expression by enhancing YAP gene transcription. In another example, TCPOBOP is a xenobiotic mimic that activates constitutive androstane receptor to increase YAP protein levels and induce HCC. Further, it was observed that inducing YAP overexpression in a liver-specific transgenic model causes abnormal hepatocyte proliferation and suppressed apoptosis, resulting in increased liver size and HCC implicating YAP as a potential therapeutic target for HCC (Steven W Plouffe et al., 2015).

### Epithelioid Hemangioendothelioma

Epithelioid hemangioendothelioma (EHE) is a vascular tumor generally found in the lung, bone, and skin. It has been observed that YAP/TAZ chromosome translocations occur in virtually all EHE cases that strongly suggest that dysregulated YAP/TAZ fusion proteins may act as cancer drivers for EHE implicating YAP as a potential therapeutic target for EHE (Steven W Plouffe et al., 2015).

### Breast Cancer

YAP/TAZ activity has been correlated with increased risk of metastasis and reduced survival in various human breast cancer subtypes. TAZ is highly expressed in invasive breast cancer cell lines and primary breast cancers. Further, TAZ overexpression is sufficient to induce cell proliferation, transformation in breast cancer cell lines. Similarly, overexpressing YAP in breast cancer cell lines induces tumor formation and growth in xenograft experiments, and deleting YAP prevents tumor growth in an oncogene-induced breast cancer model implicating YAP as a potential therapeutic target for breast cancer (Steven W Plouffe et al., 2015).

### Lung Cancer

YAP/TAZ are both highly expressed in non-small cell lung cancer (NSCLC) in humans. Knockdown of either YAP or TAZ in NSCLC cells suppresses proliferation, invasion, and tumor growth in mice. High YAP expression is correlated with advanced stage, lymph node metastasis, and decreased survival in lung cancer. Further, it has been shown that knockdown of either YAP or TAZ is sufficient to decrease cell migration in vitro and metastasis in vivo in lung cancer implicating YAP as a potential therapeutic target for NSCLC (Steven W Plouffe et al., 2015).

### Malignant Mesothelioma

It has been observed that knocking down YAP in malignant mesothelioma cells is sufficient to inhibit cell proliferation and anchorage-independent growth implicating dysregulation of the Hippo pathway in malignant mesothelioma and YAP as a potential therapeutic target for malignant mesothelioma (Steven W Plouffe et al., 2015).

### Pancreatic Cancer

Pancreatic ductal adenocarcinoma (PDAC) often have increased YAP expression, and elevated YAP expression is correlated with poor prognosis. Further, it has been observed that YAP knockdown results in reduced proliferation and reduced anchorage-independent growth in pancreatic cancer cells suggesting YAP may play an important role in PDAC progression. It was also reported that in a mouse model expressing mutated KRAS, that deleting YAP is sufficient to prevent PDAC (Steven W Plouffe et al., 2015).

### Kaposi Sarcoma

YAP/TAZ plays a major role in Kaposi sarcoma (KS). It has been shown that tissue samples from human KS patients elevated levels of YAP/TAZ. Recently, it was shown that KSHV encodes a viral GPCR (vGPCR), activating YAP/TAZ, and that cells overexpressing vGPCR failed to grow in a xenograft mouse model when YAP/TAZ were depleted, indicating that YAP/TAZ are necessary for KSHV-induced tumorigenesis (Steven W Plouffe et al., 2015).

Uveal Melanoma 80% of Uveal Melanoma (UM) cases are characterized by activating mutations in either GNAQ or GNA11 encoding Gq or G11 respectively (Gq/11). It has been shown that that Gq/11 can activate YAP, and treating UM with Verteporfin, a drug which blocks YAP-TEAD interaction inhibits UM tumor growth in mice (Steven W Plouffe et al., 2015).

### Renal Cell Carcinoma

YAP has been implicated in renal cell carcinoma (RCC). A recent report found there is increased YAP activity in RCC and that RCC tissues show elevated levels of YAP, and knocking down YAP in RCC cell lines blocks cell proliferation and increases apoptosis (Steven W Plouffe et al., 2015).

### Colorectal Cancer

It has been observed that YAP is often overexpressed in colorectal cancer (CRC), and YAP/TAZ activity is correlated with decreased survival. In mice, inducing YAP overexpression in the intestine results in dysplasia after two days, although the intestine regenerates once induction is stopped. Further, it was observed that in knockout mice which developed adenomas after 13 weeks and polyps after 13 months, that these phenotypes were blocked by deleting YAP, indicating that these pathologies are YAP-dependent. In addition, increased YAP protein levels were observed in human CRC liver metastases and were correlated with CRC relapse (Steven W Plouffe et al., 2015).

### Multiple Myeloma

The Hippo pathway plays an important role in regulating lymphocyte apoptosis. YAP acts as a tumor suppressor in several hematological cancers, including multiple myeloma (MM), lymphoma, and leukemia (Steven W Plouffe et al., 2015).

### Nervous System Tumors

The Hippo pathway is involved in several nervous system tumors. Loss of function mutations in NF2 causes Neurofibromatosis Type 2, a genetic disorder characterized by ncreased

YAP expression and. NF2 inhibits YAP activity and loss of function mutations in NF2 results in increased YAP accumulation, so loss of NF2 and subsequent tumor growth could be due to aberrant YAP activity. In the central nervous system, NF2 expression is also significantly reduced in human malignant gliomas, and expression of NF2 has been shown to inhibit human glioma growth both in vitro and in vivo. Likewise, YAP is highly expressed in many human brain tumors including infiltrating gliomas, and YAP overexpression promotes glioblastoma growth in vitro (Steven W Plouffe et al., 2015).

The methods and compounds will typically be used in therapy for human subjects. However, they may also be used to treat similar or identical indications in other animal subjects.

In certain embodiments, the patient is 60 years or older and relapsed after a first line cancer therapy. In certain embodiments, the patient is 18 years or older and is relapsed or refractory after a second line cancer therapy. In certain embodiments, the patient is 60 years or older and is primary refractory to a first line cancer therapy. In certain embodiments, the patient is 70 years or older and is previously untreated. In certain embodiments, the patient is 70 years or older and is ineligible and/or unlikely to benefit from cancer therapy.

In certain embodiments, the therapeutically effective amount used in the methods provided herein is at least 10 mg per day. In certain embodiments, the therapeutically effective amount is 10, 50, 90, 100, 135, 150, 200, 250, 300, 350, 400, 450, 500, 600, 700, 800, 900, 1000, 1200, 1300, 1400, 1500, 1600, 1700, 1800, 1900, 2000, 2200, or 2500 mg per day. In other embodiments, the therapeutically effective amount is 10, 50, 90, 100, 135, 150, 200, 250, 300, 350, 400, 450, 500, 600, 700, 800, 900, 1000, 1200, 1300, 1400, 1500, 1600, 1700, 1800, 1900, 2000, 2200, 2500, 3000, 3500, 4000, 4500, or 5000 mg per day or more. In certain embodiments, the compound is administered continuously.

In certain embodiments, provided herein is a method for treating a diseases or condition mediated by YAP or TEAD by administering to a mammal having a disease or condition at least 10, 50, 90, 100, 135, 150, 200, 250, 300, 350, 400, 450, 500, 600, 700, 800, 900, 1000, 1200, 1300, 1400, 1500, 1600, 1700, 1800, 1900, 2000, 2200, 2500, 3000, 3500, 4000, 4500, or 5000 mg per day of any of the compounds described in a compound in one of Embodiments 1-50, or a pharmaceutically acceptable salt, deuterated analog, a tautomer or a stereoisomer thereof, and wherein the compound is administered on an empty stomach.

In certain embodiments, provided herein is a method for treating a diseases or condition mediated by YAP or TEAD by administering to a mammal having a disease or condition at least 10, 50, 90, 100, 135, 150, 200, 250, 300, 350, 400, 450, 500, 600, 700, 800, 900, 1000, 1200, 1300, 1400, 1500, 1600, 1700, 1800, 1900, 2000, 2200, 2500, 3000, 3500, 4000, 4500, or 5000 mg per day of any of the compounds described in a compound in one of Embodiments 1a-47a, Embodiment, 23b, or Embodiment 40b, or a pharmaceutically acceptable salt, deuterated analog, a tautomer or a stereoisomer thereof, and wherein the compound is administered on an empty stomach.

Embodiment 53 of this disclosure relates to a method for treating a subject with a disease or condition mediated by YAP/TEAD, said method comprising administering to the subject an effective amount of a compound in any one of Embodiments 1-50, or a pharmaceutically acceptable salt, deuterated analog, a tautomer or a stereoisomer thereof, or a pharmaceutical composition in any one of Embodiments 51-52.

Embodiment 53a of this disclosure relates to a method for treating a subject with a disease or condition mediated by YAP/TEAD, said method comprising administering to the subject an effective amount of a compound in any one of Embodiments 1a-47a, Embodiment, 23b, or Embodiment 40b, or a pharmaceutically acceptable salt, deuterated analog, a tautomer or a stereoisomer thereof, or a pharmaceutical composition in any one of Embodiments 51a or 52a.

Embodiment 54 of this disclosure relates to the method of Embodiment 53, wherein the disease or condition is a cancer, a neurodegenerative disease, a heart related disorder, or a kidney-related disorder.

Embodiment 54a of this disclosure relates to the method of Embodiment 53a, wherein the disease or condition is a cancer, a neurodegenerative disease, a heart related disorder, or a kidney-related disorder.

Embodiment 55 of this disclosure relates to the method of Embodiment 53, wherein the disease or condition is polycystic kidney disease, Alzheimer's disease, arrhythmogenic cardiomyopathy, Holt-Oram syndrome, liver cancer, epithelioid hemangioendothelioma, breast cancer, lung cancer, malignant mesothelioma, pancreatic cancer, kaposi sarcoma, uveal melanoma, renal cell carcinoma, colorectal cancer, multiple myeloma, neurofibromatosis Type 2, glioma, or glioblastoma.

Embodiment 55a of this disclosure relates to the method of Embodiment 53a, wherein the disease or condition is polycystic kidney disease, Alzheimer's disease, arrhythmogenic cardiomyopathy, Holt-Oram syndrome, liver cancer, epithelioid hemangioendothelioma, breast cancer, lung cancer, malignant mesothelioma, pancreatic cancer, kaposi sarcoma, uveal melanoma, renal cell carcinoma, colorectal cancer, multiple myeloma, neurofibromatosis Type 2, glioma, or glioblastoma.

### V. Combination Therapy

YAP/TEAD modulators may be usefully combined with another pharmacologically active compound, or with two or more other pharmacologically active compounds, particularly in the treatment of cancer. In one embodiment, the composition includes any one or more compound(s) as described herein along with one or more compounds that are therapeutically effective for the same disease indication, wherein the compounds have a synergistic effect on the disease indication. In one embodiment, the composition includes any one or more compound(s) as described herein effective in treating a cancer and one or more other compounds that are effective in treating the same cancer, further wherein the compounds are synergistically effective in treating the cancer.

Embodiment 56 of this disclosure relates to the method according to any one of Embodiments 53-55, further comprising administering one or more additional therapeutic agents.

Embodiment 56 of this disclosure relates to the method according to any one of Embodiments 53a-55a, further comprising administering one or more additional therapeutic agents.

Embodiment 57 of this dislcosure relates to the method according to Embodiment 56, wherein the one or more additional therapeutic agents is one or more of i) an alkylating agent selected from adozelesin, altretamine, bizelesin, busulfan, carboplatin, carboquone, carmustine, chlorambucil, cisplatin, cyclophosphamide, dacarbazine, estramustine, fotemustine, hepsulfam, ifosfamide, improsulfan, irofulven, lomustine, mechlorethamine, melphalan, oxaliplatin, piposulfan, semustine, streptozocin, temozolomide, thiotepa, and treosulfan; ii) an antibiotic selected from bleomycin, dactinomycin, daunorubicin, doxorubicin, epirubicin, idarubicin, menogaril, mitomycin, mitoxantrone, neocarzinostatin, pentostatin, and plicamycin; iii) an antimetabolite selected from azacitidine, capecitabine, cladribine, clofarabine, cytarabine, decitabine, floxuridine, fludarabine, 5-fluorouracil, ftorafur, gemcitabine, hydroxyurea, mercaptopurine, methotrexate, nelarabine, pemetrexed, raltitrexed, thioguanine, and trimetrexate; iv) an immune checkpoint agent selected from a PD-1 inhibitor, a PD-L1 inhibitor, and an anti-CTLA4 inhibitor; v) a hormone or hormone antagonist selected from enzalutamide, abiraterone, anastrozole, androgens, buserelin, diethylstilbestrol, exemestane, flutamide, fulvestrant, goserelin, idoxifene, letrozole, leuprolide, magestrol, raloxifene, tamoxifen, and toremifene; vi) a taxane selected from DJ-927, docetaxel, TPI 287, paclitaxel and DHA-paclitaxel; vii) a retinoid selected from alitretinoin, bexarotene, fenretinide, isotretinoin, and tretinoin; viii) an alkaloid selected from etoposide, homoharringtonine, teniposide, vinblastine, vincristine, vindesine, and vinorelbine; ix) an antiangiogenic agent selected from AE-941 (GW786034, Neovastat), ABT-510, 2-methoxyestradiol, lenalidomide, and thalidomide; x) a topoisomerase inhibitor selected from amsacrine, edotecarin, exatecan, irinotecan, SN-38(7-ethyl-10-hydroxy-camptothecin), rubitecan, topotecan, and 9-aminocamptothecin; xi) a kinase inhibitor selected from erlotinib, gefitinib, flavopiridol, imatinib mesylate, lapatinib, sorafenib, sunitinib malate, 7-hydroxystaurosporine, and vatalanib; xii) a targeted signal transduction inhibitor selected from bortezomib, geldanamycin, and rapamycin; xiii) a biological response modifier selected from imiquimod, interferon-α and interleukin-2; xiv) an IDO inhibitor; xv) a chemotherapeutic agent selected from 3-AP (3-amino-2-carboxyaldehyde thiosemicarbazone), altrasentan, aminoglutethimide, anagrelide, asparaginase, bryostatin-1, cilengitide, elesclomol, eribulin mesylate, ixabepilone, lonidamine, masoprocol, mitoguanazone, oblimersen, sulindac, testolactone, tiazofurin, an mTOR inhibitor, a PI3K inhibitor, a Cdk4 inhibitor, an Akt inhibitor, a Hsp90 inhibitor, a farnesyltransferase inhibitor and an aromatase inhibitor (anastrozole letrozole exemestane); xvi) a BRAF inhibitor; xvii) a Mek inhibitor; xviii) c-Kit mutant inhibitor, xix) an EGFR inhibitor, xx) an epigenetic modulator; xxi) other adenosine axis blockade agents selected from CD39, CD38, A2AR and A2BR; or xxii) agonists of TNFA super family member; and xxiii) an anti-ErbB2 mAb.

Embodiment 57a of this dislcosure relates to the method according to Embodiment 56a, wherein the one or more additional therapeutic agents is one or more of i) an alkylating agent selected from adozelesin, altretamine, bizelesin, busulfan, carboplatin, carboquone, carmustine, chlorambucil, cisplatin, cyclophosphamide, dacarbazine, estramustine, fotemustine, hepsulfam, ifosfamide, improsulfan, irofulven, lomustine, mechlorethamine, melphalan, oxaliplatin, piposulfan, semustine, streptozocin, temozolomide, thiotepa, and treosulfan; ii) an antibiotic selected from bleomycin, dactinomycin, daunorubicin, doxorubicin, epirubicin, idarubicin, menogaril, mitomycin, mitoxantrone, neocarzinostatin, pentostatin, and plicamycin; iii) an antimetabolite selected from azacitidine, capecitabine, cladribine, clofarabine, cytarabine, decitabine, floxuridine, fludarabine, 5-fluorouracil, ftorafur, gemcitabine, hydroxyurea, mercaptopurine, methotrexate, nelarabine, pemetrexed, raltitrexed, thioguanine, and trimetrexate; iv) an immune checkpoint agent selected from a PD-1 inhibitor, a PD-L1 inhibitor, and an anti-CTLA4 inhibitor; v) a hormone or hormone antagonist selected from enzalutamide, abiraterone, anastrozole, androgens, buserelin, diethylstilbestrol, exemestane, flutamide, fulvestrant, goserelin, idoxifene, letrozole, leuprolide, magestrol, raloxifene, tamoxifen, and toremifene; vi) a taxane selected from DJ-927, docetaxel, TPI 287, paclitaxel and DHA-paclitaxel; vii) a retinoid selected from alitretinoin, bexarotene, fenretinide, isotretinoin, and tretinoin; viii) an alkaloid selected from etoposide, homoharringtonine, teniposide, vinblastine, vincristine, vindesine, and vinorelbine; ix) an antiangiogenic agent selected from AE-941 (GW786034, Neovastat), ABT-510, 2-methoxyestradiol, lenalidomide, and thalidomide; x) a topoisomerase inhibitor selected from amsacrine, edotecarin, exatecan, irinotecan, SN-38(7-ethyl-10-hydroxy-camptothecin), rubitecan, topotecan, and 9-aminocamptothecin; xi) a kinase inhibitor selected from erlotinib, gefitinib, flavopiridol, imatinib mesylate, lapatinib, sorafenib, sunitinib malate, 7-hydroxystaurosporine, and vatalanib; xii) a targeted signal transduction inhibitor selected from bortezomib, geldanamycin, and rapamycin; xiii) a biological response modifier selected from imiquimod, interferon-α and interleukin-2; xiv) an IDO inhibitor; xv) a chemotherapeutic agent selected from 3-AP (3-amino-2-carboxyaldehyde thiosemicarbazone), altrasentan, aminoglutethimide, anagrelide, asparaginase, bryostatin-1, cilengitide, elesclomol, eribulin mesylate, ixabepilone, lonidamine, masoprocol, mitoguanazone, oblimersen, sulindac, testolactone, tiazofurin, an mTOR inhibitor, a PI3K inhibitor, a Cdk4 inhibitor, an Akt inhibitor, a Hsp90 inhibitor, a farnesyltransferase inhibitor and an aromatase inhibitor (anastrozole letrozole exemestane); xvi) a BRAF inhibitor; xvii) a Mek inhibitor; xviii) c-Kit mutant inhibitor, xix) an EGFR inhibitor, xx) an epigenetic modulator; xxi) other adenosine axis blockade agents selected from CD39, CD38, A2AR and A2BR; or xxii) agonists of TNFA super family member; and xxiii) an anti-ErbB2 mAb.

In another embodiment, the present disclosure provides a method of treating a cancer in a subject in need thereof by administering to the subject an effective amount of a composition including any one or more compound(s) as described herein in combination with one or more other therapies or medical procedures effective in treating the cancer. Other therapies or medical procedures include suitable anticancer therapy (e.g. drug therapy, vaccine therapy, gene therapy, photodynamic therapy) or medical procedure (e.g. surgery, radiation treatment, hyperthermia heating, bone marrow or stem cell transplant). In one embodiment, the one or more suitable anticancer therapies or medical procedures is selected from treatment with a chemotherapeutic agent (e.g. chemotherapeutic drug), radiation treatment (e.g. x-ray, gamma-ray, or electron, proton, neutron, or alpha-particle beam), hyperthermia heating (e.g. microwave, ultrasound, radiofrequency ablation), Vaccine therapy (e.g. AFP gene hepatocellular carcinoma vaccine,

AFP adenoviral vector vaccine, AG-858, allogeneic GM-CSF-secretion breast cancer vaccine, dendritic cell peptide vaccines), gene therapy (e.g. Ad5CMV-p53 vector, adenovector encoding MDA7, adenovirus 5-tumor necrosis factor alpha), photodynamic therapy (e.g. aminolevulinic acid, motexatin lutetium), surgery, or bone marrow and stem cell transplantation.

### VI. Kits

In another aspect, the present disclosure provides kits that include one or more compounds as described in any one of Embodiments 1-50, or a pharmaceutically acceptable salt, deuterated analog, a tautomer or a stereoisomer thereof, or a pharmaceutical composition in one of Embodiments 51-52. In some embodiments, the compound or composition is packaged, e.g., in a vial, bottle, flask, which may be further packaged, e.g., within a box, envelope, or bag. The compound or composition may be approved by the U.S. Food and Drug Administration or similar regulatory agency for administration to a mammal, e.g., a human. The compound or composition may be approved for administration to a mammal, e.g., a human, for a YAP/TEAD mediated disease or condition. The kits described herein may include written instructions for use and/or other indication that the compound or composition is suitable or approved for administration to a mammal, e.g., a human, for a YAP/TEAD mediated disease or condition. The compound or composition may be packaged in unit dose or single dose form, e.g., single dose pills, capsules, or the like.

In another aspect, the present disclosure provides kits that include one or more compounds as described in any one of Embodiments 1a-47a, Embodiment, 23b, or Embodiment 40b, or a pharmaceutically acceptable salt, deuterated analog, a tautomer or a stereoisomer thereof, or a pharmaceutical composition in one of Embodiments 51a or 52a. In some embodiments, the compound or composition is packaged, e.g., in a vial, bottle, flask, which may be further packaged, e.g., within a box, envelope, or bag. The compound or composition may be approved by the U.S. Food and Drug Administration or similar regulatory agency for administration to a mammal, e.g., a human. The compound or composition may be approved for administration to a mammal, e.g., a human, for a YAP/TEAD mediated disease or condition. The kits described herein may include written instructions for use and/or other indication that the compound or composition is suitable or approved for administration to a mammal, e.g., a human, for a YAP/TEAD mediated disease or condition. The compound or composition may be packaged in unit dose or single dose form, e.g., single dose pills, capsules, or the like.

### VII. Binding Assays

The methods of the present disclosure can involve assays that are able to detect the binding of compounds to a target molecule. Such binding is at a statistically significant level, with a confidence level of at least 90%, or at least 95, 97, 98, 99% or greater confidence level that the assay signal represents binding to the target molecule, i.e., is distinguished from background. In some embodiments, controls are used to distinguish target binding from non-specific binding. A large variety of assays indicative of binding are known for different target types and can be used for this disclosure.

Binding compounds can be characterized by their effect on the activity of the target molecule. Thus, a “low activity” compound has an inhibitory concentration (IC₅₀) or effective concentration (EC₅₀) of greater than 1 μM under standard conditions. By “very low activity” is meant an IC₅₀ or EC₅₀ of above 100 μM under standard conditions. By “extremely low activity” is meant an IC₅₀ or EC₅₀ of above 1 mM under standard conditions. By “moderate activity” is meant an IC₅₀ or EC₅₀ of 200 nM to 1 μM under standard conditions. By “moderately high activity” is meant an IC₅₀ or EC₅₀ of 1 nM to 200 nM. By “high activity” is meant an IC₅₀ or EC₅₀ of below 1 nM under standard conditions. The IC₅₀ or EC₅₀ is defined as the concentration of compound at which 50% of the activity of the target molecule (e.g. enzyme or other protein) activity being measured is lost or gained relative to the range of activity observed when no compound is present. Activity can be measured using methods known to those of ordinary skill in the art, e.g., by measuring any detectable product or signal produced by occurrence of an enzymatic reaction, or other activity by a protein being measured.

By “background signal” in reference to a binding assay is meant the signal that is recorded under standard conditions for the particular assay in the absence of a test compound, molecular scaffold, or ligand that binds to the target molecule. Persons of ordinary skill in the art will realize that accepted methods exist and are widely available for determining background signal.

By “standard deviation” is meant the square root of the variance. The variance is a measure of how spread out a distribution is. It is computed as the average squared deviation of each number from its mean. For example, for the numbers 1, 2, and 3, the mean is 2 and the variance is:

$${σ}^{2} = \frac{{( 1 - 2 )}^{2} + {( 2 - 2 )}^{2} + {( 3 - 2 )}^{2}}{3} = 0.66 7 .$$

### Surface Plasmon Resonance

Binding parameters can be measured using surface plasmon resonance, for example, with a BIAcore® chip (Biacore, Japan) coated with immobilized binding components. Surface plasmon resonance is used to characterize the microscopic association and dissociation constants of reaction between an sFv or other ligand directed against target molecules. Such methods are generally described in the following references which are incorporated herein by reference. Vely F. et al., (2000) BIAcore® analysis to test phosphopeptide-SH2 domain interactions, Methods in Molecular Biology. 121:313-21; Liparoto et al., (1999) Biosensor analysis of the interleukin-2 receptor complex, Journal of Molecular Recognition. 12:316-21; Lipschultz et al., (2000) Experimental design for analysis of complex kinetics using surface plasmon resonance, Methods. 20(3): 310-8; Malmqvist., (1999) BIACORE: an affinity biosensor system for characterization of biomolecular interactions, Biochemical Society Transactions 27:335-40; Alfthan, (1998) Surface plasmon resonance biosensors as a tool in antibody engineering, Biosensors & Bioelectronics. 13:653-63; Fivash et al., (1998) BIAcore for macromolecular interaction, Current Opinion in Biotechnology. 9:97-101; Price et al.; (1998) Summary report on the ISOBM TD-4 Workshop: analysis of 56 monoclonal antibodies against the MUC1 mucin. Tumour Biology 19 Suppl 1:1-20; Malmqvist et al, (1997) Biomolecular interaction analysis: affinity biosensor technologies for functional analysis of proteins, Current Opinion in Chemical Biology. 1:378-83; O'Shannessy et al., (1996) Interpretation of deviations from pseudo-first-order kinetic behavior in the characterization of ligand binding by biosensor technology, Analytical Biochemistry. 236:275-83; Malmborg et al., (1995) BIAcore as a tool in antibody engineering, Journal of Immunological Methods. 183:7-13; Van Regenmortel, (1994) Use of biosensors to characterize recombinant proteins, Developments in Biological Standardization. 83:143-51; and O'Shannessy, (1994) Determination of kinetic rate and equilibrium binding constants for macromolecular interactions: a critique of the surface plasmon resonance literature, Current Opinions in Biotechnology. 5:65-71.

BIAcore® uses the optical properties of surface plasmon resonance (SPR) to detect alterations in protein concentration bound to a dextran matrix lying on the surface of a gold/glass sensor chip interface, a dextran biosensor matrix. In brief, proteins are covalently bound to the dextran matrix at a known concentration and a ligand for the protein is injected through the dextran matrix. Near infrared light, directed onto the opposite side of the sensor chip surface is reflected and also induces an evanescent wave in the gold film, which in turn, causes an intensity dip in the reflected light at a particular angle known as the resonance angle. If the refractive index of the sensor chip surface is altered (e.g. by ligand binding to the bound protein) a shift occurs in the resonance angle. This angle shift can be measured and is expressed as resonance units (RUs) such that 1000 RUs is equivalent to a change in surface protein concentration of 1 ng/mm2. These changes are displayed with respect to time along the y-axis of a sensorgram, which depicts the association and dissociation of any biological reaction.

### High Throughput Screening (HTS) Assays

HTS typically uses automated assays to search through large numbers of compounds for a desired activity. Typically HTS assays are used to find new drugs by screening for chemicals that act on a particular enzyme or molecule. For example, if a chemical inactivates an enzyme it might prove to be effective in preventing a process in a cell which causes a disease. High throughput methods enable researchers to assay thousands of different chemicals against each target molecule very quickly using robotic handling systems and automated analysis of results.

As used herein, “high throughput screening” or “HTS” refers to the rapid in vitro screening of large numbers of compounds (libraries); generally tens to hundreds of thousands of compounds, using robotic screening assays. Ultra-high-throughput Screening (uHTS) generally refers to the high-throughput screening accelerated to greater than 100,000 tests per day.

To achieve high-throughput screening, it is advantageous to house samples on a multicontainer carrier or platform. A multicontainer carrier facilitates measuring reactions of a plurality of candidate compounds simultaneously. Multi-well microplates may be used as the carrier. Such multi-well microplates, and methods for their use in numerous assays, are both known in the art and commercially available.

Screening assays may include controls for purposes of calibration and confirmation of proper manipulation of the components of the assay. Blank wells that contain all of the reactants but no member of the chemical library are usually included. As another example, a known inhibitor (or activator) of an enzyme for which modulators are sought, can be incubated with one sample of the assay, and the resulting decrease (or increase) in the enzyme activity used as a comparator or control. It will be appreciated that modulators can also be combined with the enzyme activators or inhibitors to find modulators which inhibit the enzyme activation or repression that is otherwise caused by the presence of the known the enzyme modulator.

### Measuring Enzymatic and Binding Reactions During Screening Assays

Techniques for measuring the progression of enzymatic and binding reactions, e.g., in multicontainer carriers, are known in the art and include, but are not limited to, the following.

Spectrophotometric and spectrofluorometric assays are well known in the art. Examples of such assays include the use of colorimetric assays for the detection of peroxides, as described in Gordon, A. J. and Ford, R. A., (1972) The Chemist's Companion: A Handbook Of Practical Data, Techniques, And References, John Wiley and Sons, N.Y., Page 437.

Fluorescence spectrometry may be used to monitor the generation of reaction products. Fluorescence methodology is generally more sensitive than the absorption methodology. The use of fluorescent probes is well known to those skilled in the art. For reviews, see Bashford et al., (1987) Spectrophotometry and Spectrofluorometry: A Practical Approach, pp. 91-114, IRL Press Ltd.; and Bell, (1981) Spectroscopy In Biochemistry, Vol. I, pp. 155-194, CRC Press.

In spectrofluorometric methods, enzymes are exposed to substrates that change their intrinsic fluorescence when processed by the target enzyme. Typically, the substrate is nonfluorescent and is converted to a fluorophore through one or more reactions. As a non-limiting example, SMase activity can be detected using the Amplex® Red reagent (Molecular Probes, Eugene, OR). In order to measure sphingomyelinase activity using Amplex® Red, the following reactions occur. First, SMase hydrolyzes sphingomyelin to yield ceramide and phosphorylcholine. Second, alkaline phosphatase hydrolyzes phosphorylcholine to yield choline. Third, choline is oxidized by choline oxidase to betaine. Finally, H₂O₂, in the presence of horseradish peroxidase, reacts with Amplex® Red to produce the fluorescent product, Resorufin, and the signal therefrom is detected using spectrofluorometry.

Fluorescence polarization (FP) is based on a decrease in the speed of molecular rotation of a fluorophore that occurs upon binding to a larger molecule, such as a receptor protein, allowing for polarized fluorescent emission by the bound ligand. FP is empirically determined by measuring the vertical and horizontal components of fluorophore emission following excitation with plane polarized light. Polarized emission is increased when the molecular rotation of a fluorophore is reduced. A fluorophore produces a larger polarized signal when it is bound to a larger molecule (i.e. a receptor), slowing molecular rotation of the fluorophore. The magnitude of the polarized signal relates quantitatively to the extent of fluorescent ligand binding. Accordingly, polarization of the “bound” signal depends on maintenance of high affinity binding.

FP is a homogeneous technology and reactions are very rapid, taking seconds to minutes to reach equilibrium. The reagents are stable, and large batches may be prepared, resulting in high reproducibility. Because of these properties, FP has proven to be highly automatable, often performed with a single incubation with a single, premixed, tracer-receptor reagent. For a review, see Owicki et al., (1997), Application of Fluorescence Polarization Assays in High-Throughput Screening, Genetic Engineering News, 17:27.

FP is particularly desirable since its readout is independent of the emission intensity (Checovich, W. J., et al., (1995) Nature 375:254-256; Dandliker, W. B., et al., (1981) Methods in Enzymology 74:3-28) and is thus insensitive to the presence of colored compounds that quench fluorescence emission. FP and FRET (see below) are well-suited for identifying compounds that block interactions between sphingolipid receptors and their ligands. See, for example, Parker et al., (2000) Development of high throughput screening assays using fluorescence polarization: nuclear receptor-ligand-binding and kinase/phosphatase assays, J Biomol Screen 5:77-88.

Fluorophores derived from sphingolipids that may be used in FP assays are commercially available. For example, Molecular Probes (Eugene, OR) currently sells sphingomyelin and one ceramide flurophores. These are, respectively, N-(4,4-difluoro-5,7-dimethyl-4-bora-3a,4a-diaza -s-indacene-3-pentanoyl) sphingosyl phosphocholine (BODIPY® FL C₅-sphingomyelin); N-(4,4-difluoro-5,7-dimethyl-4-bora-3a,4a-diaza -s-indacene-3-dodecanoyl) sphingosyl phosphocholine (BODIPY® FL C₁₂-sphingomyelin); and N-(4,4-difluoro-5,7-dimethyl-4-bora-3a,4a-diaza -s-indacene-3-pentanoyl) sphingosine (BODIPY® FL C₅-ceramide). U.S. Pat. No. 4,150,949, (Immunoassay for gentamicin), discloses fluorescein-labelled gentamicins, including fluoresceinthiocarbanyl gentamicin. Additional fluorophores may be prepared using methods well known to the skilled artisan.

Exemplary normal- and-polarized fluorescence readers include the POLARION® fluorescence polarization system (Tecan AG, Hombrechtikon, Switzerland). General multiwell plate readers for other assays are available, such as the VERSAMAX® reader and the SPECTRAMAX® multiwell plate spectrophotometer (both from Molecular Devices).

Fluorescence resonance energy transfer (FRET) is another useful assay for detecting interaction and has been described. See, e.g., Heim et al., (1996) Curr. Biol. 6:178-182; Mitra et al., (1996) Gene 173:13-17; and Selvin et al., (1995) Meth. Enzymol. 246:300-345. FRET detects the transfer of energy between two fluorescent substances in close proximity, having known excitation and emission wavelengths. As an example, a protein can be expressed as a fusion protein with green fluorescent protein (GFP). When two fluorescent proteins are in proximity, such as when a protein specifically interacts with a target molecule, the resonance energy can be transferred from one excited molecule to the other. As a result, the emission spectrum of the sample shifts, which can be measured by a fluorometer, such as a fMAX multiwell fluorometer (Molecular Devices, Sunnyvale Calif.).

Scintillation proximity assay (SPA) is a particularly useful assay for detecting an interaction with the target molecule. SPA is widely used in the pharmaceutical industry and has been described (Hanselman et al., (1997) J. Lipid Res. 38:2365-2373; Kahl et al., (1996) Anal. Biochem. 243:282-283; Undenfriend et al., (1987) Anal. Biochem. 161:494-500). See also U.S. Pat. Nos. 4,626,513 and 4,568,649, and European Patent No. 0,154,734. One commercially available system uses FLASHPLATE® scintillant-coated plates (NEN Life Science Products, Boston, MA).

The target molecule can be bound to the scintillator plates by a variety of well-known means. Scintillant plates are available that are derivatized to bind to fusion proteins such as GST, His6 or Flag fusion proteins. Where the target molecule is a protein complex or a multimer, one protein or subunit can be attached to the plate first, then the other components of the complex added later under binding conditions, resulting in a bound complex.

In a typical SPA assay, the gene products in the expression pool will have been radiolabeled and added to the wells, and allowed to interact with the solid phase, which is the immobilized target molecule and scintillant coating in the wells. The assay can be measured immediately or allowed to reach equilibrium. Either way, when a radiolabel becomes sufficiently close to the scintillant coating, it produces a signal detectable by a device such as a TOPCOUNT NXT® microplate scintillation counter (Packard BioScience Co., Meriden Conn.). If a radiolabeled expression product binds to the target molecule, the radiolabel remains in proximity to the scintillant long enough to produce a detectable signal.

In contrast, the labeled proteins that do not bind to the target molecule, or bind only briefly, will not remain near the scintillant long enough to produce a signal above background. Any time spent near the scintillant caused by random Brownian motion will also not result in a significant amount of signal. Likewise, residual unincorporated radiolabel used during the expression step may be present, but will not generate significant signal because it will be in solution rather than interacting with the target molecule. These non-binding interactions will therefore cause a certain level of background signal that can be mathematically removed. If too many signals are obtained, salt or other modifiers can be added directly to the assay plates until the desired specificity is obtained (Nichols et al., (1998) Anal. Biochem. 257:112-119).

### General Synthesis

The compounds may be prepared using the methods disclosed herein and routine modifications thereof, which will be apparent given the disclosure herein and methods well known in the art. Conventional and well-known synthetic methods may be used in addition to the teachings herein. The synthesis of typical compounds described herein may be accomplished as described in the following examples. If available, reagents may be purchased commercially, e.g., from Sigma Aldrich or other chemical suppliers.

The compounds of this disclosure can be prepared from readily available starting materials using, for example, the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein.

The compounds of this disclosure may contain one or more asymmetric or chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, supercritical fluid chromathography, chiral seed crystals, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka —Chemce or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

It will also be appreciated that in each of the schemes, the addition of any substituent may result in the production of a number of isomeric products (including, but not limited to, enantiomers or one or more diastereomers) any or all of which may be isolated and purified using conventional techniques. When enantiomerically pure or enriched compounds are desired, chiral chromatography and/or enantiomerically pure or enriched starting materials may be employed as conventionally used in the art or as described in the Examples.

Scheme 1 provides exemplary synthetic routes for the synthesis of compounds provided herein (e.g., compounds of Formula (Ia)).

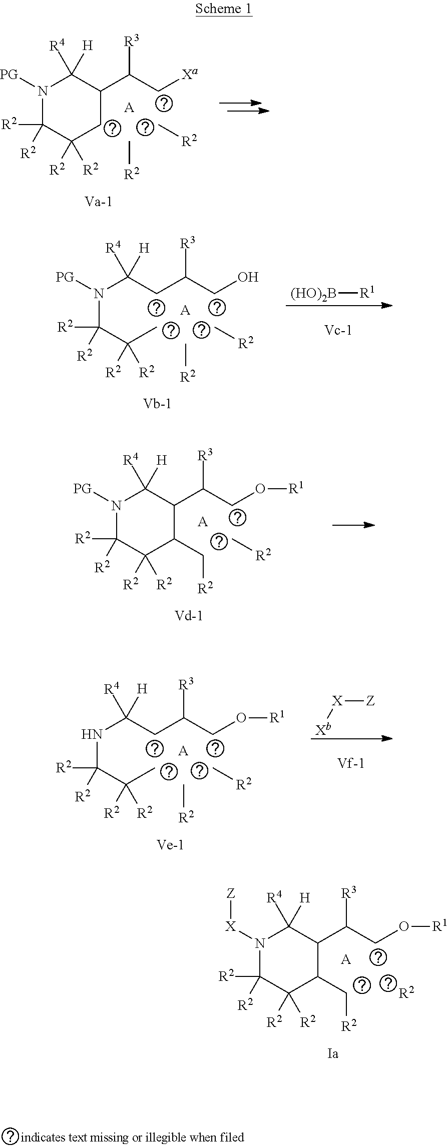

In Scheme 1, R¹, R², R³, R⁴, X, and Z are as defined herein, and PG, X^{a}, and X^{b} are defined below.

In Scheme 1, Compound Va-1, wherein X^{a} may be a suitable leaving group for a cross coupling reaction (e.g., a halo, such as a bromo, chloro, iodo) and PG may be a suitable amine protecting group (e.g., tert-butyloxycarbonyl (BOC), carbobenzyloxy (Cbz), 9-fluorenylmethyloxycarbonyl (Fmoc), and the like), may be converted to Compound Vb-1 under conditions known in the art; for example, Compound Va-1 may be combined with bis(pinacolato)diboron under Miyaura borylation conditions, including a coupling agent (e.g., Pd(dppf)Cl₂ and the like) and a base (e.g., potassium acetate, triethylamine, etc.) in a suitable solvent (e.g., dioxane and the like), followed by oxidation of the resultant boronic ester to Compound Vb-1 under oxidizing conditions, including an oxidant (e.g., hydrogen peroxide and the like) and a base (e.g., sodium hydroxide, and the like).

Conversion of Compound Vb-1 to Compound Vd-1 can be accomplished via standard coupling conditions. For example, Compound Vb-1 may be reacted with Compound Vc-1 under conditions known in the art for example, under Chan-Lam coupling conditions, including a catalyst (e.g., Cu (OAc) 2 and the like) and a base (e.g., triethylamine, pyridine, etc.) in a suitable solvent (e.g., dichloromethane, and the like), to form Compound Vd-1.

Compound Ve-1 may be synthesized by deprotecting Compound Vd under appropriate deprotection conditions, such as treatment with a strong acid (e.g., TFA, HCl) in a suitable solvent (e.g., dichloromethane), when PG is BOC. Other protecting groups may be removed using standard deprotecting conditions known to those of skill in the art.

Compound Ve-1 may then be reacted with Compound Vf-1 (wherein X^{b} may be OH or LG) to provide compounds of Formula I under standard conditions. For example, when a Compound Vf-1 is HO—(CH₂)ₙ—C(O)—Z (wherein n is as described herein), standard conditions for amide bond forming reactions between Compound Ve-1 and Compound Vf-1 may be used; these include, for example, a coupling agent for forming an amide bond (e.g., HATU, HBTU, EDC, DCC, and the like), a base (e.g., diisopropylethylamine, triethylamine, pyridine, etc.), and a suitable solvent (e.g., a polar aprotic solvent such as DMF or pyridine). In another non-limiting example, when a Compound Vf-1 is LG-(CH₂)ₘ—SO₂—Z, wherein m is as defined herein and LG is a suitable leaving group (including but not limited to a halo, such as chloro, bromo, and iodo), standard conditions for the formation of sulfonamide compounds of Formula (Ia) from Compound Ve-1 may be used; these include, for example, an optional coupling agent (e.g., HATU and the like), a base (e.g., diisopropylethylamine, triethylamine, etc.), and a suitable solvent (e.g., a polar aprotic solvent such as DMF or dichloromethane). In another non-limiting example, when a Compound Vf-1 is X^{b}-Z, wherein X^{b} is halo and Z is a 5-6 membered heteroaryl (i.e. X is a bond), standard conditions known in the art for palladium-catalyzed coupling may be used, including but not limited to Buchwald type reactions, to achieve compounds of Formula (Ia).

Compounds of Formula (Ia) can optionally be further derivatized at Z. For example, for the formation of sulfonamides from free amines on the substituent Z, conditions for the formation of sulfonamide compounds of Formula (Ia) include treatment with a sulfonyl halide (e.g., chloro, bromo, and the like), an optional coupling agent (e.g., HATU and the like), a base (e.g., diisopropylethylamine, triethylamine), and a suitable solvent (e.g., a polar aprotic solvent such as DMF or dichloromethane).

Scheme 2 provides further exemplary synthetic routes for the synthesis of compounds provided herein (e.g., compounds of Formula (Ib)).

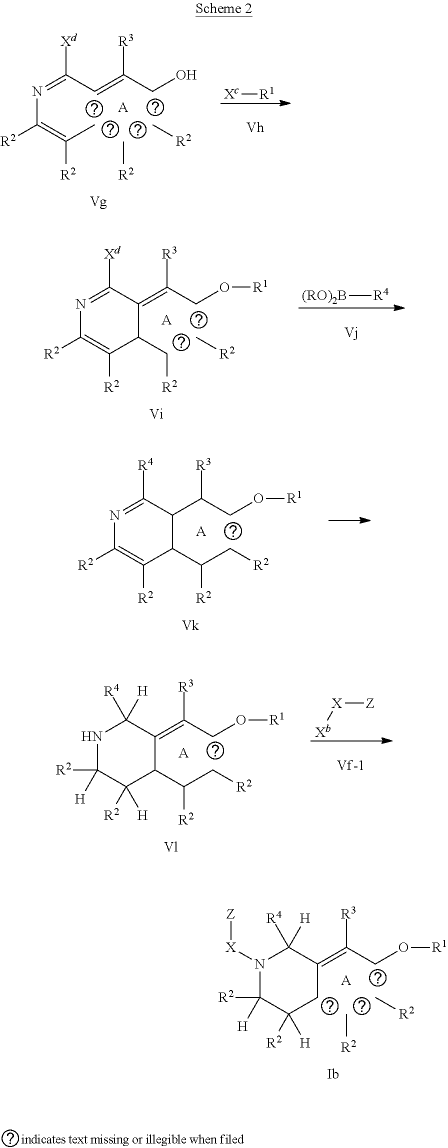

In Scheme 2, R¹, R², R³, R⁴, X, and Z are as defined herein, and R, X^{b}, X^{c}, and X^{d} are defined below.

In Scheme 2, Compound Vg may be converted to Compound Vi (wherein X^{d} may be a suitable metal-catalyzed cross coupling partner (e.g., a halo, such as a chloro, bromo, or iodo, or a pseudohalide, such as triflate and the like)) by reacting Compound Vg with Compound Vh, wherein X_{c} may be a suitable halide (e.g., iodo, bromo, chloro), under conditions known in the art, such as Ullmann-type copper-catalyzed nucleophilic aromatic susbstitution conditions (including a copper (I) catalyst (e.g., CuI and the like), an appropriate ligand (e.g., dimethylglycine and the like), and a base (e.g., cesium carbonate, potassium phosphate, etc.) in a suitable solvent (e.g., a polar aprotic solvent such as DMF, DMSO, acetonitrile, or the like).

Compound Vi may be converted to Compound Vk by reacting with Compound Vj, wherein R may be hydrogen, or the two Rs, together with oxygens to which they are attached, form a cyclic boronic ester, such as pinacol boronic ester. The reaction of Compound Vi with Compound Vj occurs under suitable conditions for cross coupling, for example, conditions described herein, or conditions known in the art. Non-limiting examples of suitable cross coupling conditions include a palladium catalyst (e.g., Pd (PPh3) 2Cl₂, Pd (PPh3) 4, and the like), a base (e.g., sodium carbonate), and a suitable solvent (e.g., dioxane/water). Compound Vk may also be formed by employing other suitable metal-catalyzed cross coupling reactions between Compound Vi and Compound Vj, including but not limited to Stille coupling (wherein Compound Vj contains an organostannane (e.g., tributyltin, triphenyltin, and the like) in place of the boronic acid or ester), Hiyama coupling (wherein Compound Vj contains an organosilane (e.g., trimethylsilane, triethylsilane, and the like) in place of the boronic acid or ester), and Negishi coupling (wherein Compound Vj contains a zinc halide (e.g., chloro, bromo, iodo) in place of the boronic acid or ester), with the coupling conditions necessary for each well know to those of skill in the art.

Compound Vk may then be converted to Compound Vl under catalytic hydrogenation conditions, such as with a metal catalyst (e.g., palladium on carbon, platinum on carbon, and the like) and a suitable solvent (e.g., methanol, etc.), under an atmosphere of hydrogen gas.

Compound Vl may then be reacted with Compound Vf-1 (wherein X^{b} may be OH or LG) to provide compounds of Formula Ib under standard conditions. For example, when a Compound Vf-1 is HO—(CH₂)ₙ—C(O)—Z (wherein n is as described herein), standard conditions for amide bond forming reactions between Compound Vl and Compound Vf-1 may be used; these include, for example, a coupling agent for forming an amide bond (e.g., HATU, HBTU, EDC, DCC, and the like), a base (e.g., diisopropylethylamine, triethylamine, pyridine, etc.), and a suitable solvent (e.g., a polar aprotic solvent such as DMF or pyridine). In another non-limiting example, when a Compound Vf-1 is LG-(CH₂)ₘ—SO₂-Z, wherein m is as defined herein and LG is a suitable leaving group (including but not limited to a halo, such as chloro, bromo, and iodo), standard conditions for the formation of sulfonamide compounds of Formula Ib from Compound Vl may be used; these include, for example, an optional coupling agent (e.g., HATU and the like), a base (e.g., diisopropylethylamine, triethylamine, etc.), and a suitable solvent (e.g., a polar aprotic solvent such as DMF or dichloromethane). In another non-limiting example, when a Compound Vf-1 is X^{b}-Z, wherein X^{b} is halo and Z is a 5-6 membered heteroaryl (i.e. X is a bond), standard conditions known in the art for palladium-catalyzed coupling may be used, including but not limited to Buchwald type reactions, to achieve compounds of Formula I′.

Compounds of Formula Ib can optionally be further derivatized at Z, such as described herein, for example, for compounds of Formula Ia.

Scheme 3 provides exemplary synthetic routes for the synthesis of compounds provided herein (e.g., compounds of Formula (Ic) and Formula (Id)).

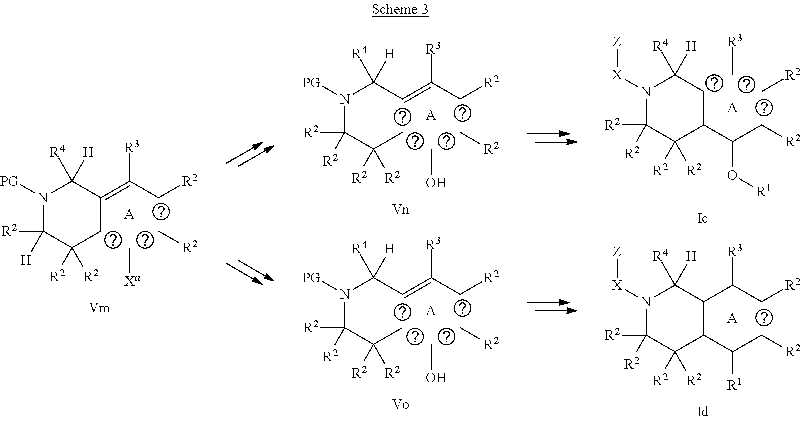

In Scheme 3, R¹, R², R³, R⁴, X, and Z are as defined herein, and PG and X^{a} are defined below.

In Scheme 3, Compound Vm, wherein X^{a} may be a suitable leaving group for a cross coupling reaction (e.g., a halo, such as a bromo, chloro, iodo) and PG may be a suitable amine protecting group (e.g., tert-butyloxycarbonyl (BOC), carbobenzyloxy (Cbz), 9-fluorenylmethyloxycarbonyl (Fmoc), and the like), may be converted to Compound Vn under conditions known in the art; for example, Compound Vm may be combined with bis(pinacolato)diboron under Miyaura borylation conditions, including a coupling agent (e.g., Pd(dppf)Cl₂ and the like) and a base (e.g., potassium acetate, triethylamine, etc.) in a suitable solvent (e.g., dioxane and the like), followed by oxidation of the resultant boronic ester to Compound Vn under oxidizing conditions, including an oxidant (e.g., hydrogen peroxide and the like) and a base (e.g., sodium hydroxide, and the like). Derivation of Compound Vn to form Compound Ic can be performed as described herein, including but not limited to Scheme 1.

Compound Vm, wherein X^{a} may be a suitable leaving group for a cross coupling reaction (e.g., a halo or triflate), may also be converted to Compound Vo via standard cross-coupling conditions, including but not limited to Suzuki coupling conditions (e.g., in the presence of an appropriate palladium calayst and with an appropriate boronic ester, etc.). Deprotection of Compound Vo and subsequent introduction of-X-Z according to methods as described herein (including but not limited to Scheme 1) affords Compound Id.

Scheme 4 provides further exemplary synthetic routes for the synthesis of compounds provided herein (e.g., compounds of Formula (Ie)).

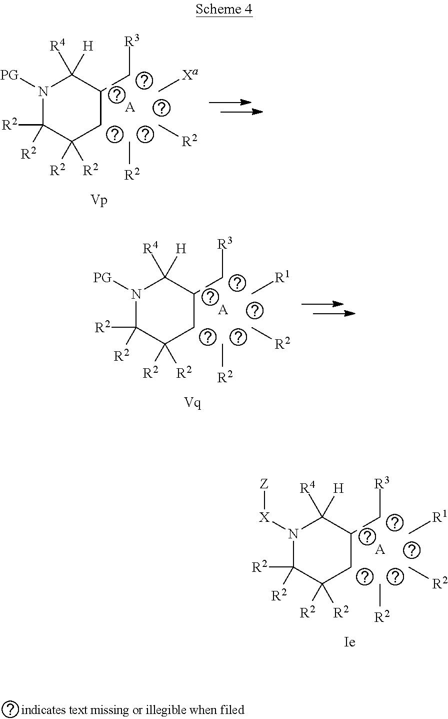

In Scheme 4, R¹, R², R³, R⁴, X, and Z are as defined herein, and PG and X^{a} are defined below.

In Scheme 4, Compound Vp, wherein X^{a} may be a suitable leaving group for a cross coupling reaction (e.g., a halo or triflate), may be converted to Compound Vq via standard cross-coupling conditions, including but not limited to Suzuki coupling conditions (e.g., in the presence of an appropriate palladium calayst and with an appropriate boronic ester, etc.). Deprotection of Compound Vq and subsequent introduction of X-Z according to methods as described herein (including but not limited to Scheme 1) affords Compound Ie.

Alternatively, the introduction of X-Z to a deprotected analog of Compound Vp according to standard methods known in the art and/or methods as described herein, followed by introduction of R¹ according to standard methods known in the art and/or methods as described herein can afford Compound Ie.

A person of skill in the art will appreciate that any of Compounds Va-1, Vb-1, Vc-1, Vd-1, Ve-1, Vf-1, Vg, Vh, Vi, Vj, Vk, VI, Vm, Vn, Vo, Vp, or Vq may be available from a commercial supplier for a particular embodiment. Alternative syntheses of Compounds Va-1, Vb-1, Vc-1, Vd-1, Ve-1, Vf-1, Vg, Vh, Vi, Vj, Vk, VI, Vm, Vn, Vo, Vp, or Vq may be as described herein or as known to those of skill in the art.

Compounds of the present disclosure may be synthesized in accordance with the examples described below. The examples may be altered by substitution of the starting materials with other materials having similar structures to result in corresponding products. The structure of the desired product will generally make apparent to a person of skill in the art the required starting materials.

### Synthesis of Intermediate 1

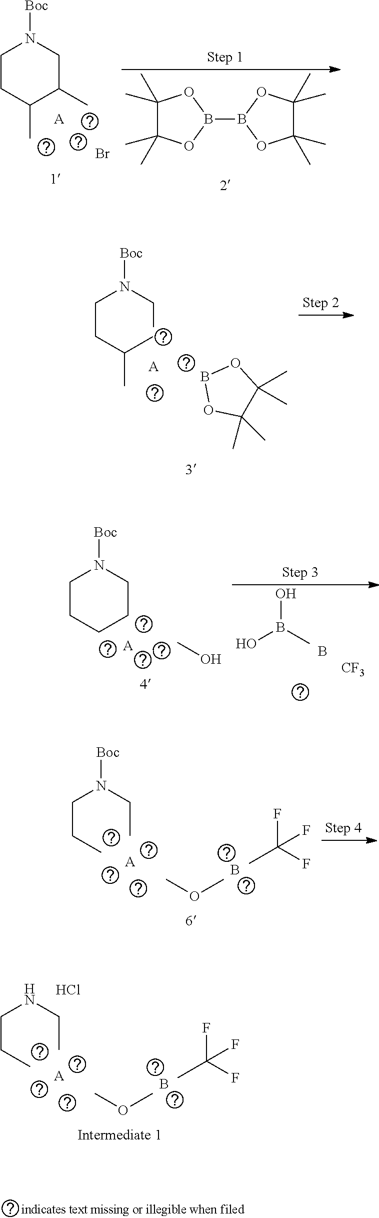

### Step 1: Preparation of tert-Butyl 7-(4,4,5,5-tetramethyl-1,3,2-dioxaborolan-2-yl)-3,4-dihydroisoquinoline-2(1H)-carboxylate (3′)

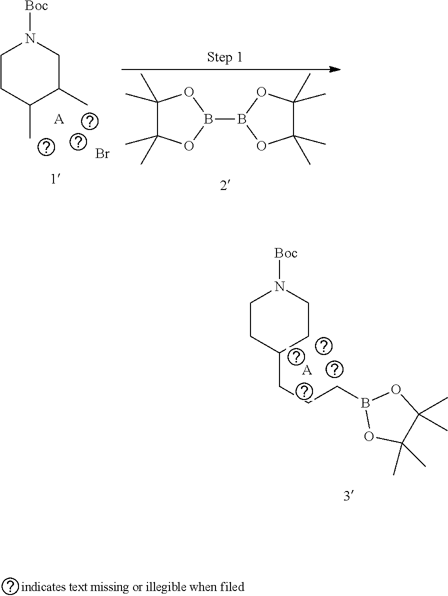

To a solution of tert-butyl 7-bromo-3,4-dihydro-1H-isoquinoline-2-carboxylate (compound 1′, 53 g, 169.76 mmol, 1 eq) in dioxane (500 mL) was added 4,4,5,5-tetramethyl-2-(4,4,5,5-tetramethyl-1,3,2-dioxaborolan-2-yl)-1,3,2-dioxaborolane (compound 2′, 64.66 g, 254.64 mmol, 1.5 eq), Pd(dppf)Cl₂(6.21 g, 8.49 mmol, 0.05 eq), and AcOK (49.98 g, 509.29 mmol, 3 eq). The mixture was stirred at 90° C. under N₂ for 15 hrs. LC-MS showed the reaction completed. The reaction mixture was diluted with water (500 mL) and extracted with EtOAc (500 mL×2). The combined organic layers were washed with brine (500 mL), dried over Na₂SO₄, filtered and concentrated under reduced pressure to give a residue. The residue was purified by column chromatography (SiO₂, Petroleum ether/Ethyl acetate=100/0 to 10/1) to give compound 3′. LC-MS (ESI): RT=1.50 min, mass calcd. for C₂₀H₃₀BNO₄ 359.23 m/z, found 305.10 [M-56+H]⁺. Method: the gradient was 30-100% B in 1.5 min, 100-100% B in 1.0 min, 100-30% B in 0.01 min. (0.3 mL/min flow rate). Mobile phase A was 0.01% CF3COOH in water, mobile phase B was 0.01% CF3COOH in CH₃CN. The column used for the chromatography is a Chromolith Flash waters-BEH-C18 1.7 um, 2.1×50 mm column. Detection methods are diode array (waters-UPLC-PDA) as well as positive electrospray ionization (waters-SQD-MS). 1H NMR (400 MHZ, CDCl₃) δ1.32 (s, 12H), 1.46 (s, 9H), 2.83 (s, 2H), 3.62 (s, 2 H), 4.56 (s, 2H), 7.12 (d, J=7.46 Hz, 1H), 7.51-7.60 (m, 2H).

### Step 2: Preparation of tert-Butyl 7-hydroxy-3,4-dihydroisoquinoline-2(1H)-carboxylate (4′)

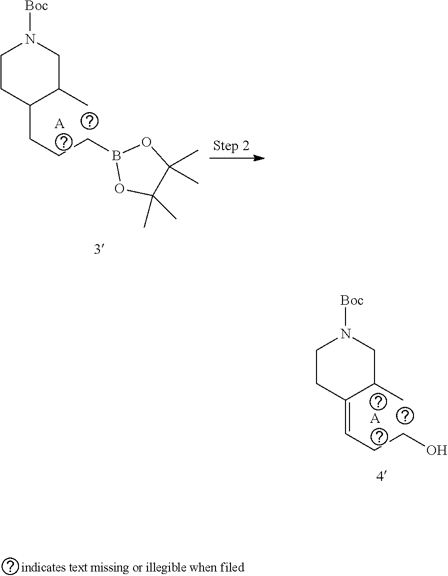

The reaction was performed in three batches: 63 g/71 g/71 g of compound 3′, which were combined upon completion. NaOH (6 M, 116.90 mL, 5 eq) was added dropwise to a solution of tert-butyl 7-(4,4,5,5-tetramethyl-1,3,2-dioxaborolan-2-yl)-3,4-dihydro-1H-isoquinoline-2-carboxylate (compound 3′, 63 g, 140.29 mmol, 80% purity, 1 eq) in DCM (600 mL) at 0° C. The resultant mixture was stirred at 0° C. for 10 min before treating with H₂O₂ (135.18 g, 1.19 mol, 114.56 mL, 30% purity, 8.5 eq) in portions at 0° C. under N₂. The resultant mixture was stirred for 16 hrs with gradual warming to room-temperature. LC-MS showed the reaction completed. The reaction was quenched with Na₂S203(600 mL). The mixture was stirred for 1 hr, then adjusted pH to ˜6. Then the mixture was diluted with water (600 mL) and extracted with DCM (600 mL*3). The combined organic layers were washed with brine (200 mL), dried over Na₂SO₄, filtered and concentrated under reduced pressure to give a residue. The residues of three batches were combined. The residue was purified by column chromatography (SiO₂, Petroleum ether/Ethyl acetate=100/0 to 4/1) to give compound 4′. LC-MS (ESI): RT=1.13 min, mass calcd. for C₁₄H₁₉NO₃ 249.14 m/z, found 195.2 [M-56+H]⁺. Method: the gradient was 30-100% B in 1.5 min, 100-100% B in 1.0 min, 100-30% B in 0.01 min. (0.3 mL/min flow rate). Mobile phase A was 0.01% CF₃COOH in water, mobile phase B was 0.01% CF₃COOH in CH₃CN. The column used for the chromatography is a Chromolith Flash waters-BEH-C18 1.7 um, 2.1×50 mm column. Detection methods are diode array (waters-UPLC-PDA) as well as positive electrospray ionization (waters-SQD-MS). 1H NMR (400 MHZ, CDCl₃) δ1.49 (s, 9H), 2.73 (t, J=5.8 Hz, 2H), 3.61 (t, J-6.0 Hz, 2H), 4.50 (s, 2H), 6.61-6.73 (m, 2H), 6.96 (d, J=8.20 Hz, 1H).

### Step 3: Preparation of tert-Butyl 7-(4-(trifluoromethyl) phenoxy)-3,4-dihydroisoquinoline-2(1H)-carboxylate (6′)

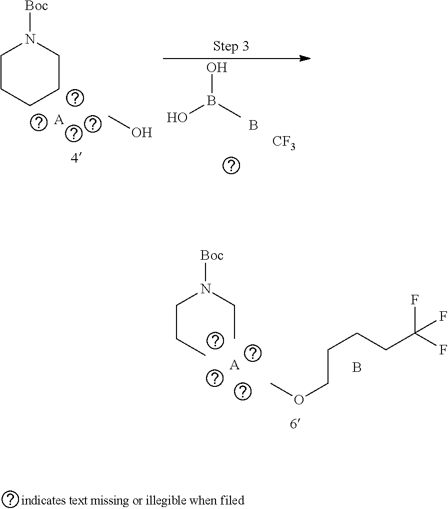

To a solution of tert-butyl 7-hydroxy-3,4-dihydro-1H-isoquinoline-2-carboxylate (compound 4′, 80 g, 320.89 mmol, 1 eq), (4-(trifluoromethyl)phenyl) boronic acid (compound 5′, 79.84 g, 420.37 mmol, 1.31 eq), TEA (129.88 g, 1.28 mol, 178.66 mL, 4 eq), and 4A molecular sieve (80 g) in DCM (1000 mL), was added Cu (OAc) 2(17.49 g, 96.27 mmol, 0.3 eq). The mixture was purged with O₂ for 5 min, then stirred at 40° C. for 16 hrs under 02. LC-MS showed some of compound 4′ remained and the desired compound was detected. The mixture was filtered. The filtrate was diluted with water (1000 mL) and extracted with DCM (500 mL×2). The combined organic layers were washed with brine (500 mL), dried over Na₂SO₄, filtered and concentrated under reduced pressure to give a residue. The residue was purified by column chromatography (SiO₂, Petroleum ether/Ethyl acetate=100/1 to 5/1) to give crude compound 6′. The combined crude tert-butyl 7-[4-(trifluoromethyl) phenoxy]-3,4-dihydro-1H-isoquinoline-2-carboxylate was re-crystallized from PE: EA (10:1) to give compound 6′. LC-MS (ESI): RT=1.60 min, mass calcd. for C₂₁H₂₂F3NO₃ 393.16 m/z, found 337.80 [M-56+H]⁺. Method: the gradient was 30-100% B in 1.5 min, 100-100% B in 1.0 min, 100-30% B in 0.01 min. (0.3 mL/min flow rate). Mobile phase A was 0.01% CF₃COOH in water, mobile phase B was 0.01% CF₃COOH in CH₃CN. The column used for the chromatography is a Chromolith Flash waters-BEH-C18 1.7 um, 2.1×50 mm column. Detection methods are diode array (waters-UPLC-PDA) as well as positive electrospray ionization (waters-SQD-MS). 1H NMR (400 MHZ, CDCl₃) δ1.49 (s, 9H), 2.83 (t, J=5.44 Hz, 2H), 3.66 (t, J=5.20 Hz, 2H), 4.55 (s, 2H), 6.77-6.90 (m, 2H), 7.02 (d, J=8.6 Hz, 2H), 7.15 (d, J=8.2 Hz, 1H), 7.56 (d, J=8.68 Hz, 2H).

### Step 4: Preparation of 7-(4-(Trifluoromethyl) phenoxy)-1,2,3,4-tetrahydroisoquinoline hydrochloride (Intermediate 1)

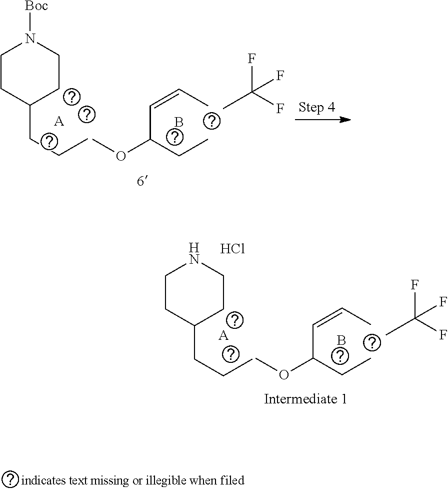

A mixture of tert-butyl 7-[4-(trifluoromethyl) phenoxy]-3,4-dihydro-1H-isoquinoline-2-carboxylate (compound 6′, 35 g, 88.97 mmol, 1 eq), HCl/dioxane (4 M, 111.21 mL, 5 eq), in DCM (400 mL) was stirred at 25° C. for 2 hrs. LC-MS showed the reaction completed. The mixture was concentrated by evaporation under reduced pressure to give Intermediate 1HCI, which was used in the next step without purification. LC-MS (ESI): RT=0.48 min, mass calcd. for C₁₆H₁₄F₃NO 293.10 m/z, found 295.5 [M+H]⁺. Method: the gradient was 5-95% B in 5 min, 95-95% B in 1.0 min, 95-5% B in 0.01 min. (1.0 mL/min flow rate). Mobile phase A was 0.01% CF₃COOH in water, mobile phase B was 0.01% CF₃COOH in CH₃CN. The column used for the chromatography is a Chromolith Flash waters-BEH-C18 1.7 um, 2.1×50 mm column. Detection methods are diode array (waters-UPLC-PDA) as well as positive electrospray ionization (waters-SQD-MS).

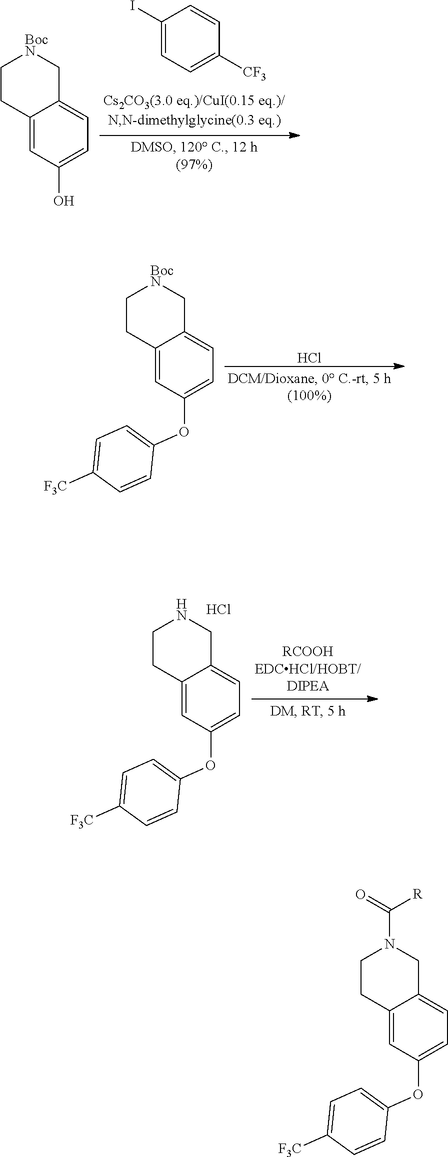

### General Procedure for the Synthesis of 6-Position Ether-Linked Intermediates

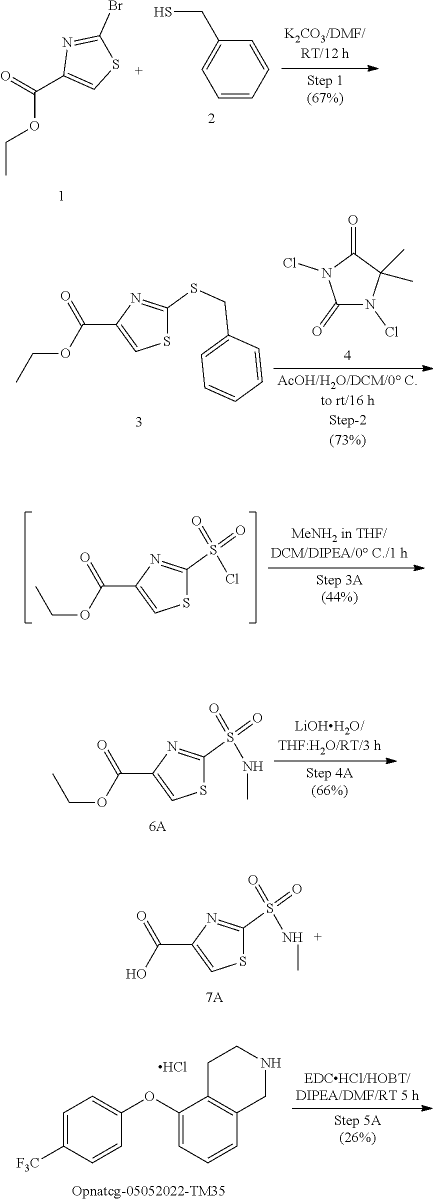

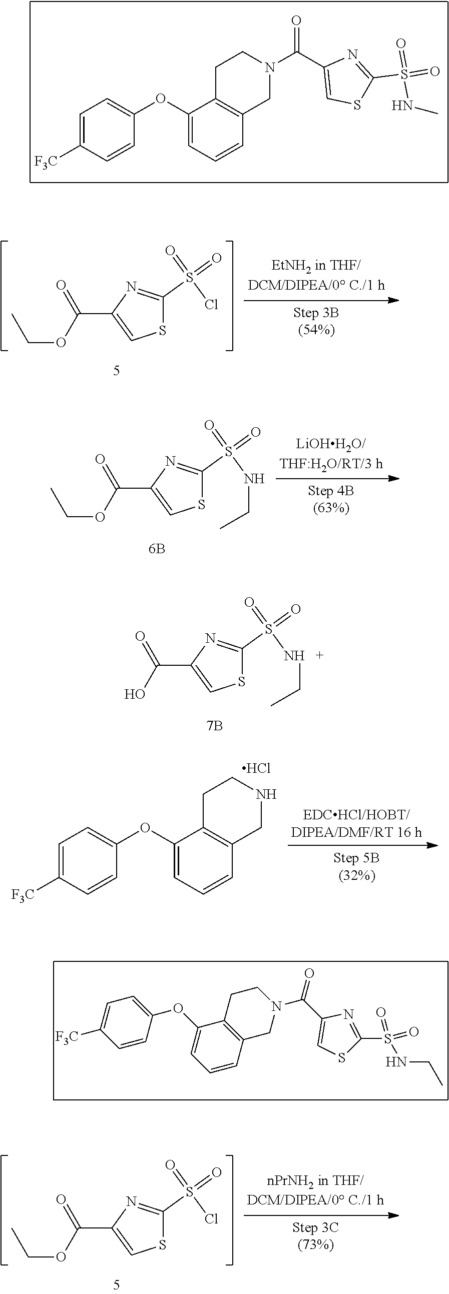

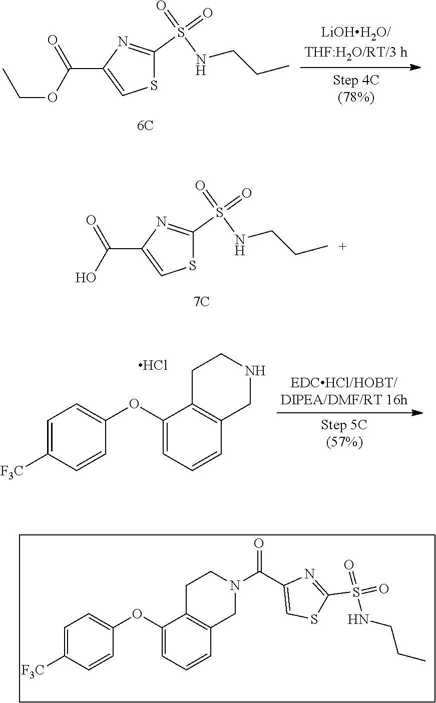

### Synthesis procedures for the sulfonamide analogues

### Intermediate 3

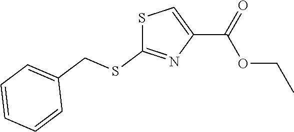 [SMILES: CCOC(=O)c1csc(SCc2ccccc2)n1]

### ethyl 2-(benzylthio) thiazole-4-carboxylate

Step-1: To a solution of ethyl 2-bromothiazole-4-carboxylate (1) (1 g, 4.24 mmol) in DMF (15 mL) was added K₂CO3(1.17 g, 8.47 mmol) in a single portion, stirred for 5 min and phenylmethanethiol (2) (0.5 mL, 4.24 mmol) was added and stirred for 12 h at room temperature. After completion, the reaction was diluted with ethyl acetate and quenched with water and saturated sodium bicarbonate. The layers were separated, and the organic part was evaporated to get crude, which was purified by combi-flash chromatography (15-20% ethyl acetate in hexane) to get ethyl 2-(benzylthio) thiazole-4-carboxylate (Intermediate 3) as white gum. (800 mg, 67%); MS (m/z): 280.1 [M+H].

### Intermediate 5

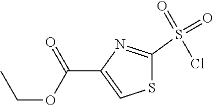

### ethyl 2-(chlorosulfonyl) thiazole-4-carboxylate

Step-2: To a solution of ethyl 2-(benzylthio) thiazole-4-carboxylate (Intermediate 3) (1.5 g, 5.37 mmol) in DCM (15 mL), Acetic acid (1.5 mL) and water (3 mL) was added 1,3-dichloro-5,5-dimethyl-imidazolidine-2,4-dione (3.2 g, 16.1 mmol) portionwise with stirring at 0° C. The mixture was stirred at room temperature for 16 h. Then the mixture was quenched with water and extracted with DCM. The combined organic layers were washed with sodium bicarbonate, dried over anhydrous magnesium sulfate, filtered and concentrated under reduced pressure to give a residue that was triturated with 3 volumes of pentane and filtered to give ethyl 2-(chlorosulfonyl) thiazole-4-carboxylate (Intermediate 5) as off white solid. (1000 mg, 73%); Confirmed by 1H-NMR.

### Intermediate 6A

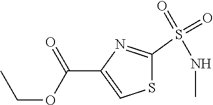

### ethyl 2-(N-methylsulfamoyl) thiazole-4-carboxylate

Step 3A: To a solution of ethyl 2-(chlorosulfonyl) thiazole-4-carboxylate (Intermediate 5) (500 mg, 1.95 mmol) in DCM (10 mL) at 0° C., was added MeNH2(2M in THF) (3 mL, 5.87 mmol) dropwise, followed by addition of DIPEA (0.7 mL, 3.9 mmol) and the reaction was stirred at 0° C. for 1 h. TLC and LCMS of crude showed formation of desired mass. The reaction was diluted with DCM and washed with saturated bicarbonate solution, water and brine. Dried over anhydrous sodium sulfate and concentrated in vacuum to get crude (˜270 mg). Crude was purified by flash chromatography (SiO2, 30-40% ethyl acetate-hexane) to get desired compound ethyl 2-(N-methylsulfamoyl) thiazole-4-carboxylate (Intermediate 6A) as off white solid. (215 mg, 44%); MS (m/z): 251.2 [M+H].

### Intermediate 6B

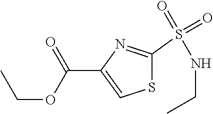 [SMILES: CCNS(=O)(=O)c1nc(C(=O)OCC)cs1]

Step 3B: To a solution of ethyl 2-(chlorosulfonyl) thiazole-4-carboxylate (Intermediate 5) (500 mg, 1.96 mmol) in DCM (10 mL) at 0° C., was added ethylamine (2M in THF) (3 mL, 5.87 mmol) dropwise, followed by addition of DIPEA (0.7 mL, 3.9 mmol) and the reaction mixture was stirred at 0° C. for 1h. TLC and LCMS of crude reaction mixture showed formation of desired mass. The reaction mixture was diluted with DCM and washed with saturated bicarbonate solution, water and brine. Dried over anhydrous sodium sulfate and concentrated in vacuum to get crude (˜350 mg). Crude was purified by flash chromatography (SiO2, 30-40% ethyl acetate-hexane) to get desired compound ethyl 2-(N-ethylsulfamoyl) thiazole-4-carboxylate (Intermediate 6B) as off white solid. (280 mg, 54%); MS (m/z): 265.3 [M+H].

### Intermediate 6C

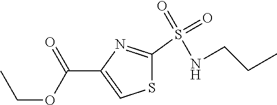 [SMILES: CCCNS(=O)(=O)c1nc(C(=O)OCC)cs1]

### ethyl 2-(N-propylsulfamoyl) thiazole-4-carboxylate

Step 3C: To a solution of ethyl 2-(chlorosulfonyl) thiazole-4-carboxylate (Intermediate 5) (500 mg, 1.95 mmol) in DCM (10 mL) at 0° C., was added propylamine (0.3 mL, 3.9 mmol; diluted in 1 mL DCM) dropwise and the reaction mixture was stirred at 0° C., followed by addition of DIPEA (0.7 mL, 3.9 mmol) and the reaction mixture was stirred at 0° C. for 1h. TLC and LCMS of crude reaction mixture showed formation of desired mass. The reaction was diluted with DCM and washed with saturated bicarbonate solution, water and brine. Dried over anhydrous sodium sulfate and concentrated in vacuum to get crude (˜550 mg). Crude was purified by flash chromatography

(SiO2, 30-40% ethyl acetate-hexane) to get desired compound ethyl 2-(N-propylsulfamoyl) thiazole-4-carboxylate (Intermediate 6C) as off white solid. (400 mg, 73%); MS (m/z): 278.6 [M+H].

### Intermediate 7A

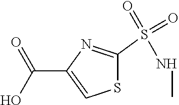

### 2-(N-methylsulfamoyl) thiazole-4-carboxylic acid

Step 4A: Starting material ethyl 2-(N-methylsulfamoyl) thiazole-4-carboxylate (Intermediate 6A) (170 mg, 0.68 mmol) was dissolved in THF/water (3:1) (4 mL) and then lithiumhydroxide monohydrate (86 mg, 2.0 mmol) was added and the reaction was stirred in room temperature for 3 h. After completion, LCMS and TLC of crude reaction material showed consumption of starting material and formation of desired mass. The reaction mixture was evaporated, dissolved in minimum volume of water, neutralized with 6N HCl carefully at 0° C. and filtered to collect crude solid 2-(N-methylsulfamoyl) thiazole-4-carboxylic acid (Intermediate 7A) as white solid, dried under vacuum for further use. (100 mg, 66%); MS (m/z): 221 [M+H].

### Intermediate 7R

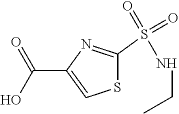

### 2-(N-ethylsulfamoyl) thiazole-4-carboxylic acid

Step-4B: Starting material ethyl 2-(N-ethylsulfamoyl) thiazole-4-carboxylate (Intermediate 6B) (230 mg, 0.87 mmol) was dissolved in THF/water (3:1) (4 mL) and then lithium hydroxide monohydrate (110 mg, 2.61 mmol) was added and the reaction was stirred in room temperature for 3 h. After completion, LCMS and TLC of crude reaction material showed consumption of starting material and formation of desired mass. The reaction mixture was evaporated, dissolved in minimum volume of water, neutralized with 6N HCl carefully at 0° C. and filtered to collect crude solid 2-(N-ethylsulfamoyl) thiazole-4-carboxylic acid (Intermediate 7B), dried under vacuum for further use. (130 mg, 63%); MS (m/z): 235.0 [M+H].

### Intermediate 7C

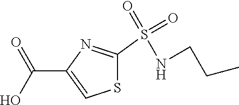

### 2-(N-propylsulfamoyl) thiazole-4-carboxylic acid

Step 4C: Starting material ethyl 2-(N-propylsulfamoyl) thiazole-4-carboxylate (Intermediate 6C) (100 mg, 0.36 mmol) was dissolved in THF/water (3:1) (4 mL) and then lithiumhydroxide monohydrate (45 mg, 1.07 mmol) was added and the reaction was stirred in room temperature for 3 h. After completion, LCMS and TLC of crude reaction material showed consumption of starting material and formation of desired mass. The reaction mixture was evaporated, dissolved in minimum volume of water, neutralized with 6N HCl carefully at 0° C. and filtered to collect crude solid 2-(N-propylsulfamoyl) thiazole-4-carboxylic acid (Intermediate 7C) as white solid, dried under vacuum for further use. (70 mg, 78%); MS (m/z): 249.2 [M−H].

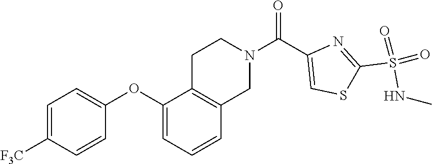 [SMILES: CNS(=O)(=O)c1nc(C(=O)N2CCc3c(cccc3Oc3ccc(C(F)(F)F)cc3)C2)cs1]

### N-methyl-4-(5-(4-(trifluoromethyl) phenoxy)-1,2,3,4-tetrahydroisoquinoline-2-carbonyl) thiazole-2-sulfonamide

Step-5A: Starting material 2-(N-methylsulfamoyl) thiazole-4-carboxylic acid (Intermediate 7A) (80 mg, 0.36 mmol) was dissolved in DMF (3 mL), to this solution EDC.HCl (103 mg, 0.54 mmol), HOBT (73 mg, 0.54 mmol) and DIPEA (0.3 mL, 1.8 mmol) were added sequentially at 0° C. and the resulting mixture was stirred for 10 minutes. To this reaction mixture, 5-(4-(trifluoromethyl) phenoxy)-1,2,3,4-tetrahydroisoquinoline hydrochloride (105 mg, 0.36 mmol) was added and the reaction mixture was allowed to stir for 5 h at room temperature. After completion of the reaction, TLC and LCMS of crude compound showed formation of desired mass. The reaction mixture was diluted with cold water and extracted with ethyl acetate (2×25 mL), washed with water (20 mL) and brine (20 mL), dried over anhydrous sodium sulphate and concentrated under vacuum to get crude (˜130 mg, ˜79% product enrichment according to LCMS, gum). Crude was purified by prep-HPLC method to get desired N-methyl-4-(5-(4-(trifluoromethyl) phenoxy)-1,2,3,4-tetrahydroisoquinoline-2-carbonyl) thiazole-2-sulfonamide as white solid. (47 mg, 26%); MS (m/z): 498 [M+H].

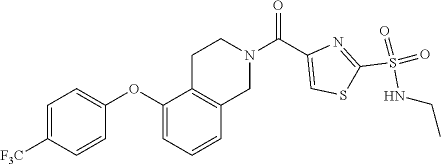 [SMILES: CCNS(=O)(=O)c1nc(C(=O)N2CCc3c(cccc3Oc3ccc(C(F)(F)F)cc3)C2)cs1]

### N-ethyl-4-(5-(4-(trifluoromethyl) phenoxy)-1,2,3,4-tetrahydroisoquinoline-2-carbonyl) thiazole-2-sulfonamide

Step-5B: Starting material 2-(N-methylsulfamoyl) thiazole-4-carboxylic acid (Intermediate 7A) (85 mg, 0.36 mmol) was dissolved in DMF (3 mL), to this solution EDC.HCl (103 mg, 0.54 mmol), HOBT (73 mg, 0.54 mmol) and DIPEA (0.3 mL, 1.8 mmol) were added sequentially at 0° C. and the resulting mixture was stirred for 10 minutes. To this reaction mixture, 5-(4-(trifluoromethyl) phenoxy)-1,2,3,4-tetrahydroisoquinoline hydrochloride (Opnatcg-05052022-TM35) (105 mg, 0.36 mmol) was added and the reaction mixture was allowed to stir for 5 h at room temperature. After completion of the reaction, TLC and LCMS of crude compound showed formation of desired mass. The reaction mixture was diluted with cold water and extracted with ethyl acetate (2×25 mL), washed with water (20 mL) and brine (20 mL), dried over anhydrous sodium sulphate and concentrated under vacuum to get crude (˜150 mg, ˜82% product enrichment according to LCMS, gum). Crude was purified by prep-HPLC method to get desired N-ethyl-4-(5-(4-(trifluoromethyl) phenoxy)-1,2,3,4-tetrahydroisoquinoline-2-carbonyl) thiazole-2-sulfonamide (Target Opnatcg-24-TM4) as white solid. (60 mg, 32%); MS (m/z): 512 [M+H].

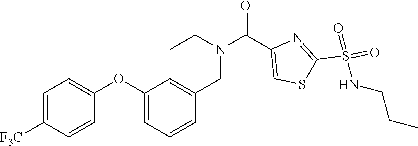 [SMILES: CCCNS(=O)(=O)c1nc(C(=O)N2CCc3c(cccc3Oc3ccc(C(F)(F)F)cc3)C2)cs1]

### N-propyl-4-(5-(4-(trifluoromethyl) phenoxy)-1,2,3,4-tetrahydroisoquinoline-2-carbonyl) thiazole-2-sulfonamide

Step 5C: Starting material 2-(N-methylsulfamoyl) thiazole-4-carboxylic acid (Intermediate 7A) (60 mg, 0.24 mmol) was dissolved in DMF (3 mL), to this solution EDC.HCl (69 mg, 0.36 mmol), HOBT (48 mg, 0.36 mmol) and DIPEA (0.2 mL, 1.2 mmol) were added sequentially at 0° C. and the resulting mixture was stirred for 10 minutes. To this reaction mixture, 5-(4-(trifluoromethyl) phenoxy)-1,2,3,4-tetrahydroisoquinoline hydrochloride (70 mg, 0.24 mmol) was added and the reaction mixture was allowed to stir for 5 h at room temperature. After completion of the reaction, TLC and LCMS of crude compound showed formation of desired mass. The reaction mixture was diluted with cold water and extracted with ethyl acetate (2×25 mL), washed with water (20 mL) and brine (20 mL), dried over anhydrous sodium sulphate and concentrated under vacuum to get crude (˜130 mg, ˜90% product enrichment according to LCMS, gum). Crude was purified by prep-HPLC method to get desired N-propyl-4-(5-(4-(trifluoromethyl) phenoxy)-1,2,3,4-tetrahydroisoquinoline-2-carbonyl) thiazole-2-sulfonamide as white solid. (72 mg, 57%); MS (m/z): 526 [M+H].

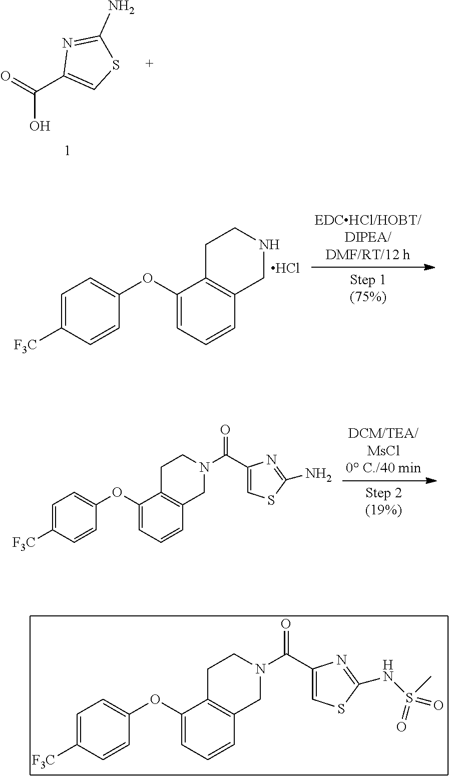

### Intermediate 24-TM9A

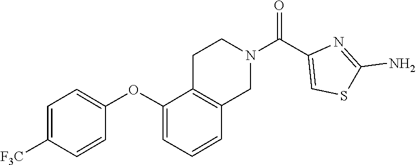 [SMILES: Nc1nc(C(=O)N2CCc3c(cccc3Oc3ccc(C(F)(F)F)cc3)C2)cs1]

### (2-aminothiazol-4-yl) (5-(4-(trifluoromethyl) phenoxy)-3,4-dihydroisoquinolin-2(1H)-yl)methanone

Step 1: To a stirred solution of 2-aminothiazole-4-carboxylic acid (1) (80 mg, 0.63 mmol) in DMF (1 mL), EDC.HCl (146 mg, 0.94 mmol), HOBT (127 mg, 0.94 mmol) and DIPEA (0.3 mL, 2.20 mmol) were added and the resulting mixture was stirred for 10 min. To this reaction mixture 5-(4-(trifluoromethyl) phenoxy)-1,2,3,4-tetrahydroisoquinoline hydrochloride (184 mg, 0.63 mmol) was added and the reaction mixture was allowed to stir for 12 h at room temperature. After completion of the reaction, ice was added to the reaction mixture and was extracted with ethyl acetate (2×50 mL), washed with brine (25 mL), dried over anhydrous sodium sulphate and concentrated under reduced pressure. Crude was purified by combi flash column chromatography (eluted at ˜70% Ethyl acetate in Hexane) to get (2-aminothiazol-4-yl) (5-(4-(trifluoromethyl) phenoxy)-3,4-dihydroisoquinolin-2(1H)-yl)methanone (200 mg, 75%) (24-TM9A) as yellow gum. MS (m/z): 420.0 [M+H].

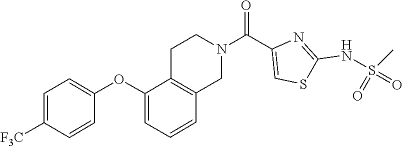 [SMILES: CS(=O)(=O)Nc1nc(C(=O)N2CCc3c(cccc3Oc3ccc(C(F)(F)F)cc3)C2)cs1]

### N-(4-(5-(4-(trifluoromethyl) phenoxy)-1,2,3,4-tetrahydroisoquinoline-2-carbonyl) thiazol-2-yl)methane sulfonamide

Step 2: To a stirred solution of (2-aminothiazol-4-yl) (5-(4-(trifluoromethyl) phenoxy)-3,4-dihydroisoquinolin-2(1H)-yl)methanone (24-TM9A) (150 mg, 0.35 mmol) in DCM (1 mL) at 0° C., was added TEA (0.1 mL, 1.07 mmol), followed by methanesulfonyl chloride (83 μL, 1.07 mmol). The reaction mixture was stirred at 0° C. for 40 min. After completion the reaction mixture was diluted with DCM (50 mL), washed with saturated bicarbonate solution (20 mL) and brine (15 mL), dried over anhydrous sodium sulfate. After concentration, after initial purification by combi flash column chromatography (eluted ˜20% Methanol in DCM), it was purified by Prep-HPLC purification method to get N-(4-(5-(4-(trifluoromethyl) phenoxy)-1,2,3,4-tetrahydroisoquinoline-2-carbonyl) thiazol-2-yl)methanesulfonamide (35 mg, 19%) as white solid. MS (m/z): 498 0 [M+H].

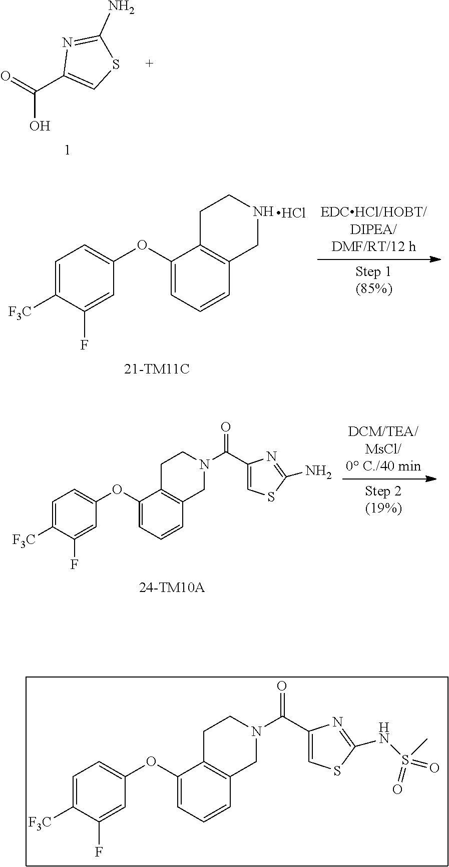

### Intermediate 24-TM10A

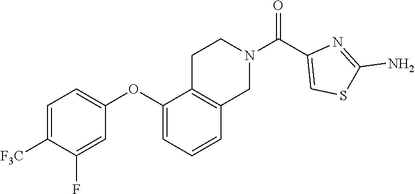 [SMILES: Nc1nc(C(=O)N2CCc3c(cccc3Oc3ccc(C(F)(F)F)c(F)c3)C2)cs1]

### (2-aminothiazol-4-yl) (5-(3-fluoro-4-(trifluoromethyl) phenoxy)-3,4-dihydroisoquinolin-2(1H)-yl)methanone

Step 1: To a stirred solution of 2-aminothiazole-4-carboxylic acid (1) (70 mg, 0.34 mmol) in DMF (1 mL), EDC.HCl (80 mg, 0.52 mmol), HOBT (70 mg, 0.52 mmol), and DIPEA (0.2 mL, 1.21 mmol) were added and the resulting mixture was stirred for 10 min. To this reaction mixture 5-(3-fluoro-4-(trifluoromethyl) phenoxy)-1,2,3,4-tetrahydroisoquinoline hydrochloride (21-TM11C) (107 mg, 0.34 mmol) was added and the reaction mixture was allowed to stir for 12 h at room temperature. After completion of the reaction, ice was added to the reaction mixture and was extracted with ethyl acetate (2×50 mL), washed with brine (25 mL), dried over anhydrous sodium sulphate and concentrated under reduced pressure. Crude was purified by combi flash column chromatography (eluted at ˜70% Ethyl acetate in Hexane) to get (2-aminothiazol-4-yl) (5-(3-fluoro-4-(trifluoromethyl) phenoxy)-3,4-dihydroisoquinolin-2(1H)-yl)methanone (130 mg, 85%) (24-TM10A) as yellow gum. MS (m/z): 438.0 [M+H].

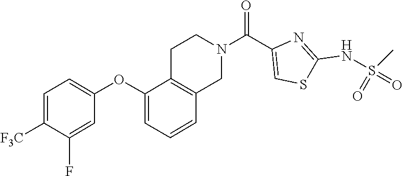 [SMILES: CS(=O)(=O)Nc1nc(C(=O)N2CCc3c(cccc3Oc3ccc(C(F)(F)F)c(F)c3)C2)cs1]

### N-(4-(5-(3-fluoro-4-(trifluoromethyl) phenoxy)-1,2,3,4-tetrahydroisoquinoline-2-carbonyl) thiazol-2-yl)methanesulfonamide

Step 2: To a stirred solution of (2-aminothiazol-4-yl) (5-(3-fluoro-4-(trifluoromethyl) phenoxy)-3,4-dihydroisoquinolin-2(1H)-yl)methanone (24-TM10A) (100 mg, 0.23 mmol) in DCM (1 mL) at 0° C., was added TEA (99 μL, 0.71 mmol), followed by Methanesulfonyl chloride (55 μL, 0.71 mmol). The reaction mixture was stirred at 0° C. for 40 min. After completion the reaction mixture was diluted with DCM (50 mL), washed with saturated bicarbonate solution (20 mL) and brine (15 mL), dried over anhydrous sodium sulfate. After concentration, after initial purification by combi flash column chromatography (eluted ˜20% Methanol in DCM), it was purified by Prep-HPLC purification method to get N-(4-(5-(3-fluoro-4-(trifluoromethyl) phenoxy)-1,2,3,4-tetrahydroisoquinoline-2-carbonyl) thiazol-2-yl)methanesulfonamide (23 mg, 19%) as white solid. MS (m/z): 516 0 [M+H].

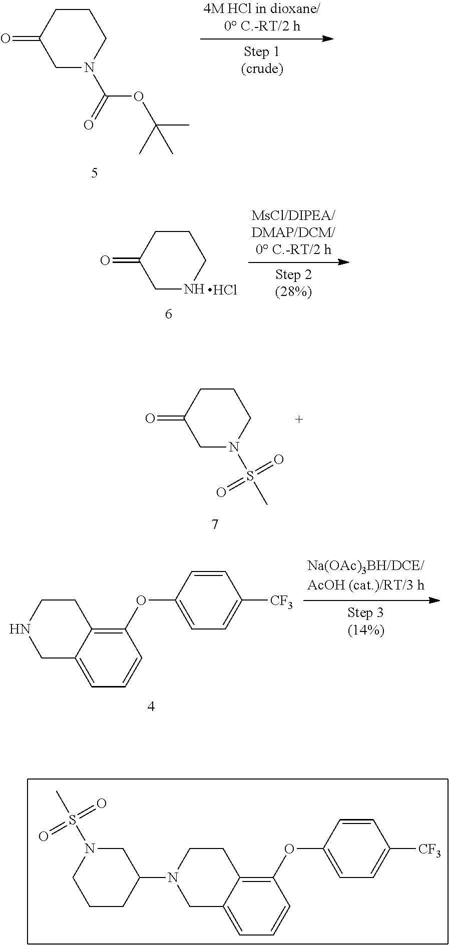

### Intermediate-6

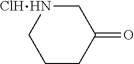

### piperidin-3-one hydrochloride

Step 1: To a stirred solution of tert-butyl 3-oxopiperidine-1-carboxylate (5) (250 mg, 1.25 mmol) in dichloromethane (3 mL) was added 4M dioxane-HCl (1.0 mL) at 0° C. The reaction mixture was allowed to stir for 2 h at room temperature under argon atmosphere. After completion of the reaction, the reaction mixture was concentrated under reduced pressure and triturated with n-pentane and ether to provide the desire compound piperidin-3-one hydrochloride (Intermediate 8) (120.0 mg) as crude. This crude compound has used in the next step without further purification. MS (m/z): 99.9 [M+H].

### Intermediate-7

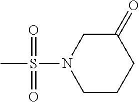

### 1-(methylsulfonyl) piperidin-3-one

Step 2: To a stirred solution of piperidin-3-one hydrochloride (6) (190 mg, 1.40 mmol) in dichloromethane (3 mL) was added DIPEA (0.76 mL, 4.22 mmol), catalytic amount DMAP. After stirring for 30 min at 0° C., methanesulfonyl chloride (0.16 mL, 2.11 mmol) was added in the reaction and further stirred for 2 h. After completion, reaction mixture was quenched with cold water (10 mL), made the reaction mixture basic using saturated sodium bicarbonate solution (5 mL), and extracted the with dichloromethane (3×50 mL), dried over anhydrous sodium sulfate, concentrated in vacuo to get crude and the crude compound was purified by combi-flash chromatography to afford 1-(methylsulfonyl) piperidin-3-one (7) (70 mg, 28%) as white solid. MS (m/z): 177.1 [M+H].

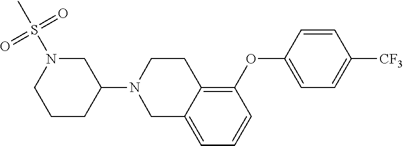 [SMILES: CS(=O)(=O)N1CCCC(N2CCc3c(cccc3Oc3ccc(C(F)(F)F)cc3)C2)C1]

### 2-(1-(methylsulfonyl) piperidin-3-yl)-5-(4-(trifluoromethyl) phenoxy)-1,2,3,4-tetrahydroisoquinoline

Step 3: To a stirred solution of 5-(4-(trifluoromethyl) phenoxy)-1,2,3,4-tetrahydroisoquinoline (4) (300 mg, 1.02 mmol) and 1-(methylsulfonyl) piperidin-3-one (7) (272 mg, 1.54 mmol) in DCE was added catalytic amount of acetic acid at room temperature. After stirring for 5 min, sodium triacetoxyborohydride (868 mg, 4.1 mmol) was added in the reaction mixture and stirred for 3 h. After completion, reaction mixture was quenched with sat NaHCO₃ solution (10 mL), diluted with EtOAc (300 mL), washed the organic part with water (2×50 mL), dried the organic part over anhydrous sodium sulfate, concentrated under vacuo to get the crude and the crude was purified by prep-HPLC purification to afford 2-(1-(methylsulfonyl) piperidin-3-yl)-5-(4-(trifluoromethyl) phenoxy)-1,2,3,4-tetrahydroisoquinoline (Opnatcg-08-TM7) (66 mg, 14%) as white solid. MS (m/z): 455.4 [M+H].

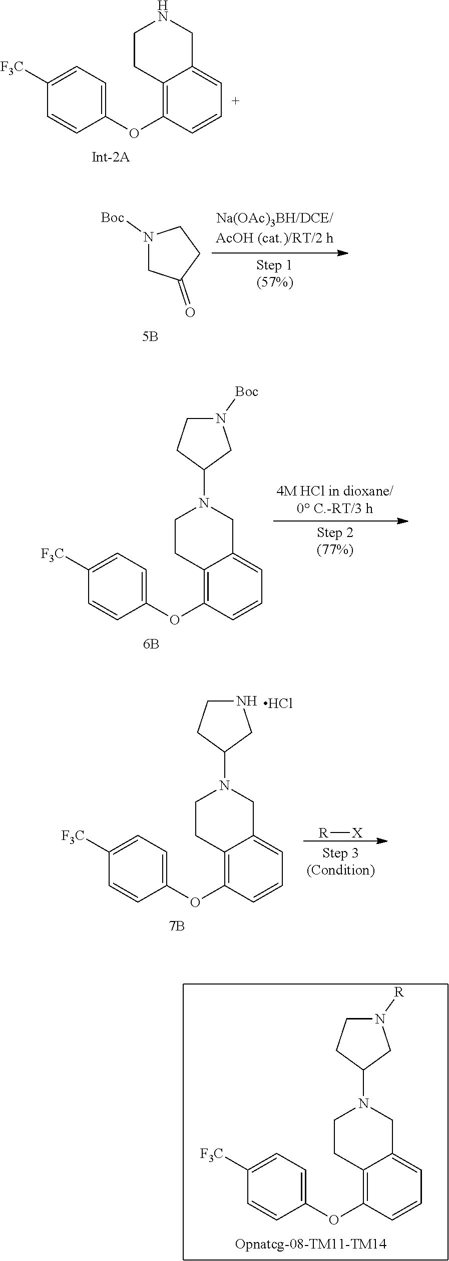

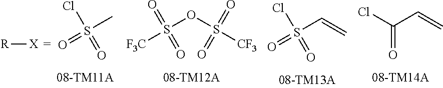

### Intermediate 6B

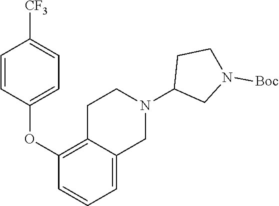 [SMILES: CC(C)(C)OC(=O)N1CCC(N2CCc3c(cccc3Oc3ccc(C(F)(F)F)cc3)C2)C1]

### tert-butyl 3-(5-(4-(trifluoromethyl) phenoxy)-3,4-dihydroisoquinolin-2(1H)-yl) pyrrolidine-1-carboxylate

Step-1: To a stirred solution of 5-(4-(trifluoromethyl) phenoxy)-1,2,3,4-tetrahydroisoquinoline (Int-2A, 500 mg, 1.70 mmol) and tert-butyl 3-oxopyrrolidine-1-carboxylate (5B) (474 mg, 2.56 mmol) in 1,2-dichloroethane (10 mL), catalytic amount of acetic acid (10 mL) was added and the resulting reaction mixture was stirred for 5 minutes at room temperature. To this reaction mixture, was added sodium triacetoxyborohydride (1.45 g, 6.82 mmol.) and the resulting reaction mixture was stirred at room temperature for 2 h. LCMS of crude reaction mixture showed formation of desired mass. Crude reaction mixture was diluted with DCM (100 mL), washed with saturated sodium bicarbonate solution (2×50 mL), saturated brine solution (50 mL) and dried over anhydrous sodium sulfate, filtered and evaporated under reduced pressure to obtain crude reaction mixture. Crude was purified by flash chromatography (30% EtOAc in Hexane) to get tert-butyl 3-(5-(4-(trifluoromethyl) phenoxy)-3,4-dihydroisoquinolin-2(1H)-yl) pyrrolidine-1-carboxylate (6B) (450 mg, 57%) as white solid. MS (m/z): 463.6 [M+H].

### Intermediate 7B

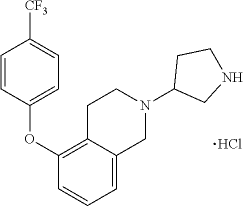

### 2-(pyrrolidin-3-yl)-5-(4-(trifluoromethyl) phenoxy)-1,2,3,4-tetrahydroisoquinoline hydrochloride

Step-2: tert-butyl 3-(5-(4-(trifluoromethyl) phenoxy)-3,4-dihydroisoquinolin-2(1H)-yl) pyrrole dine-1-carboxylate (6B) (300 mg, 0.65 mmol) was dissolved in DCM (5 mL) and to this 4M HCl in dioxane (0.5 mL, 1.95 mmol) was added in ice cold condition and then the reaction mixture was stirred at room temperature for 3 h. After completion of the reaction, crude LCMS showed formation of desired product mass. Reaction mixture was evaporated to dryness, triturated with ether and pentane for three times and dried under reduced pressure to get 2-(pyrrolidin-3-yl)-5-(4-(trifluoromethyl) phenoxy)-1,2,3,4-tetrahydroisoquinoline hydro chloride (Intermediate 7B) (200 mg, 77%) as pale brown solid. MS (m/z): 362.8 [M+H].

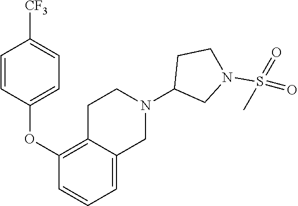 [SMILES: CS(=O)(=O)N1CCC(N2CCc3c(cccc3Oc3ccc(C(F)(F)F)cc3)C2)C1]

### 2-(1-(methylsulfonyl) pyrrolidin-3-yl)-5-(4-(trifluoromethyl) phenoxy)-1,2,3,4-tetrahydroisoquinoline

Step-3: To a stirred solution of 2-(pyrrolidin-3-yl)-5-(4-(trifluoromethyl) phenoxy)-1,2,3,4-tetrahydroisoquinoline hydrochloride (7B) (100 mg, 0.25 mmol) in dry DCM (5 mL) at 0° C., was added triethylamine (0.1 mL, 0.82 mmol), followed by mesyl chloride (32 mL, 0.41 mmol) and the reaction mixture was stirred at 0° C. to room temperature over 1 h. After completion, as monitored by TLC and LCMS of crude reaction mixture, the reaction mixture was diluted with DCM (20 mL), washed with saturated sodium bicarbonate solution (10 mL), saturated brine solution (10 mL) and dried over anhydrous sodium sulfate. After concentration, crude was purified by prep-HPLC purification method to get desired 2-(1-(methylsulfonyl) pyrrolidin-3-yl)-5-(4-(trifluoromethyl) phenoxy)-1,2,3,4-tetrahydroisoquinoline (72 mg, 59%) as off white solid. MS (m/z): 441.2 [M+H].

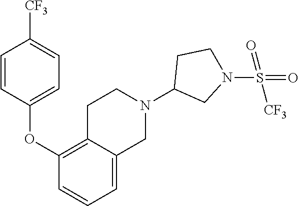 [SMILES: O=S(=O)(N1CCC(N2CCc3c(cccc3Oc3ccc(C(F)(F)F)cc3)C2)C1)C(F)(F)F]

### 5-(4-(trifluoromethyl) phenoxy)-2-(1-((trifluoromethyl) sulfonyl) pyrrolidin-3-yl)-1,2,3,4-tetrahydroisoquinoline

Step-3: To a stirred solution of 2-(pyrrolidin-3-yl)-5-(4-(trifluoromethyl) phenoxy)-1,2,3,4-tetrahydroisoquinoline hydrochloride (7B) (100 mg, 0.25 mmol), in dry DCM (4 mL) at 0° C., was added N,N-diisopropylethylamine (0.14 mL, 0.82 mmol) and catalytic amount of DMAP, followed by slow addition of triflic anhydride (46 mL, 0.27 mmol) in dry DCM (1 mL). The reaction mixture was stirred at 0° C. to room temperature over 1 h. After completion, as monitored by TLC and LCMS of crude reaction mixture, the reaction mixture was diluted with DCM (20 mL), washed with saturated sodium bicarbonate solution (10 mL), saturated brine solution (10 mL) and dried over anhydrous sodium sulfate. After concentration, crude was purified by prep-HPLC purification method to get desired 5-(4-(trifluoromethyl) phenoxy)-2-(1-((trifluoromethyl) sulfonyl) pyrrolidin-3-yl)-1,2,3,4-tetrahydroisoquino line (55 mg, 40%) as white solid. MS (m/z): 495.1 [M+H].

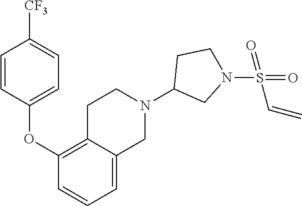 [SMILES: C=CS(=O)(=O)N1CCC(N2CCc3c(cccc3Oc3ccc(C(F)(F)F)cc3)C2)C1]

### 5-(4-(trifluoromethyl) phenoxy)-2-(1-(vinylsulfonyl) pyrrolidin-3-yl)-1,2,3,4-tetrahydroiso quinoline

Step-3: To a stirred solution of 2-(pyrrolidin-3-yl)-5-(4-(trifluoromethyl) phenoxy)-1,2,3,4-tetrahydro isoquinoline hydrochloride (7B) (100 mg, 0.25 mmol) in dry DCM (4 mL) at −10° C., was added N,N-diisopropylethylamine (0.15 mL, 0.82 mmol) and catalytic amount of DMAP, followed by slow addition of vinylsulfonyl chloride stock solution (a stock solution of 0.1 mL of vinylsulfonyl chloride and 0.9 mL of dry DCM was prepared, from this stock solution 0.37 mL) to the reaction mixture at −10° C. Reaction mixture was stirred at −10° C. for 40 min. After completion, as monitored by TLC (5% MeOH-DCM) and LCMS of crude, reaction mixture was diluted with DCM (5 mL). Crude reaction mixture was concentrated and initially purified by flash chromatography (flash silica, 230-400 mesh, 0-2% MeOH-DCM as eluent) to get ˜70 mg crude desired compound. This crude compound, after final prep-HPLC purification yielded desired 5-(4-(trifluoromethyl) phenoxy)-2-(1-(vinylsulfonyl) pyrrolidin-3-yl)-1,2,3,4-tetrahydroisoquinoline (26 mg, 21%) as white solid. MS (m/z): 441.2 [M+H].

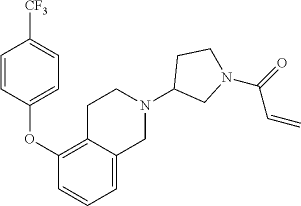 [SMILES: C=CC(=O)N1CCC(N2CCc3c(cccc3Oc3ccc(C(F)(F)F)cc3)C2)C1]

### 1-(3-(5-(4-(trifluoromethyl)phenoxy)-3,4-dihydroisoquinolin-2(1H)-yl)pyrrolidin-1-yl)prop-2-en-1-one

Step-3: To a stirred solution of 2-(pyrrolidin-3-yl)-5-(4-(trifluoromethyl) phenoxy)-1,2,3,4-tetrahydroisoquinoline hydrochloride (7B) (100 mg, 0.29 mmol) in dry DCM (4 mL) at −10° C., was added triethylamine (0.12 mL, 0.86 mmol), followed by slow addition of acryloyl chloride stock solution (a stock solution of 0.1 mL of acryloyl chloride and 0.9 mL of dry DCM was prepared, from this stock solution 0.33 mL, 0.43 mmol.) was added dropwise to the reaction mixture at −10° C. Reaction mixture was stirred at −10° C. for 40 min. After completion, as monitored by TLC (5% MeOH-DCM) and LCMS of crude, reaction mixture was diluted with DCM (5 mL). Crude reaction mixture was concentrated and initially purified by flash chromatography (flash silica, 230-400 mesh, 0-2% MeOH-DCM as eluent) to get ˜80 mg crude desired compound. This crude compound after final prep-HPLC purification yielded desired 1-(3-(5-(4-(trifluoromethyl) phenoxy)-3,4-dihydroisoquinolin-2(1H)-yl) pyrrolidin-1-yl) prop-2-en-1-one (30 mg, 26%) as off white solid. MS (m/z): 417.1 [M+H].

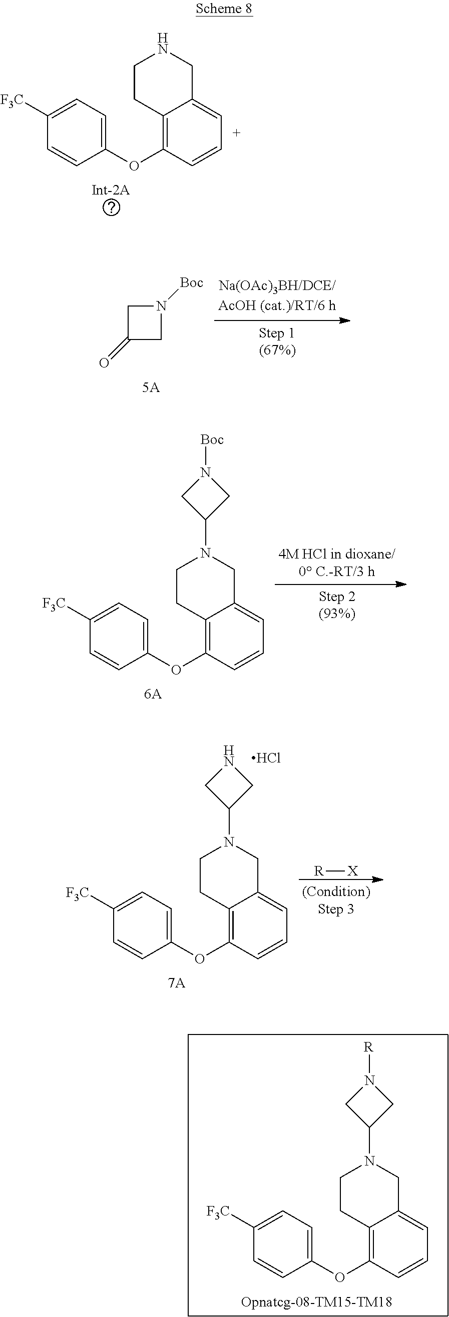

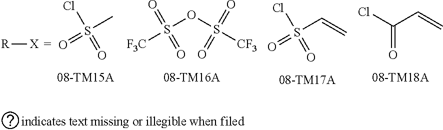

### Intermediate 6A

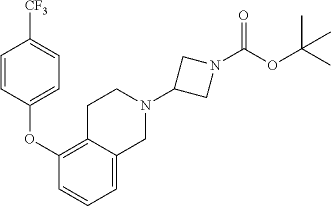 [SMILES: CC(C)(C)OC(=O)N1CC(N2CCc3c(cccc3Oc3ccc(C(F)(F)F)cc3)C2)C1]

### tert-butyl 3-(5-(4-(trifluoromethyl) phenoxy)-3,4-dihydroisoquinolin-2(1H)-yl) azetidine-1-carboxylate

Step-1: To a stirred solution of 5-(4-(trifluoromethyl) phenoxy)-1,2,3,4-tetrahydroisoquinoline (Int-2A, 500 mg, 1.70 mmol) and tert-butyl 3-oxoazetidine-1-carboxylate (5A) (438 mg, 2.56 mmol) in 1,2-dichloroethane solvent (10 mL), catalytic amount of acetic acid (10 mL) was added and the resulting reaction mixture was stirred for 5 minutes at room temperature. To this reaction mixture, was added sodium triacetoxyborohydride (1.45 g, 6.82 mmol.) and the resulting reaction mixture was stirred at room temperature for 2 h. LCMS of crude reaction mixture showed formation of desired mass. Crude reaction mixture was diluted with DCM (100 mL), washed with saturated sodium bicarbonate solution (50 mL) two times, saturated brine solution (50 mL) and dried over anhydrous sodium sulfate, filtered and evaporated under reduced pressure to obtain crude reaction mixture. Crude was purified by flash chromatography (35-40% EtOAc-Hexane) to get tert-butyl 3-(5-(4-(trifluoromethyl) phenoxy)-3,4-dihydroisoquinolin-2(1H)-yl) azetidine-1-carboxylate (6A) (510 mg, 67%) as off-white solid. MS (m/z): 448.8 [M+H].

### Intermediate 7A

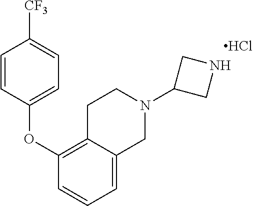 [SMILES: FC(F)(F)c1ccc(Oc2cccc3c2CCN(C2CNC2)C3)cc1 ; Cl]

### 2-(azetidin-3-yl)-5-(4-(trifluoromethyl) phenoxy)-1,2,3,4-tetrahydroisoquinoline hydrochloride

Step-2: tert-butyl 3-(5-(4-(trifluoromethyl) phenoxy)-3,4-dihydroisoquinolin-2(1H)-yl) azetidine-1-carboxylate (6A) (400 mg, 0.89 mmol) was dissolved in 1,4-Dioxane (4 mL) and to this 4M HC in dioxane (2.2 mL, 8.92 mmol) was added in ice cold condition and then the reaction mixture was stirred at room temperature for 3 h. After completion of the reaction, crude LCMS showed formation of desired product mass. Reaction mixture was evaporated to dryness, triturated with ether and pentane for three times and dried under reduced pressure to get 2-(azetidin-3-yl)-5-(4-(trifluoromethyl) phenoxy)-1,2,3,4-tetrahydroisoquinoline hydrochloride (7A) (290 mg, 93%) as off white solid. MS (m/z): 349.3 [M+H].

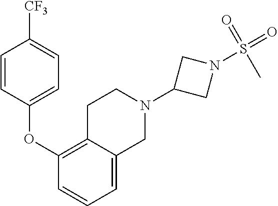 [SMILES: CS(=O)(=O)N1CC(N2CCc3c(cccc3Oc3ccc(C(F)(F)F)cc3)C2)C1]

### 2-(1-(methylsulfonyl) azetidin-3-yl)-5-(4-(trifluoromethyl) phenoxy)-1,2,3,4-tetrahydroisoquinoline

Step-3: To a stirred solution of 2-(azetidin-3-yl)-5-(4-(trifluoromethyl) phenoxy)-1,2,3,4-tetrahydroisoquinoline hydrochloride (7A) (70 mg, 0.20 mmol) in dry DCM (5 mL) at 0° C., was added triethylamine (84 mL, 0.60 mmol), followed by mesyl chloride (08-TM15A) (23 mL, 0.30 mmol) and the reaction mixture was stirred at 0° C. to room temperature over 1 h. After completion, as monitored by TLC and LCMS of crude reaction mixture, the reaction mixture was diluted with DCM (20 mL), washed with saturated sodium bicarbonate solution (10 mL), saturated brine solution (10 mL) and dried over anhydrous sodium sulfate. After concentration, crude was purified by prep-HPLC purification method to get desired 2-(1-(methylsulfonyl) azetidin-3-yl)-5-(4-(trifluoromethyl) phenoxy)-1,2,3,4-tetrahydroisoquinoline (40 mg, 46%) as off white sticky solid. MS (m/z): 427.1 [M+H].

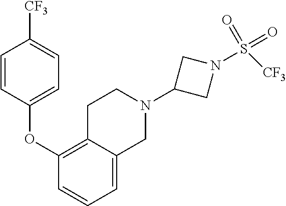 [SMILES: O=S(=O)(N1CC(N2CCc3c(cccc3Oc3ccc(C(F)(F)F)cc3)C2)C1)C(F)(F)F]

### 5-(4-(trifluoromethyl) phenoxy)-2-(1-((trifluoromethyl) sulfonyl) azetidin-3-yl)-1,2,3,4-tetra hydroiso quinoline

Step-3: To a stirred solution of 2-(azetidin-3-yl)-5-(4-(trifluoromethyl) phenoxy)-1,2,3,4-tetrahydroisoquinoline hydrochloride (7A) (100 mg, 0.29 mmol) in dry DCM (5 mL), at 0° C., was added N,N-diisopropylethylamine (0.15 mL, 0.86 mmol) and catalytic amount of DMAP, followed by slow addition of triflic anhydride (08-TM16A) (72 mL, 0.43 mmol) in dry DCM (1 mL). The reaction mixture was stirred at 0° C. to room temperature over 1 h. After completion, as monitored by TLC and LCMS of crude reaction mixture, the reaction mixture was diluted with DCM (20 mL), washed with saturated sodium bicarbonate solution (10 mL), saturated brine solution (10 mL) and dried over anhydrous sodium sulfate. After concentration, crude was purified by prep-HPLC purification method to get desired 5-(4-(trifluoromethyl) phenoxy)-2-(1-((trifluoromethyl) sulfonyl) azetidin-3-yl)-1,2,3,4-tetrahydroisoquinoline (55 mg, 34%) as light brown gum. MS (m/z): 481.1 [M+H].

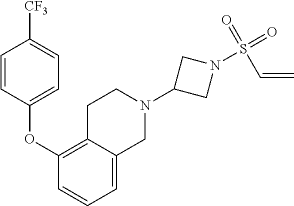 [SMILES: C=CS(=O)(=O)N1CC(N2CCc3c(cccc3Oc3ccc(C(F)(F)F)cc3)C2)C1]

### 5-(4-(trifluoromethyl) phenoxy)-2-(1-(vinylsulfonyl) azetidin-3-yl)-1,2,3,4-tetrahydroiso quinoline

Step-3: To a stirred solution of 2-(azetidin-3-yl)-5-(4-(trifluoromethyl) phenoxy)-1,2,3,4-tetrahydroisoquinoline hydrochloride (7A) (100 mg, 0.29 mmol) in dry DCM (4 mL) at −10° C., was added N,N-diisopropylethylamine (0.14 mL, 0.82 mmol) and catalytic amount of DMAP, followed by slow addition of vinylsulfonyl chloride (08-TM17A) stock solution (a stock solution of 0.1 mL of vinylsulfonyl chloride and 0.9 mL of dry DCM was prepared, from this stock solution 0.37 mL, 0.41 mmol.) to the reaction mixture at −10° C. Reaction mixture was stirred at −10° C. for 40 min. After completion, as monitored by TLC (5% MeOH-DCM).and LCMS of crude, reaction mixture was diluted with DCM (5 mL). Crude reaction mixture was concentrated and initially purified by flash chromatography (flash silica, 230-400 mesh, 0-2% MeOH-DCM as eluent) to get ˜80 mg of crude desired compound. This compound, after final prep-HPLC purification yielded desired 5-(4-(trifluoromethyl) phenoxy)-2-(1-(vinylsulfonyl) azetidin-3-yl)-1,2,3,4-tetrahydroisoquinoline (16 mg, 13%) as off white sticky solid. MS (m/z): 439.1 [M+H].

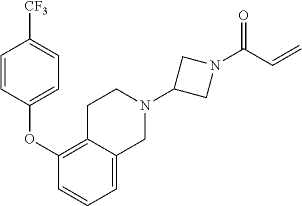 [SMILES: C=CC(=O)N1CC(N2CCc3c(cccc3Oc3ccc(C(F)(F)F)cc3)C2)C1]

### 1-(3-(5-(4-(trifluoromethyl) phenoxy)-3,4-dihydroisoquinolin-2(1H)-yl) azetidin-1-yl) prop-2-en-1-one

Step-3: To a stirred solution of 2-(azetidin-3-yl)-5-(4-(trifluoromethyl) phenoxy)-1,2,3,4-tetrahydroisoquinoline hydrochloride (7B) (100 mg, 0.29 mmol) in dry DCM (4 mL) at −10° C., was added triethylamine (0.12 mL, 0.86 mmol), followed by slow addition of acryloyl chloride stock solution (a stock solution of 0.1 mL of acryloyl chloride (08-TM18A) and 0.9 mL of dry DCM was prepared, from this stock solution 0.35 mL) was added dropwise to the reaction mixture at −10° C. Reaction mixture was stirred at −10° C. for 40 min. After completion, as monitored by TLC (5% MeOH-DCM) and LCMS of crude, reaction mixture was diluted with DCM (5 mL). Crude reaction mixture was concentrated and initially purified by flash chromatography (flash silica, 230-400 mesh, 0-2% MeOH-DCM as eluent) to get ˜ 80 mg desired crude compound. This crude compound, after final prep-HPLC purification yielded desired 1-(3-(5-(4-(trifluoromethyl) phenoxy)-3,4-dihydroisoquinolin-2(1H)-yl) azetidin-1-yl) prop-2-en-1-one (30 mg, 26%) as off white solid. MS (m/z): 403.4 [M+H].

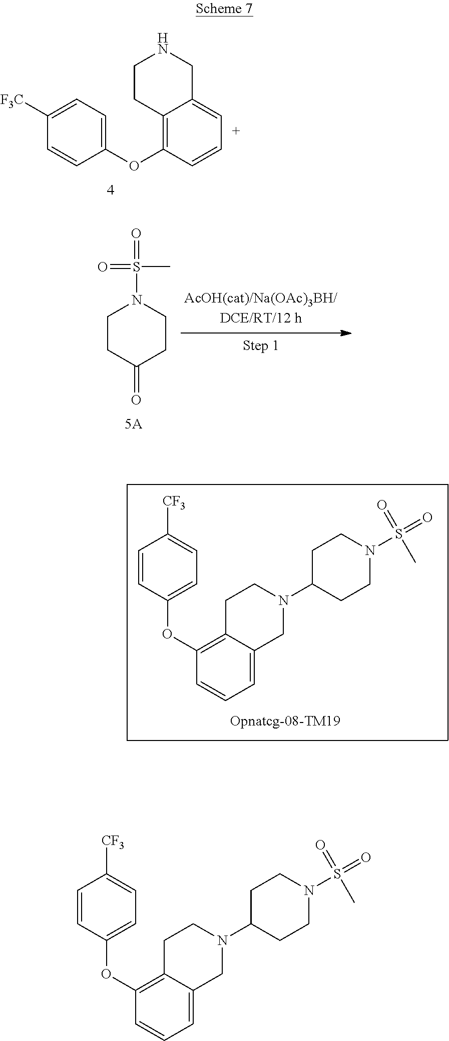 [SMILES: FC(F)(F)c1ccc(Oc2cccc3c2CCNC3)cc1 ; CS(=O)(=O)N1CCC(=O)CC1 ; CS(=O)(=O)N1CCC(N2CCc3c(cccc3Oc3ccc(C(F)(F)F)cc3)C2)CC1 ; CS(=O)(=O)N1CCC(N2CCc3c(cccc3Oc3ccc(C(F)(F)F)cc3)C2)CC1]

### 2-(1-(methylsulfonyl) piperidin-4-yl)-5-(4-(trifluoromethyl) phenoxy)-1,2,3,4-tetrahydroisoquinoline

Step-1: To a stirred solution of 5-(4-(trifluoromethyl) phenoxy)-1,2,3,4-tetrahydroisoquinoline (4) (50.mg, 0.17 mmol) in DCE (2 mL) were added 1-(methylsulfonyl) piperidin-4-one (5A) (60 mg, 0.34 mmol) and acetic acid (0.05 mL) and the reaction mixture was allowed to stir at room temperature for 5 min, then sodium triacetoxyborohydride (180 mg, 0.85 mmol) was added to it and was stirred for another 12 h at room temperature. After completion of reaction, reaction mixture was concentrated to dryness, quenched with water (1×50 mL) and extracted with 10% MeOH in DCM (3×50 mL). The organic layers were washed with water (3×50 mL), brine (500 mL), dried over anhydrous sodium sulfate. The organic layer was concentrated and the resulted crude was purified by Prep-HPLC purification method to get the desired product 2-(1-(methylsulfonyl) piperidin-4-yl)-5-(4-(trifluoromethyl) phenoxy)-1,2,3,4-tetrahydroisoquinoline (15 mg, 20%) as white solid. MS (m/z): 455.1 [M+H].

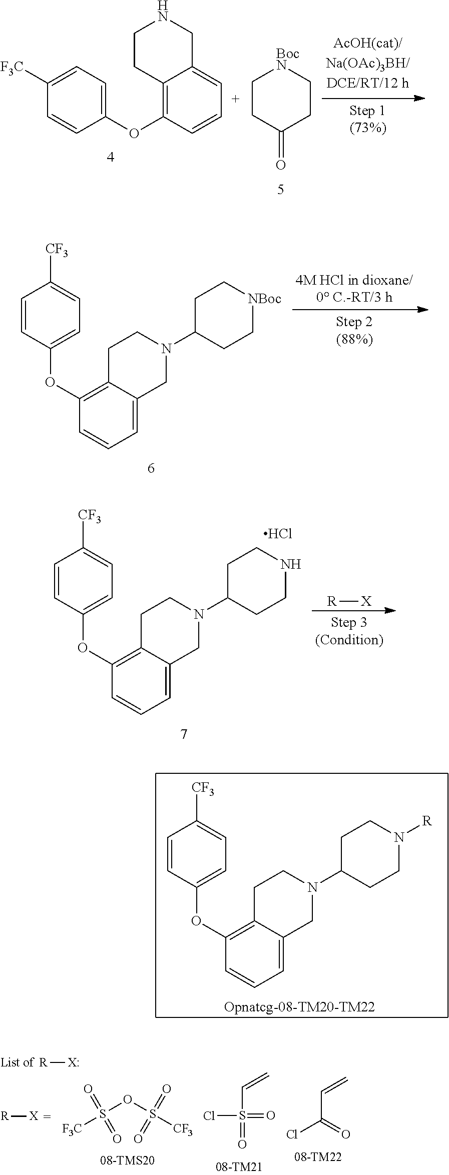

### Intermediate 6

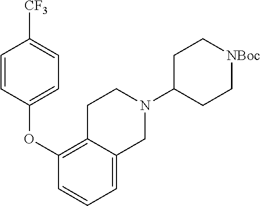 [SMILES: CC(C)(C)OC(=O)N1CCC(N2CCc3c(cccc3Oc3ccc(C(F)(F)F)cc3)C2)CC1]

### tert-butyl 4-(5-(4-(trifluoromethyl) phenoxy)-3,4-dihydroisoquinolin-2(1H)-yl) piperidine-1-carboxylate

Step-1: To a stirred solution of 5-(4-(trifluoromethyl) phenoxy)-1,2,3,4-tetrahydroisoquinoline (4) (500 mg, 1.71 mmol) in DCE (2 mL) were added tert-butyl 4-oxopiperidine-1-carboxylate (679 mg, 3.41 mmol) and acetic acid (0.05 mL) and the reaction mixture was allowed to stir at room temperature for 5 min, then sodium triacetoxyborohydride (1.8 g, 8.53 mmol) was added to it and continuously stir for another 12 h at room temperature. The reaction mixture was concentrated to dryness, quenched with water (1×50 mL) and was extracted in 10% MeOH in DCM (3×50 mL). The organic layers were washed with water (3×50 mL), brine (500 mL), dried over anhydrous sodium sulfate. The organic layer was concentrated and the resulted crude mass was purified by combi flash column chromatography, eluting with 40-45% EtOAc/Hexane to get the desired product tert-butyl 4-(5-(4-(trifluoromethyl) phenoxy)-3,4-dihydroisoquinolin-2(1H)-yl) piperidine-1-carboxylate (6) (600 mg, 73%) as white solid. MS (m/z): 477.2 [M +H].

### Intermediate 7

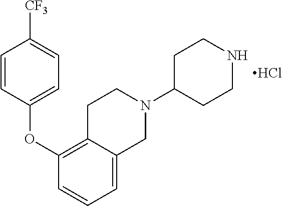 [SMILES: FC(F)(F)c1ccc(Oc2cccc3c2CCN(C2CCNCC2)C3)cc1 ; Cl]

### 2-(piperidin-4-yl)-5-(4-(trifluoromethyl) phenoxy)-1,2,3,4-tetrahydroisoquinoline hydrochloride

Step-2: tert-butyl 4-(5-(4-(trifluoromethyl) phenoxy)-3,4-dihydroisoquinolin-2(1H)-yl)piperidine-1-carboxylate (6) (500 mg, 1.05 mmol) was dissolved in DCM (5 mL). To this 4M HCl in dioxane (2 mL) was added in ice-cold condition and then the reaction mixture was stirred at room temperature for 3 h. After completion of the reaction, the reaction mixture was evaporated under reduced pressure to get a solid mass. This solid mass was triturated with ether and pentane for three times to get 2-(piperidin-4-yl)-5-(4-(trifluoromethyl) phenoxy)-1,2,3,4-tetrahydroisoquinoline hydrochloride (7) (350 mg, 88%) as white solid. MS (m/z): 377.2 [M+H].

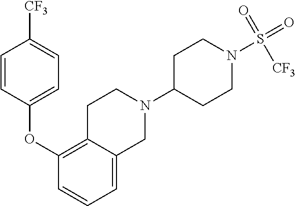 [SMILES: O=S(=O)(N1CCC(N2CCc3c(cccc3Oc3ccc(C(F)(F)F)cc3)C2)CC1)C(F)(F)F]

### 5-(4-(trifluoromethyl) phenoxy)-2-(1-((trifluoromethyl) sulfonyl) piperidin-4-yl)-1,2,3,4-tetrahydroisoquinoline

Step-3: To a stirred solution of 2-(piperidin-4-yl)-5-(4-(trifluoromethyl) phenoxy)-1,2,3,4-tetrahydro isoquinoline hydrochloride (7) (70 mg, 0.17 mmol) in DCM (4 mL) at 0° C., was added DIPEA (0.12 mL, 0.52 mmol), followed by triflic anhydride (08-TM20) (58 μL, 0.34 mmol). The reaction mixture was stirred at 0° C. until reaches slowly at room temperature. After completion the reaction mixture was diluted with DCM (50 mL), washed with saturated bicarbonate solution (20 mL) and brine (15 mL), dried over anhydrous sodium sulfate. After concentration, crude was purified by Prep-HPLC purification method to get 5-(4-(trifluoromethyl) phenoxy)-2-(1-((trifluoromethyl) sulfonyl) piperidin-4-yl)-1,2,3,4-tetrahydroisoquinoline (15 mg, 17%) as light brown sticky solid. MS (m/z): 509.3 [M+H].

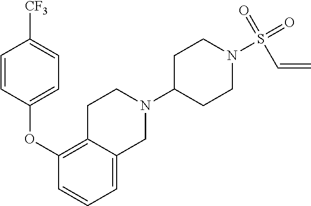 [SMILES: C=CS(=O)(=O)N1CCC(N2CCc3c(cccc3Oc3ccc(C(F)(F)F)cc3)C2)CC1]

### 5-(4-(trifluoromethyl) phenoxy)-2-(1-(vinylsulfonyl) piperidin-4-yl)-1,2,3,4-tetrahydroisoquinoline

Step-4: To a stirred solution of 2-(piperidin-4-yl)-5-(4-(trifluoromethyl) phenoxy)-1,2,3,4-tetrahydroisoquinoline hydrochloride (7) (100 mg, 0.26 mmol) in dry DCM (2 mL) at 0° C., was added DIPEA (0.1 mL, 0.79 mmol), followed by addition of catalytic amount of DMAP. To this reaction mixture, vinylsulfonyl chloride (08-TM20) (40 μL, 0.53 mmol) in DCM at 0° C. was added dropwise. Reaction mixture was allowed to stir at 0° C. to RT for 30 min. Reaction mixture was diluted with DCM (5 mL), After concentration, crude was initially purified by combi-flash chromatography (0-3% MeOH-DCM) and after Prep-HPLC purification provided 5-(4-(trifluoromethyl) phenoxy)-2-(1-(vinylsulfonyl) piperidin-4-yl)-1,2,3,4-tetrahydroisoquinoline (Opnatcg-08-TM21) (6 mg, 6%) as white solid. MS (m/z): 467.2 [M+H].

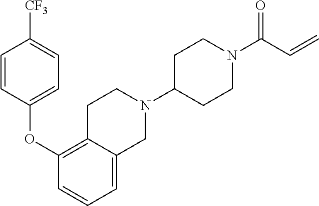 [SMILES: C=CC(=O)N1CCC(N2CCc3c(cccc3Oc3ccc(C(F)(F)F)cc3)C2)CC1]

### 1-(4-(5-(4-(trifluoromethyl) phenoxy)-3,4-dihydroisoquinolin-2(1H)-yl) piperidin-1-yl) prop-2-en-1-one

Step-5: To a stirred solution of 2-(piperidin-4-yl)-5-(4-(trifluoromethyl) phenoxy)-1,2,3,4-tetrahydroisoquinoline hydrochloride (7) (70 mg, 0.18 mmol) in dry DCM (2 mL) at −20° C., was added DIPEA (0.1 mL, 0.56 mmol), followed by dropwise addition of acryloyl chloride (08-TM21) (20 μL, 0.37 mmol) in dry DCM. The reaction mixture was stirred at −20° C. for 30 min. After completion, reaction mixture was diluted with DCM (25 mL). After concentration crude was initially purified by combi-flash chromatography (0-3% MeOH-DCM) and after Prep-HPLC purification provided 1-(4-(5-(4-(trifluoromethyl) phenoxy)-3,4-dihydroisoquinolin-2(1H)-yl) piperidin-1-yl) prop-2-en-1-one (8 mg, 10%) as off white sticky solid. MS (m/z): 431 [M+H].

All compounds in Table 1 listed below can be made according to the synthetic examples described in this disclosure, and by making any necessary substitutions of starting materials that the skilled artisan would be able to obtain either commercially or otherwise.

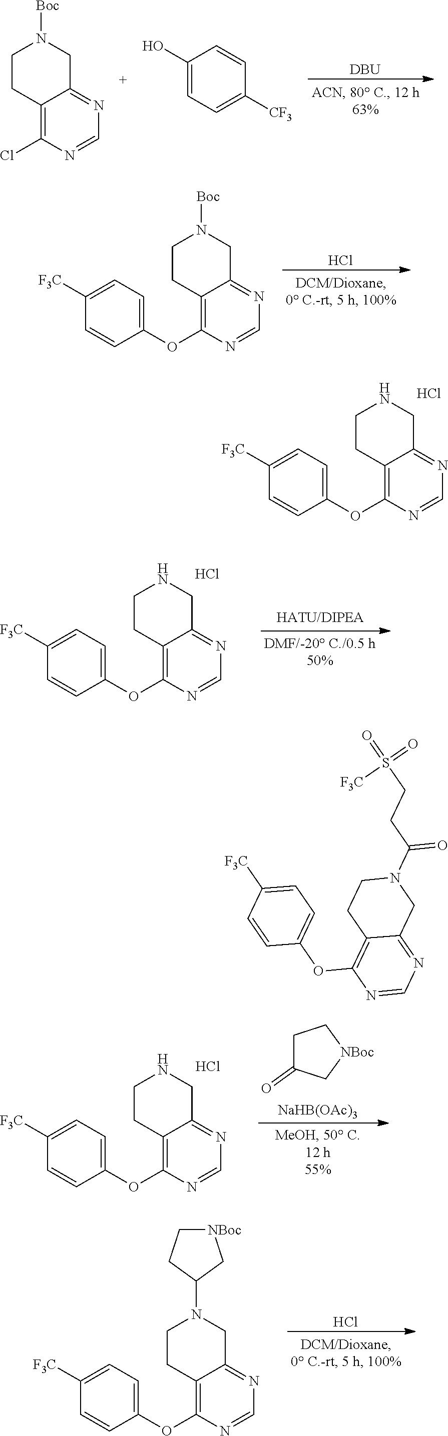

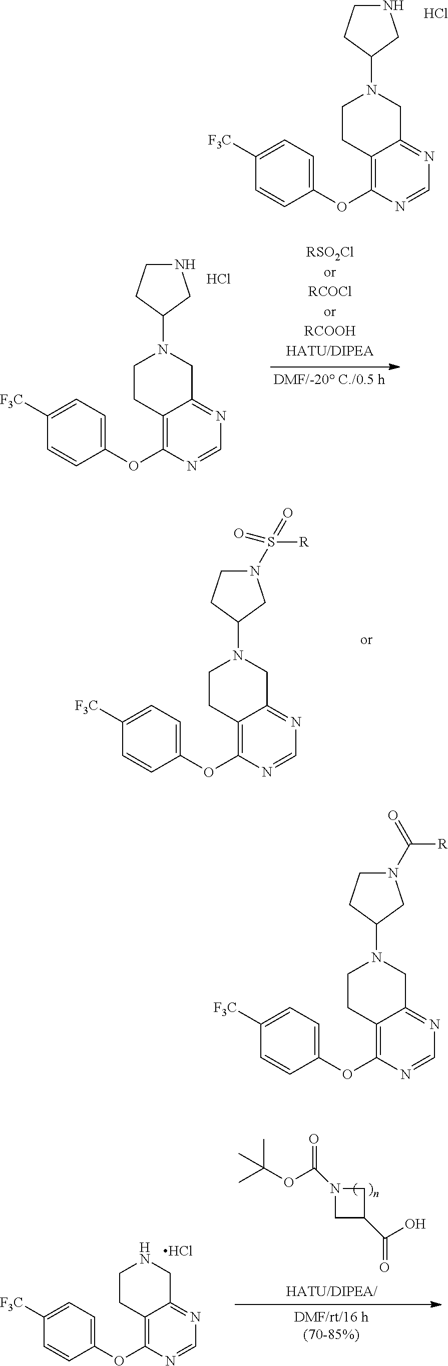

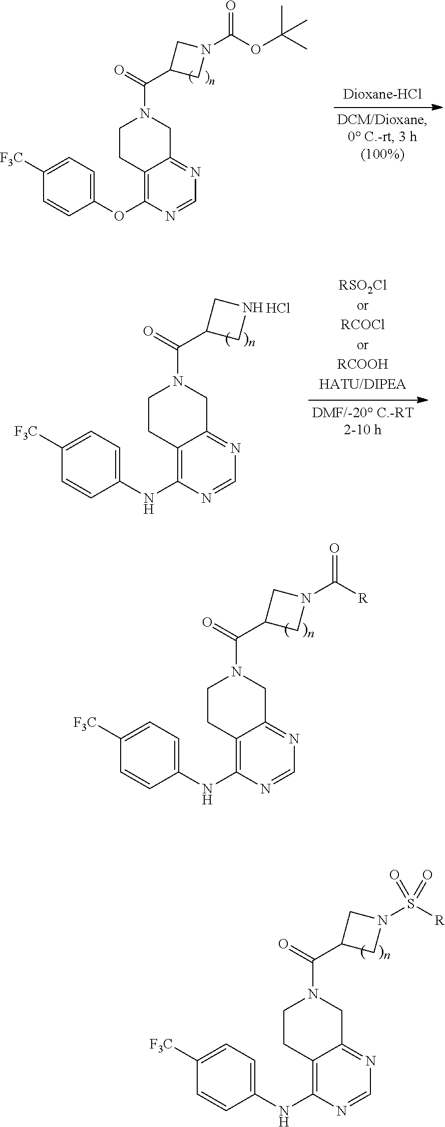

TABLE 1

| O # | Structure | Name |
|---|---|---|
| O-0001 | 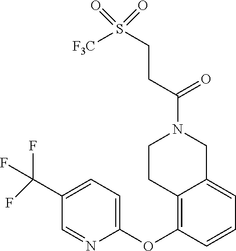 [SMILES: O=C(CCS(=O)(=O)C(F)(F)F)N1CCc2c(cccc2Oc2ccc(C(F)(F)F)cn2)C1] | 1-(5-((5-(trifluoromethyl)pyridin-2-yl)oxy)- 3,4-dihydroisoquinolin-2(1H)-yl)-3- ((trifluoromethyl)sulfonyl)propan-1-one |
| O-0002 | 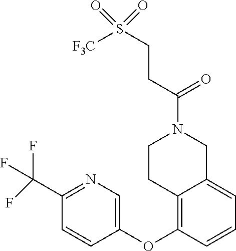 [SMILES: O=C(CCS(=O)(=O)C(F)(F)F)N1CCc2c(cccc2Oc2ccc(C(F)(F)F)nc2)C1] | 1-(5-((6-(trifluoromethyl)pyridin-3-yl)oxy)-3,4- dihydroisoquinolin-2(1H)-yl)-3- ((trifluoromethyl)sulfonyl)propan-1-one |
| O-0003 | 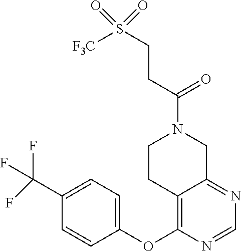 [SMILES: O=C(CCS(=O)(=O)C(F)(F)F)N1CCc2c(ncnc2Oc2ccc(C(F)(F)F)cc2)C1] | 1-(4-(4-(trifluoromethyl)phenoxy)-5,8- dihydropyrido[3,4-d]pyrimidin-7(6H)-yl)-3- ((trifluoromethyl)sulfonyl)propan-1-one |
| O-0004 | 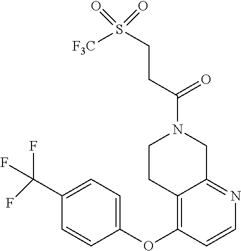 [SMILES: O=C(CCS(=O)(=O)C(F)(F)F)N1CCc2c(Oc3ccc(C(F)(F)F)cc3)ccnc2C1] | 1-(4-(4-(trifluoromethyl)phenoxy)-5,8-dihydro- 1,7-naphthyridin-7(6H)-yl)-3- ((trifluoromethyl)sulfonyl)propan-1-one |
| O-0005 | 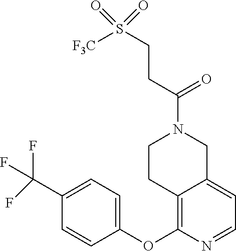 [SMILES: O=C(CCS(=O)(=O)C(F)(F)F)N1CCc2c(ccnc2Oc2ccc(C(F)(F)F)cc2)C1] | 1-(5-(4-(trifluoromethyl)phenoxy)-3,4-dihydro- 2,6-naphthyridin-2(1H)-yl)-3- ((trifluoromethyl)sulfonyl)propan-1-one |
| O-0006 | 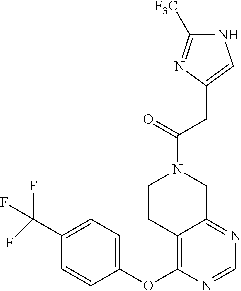 [SMILES: O=C(Cc1c[nH]c(C(F)(F)F)n1)N1CCc2c(ncnc2Oc2ccc(C(F)(F)F)cc2)C1] | 2-(2-(trifluoromethyl)-1H-imidazol-4-yl)-1-(4- (4-(trifluoromethyl)phenoxy)-5,8- dihydropyrido[3,4-d]pyrimidin-7(6H)-yl)ethan- 1-one |
| O-0007 | 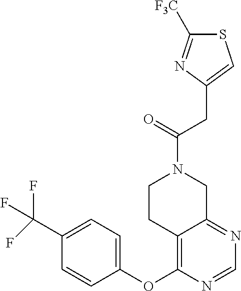 [SMILES: O=C(Cc1csc(C(F)(F)F)n1)N1CCc2c(ncnc2Oc2ccc(C(F)(F)F)cc2)C1] | 1-(4-(4-(trifluoromethyl)phenoxy)-5,8- dihydropyrido[3,4-d]pyrimidin-7(6H)-yl)-2-(2- (trifluoromethyl)thiazol-4-yl)ethan-1-one |
| O-0008 | 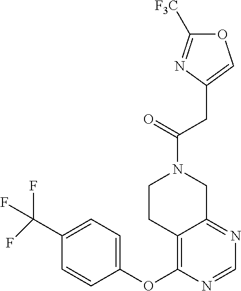 [SMILES: O=C(Cc1coc(C(F)(F)F)n1)N1CCc2c(ncnc2Oc2ccc(C(F)(F)F)cc2)C1] | 2-(2-(trifluoromethyl)oxazol-4-yl)-1-(4-(4- (trifluoromethyl)phenoxy)-5,8- dihydropyrido[3,4-d]pyrimidin-7(6H)-yl)ethan- 1-one |
| O-0009 | 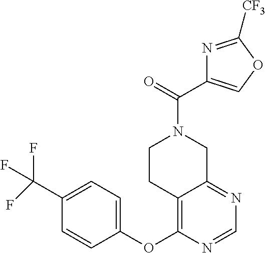 [SMILES: O=C(c1coc(C(F)(F)F)n1)N1CCc2c(ncnc2Oc2ccc(C(F)(F)F)cc2)C1] | (2-(trifluoromethyl)oxazol-4-yl)(4-(4- (trifluoromethyl)phenoxy)-5,8- dihydropyrido[3,4-d]pyrimidin-7(6H)- yl)methanone |
| O-0010 | 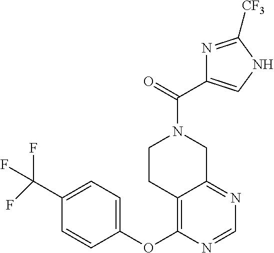 [SMILES: O=C(c1c[nH]c(C(F)(F)F)n1)N1CCc2c(ncnc2Oc2ccc(C(F)(F)F)cc2)C1] | (2-(trifluoromethyl)-1H-imidazol-4-yl)(4-(4- (trifluoromethyl)phenoxy)-5,8- dihydropyrido[3,4-d]pyrimidin-7(6H)- yl)methanone |
| O-0011 | 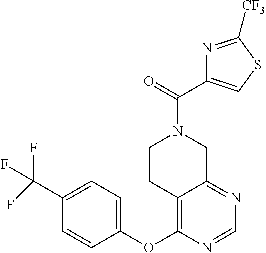 [SMILES: O=C(c1csc(C(F)(F)F)n1)N1CCc2c(ncnc2Oc2ccc(C(F)(F)F)cc2)C1] | (4-(4-(trifluoromethyl)phenoxy)-5,8- dihydropyrido[3,4-d]pyrimidin-7(6H)-yl)(2- (trifluoromethyl)thiazol-4-yl)methanone |
| O-0012 | 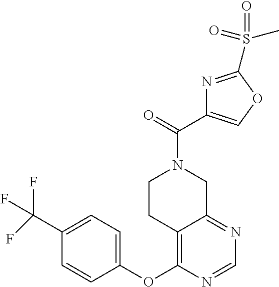 [SMILES: CS(=O)(=O)c1nc(C(=O)N2CCc3c(ncnc3Oc3ccc(C(F)(F)F)cc3)C2)co1] | (2-(methylsulfonyl)oxazol-4-yl)(4-(4- (trifluoromethyl)phenoxy)-5,8- dihydropyrido[3,4-d]pyrimidin-7(6H)- yl)methanone |
| O-0013 | 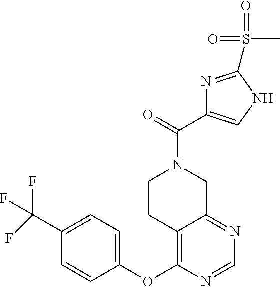 [SMILES: CS(=O)(=O)c1nc(C(=O)N2CCc3c(ncnc3Oc3ccc(C(F)(F)F)cc3)C2)c[nH]1] | (2-(methylsulfonyl)-1H-imidazol-4-yl)(4-(4- (trifluoromethyl)phenoxy)-5,8- dihydropyrido[3,4-d]pyrimidin-7(6H)- yl)methanone |
| O-0014 | 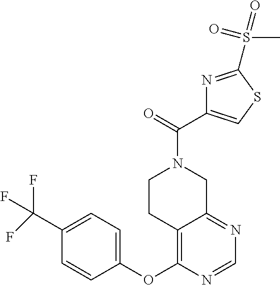 [SMILES: CS(=O)(=O)c1nc(C(=O)N2CCc3c(ncnc3Oc3ccc(C(F)(F)F)cc3)C2)cs1] | (2-(methylsulfonyl)thiazol-4-yl)(4-(4- (trifluoromethyl)phenoxy)-5,8- dihydropyrido[3,4-d]pyrimidin-7(6H)- yl)methanone |
| O-0015 | 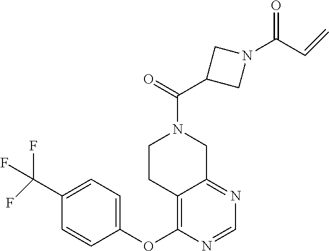 [SMILES: C=CC(=O)N1CC(C(=O)N2CCc3c(ncnc3Oc3ccc(C(F)(F)F)cc3)C2)C1] | 1-(3-(4-(4-(trifluoromethyl)phenoxy)-5,6,7,8- tetrahydropyrido[3,4-d]pyrimidine-7- carbonyl)azetidin-1-yl)prop-2-en-1-one |
| O-0016 | 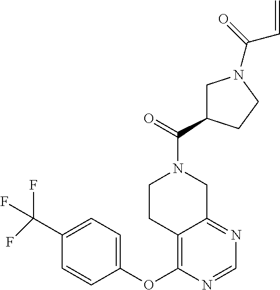 [SMILES: C=CC(=O)N1CC[C@@H](C(=O)N2CCc3c(ncnc3Oc3ccc(C(F)(F)F)cc3)C2)C1] | (R)-1-(3-(4-(4-(trifluoromethyl)phenoxy)- 5,6,7,8-tetrahydropyrido[3,4-d]pyrimidine-7- carbonyl)pyrrolidin-1-yl)prop-2-en-1-one |
| O-0017 | 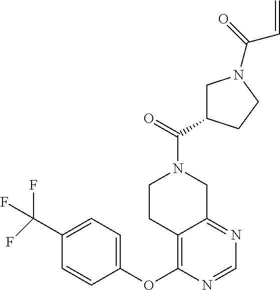 [SMILES: C=CC(=O)N1CC[C@H](C(=O)N2CCc3c(ncnc3Oc3ccc(C(F)(F)F)cc3)C2)C1] | (S)-1-(3-(4-(4-(trifluoromethyl)phenoxy)- 5,6,7,8-tetrahydropyrido[3,4-d]pyrimidine-7- carbonyl)pyrrolidin-1-yl)prop-2-en-1-one |
| O-0018 | 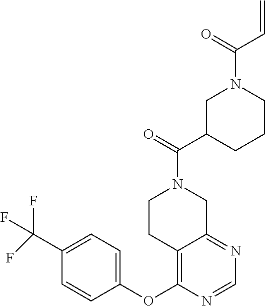 [SMILES: C=CC(=O)N1CCCC(C(=O)N2CCc3c(ncnc3Oc3ccc(C(F)(F)F)cc3)C2)C1] | 1-(3-(4-(4-(trifluoromethyl)phenoxy)-5,6,7,8- tetrahydropyrido[3,4-d]pyrimidine-7- carbonyl)piperidin-1-yl)prop-2-en-1-one |
| O-0019 | 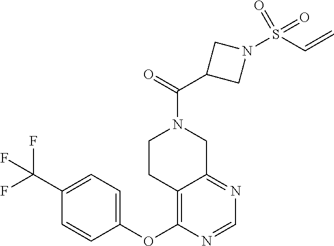 [SMILES: C=CS(=O)(=O)N1CC(C(=O)N2CCc3c(ncnc3Oc3ccc(C(F)(F)F)cc3)C2)C1] | (4-(4-(trifluoromethyl)phenoxy)-5,8- dihydropyrido[3,4-d]pyrimidin-7(6H)-yl)(1- (vinylsulfonyl)azetidin-3-yl)methanone |
| O-0020 | 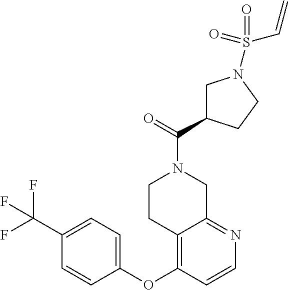 [SMILES: C=CS(=O)(=O)N1CC[C@@H](C(=O)N2CCc3c(Oc4ccc(C(F)(F)F)cc4)ccnc3C2)C1] | (R)-(4-(4-(trifluoromethyl)phenoxy)-5,8- dihydropyrido[3,4-d]pyrimidin-7(6H)-yl)(1- (vinylsulfonyl)pyrrolidin-3-yl)methanone |
| O-0021 | 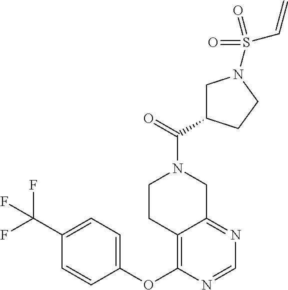 [SMILES: C=CS(=O)(=O)N1CC[C@H](C(=O)N2CCc3c(ncnc3Oc3ccc(C(F)(F)F)cc3)C2)C1] | (S)-(4-(4-(trifluoromethyl)phenoxy)-5,8- dihydropyrido[3,4-d]pyrimidin-7(6H)-yl)(1- (vinylsulfonyl)pyrrolidin-3-yl)methanone |
| O-0022 | 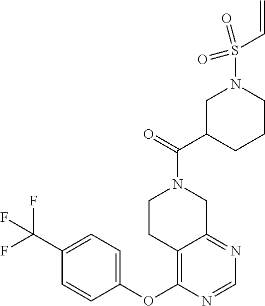 [SMILES: C=CS(=O)(=O)N1CCCC(C(=O)N2CCc3c(ncnc3Oc3ccc(C(F)(F)F)cc3)C2)C1] | (4-(4-(trifluoromethyl)phenoxy)-5,8- dihydropyrido[3,4-d]pyrimidin-7(6H)-yl)(1- (vinylsulfonyl)piperidin-3-yl)methanone |
| O-0023 | 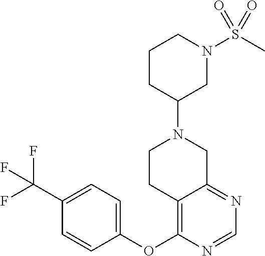 [SMILES: CS(=O)(=O)N1CCCC(N2CCc3c(ncnc3Oc3ccc(C(F)(F)F)cc3)C2)C1] | 7-(1-(methylsulfonyl)piperidin-3-yl)-4-(4- (trifluoromethyl)phenoxy)-5,6,7,8- tetrahydropyrido[3,4-d]pyrimidine |
| O-0024 | 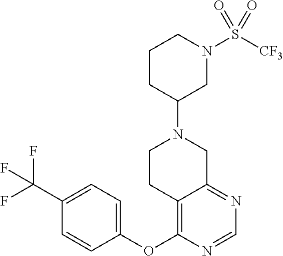 [SMILES: O=S(=O)(N1CCCC(N2CCc3c(ncnc3Oc3ccc(C(F)(F)F)cc3)C2)C1)C(F)(F)F] | 4-(4-(trifluoromethyl)phenoxy)-7-(1- ((trifluoromethyl)sulfonyl)piperidin-3-yl)- 5,6,7,8-tetrahydropyrido[3,4-d]pyrimidine |
| O-0025 | 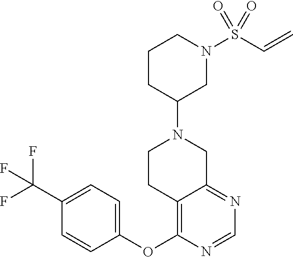 [SMILES: C=CS(=O)(=O)N1CCCC(N2CCc3c(ncnc3Oc3ccc(C(F)(F)F)cc3)C2)C1] | 4-(4-(trifluoromethyl)phenoxy)-7-(1- (vinylsulfonyl)piperidin-3-yl)-5,6,7,8- tetrahydropyrido[3,4-d]pyrimidine |
| O-0026 | 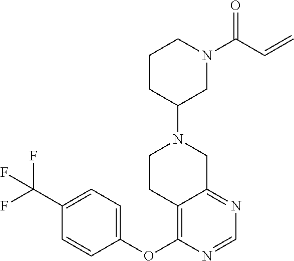 [SMILES: C=CC(=O)N1CCCC(N2CCc3c(ncnc3Oc3ccc(C(F)(F)F)cc3)C2)C1] | 1-(3-(4-(4-(trifluoromethyl)phenoxy)-5,8- dihydropyrido[3,4-d]pyrimidin-7(6H)- yl)piperidin-1-yl)prop-2-en-1-one |
| O-0027 | 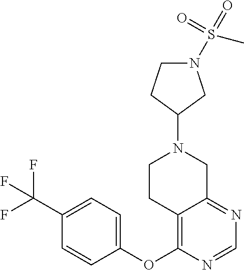 [SMILES: CS(=O)(=O)N1CCC(N2CCc3c(ncnc3Oc3ccc(C(F)(F)F)cc3)C2)C1] | 7-(1-(methylsulfonyl)pyrrolidin-3-yl)-4-(4- (trifluoromethyl)phenoxy)-5,6,7,8- tetrahydropyrido[3,4-d]pyrimidine |
| O-0028 | 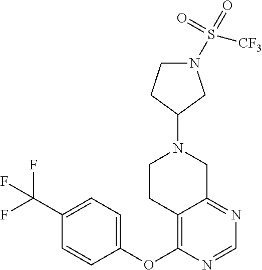 [SMILES: O=S(=O)(N1CCC(N2CCc3c(ncnc3Oc3ccc(C(F)(F)F)cc3)C2)C1)C(F)(F)F] | 4-(4-(trifluoromethyl)phenoxy)-7-(1- ((trifluoromethyl)sulfonyl)pyrrolidin-3-yl)- 5,6,7,8-tetrahydropyrido[3,4-d]pyrimidine |
| O-0029 | 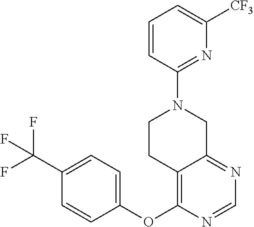 [SMILES: FC(F)(F)c1ccc(Oc2ncnc3c2CCN(c2cccc(C(F)(F)F)n2)C3)cc1] | 4-(4-(trifluoromethyl)phenoxy)-7-(6- (trifluoromethyl)pyridin-2-yl)-5,6,7,8- tetrahydropyrido[3,4-d]pyrimidine |
| O-0030 | 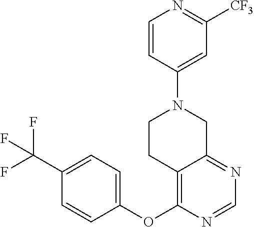 [SMILES: FC(F)(F)c1ccc(Oc2ncnc3c2CCN(c2ccnc(C(F)(F)F)c2)C3)cc1] | 4-(4-(trifluoromethyl)phenoxy)-7-(2- (trifluoromethyl)pyridin-4-yl)-5,6,7,8- tetrahydropyrido[3,4-d]pyrimidine |
| O-0031 | 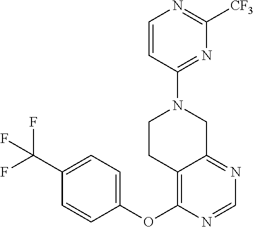 [SMILES: FC(F)(F)c1ccc(Oc2ncnc3c2CCN(c2ccnc(C(F)(F)F)n2)C3)cc1] | 4-(4-(trifluoromethyl)phenoxy)-7-(2- (trifluoromethyl)pyrimidin-4-yl)-5,6,7,8- tetrahydropyrido[3,4-d]pyrimidine |
| O-0032 | 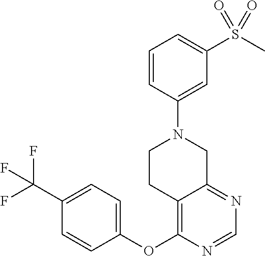 [SMILES: CS(=O)(=O)c1cccc(N2CCc3c(ncnc3Oc3ccc(C(F)(F)F)cc3)C2)c1] | 7-(3-(methylsulfonyl)phenyl)-4-(4- (trifluoromethyl)phenoxy)-5,6,7,8- tetrahydropyrido[3,4-d]pyrimidine |
| O-0033 | 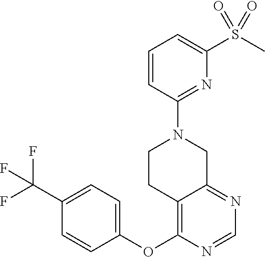 [SMILES: CS(=O)(=O)c1cccc(N2CCc3c(ncnc3Oc3ccc(C(F)(F)F)cc3)C2)n1] | 7-(6-(methylsulfonyl)pyridin-2-yl)-4-(4- (trifluoromethyl)phenoxy)-5,6,7,8- tetrahydropyrido[3,4-d]pyrimidine |
| O-0034 | 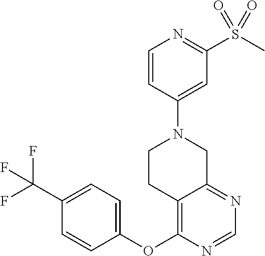 [SMILES: CS(=O)(=O)c1cc(N2CCc3c(ncnc3Oc3ccc(C(F)(F)F)cc3)C2)ccn1] | 7-(2-(methylsulfonyl)pyridin-4-yl)-4-(4- (trifluoromethyl)phenoxy)-5,6,7,8- tetrahydropyrido[3,4-d]pyrimidine |
| O-0035 | 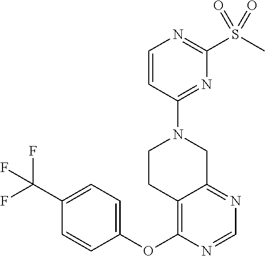 [SMILES: CS(=O)(=O)c1nccc(N2CCc3c(ncnc3Oc3ccc(C(F)(F)F)cc3)C2)n1] | 7-(2-(methylsulfonyl)pyrimidin-4-yl)-4-(4- (trifluoromethyl)phenoxy)-5,6,7,8- tetrahydropyrido[3,4-d]pyrimidine |
| O-0036 | 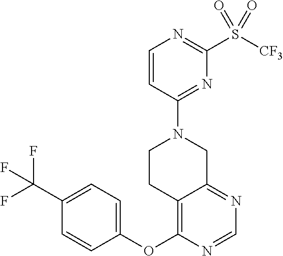 [SMILES: O=S(=O)(c1nccc(N2CCc3c(ncnc3Oc3ccc(C(F)(F)F)cc3)C2)n1)C(F)(F)F] | 4-(4-(trifluoromethyl)phenoxy)-7-(2- ((trifluoromethyl)sulfonyl)pyrimidin-4-yl)- 5,6,7,8-tetrahydropyrido[3,4-d]pyrimidine |
| O-0037 | 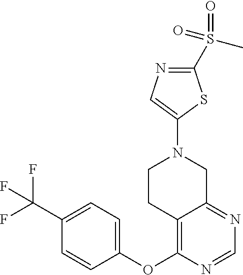 [SMILES: CS(=O)(=O)c1ncc(N2CCc3c(ncnc3Oc3ccc(C(F)(F)F)cc3)C2)s1] | 2-(methylsulfonyl)-5-(4-(4- (trifluoromethyl)phenoxy)-5,8- dihydropyrido[3,4-d]pyrimidin-7(6H)- yl)thiazole |
| O-0038 | 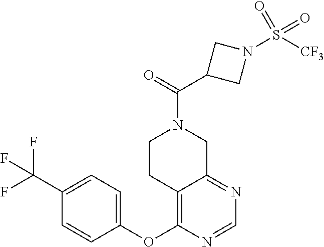 [SMILES: O=C(C1CN(S(=O)(=O)C(F)(F)F)C1)N1CCc2c(ncnc2Oc2ccc(C(F)(F)F)cc2)C1] | (4-(4-(trifluoromethyl)phenoxy)-5,8- dihydropyrido[3,4-d]pyrimidin-7(6H)-yl)(1- ((trifluoromethyl)sulfonyl)azetidin-3- yl)methanone |
| O-0039 | 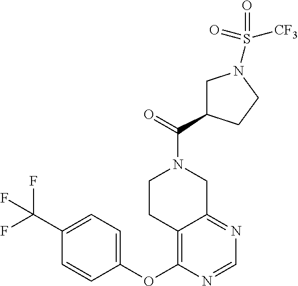 [SMILES: O=C([C@@H]1CCN(S(=O)(=O)C(F)(F)F)C1)N1CCc2c(ncnc2Oc2ccc(C(F)(F)F)cc2)C1] | (R)-(4-(4-(trifluoromethyl)phenoxy)-5,8- dihydropyrido[3,4-d]pyrimidin-7(6H)-yl)(1- ((trifluoromethyl)sulfonyl)pyrrolidin-3- yl)methanone |
| O-0040 | 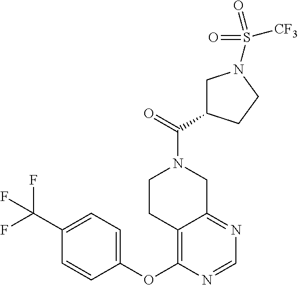 [SMILES: O=C([C@H]1CCN(S(=O)(=O)C(F)(F)F)C1)N1CCc2c(ncnc2Oc2ccc(C(F)(F)F)cc2)C1] | (S)-(4-(4-(trifluoromethyl)phenoxy)-5,8- dihydropyrido[3,4-d]pyrimidin-7(6H)-yl)(1- ((trifluoromethyl)sulfonyl)pyrrolidin-3- yl)methanone |
| O-0041 | 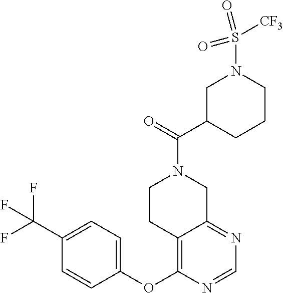 [SMILES: O=C(C1CCCN(S(=O)(=O)C(F)(F)F)C1)N1CCc2c(ncnc2Oc2ccc(C(F)(F)F)cc2)C1] | (4-(4-(trifluoromethyl)phenoxy)-5,8- dihydropyrido[3,4-d]pyrimidin-7(6H)-yl)(1- ((trifluoromethyl)sulfonyl)piperidin-3- yl)methanone |
| O-0042 | 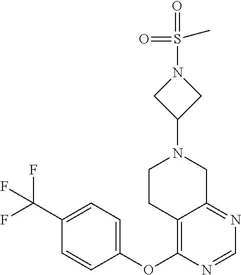 [SMILES: CS(=O)(=O)N1CC(N2CCc3c(ncnc3Oc3ccc(C(F)(F)F)cc3)C2)C1] | 5-(4-(trifluoromethyl)phenoxy)-2-(1- (vinylsulfonyl)pyrrolidin-3-yl)-1,2,3,4- tetrahydroisoquinoline |
| O-0043 | 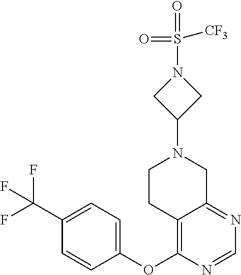 [SMILES: O=S(=O)(N1CC(N2CCc3c(ncnc3Oc3ccc(C(F)(F)F)cc3)C2)C1)C(F)(F)F] | 4-(4-(trifluoromethyl)phenoxy)-7-(1- ((trifluoromethyl)sulfonyl)azetidin-3-yl)-5,6,7,8- tetrahydropyrido[3,4-d]pyrimidine |
| O-0044 | 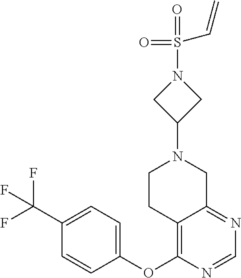 [SMILES: C=CS(=O)(=O)N1CC(N2CCc3c(ncnc3Oc3ccc(C(F)(F)F)cc3)C2)C1] | 4-(4-(trifluoromethyl)phenoxy)-7-(1- (vinylsulfonyl)azetidin-3-yl)-5,6,7,8- tetrahydropyrido[3,4-d]pyrimidine |
| O-0045 | 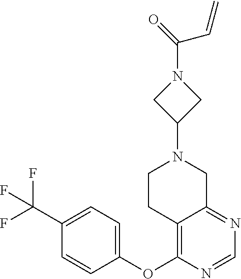 [SMILES: C=CC(=O)N1CC(N2CCc3c(ncnc3Oc3ccc(C(F)(F)F)cc3)C2)C1] | 1-(3-(4-(4-(trifluoromethyl)phenoxy)-5,8- dihydropyrido[3,4-d]pyrimidin-7(6H)- yl)azetidin-1-yl)prop-2-en-1-one |
| O-0046 | 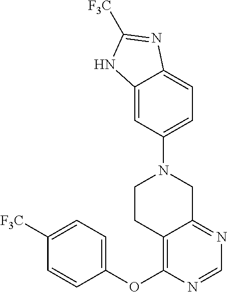 [SMILES: FC(F)(F)c1ccc(Oc2ncnc3c2CCN(c2ccc4nc(C(F)(F)F)[nH]c4c2)C3)cc1] | 7-(2-(trifluoromethyl)-1H-benzo[d]imidazol-6- yl)-4-(4-(trifluoromethyl)phenoxy)-5,6,7,8- tetrahydropyrido[3,4-d]pyrimidine |
| O-0047 | 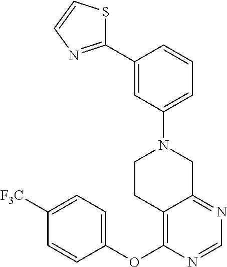 [SMILES: FC(F)(F)c1ccc(Oc2ncnc3c2CCN(c2cccc(-c4nccs4)c2)C3)cc1] | 2-(3-(4-(4-(trifluoromethyl)phenoxy)-5,8- dihydropyrido[3,4-d]pyrimidin-7(6H)- yl)phenyl)thiazole |
| O-0048 | 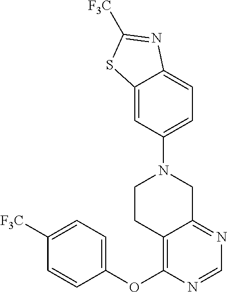 [SMILES: FC(F)(F)c1ccc(Oc2ncnc3c2CCN(c2ccc4nc(C(F)(F)F)sc4c2)C3)cc1] | 2-(trifluoromethyl)-6-(4-(4- (trifluoromethyl)phenoxy)-5,8- dihydropyrido[3,4-d]pyrimidin-7(6H)- yl)benzo[d]thiazole |
| O-0049 | 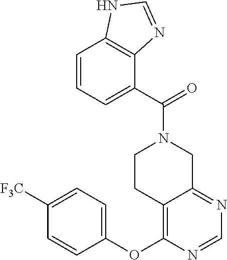 [SMILES: O=C(c1cccc2[nH]cnc12)N1CCc2c(ncnc2Oc2ccc(C(F)(F)F)cc2)C1] | (1H-benzo[d]imidazol-4-yl)(4-(4- (trifluoromethyl)phenoxy)-5,8- dihydropyrido[3,4-d]pyrimidin-7(6H)- yl)methanone |
| O-0050 | 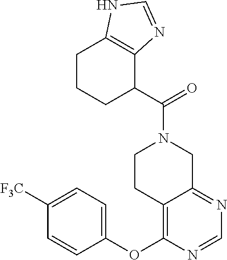 [SMILES: O=C(C1CCCc2[nH]cnc21)N1CCc2c(ncnc2Oc2ccc(C(F)(F)F)cc2)C1] | (4,5,6,7-tetrahydro-1H-benzo[d]imidazol-4- yl)(4-(4-(trifluoromethyl)phenoxy)-5,8- dihydropyrido[3,4-d]pyrimidin-7(6H)- yl)methanone |
| O-0051 | 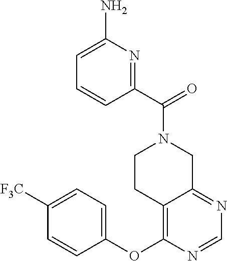 [SMILES: Nc1cccc(C(=O)N2CCc3c(ncnc3Oc3ccc(C(F)(F)F)cc3)C2)n1] | (6-aminopyridin-2-yl)(4-(4- (trifluoromethyl)phenoxy)-5,8- dihydropyrido[3,4-d]pyrimidin-7(6H)- yl)methanone |
| O-0052 | 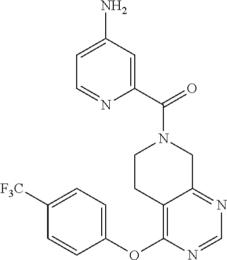 [SMILES: Nc1ccnc(C(=O)N2CCc3c(ncnc3Oc3ccc(C(F)(F)F)cc3)C2)c1] | (4-aminopyridin-2-yl)(4-(4- (trifluoromethyl)phenoxy)-5,8- dihydropyrido[3,4-d]pyrimidin-7(6H)- yl)methanone |
| O-0053 | 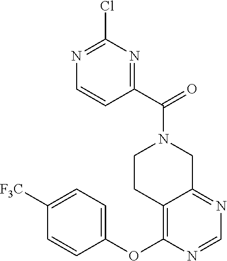 [SMILES: O=C(c1ccnc(Cl)n1)N1CCc2c(ncnc2Oc2ccc(C(F)(F)F)cc2)C1] | (2-chloropyrimidin-4-yl)(4-(4- (trifluoromethyl)phenoxy)-5,8- dihydropyrido[3,4-d]pyrimidin-7(6H)- yl)methanone |
| O-0054 | 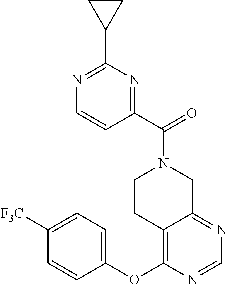 [SMILES: O=C(c1ccnc(C2CC2)n1)N1CCc2c(ncnc2Oc2ccc(C(F)(F)F)cc2)C1] | (2-cyclopropylpyrimidin-4-yl)(4-(4- (trifluoromethyl)phenoxy)-5,8- dihydropyrido[3,4-d]pyrimidin-7(6H)- yl)methanone |
| O-0055 | 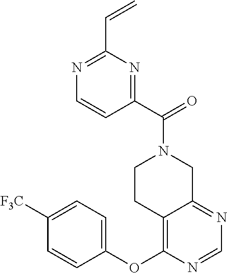 [SMILES: C=Cc1nccc(C(=O)N2CCc3c(ncnc3Oc3ccc(C(F)(F)F)cc3)C2)n1] | (4-(4-(trifluoromethyl)phenoxy)-5,8- dihydropyrido[3,4-d]pyrimidin-7(6H)-yl)(2- vinylpyrimidin-4-yl)methanone |
| O-0056 | 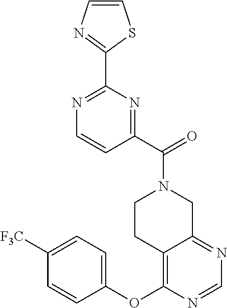 [SMILES: O=C(c1ccnc(-c2nccs2)n1)N1CCc2c(ncnc2Oc2ccc(C(F)(F)F)cc2)C1] | (2-(thiazol-2-yl)pyrimidin-4-yl)(4-(4- (trifluoromethyl)phenoxy)-5,8- dihydropyrido[3,4-d]pyrimidin-7(6H)- yl)methanone |
| O-0057 | 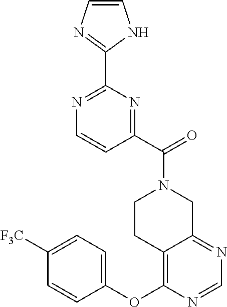 [SMILES: O=C(c1ccnc(-c2ncc[nH]2)n1)N1CCc2c(ncnc2Oc2ccc(C(F)(F)F)cc2)C1] | (2-(1H-imidazol-2-yl)pyrimidin-4-yl)(4-(4- (trifluoromethyl)phenoxy)-5,8- dihydropyrido[3,4-d]pyrimidin-7(6H)- yl)methanone |
| O-0058 | 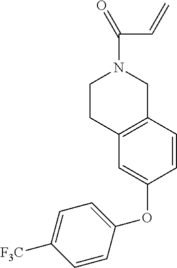 [SMILES: C=CC(=O)N1CCc2cc(Oc3ccc(C(F)(F)F)cc3)ccc2C1] | 1-(6-(4-(trifluoromethyl)phenoxy)-3,4- dihydroisoquinolin-2(1H)-yl)prop-2-en-1-one |
| O-0059 | 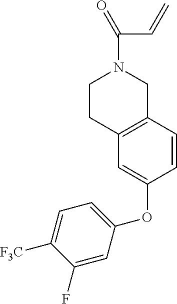 [SMILES: C=CC(=O)N1CCc2cc(Oc3ccc(C(F)(F)F)c(F)c3)ccc2C1] | 1-(6-(3-fluoro-4-(trifluoromethyl)phenoxy)-3,4- dihydroisoquinolin-2(1H)-yl)prop-2-en-1-one |
| O-0060 | 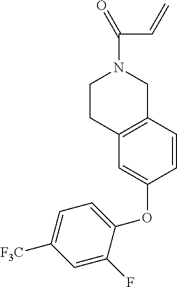 [SMILES: C=CC(=O)N1CCc2cc(Oc3ccc(C(F)(F)F)cc3F)ccc2C1] | 1-(6-(2-fluoro-4-(trifluoromethyl)phenoxy)-3,4- dihydroisoquinolin-2(1H)-yl)prop-2-en-1-one |
| 0-0061 | 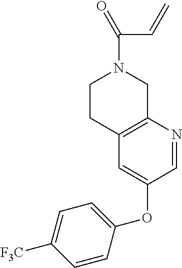 [SMILES: C=CC(=O)N1CCc2cc(Oc3ccc(C(F)(F)F)cc3)cnc2C1] | 1-(3-(4-(trifluoromethyl)phenoxy)-5,8-dihydro-1,7- naphthyridin-7(6H)-yl)prop-2-en-1-one |
| O-0062 | 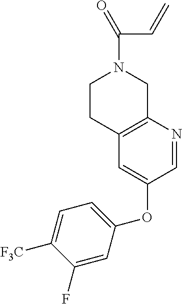 [SMILES: C=CC(=O)N1CCc2cc(Oc3ccc(C(F)(F)F)c(F)c3)cnc2C1] | 1-(3-(3-fluoro-4-(trifluoromethyl)phenoxy)-5,8- dihydro-1,7-naphthyridin-7(6H)-yl)prop-2-en-1-one |
| O-0063 | 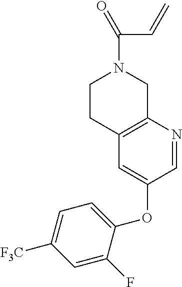 [SMILES: C=CC(=O)N1CCc2cc(Oc3ccc(C(F)(F)F)cc3F)cnc2C1] | 1-(3-(2-fluoro-4-(trifluoromethyl)phenoxy)-5,8- dihydro-1,7-naphthyridin-7(6H)-yl)prop-2-en-1-one |
| O-0064 | 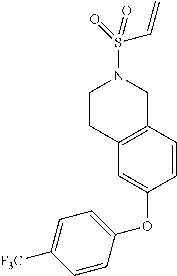 [SMILES: C=CS(=O)(=O)N1CCc2cc(Oc3ccc(C(F)(F)F)cc3)ccc2C1] | 6-(4-(trifluoromethyl)phenoxy)-2-(vinylsulfonyl)- 1,2,3,4-tetrahydroisoquinoline |
| O-0065 | 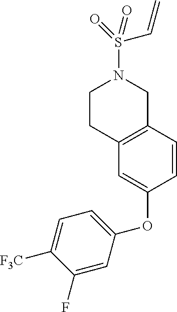 [SMILES: C=CS(=O)(=O)N1CCc2cc(Oc3ccc(C(F)(F)F)c(F)c3)ccc2C1] | 6-(3-fluoro-4-(trifluoromethyl)phenoxy)-2- (vinylsulfonyl)-1,2,3,4-tetrahydroisoquinoline |
| O-0066 | 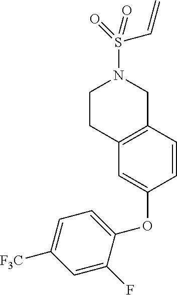 [SMILES: C=CS(=O)(=O)N1CCc2cc(Oc3ccc(C(F)(F)F)cc3F)ccc2C1] | 6-(2-fluoro-4-(trifluoromethyl)phenoxy)-2- (vinylsulfonyl)-1,2,3,4-tetrahydroisoquinoline |
| O-0067 | 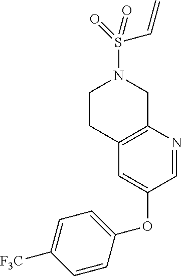 [SMILES: C=CS(=O)(=O)N1CCc2cc(Oc3ccc(C(F)(F)F)cc3)cnc2C1] | 3-(4-(trifluoromethyl)phenoxy)-7-(vinylsulfonyl)- 5,6,7,8-tetrahydro-1,7-naphthyridine |
| O-0068 | 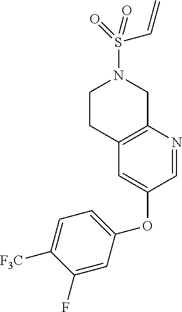 [SMILES: C=CS(=O)(=O)N1CCc2cc(Oc3ccc(C(F)(F)F)c(F)c3)cnc2C1] | 3-(3-fluoro-4-(trifluoromethyl)phenoxy)-7- (vinylsulfonyl)-5,6,7,8-tetrahydro-1,7-naphthyridine |
| O-0069 | 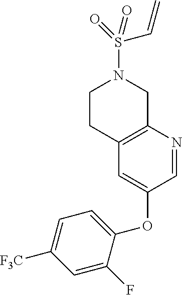 [SMILES: C=CS(=O)(=O)N1CCc2cc(Oc3ccc(C(F)(F)F)cc3F)cnc2C1] | 3-(2-fluoro-4-(trifluoromethyl)phenoxy)-7- (vinylsulfonyl)-5,6,7,8-tetrahydro-1,7-naphthyridine |
| 0-0070 | 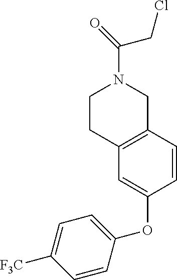 [SMILES: O=C(CCl)N1CCc2cc(Oc3ccc(C(F)(F)F)cc3)ccc2C1] | 2-chloro-1-(6-(4-(trifluoromethyl)phenoxy)-3,4- dihydroisoquinolin-2(1H)-yl)ethan-1-one |
| O-0071 | 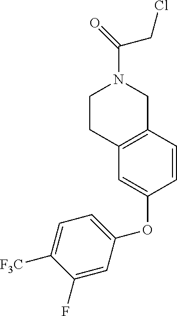 [SMILES: O=C(CCl)N1CCc2cc(Oc3ccc(C(F)(F)F)c(F)c3)ccc2C1] | 2-chloro-1-(6-(3-fluoro-4-(trifluoromethyl)phenoxy)- 3,4-dihydroisoquinolin-2(1H)-yl)ethan-1-one |
| O-0072 | 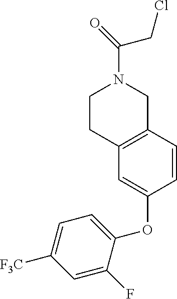 [SMILES: O=C(CCl)N1CCc2cc(Oc3ccc(C(F)(F)F)cc3F)ccc2C1] | 2-chloro-1-(6-(2-fluoro-4-(trifluoromethyl)phenoxy)- 3,4-dihydroisoquinolin-2(1H)-yl)ethan-1-one |
| O-0073 | 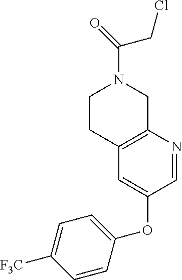 [SMILES: O=C(CCl)N1CCc2cc(Oc3ccc(C(F)(F)F)cc3)cnc2C1] | 2-chloro-1-(3-(4-(trifluoromethyl)phenoxy)-5,8- dihydro-1,7-naphthyridin-7(6H)-yl)ethan-1-one |
| O-0074 | 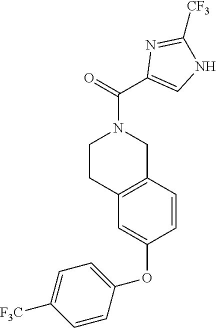 [SMILES: O=C(c1c[nH]c(C(F)(F)F)n1)N1CCc2cc(Oc3ccc(C(F)(F)F)cc3)ccc2C1] | (2-(trifluoromethyl)-1H-imidazol-4-yl)(6-(4- (trifluoromethyl)phenoxy)-3,4-dihydroisoquinolin- 2(1H)-yl)methanone |
| O-0075 | 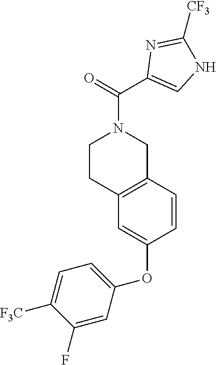 [SMILES: O=C(c1c[nH]c(C(F)(F)F)n1)N1CCc2cc(Oc3ccc(C(F)(F)F)c(F)c3)ccc2C1] | (6-(3-fluoro-4-(trifluoromethyl)phenoxy)-3,4- dihydroisoquinolin-2(1H)-yl)(2-(trifluoromethyl)- 1H-imidazol-4-yl)methanone |
| O-0076 | 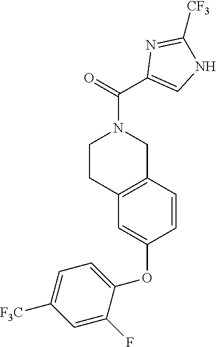 [SMILES: O=C(c1c[nH]c(C(F)(F)F)n1)N1CCc2cc(Oc3ccc(C(F)(F)F)cc3F)ccc2C1] | (6-(2-fluoro-4-(trifluoromethyl)phenoxy)-3,4- dihydroisoquinolin-2(1H)-yl)(2-(trifluoromethyl)- 1H-imidazol-4-yl)methanone |
| 0-0077 | 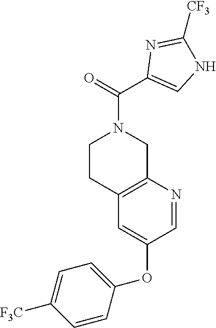 [SMILES: O=C(c1c[nH]c(C(F)(F)F)n1)N1CCc2cc(Oc3ccc(C(F)(F)F)cc3)cnc2C1] | (2-(trifluoromethyl)-1H-imidazol-4-yl)(3-(4- (trifluoromethyl)phenoxy)-5,8-dihydro-1,7- naphthyridin-7(6H)-yl)methanone |
| O-0078 | 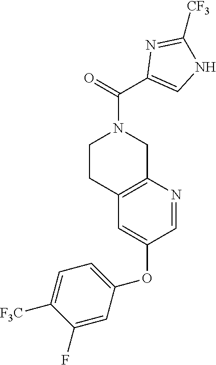 [SMILES: O=C(c1c[nH]c(C(F)(F)F)n1)N1CCc2cc(Oc3ccc(C(F)(F)F)c(F)c3)cnc2C1] | (3-(3-fluoro-4-(trifluoromethyl)phenoxy)-5,8- dihydro-1,7-naphthyridin-7(6H)-yl)(2- (trifluoromethyl)-1H-imidazol-4-yl)methanone |
| O-0079 | 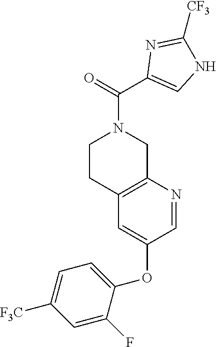 [SMILES: O=C(c1c[nH]c(C(F)(F)F)n1)N1CCc2cc(Oc3ccc(C(F)(F)F)cc3F)cnc2C1] | (3-(2-fluoro-4-(trifluoromethyl)phenoxy)-5,8- dihydro-1,7-naphthyridin-7(6H)-yl)(2- (trifluoromethyl)-1H-imidazol-4-yl)methanone |
| O-0080 | 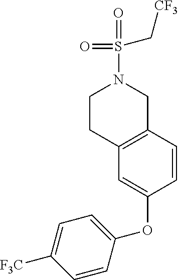 [SMILES: O=S(=O)(CC(F)(F)F)N1CCc2cc(Oc3ccc(C(F)(F)F)cc3)ccc2C1] | 2-((2,2,2-trifluoroethyl)sulfonyl)-6-(4- (trifluoromethyl)phenoxy)-1,2,3,4- tetrahydroisoquinoline |
| O-0081 | 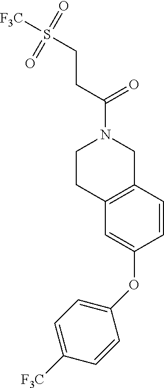 [SMILES: O=C(CCS(=O)(=O)C(F)(F)F)N1CCc2cc(Oc3ccc(C(F)(F)F)cc3)ccc2C1] | 1-(6-(4-(trifluoromethyl)phenoxy)-3,4- dihydroisoquinolin-2(1H)-yl)-3- ((trifluoromethyl)sulfonyl)propan-1-one |
| O-0082 | 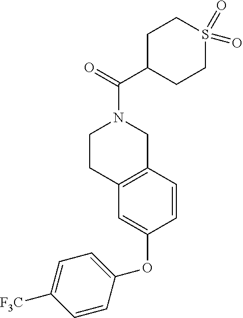 [SMILES: O=C(C1CCS(=O)(=O)CC1)N1CCc2cc(Oc3ccc(C(F)(F)F)cc3)ccc2C1] | (1,1-dioxidotetrahydro-2H-thiopyran-4-yl)(6-(4- (trifluoromethyl)phenoxy)-3,4-dihydroisoquinolin- 2(1H)-yl)methanone |
| O-0083 | 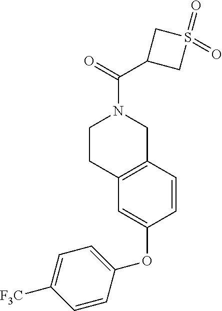 [SMILES: O=C(C1CS(=O)(=O)C1)N1CCc2cc(Oc3ccc(C(F)(F)F)cc3)ccc2C1] | (1,1-dioxidothietan-3-yl)(6-(4- (trifluoromethyl)phenoxy)-3,4-dihydroisoquinolin- 2(1H)-yl)methanone |
| O-0084 | 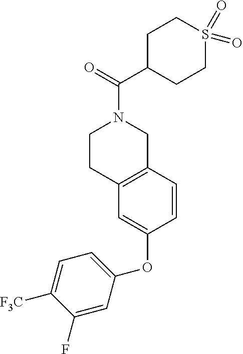 [SMILES: O=C(C1CCS(=O)(=O)CC1)N1CCc2cc(Oc3ccc(C(F)(F)F)c(F)c3)ccc2C1] | (1,1-dioxidotetrahydro-2H-thiopyran-4-yl)(6-(3- fluoro-4-(trifluoromethyl)phenoxy)-3,4- dihydroisoquinolin-2(1H)-yl)methanone |
| O-0085 | 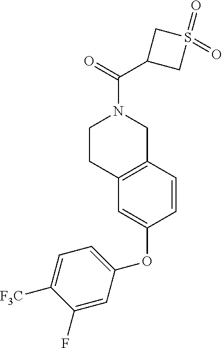 [SMILES: O=C(C1CS(=O)(=O)C1)N1CCc2cc(Oc3ccc(C(F)(F)F)c(F)c3)ccc2C1] | (1,1-dioxidothietan-3-yl)(6-(3-fluoro-4- (trifluoromethyl)phenoxy)-3,4-dihydroisoquinolin- 2(1H)-yl)methanone |
| O-0086 | 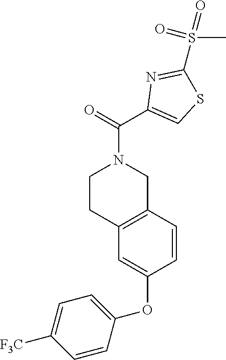 [SMILES: CS(=O)(=O)c1nc(C(=O)N2CCc3cc(Oc4ccc(C(F)(F)F)cc4)ccc3C2)cs1] | (2-(methylsulfonyl)thiazol-4-yl)(6-(4- (trifluoromethyl)phenoxy)-3,4-dihydroisoquinolin- 2(1H)-yl)methanone |
| O-0087 | 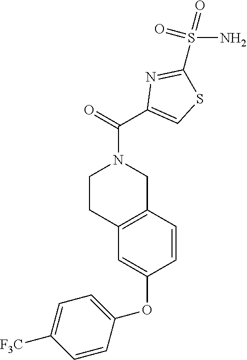 [SMILES: NS(=O)(=O)c1nc(C(=O)N2CCc3cc(Oc4ccc(C(F)(F)F)cc4)ccc3C2)cs1] | 4-(6-(4-(trifluoromethyl)phenoxy)-1,2,3,4- tetrahydroisoquinoline-2-carbonyl)thiazole-2- sulfonamide |
| O-0088 | 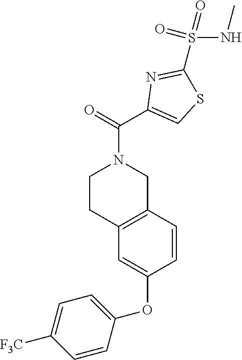 [SMILES: CNS(=O)(=O)c1nc(C(=O)N2CCc3cc(Oc4ccc(C(F)(F)F)cc4)ccc3C2)cs1] | N-methyl-4-(6-(4-(trifluoromethyl)phenoxy)-1,2,3,4- tetrahydroisoquinoline-2-carbonyl)thiazole-2- sulfonamide |
| O-0089 | 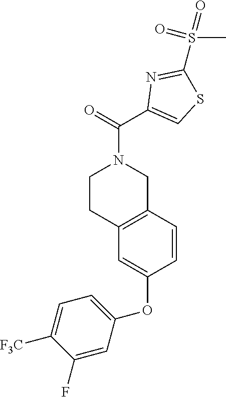 [SMILES: CS(=O)(=O)c1nc(C(=O)N2CCc3cc(Oc4ccc(C(F)(F)F)c(F)c4)ccc3C2)cs1] | (6-(3-fluoro-4-(trifluoromethyl)phenoxy)-3,4- dihydroisoquinolin-2(1H)-yl)(2- (methylsulfonyl)thiazol-4-yl)methanone |
| O-0090 | 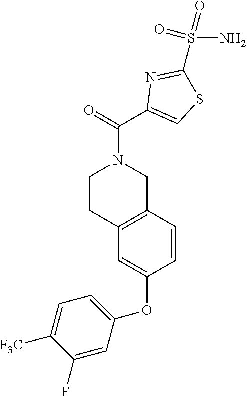 [SMILES: NS(=O)(=O)c1nc(C(=O)N2CCc3cc(Oc4ccc(C(F)(F)F)c(F)c4)ccc3C2)cs1] | 4-(6-(3-fluoro-4-(trifluoromethyl)phenoxy)-1,2,3,4- tetrahydroisoquinoline-2-carbonyl)thiazole-2- sulfonamide |
| O-0091 | 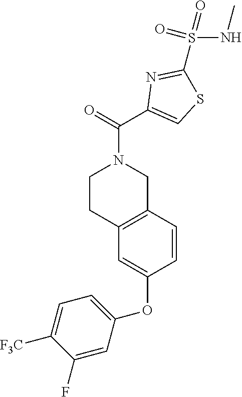 [SMILES: CNS(=O)(=O)c1nc(C(=O)N2CCc3cc(Oc4ccc(C(F)(F)F)c(F)c4)ccc3C2)cs1] | 4-(6-(3-fluoro-4-(trifluoromethyl)phenoxy)-1,2,3,4- tetrahydroisoquinoline-2-carbonyl)-N- methylthiazole-2-sulfonamide |
| O-0092 | 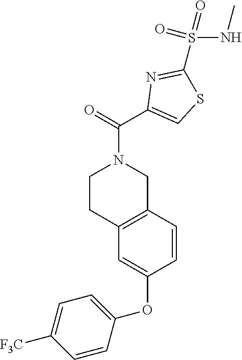 [SMILES: CNS(=O)(=O)c1nc(C(=O)N2CCc3cc(Oc4ccc(C(F)(F)F)cc4)ccc3C2)cs1] | N-methyl-4-(6-(4-(trifluoromethyl)phenoxy)-1,2,3,4- tetrahydroisoquinoline-2-carbonyl)thiazole-2- sulfonamide |
| O-0093 | 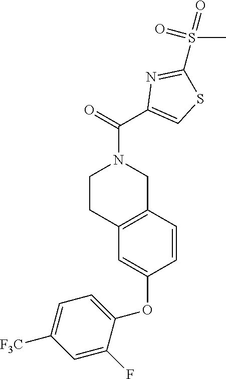 [SMILES: CS(=O)(=O)c1nc(C(=O)N2CCc3cc(Oc4ccc(C(F)(F)F)cc4F)ccc3C2)cs1] | (6-(2-fluoro-4-(trifluoromethyl)phenoxy)-3,4- dihydroisoquinolin-2(1H)-yl)(2- (methylsulfonyl)thiazol-4-yl)methanone |
| O-0094 | 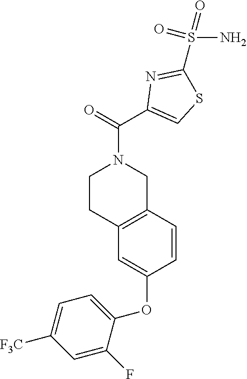 [SMILES: NS(=O)(=O)c1nc(C(=O)N2CCc3cc(Oc4ccc(C(F)(F)F)cc4F)ccc3C2)cs1] | 4-(6-(2-fluoro-4-(trifluoromethyl)phenoxy)-1,2,3,4- tetrahydroisoquinoline-2-carbonyl)thiazole-2- sulfonamide |
| O-0095 | 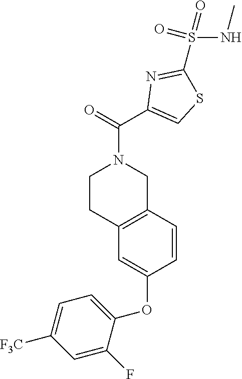 [SMILES: CNS(=O)(=O)c1nc(C(=O)N2CCc3cc(Oc4ccc(C(F)(F)F)cc4F)ccc3C2)cs1] | 4-(6-(2-fluoro-4-(trifluoromethyl)phenoxy)-1,2,3,4- tetrahydroisoquinoline-2-carbonyl)-N- methylthiazole-2-sulfonamide |
| 0-0096 | 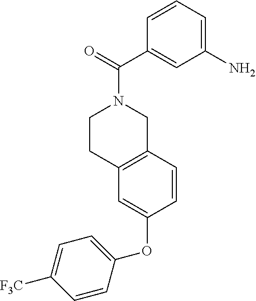 [SMILES: Nc1cccc(C(=O)N2CCc3cc(Oc4ccc(C(F)(F)F)cc4)ccc3C2)c1] | (3-aminophenyl)(6-(4-(trifluoromethyl)phenoxy)- 3,4-dihydroisoquinolin-2(1H)-yl)methanone |
| O-0097 | 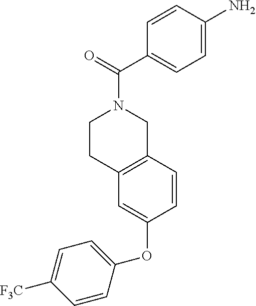 [SMILES: Nc1ccc(C(=O)N2CCc3cc(Oc4ccc(C(F)(F)F)cc4)ccc3C2)cc1] | (4-aminophenyl)(6-(4-(trifluoromethyl)phenoxy)- 3,4-dihydroisoquinolin-2(1H)-yl)methanone |
| O-0098 | 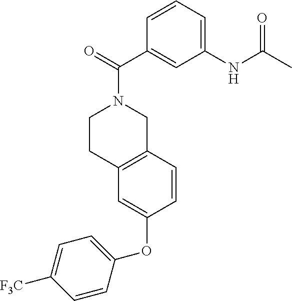 [SMILES: CC(=O)Nc1cccc(C(=O)N2CCc3cc(Oc4ccc(C(F)(F)F)cc4)ccc3C2)c1] | N-(3-(6-(4-(trifluoromethyl)phenoxy)-1,2,3,4- tetrahydroisoquinoline-2-carbonyl)phenyl)acetamide |
| 0-0099 | 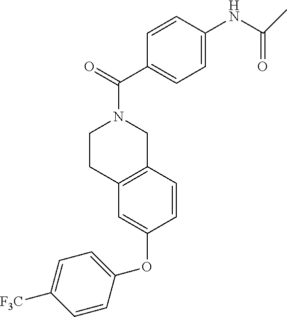 [SMILES: CC(=O)Nc1ccc(C(=O)N2CCc3cc(Oc4ccc(C(F)(F)F)cc4)ccc3C2)cc1] | N-(4-(6-(4-(trifluoromethyl)phenoxy)-1,2,3,4- tetrahydroisoquinoline-2-carbonyl)phenyl)acetamide |
| O-0100 | 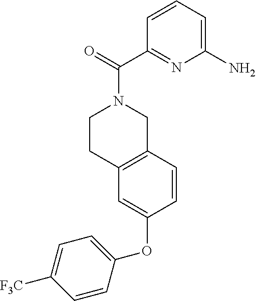 [SMILES: Nc1cccc(C(=O)N2CCc3cc(Oc4ccc(C(F)(F)F)cc4)ccc3C2)n1] | (6-aminopyridin-2-yl)(6-(4- (trifluoromethyl)phenoxy)-3,4-dihydroisoquinolin- 2(1H)-yl)methanone |
| 0-0101 | 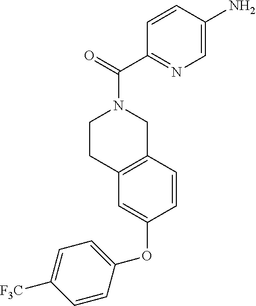 [SMILES: Nc1ccc(C(=O)N2CCc3cc(Oc4ccc(C(F)(F)F)cc4)ccc3C2)nc1] | (5-aminopyridin-2-yl)(6-(4- (trifluoromethyl)phenoxy)-3,4-dihydroisoquinolin- 2(1H)-yl)methanone |
| O-0102 | 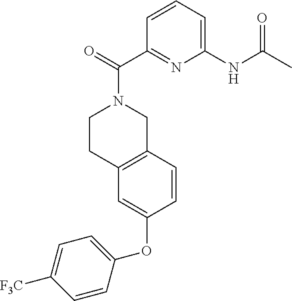 [SMILES: CC(=O)Nc1cccc(C(=O)N2CCc3cc(Oc4ccc(C(F)(F)F)cc4)ccc3C2)n1] | N-(6-(6-(4-(trifluoromethyl)phenoxy)-1,2,3,4- tetrahydroisoquinoline-2-carbonyl)pyridin-2- yl)acetamide |
| O-0103 | 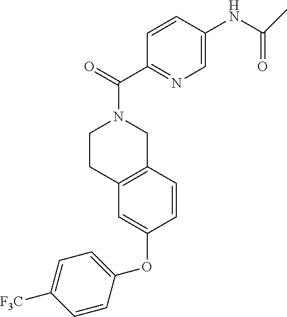 [SMILES: CC(=O)Nc1ccc(C(=O)N2CCc3cc(Oc4ccc(C(F)(F)F)cc4)ccc3C2)nc1] | N-(6-(6-(4-(trifluoromethyl)phenoxy)-1,2,3,4- tetrahydroisoquinoline-2-carbonyl)pyridin-3- yl)acetamide |
| O-0104 | 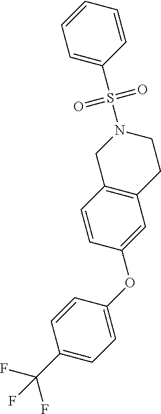 [SMILES: O=S(=O)(c1ccccc1)N1CCc2cc(Oc3ccc(C(F)(F)F)cc3)ccc2C1] | 2-(phenylsulfonyl)-6-(4-(trifluoromethyl)phenoxy)- 1,2,3,4-tetrahydroisoquinoline |
| O-0105 | 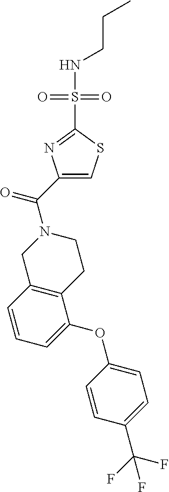 [SMILES: CCCNS(=O)(=O)c1nc(C(=O)N2CCc3c(cccc3Oc3ccc(C(F)(F)F)cc3)C2)cs1] | N-propyl-4-(5-(4-(trifluoromethyl)phenoxy)-1,2,3,4- tetrahydroisoquinoline-2-carbonyl)thiazole-2- sulfonamide |
| O-0106 | 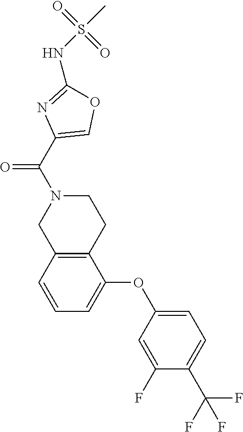 [SMILES: CS(=O)(=O)Nc1nc(C(=O)N2CCc3c(cccc3Oc3ccc(C(F)(F)F)c(F)c3)C2)co1] | N-(4-(5-(3-fluoro-4-(trifluoromethyl)phenoxy)- 1,2,3,4-tetrahydroisoquinoline-2-carbonyl)oxazol-2- yl)methanesulfonamide |
| O-0107 | 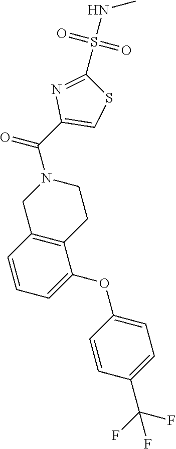 [SMILES: CNS(=O)(=O)c1nc(C(=O)N2CCc3c(cccc3Oc3ccc(C(F)(F)F)cc3)C2)cs1] | N-methyl-4-(5-(4-(trifluoromethyl)phenoxy)-1,2,3,4- tetrahydroisoquinoline-2-carbonyl)thiazole-2- sulfonamide |
| O-0108 | 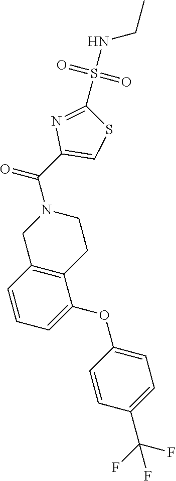 [SMILES: CCNS(=O)(=O)c1nc(C(=O)N2CCc3c(cccc3Oc3ccc(C(F)(F)F)cc3)C2)cs1] | N-ethyl-4-(5-(4-(trifluoromethyl)phenoxy)-1,2,3,4- tetrahydroisoquinoline-2-carbonyl)thiazole-2- sulfonamide |
| O-0109 | 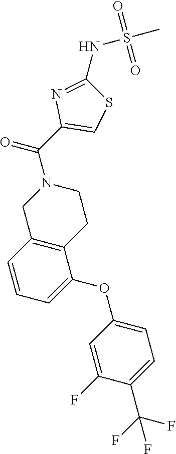 [SMILES: CS(=O)(=O)Nc1nc(C(=O)N2CCc3c(cccc3Oc3ccc(C(F)(F)F)c(F)c3)C2)cs1] | N-(4-(5-(3-fluoro-4-(trifluoromethyl)phenoxy)- 1,2,3,4-tetrahydroisoquinoline-2-carbonyl)thiazol-2- yl)methanesulfonamide |
| O-0110 | 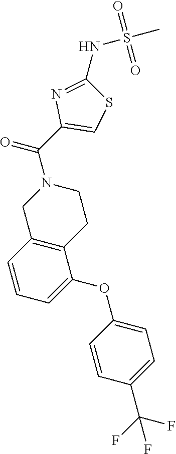 [SMILES: CS(=O)(=O)Nc1nc(C(=O)N2CCc3c(cccc3Oc3ccc(C(F)(F)F)cc3)C2)cs1] | N-(4-(5-(4-(trifluoromethyl)phenoxy)-1,2,3,4- tetrahydroisoquinoline-2-carbonyl)thiazol-2- yl)methanesulfonamide |
| 0-0111 | 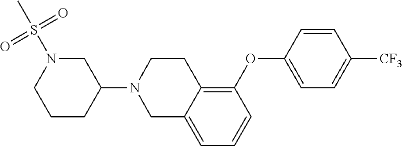 [SMILES: CS(=O)(=O)N1CCCC(N2CCc3c(cccc3Oc3ccc(C(F)(F)F)cc3)C2)C1] | 2-(1-(methylsulfonyl)piperidin-3-yl)-5-(4- (trifluoromethyl)phenoxy)-1,2,3,4- tetrahydroisoquinoline |
| O-0112 | 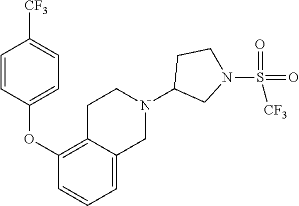 [SMILES: O=S(=O)(N1CCC(N2CCc3c(cccc3Oc3ccc(C(F)(F)F)cc3)C2)C1)C(F)(F)F] | 5-(4-(trifluoromethyl)phenoxy)-2-(1- ((trifluoromethyl)sulfonyl)pyrrolidin-3-yl)- 1,2,3,4-tetrahydroisoquinoline. |
| O-0113 | 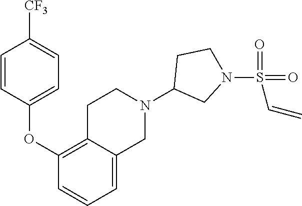 [SMILES: C=CS(=O)(=O)N1CCC(N2CCc3c(cccc3Oc3ccc(C(F)(F)F)cc3)C2)C1] | 5-(4-(trifluoromethyl)phenoxy)-2-(1- (vinylsulfonyl)pyrrolidin-3-yl)-1,2,3,4- tetrahydroisoquinoline |
| O-0114 | 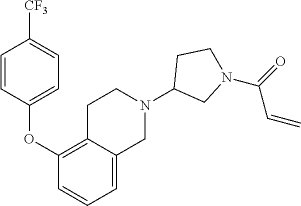 [SMILES: C=CC(=O)N1CCC(N2CCc3c(cccc3Oc3ccc(C(F)(F)F)cc3)C2)C1] | 1-(3-(5-(4-(trifluoromethyl)phenoxy)-3,4- dihydroisoquinolin-2(1H)-yl)pyrrolidin-1- yl)prop-2-en-1-one |
| O-0115 | 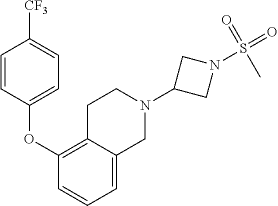 [SMILES: CS(=O)(=O)N1CC(N2CCc3c(cccc3Oc3ccc(C(F)(F)F)cc3)C2)C1] | 2-(1-(methylsulfonyl)azetidin-3-yl)-5-(4- (trifluoromethyl)phenoxy)-1,2,3,4-tetrahydroiso- quinoline |
| O-0116 | 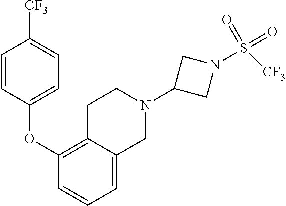 [SMILES: O=S(=O)(N1CC(N2CCc3c(cccc3Oc3ccc(C(F)(F)F)cc3)C2)C1)C(F)(F)F] | 5-(4-(trifluoromethyl)phenoxy)-2-(1- ((trifluoromethyl)sulfonyl)azetidin-3-yl)-1,2,3,4- tetrahydroisoquinoline |
| O-0118 | 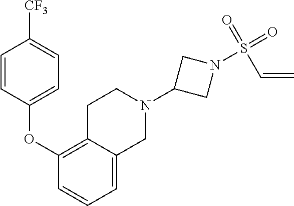 [SMILES: C=CS(=O)(=O)N1CC(N2CCc3c(cccc3Oc3ccc(C(F)(F)F)cc3)C2)C1] | 5-(4-(trifluoromethyl)phenoxy)-2-(1- (vinylsulfonyl)azetidin-3-yl)-1,2,3,4- tetrahydroisoquinoline. |
| 0-0119 | 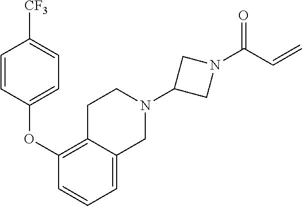 [SMILES: C=CC(=O)N1CC(N2CCc3c(cccc3Oc3ccc(C(F)(F)F)cc3)C2)C1] | 1-(3-(5-(4-(trifluoromethyl)phenoxy)-3,4- dihydroisoquinolin-2(1H)-yl)azetidin-1-yl)prop- 2-en-1-one |
| O-0120 | 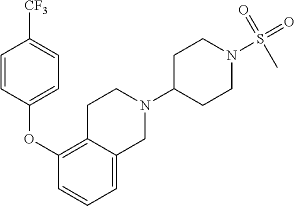 [SMILES: CS(=O)(=O)N1CCC(N2CCc3c(cccc3Oc3ccc(C(F)(F)F)cc3)C2)CC1] | 2-(1-(methylsulfonyl)piperidin-4-yl)-5-(4- (trifluoromethyl)phenoxy)-1,2,3,4- tetrahydroisoquinoline |
| 0-0121 | 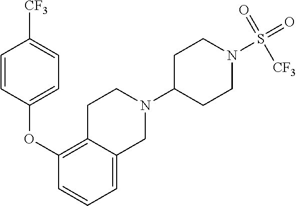 [SMILES: O=S(=O)(N1CCC(N2CCc3c(cccc3Oc3ccc(C(F)(F)F)cc3)C2)CC1)C(F)(F)F] | 5-(4-(trifluoromethyl)phenoxy)-2-(1- ((trifluoromethyl)sulfonyl)piperidin-4-yl)- 1,2,3,4-tetrahydroisoquinoline |
| 0-0122 | 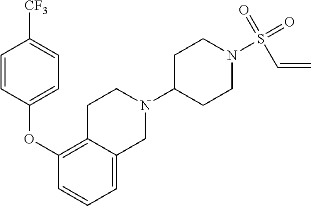 [SMILES: C=CS(=O)(=O)N1CCC(N2CCc3c(cccc3Oc3ccc(C(F)(F)F)cc3)C2)CC1] | 5-(4-(trifluoromethyl)phenoxy)-2-(1- (vinylsulfonyl)piperidin-4-yl)-1,2,3,4- tetrahydroisoquinoline |
| 0-0123 | 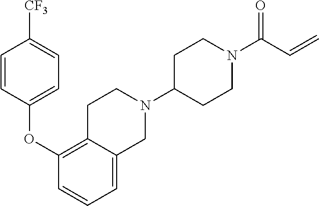 [SMILES: C=CC(=O)N1CCC(N2CCc3c(cccc3Oc3ccc(C(F)(F)F)cc3)C2)CC1] | 1-(4-(5-(4-(trifluoromethyl)phenoxy)-3,4- dihydroisoquinolin-2(1H)-yl)piperidin-1- yl)prop-2-en-1-one |
| 0-0124 | 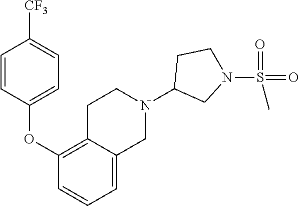 [SMILES: CS(=O)(=O)N1CCC(N2CCc3c(cccc3Oc3ccc(C(F)(F)F)cc3)C2)C1] | 5-(4-(trifluoromethyl)phenoxy)-2-(1- ((methyl)sulfonyl)pyrrolidin-3-yl)-1,2,3,4- tetrahydroisoquinoline. |

### Biological Examples

### Biological Test Methods

### Determine Inhibitor Activity Against TEAD-Dependent Transcription in a Cell-Based Reporter Assay

The human mesothelioma cell line MSTO-211H was stably transfected with a pGL4.21 plasmid containing a synthetic promoter with 12 copies of the GTIIC TEAD response element that drives the expression of a luciferase reporter gene 1 using Lipofectamine transfection reagent (Thermo Fisher). Transfected cells were selected using puromycin and single cell clones expressing this construct (referred to as MSTO-211H+12XGTIIC) were generated by limiting dilution. MSTO-211H cells was characterized by genetic alterations in key components of the Hippo pathway², resulting in up-regulation of YAP/TEAD-mediated transcription. Stable expression of the 12XGTIIC reporter construct in MSTO-211H cells resulted in constitutive luciferase expression. Treatment of these cells with inhibitors targeting TEAD resulted in decreased luciferase expression. To evaluate TEAD inhibitors, the MSTO-211H+12XGTIIC cell line was seeded in a 96-well plate in 50 μL of culture media at 1×10⁴ cells per well and incubated at 37° C. overnight. Serial dilutions of compounds (in total volume of 50 μL of culture media) were added to the cells and incubated at 37° C. for 24 hours. Each plate included cells treated with DMSO as high controls and cells treated with 20 μM of the reference compound K-975 (a known TEAD inhibitor) 3 as low controls. Cell viability was assayed by the addition of 25 μL of CellTiter-Fluor reagent (Promega) followed by a 30-minute incubation at 37° C. and quantification of the fluorescence signal (Ex400/Em505). Subsequently, luciferase expression was assayed by the addition of 25 μL of ONE-Glo reagent (Promega) followed by a 10-minute incubation at room temperature and quantification of the luminescence signal. The luminescence signal was normalized to the fluorescence signal to correct for any loss in cell viability over the 24-hour compound incubation period. The percentage of inhibition of normalized luminescence signal, indicative of compound-mediated inhibition of TEAD-dependent transcription, at individual compound concentrations relative to high and low controls was calculated. The data were analyzed by using nonlinear regression to generate IC₅₀ values for individual compounds.

### REFERENCES

1. Dupont, S. et al. Role of YAP/TAZ in mechanotransduction. Nature 474, 179-184 (2011).
2. Miyanaga, A. et al. Hippo pathway gene mutations in malignant mesothelioma: Revealed by RNA and targeted exon sequencing. Journal of Thoracic Oncology 10, 844-851(2015).
3. Kaneda, A. et al. The novel potent TEAD inhibitor, K-975, inhibits YAP1/TAZ-TEAD protein-protein interactions and exerts an anti-tumor effect on malignant pleural mesothelioma. Am J Cancer Res vol. 10(2020).

The following Table 2 provides data indicating biochemical and/or cell inhibitory activity for exemplary compounds as described herein in Table 1. In Table 2 below, activity is provided as follows: +++=0.001 μM<IC₅₀ <10 μM; ++=10 μM<IC, <100 μM, +=IC, >100 μM. The absence of data in Table 2 indicates unavailable data and is not indicative of activity.

TABLE 2

| | MSTO-211H |
|---|---|
| | GMean |
| | IC50 (uM) |
| | Cell Assay |
| O # | Reporter Cell Variant: TEAD |
| O-0002 | +++ |
| O-0003 | +++ |
| O-0058 | +++ |
| O-0064 | +++ |
| O-0070 | +++ |
| O-0074 | +++ |
| O-0104 | ++ |
| O-0105 | +++ |
| O-0106 | ++ |
| O-0107 | +++ |
| O-0108 | +++ |
| O-0109 | ++ |
| O-0110 | +++ |
| O-0111 | +++ |
| O-0112 | +++ |
| O-0113 | +++ |
| O-0114 | +++ |
| O-0115 | +++ |
| O-0116 | +++ |
| O-0117 | +++ |
| O-0118 | ++ |
| O-0119 | +++ |
| O-0120 | +++ |
| O-0121 | +++ |
| O-0122 | ++ |
| O-0123 | +++ |

All patents and other references cited herein are indicative of the level of skill of those skilled in the art to which the disclosure pertains, and are incorporated by reference in their entireties, including any tables and figures, to the same extent as if each reference had been incorporated by reference in its entirety individually.

One skilled in the art would readily appreciate that the present disclosure is well adapted to obtain the ends and advantages mentioned, as well as those inherent therein. The methods, variances, and compositions described herein as presently representative of the embodiments described herein are exemplary and are not intended as limitations on the scope of the disclosure. Changes therein and other uses will occur to those skilled in the art, which are encompassed within the spirit of the disclosure, are defined by the scope of the claims.

It will be readily apparent to one skilled in the art that varying substitutions and modifications may be made to the present disclosure described herein without departing from the scope and spirit of the disclosure. For example, variations can be made to provide additional compounds of the compounds of this disclosure and/or various methods of administration can be used. Thus, such additional embodiments are within the scope of the present disclosure and the following claims.

The present disclosure illustratively described herein suitably may be practiced in the absence of any element or elements, limitation or limitations which is not specifically described herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically described by the embodiments and optional features, modification and variation of the concepts herein described may be resorted to by those skilled in the art, and that such modifications and variations are considered to be within the scope of this disclosure as defined by the appended claims.

In addition, where features or aspects of the disclosure are described in terms grouping of alternatives, those skilled in the art will recognize that the disclosure is also thereby described in terms of any individual member or subgroup of members of the groups described herein.

Also, unless indicated to the contrary, where various numerical values are provided for embodiments, additional embodiments are described by taking any 2 different values as the endpoints of a range. Such ranges are also within the scope of the present disclosure.

Thus, additional embodiments are within the scope of the disclosure and within the following claims.

1. A compound having Formula (I): 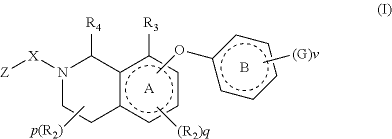
or a pharmaceutically acceptable salt, a tautomer, a stereoisomer, or a deuterated analog thereof, wherein:
ring A is phenyl or a 5- or 6-membered heteroaryl comprising 1-4 heteroatoms selected from N, O and S;
ring B is phenyl or a 5- or 6-membered heteroaryl comprising 1-4 heteroatoms selected from N, O and S,
each G is independently selected from halogen, OH, CN, alkyl optionally substituted with one or more R⁵, and alkoxy optionally substituted with one or more R⁵;
each R² is independently H, halogen, —C(O)O-alkyl, or C₁-C₃alkyl optionally substituted with 1-3 halogens, provided that not more than one R² is —C(O)O-alkyl;
R³ is H; halogen; alkenyl optionally substituted with cycloalkyl or heterocycloalkyl; heterocycloalkyl optionally substituted with —C(O)-alkyl; heterocycloalkenyl optionally substituted with C(O)-alkyl; heterocycloalkylalkyl optionally substituted with C(O)-alkyl; or heteroaryl optionally substituted with haloalkyl, cycloalkyl, or cycloalkylalkyl;
R⁴ is H; alkyl; cycloalkyl optionally substituted with haloalkyl or —C(O)-alkenyl; heterocycloalkyl optionally substituted with —C(O)-alkyl, —C(O)-alkenyl, or —C(O)-cycloalkyl; or heterocycloalkylalkyl optionally substituted with —C(O)-alkyl, —C(O)—CH₂—OH, or heteroaryl;
R⁵ is halogen or OH;
X is a bond, —(CH₂)ₘ—S(O)₂—, —(CH₂)ₙ—C(O)—, or —C(O)O—, wherein the right-hand side indicates the point of attachment to Z;
Z is —NR⁶R⁷, C₁-C₆ alkyl optionally substituted with 1-4 R⁸, C₂-C₆ alkenyl; —C(CH₃)═CH₂, —CH₂—CH═C—O, cycloalkyl optionally substituted with 1-4 R⁹, heterocycloalkyl optionally substituted with 1-4 R¹⁰, aryl optionally substituted with 1-4 R¹⁰, or heteroaryl optionally substituted with 1-4 R¹⁰,
R⁶ is hydrogen, —S(O)₂—C₁-C₃alkyl, C₁-C₆alkyl, cycloalkyl, heterocycloalkyl, aryl, or heteroaryl, wherein each C₁-C₆alkyl, cycloalkyl, heterocycloalkyl, aryl, or heteroaryl is optionally substituted with 1-3 R¹¹;
R⁷ is hydrogen or C₁-C₆alkyl; or
R⁶ and R⁷, together with the nitrogen to which they are attached, form a heterocycloalkyl;
R⁸ is halogen, —NR⁶R⁷, CN, hydroxy, alkoxy, haloalkyl, haloalkoxy, —S—C₁-C₃alkyl, —S(O)—C₁-C₅alkyl, —S(O)₂—C₁-C₅alkyl, —S(O)₂—C₁-C₅haloalkyl, —S(O)₂—NH₂, —S(O)₂(NH)—C₁-C₃alkyl, —C(O)—NH₂, —C(O)OH, —C(O)O—C₁-C₃alkyl, —C(O)C₁-C₅haloalkyl, —N(H)—C(O)—NR⁶R⁷, —N(H)—C(O)—C₁-C₃alkyl optionally substituted with —NR¹²R¹³ or CN, —N(H)—C(O)—C₂-C_{6alkenyl}, —N(H)—C(O)—C₁-C₅haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₃haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰, or
two R⁸, together with the carbon atom to which they are attached, join together to form a cycloalkyl;
each R⁹ is independently CN, —S(O)₂—C₁-C₃alkyl, or —N(C₁-₃alkyl)₂;
each R¹⁰ is independently hydroxy, halogen, CN, C₁-C₆ alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkyl, —NH—C(O)-alkenyl, N(H)—S(O)₂—C₁-C₃alkyl, —S(O)₂—C₁-C₃alkyl, —S(O)₂—C₁-C₃haloalkyl, —S(O)₂—C₂-C_{6alkenyl}, —S(O)₂—NH₂, or —S(O)₂—N(H)—C₁-C₃alkyl;
each R¹¹ is independently hydroxy, CN, alkoxy, —S(O)₂—C₁-C₃alkyl, or cycloalkyl;
each R¹² and R¹³ are independently hydrogen, C₁-C₆alkyl, or C₃-C₆cycloalkyl;
v is 0, 1, 2, 3, or 4;
p is 0, 1, 2, 3, or 4;
q is 0, 1, or 2;
n is 0, 1, or 2; and
m is 0, 1, 2, or 3;
provided that if X is —(CH₂)ₘ—S(O)₂—, —(CH₂)ₙ—C(O)—, or —C(O)O—, then ring A and/or ring B is a 5- or 6-membered heteroaryl comprising 1-4 heteroatoms selected from N, O and S when —O— is attached to Ring A at the 5 or 7 position.

2. A compound of claim 1, having Formula (Ia), (Ib) or (Ic): 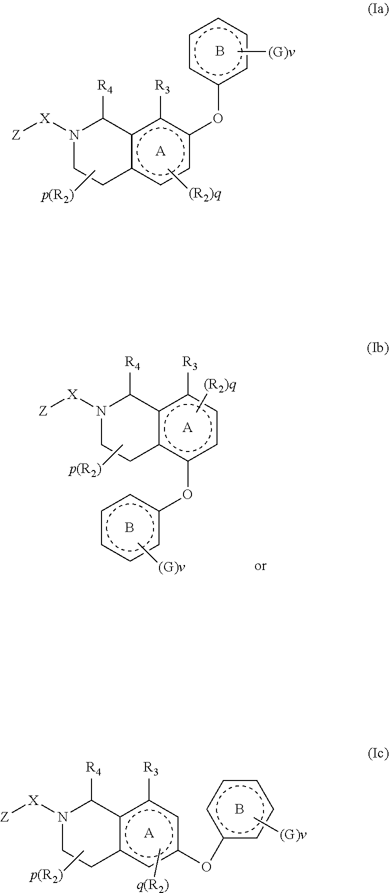
or a pharmaceutically acceptable salt, a tautomer, a stereoisomer, or a deuterated analog thereof, wherein:
ring A is phenyl or a 5- or 6-membered heteroaryl comprising 1-4 heteroatoms selected from N, O and S;
ring B is phenyl or a 5- or 6-membered heteroaryl comprising 1-4 heteroatoms selected from N, O and S,
each G is independently selected from halogen, OH, CN, alkyl optionally substituted with one or more R⁵, and alkoxy optionally substituted with one or more R⁵;
each R² is independently H, halogen, —C(O)O-alkyl, or C₁-C₃alkyl optionally substituted with 1-3 halogens, provided that not more than one R² is —C(O)O-alkyl;
R³ is H; halogen; alkenyl optionally substituted with cycloalkyl or heterocycloalkyl; heterocycloalkyl optionally substituted with —C(O)-alkyl; heterocycloalkenyl optionally substituted with C(O)-alkyl; heterocycloalkylalkyl optionally substituted with C(O)-alkyl; or heteroaryl optionally substituted with haloalkyl, cycloalkyl, or cycloalkylalkyl;
R⁴ is H; alkyl; cycloalkyl optionally substituted with haloalkyl or —C(O)-alkenyl; heterocycloalkyl optionally substituted with —C(O)-alkyl, —C(O)-alkenyl, or —C(O)-cycloalkyl; or heterocycloalkylalkyl optionally substituted with —C(O)-alkyl, —C(O)—CH₂—OH, or heteroaryl;
R⁵ is halogen or OH;
X is a bond, —(CH₂)ₘ—S(O)₂—, —(CH₂)ₙ—C(O)—, or —C(O)O—, wherein the right-hand side indicates the point of attachment to Z;
Z is —NR⁶R⁷, C₁-C₆ alkyl optionally substituted with 1-4 R⁸, C₂-C₆ alkenyl; —C(CH₃)═CH₂, —CH₂—CH═C═O, cycloalkyl optionally substituted with 1-4 R⁹, heterocycloalkyl optionally substituted with 1-4 R¹⁰, aryl optionally substituted with 1-4 R¹⁰, or heteroaryl optionally substituted with 1-4 R¹⁰;
R⁶ is hydrogen, —S(O)₂—C₁-C₃alkyl, C₁-C₃alkyl, cycloalkyl, heterocycloalkyl, aryl, or heteroaryl, wherein each C₁-C₆alkyl, cycloalkyl, heterocycloalkyl, aryl, or heteroaryl is optionally substituted with 1-3 R¹¹;
R⁷ is hydrogen or C₁-C₆alkyl; or
R⁶ and R⁷, together with the nitrogen to which they are attached, form a heterocycloalkyl;
R⁸ is halogen, —NR⁶R⁷, CN, hydroxy, alkoxy, haloalkyl, haloalkoxy, —S—C₁-C₅alkyl, —S(O)—C₁-C₃alkyl, —S(O)₂—C₁-C₃alkyl, —S(O)₂—C₁-C₅haloalkyl, —S(O)₂—NH₂, —S(O)₂ (NH)—C₁-C₅alkyl, —C(O)—NH₂, —C(O)OH, —C(O)O—C₁-C₃alkyl, —C(O)C₁-C₃haloalkyl, —N(H)—C(O)—NR⁶R⁷, —N(H)—C(O)—C₁-C₃alkyl optionally substituted with —NR¹²R¹³ or CN, —N(H)—C(O)—C₂-C₆alkenyl, —N(H)—C(O)—C₁-C₃haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₃haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰; or
two R⁸, together with the carbon atom to which they are attached, join together to form a cycloalkyl;
each R⁹ is independently CN, —S(O)₂—C₁-C₃alkyl, or —N(C₁-₃alkyl)₂;
each R¹⁰ is independently hydroxy, halogen, CN, C₁-C₆ alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkyl, —NH—C(O)-alkenyl, N(H)—S(O)₂—C₁-C₃alkyl, —S(O)₂—C₁-C₃alkyl, —S(O)₂—C₁-C₃haloalkyl, —S(O)₂—C₂-C₆alkenyl, —S(O)₂—NH₂, or —S(O)₂—N(H)—C₁-C₃alkyl;
each R¹¹ is independently hydroxy, CN, alkoxy, —S(O)₂—C₁-C₅alkyl, or cycloalkyl;
each R¹² and R¹³ are independently hydrogen, C₁-C₆alkyl, or C₃-C₆cycloalkyl;
v is 0, 1, 2, 3, or 4;
p is 0, 1, 2, 3, or 4;
qis 0, 1, or 2;
n is 0, 1, or 2; and
m is 0, 1, 2, or 3;
provided that if X is —(CH₂)ₘ—S(O)₂—, —(CH₂)ₙ—C(O)—, or —C(O)O—, then ring A and/or ring B is a 5- or 6-membered heteroaryl comprising 1-4 heteroatoms selected from N, O and S when —O— is attached to Ring A at the 5 or 7 position.

3. A compound of claim 1, wherein
ring A is phenyl or a 5- or 6-membered heteroaryl comprising 1-4 heteroatoms selected from N, O and S;
ring B is phenyl or a 5- or 6-membered heteroaryl comprising 1-4 heteroatoms selected from N, O and S,
each G is independently selected from halogen, OH, CN, alkyl optionally substituted with one or more R⁵, and alkoxy optionally substituted with one or more R⁵;
each R² is independently H, halogen, —C(O)O-alkyl, or C₁-C₃alkyl optionally substituted with 1-3 halogens, provided that not more than one R² is —C(O)O-alkyl;
R³ is H; halogen; alkenyl optionally substituted with cycloalkyl or heterocycloalkyl; heterocycloalkyl optionally substituted with —C(O)-alkyl; heterocycloalkenyl optionally substituted with C(O)-alkyl; heterocycloalkylalkyl optionally substituted with C(O)-alkyl; or heteroaryl optionally substituted with haloalkyl, cycloalkyl, or cycloalkylalkyl;
R⁴ is H; alkyl; cycloalkyl optionally substituted with haloalkyl or —C(O)-alkenyl; heterocycloalkyl optionally substituted with —C(O)-alkyl, —C(O)-alkenyl, or —C(O)-cycloalkyl; or heterocycloalkylalkyl optionally substituted with —C(O)-alkyl, —C(O)—CH₂—OH, or heteroaryl;
R⁵ is halogen or OH;
X is —(CH₂)ₘ—S(O)₂—, —(CH₂)ₙ—C(O)—, or —C(O)O—, wherein the right-hand side indicates the point of attachment to Z;
Z is —NR⁶R⁷, C₁-C₆alkyl optionally substituted with 1-4 R⁸, —C(CH₃)-CH₂, —CH₂—CH═C—O, cycloalkyl optionally substituted with 1-4 R⁹, heterocycloalkyl optionally substituted with 1-4 R¹⁰, aryl optionally substituted with 1-4 R¹⁰, or heteroaryl optionally substituted with 1-4 R¹⁰;
R⁶ is hydrogen, —S(O)₂—C₁-C₅alkyl, C₁-C₆alkyl, cycloalkyl, heterocycloalkyl, aryl, or heteroaryl, wherein each C₁-C₆alkyl, cycloalkyl, heterocycloalkyl, aryl, or heteroaryl is optionally substituted with 1-3 R¹¹;
R⁷ is hydrogen or C₁-C₆alkyl; or
R⁶ and R⁷, together with the nitrogen to which they are attached, form a heterocycloalkyl;
R⁸ is —NR⁶R⁷, CN, hydroxy, alkoxy, haloalkoxy, —S—C₁-C₃alkyl, —S(O)—C₁-C₃alkyl, —S(O)₂—C₁-C₃alkyl, —S(O)₂—C₁-C₃haloalkyl, —S(O)₂—NH₂, —S(O)(NH)—C₁-C₃alkyl, —C(O)—NH₂, —C(O)OH, —C(O)O—C₁-C₅alkyl, —C(O)C₁-C₃haloalkyl, —N(H)—C(O)—NR⁶R⁷, —N(H)—C(O)—C₁-C₅alkyl optionally substituted with —NR¹²R¹³ or CN, —N(H)—C(O)—C₂-C₆alkenyl, —N(H)—C(O)—C₁-C₃haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₃haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰; or
two R⁸, together with the carbon atom to which they are attached, join together to form a cycloalkyl;
each R⁹ is independently CN, —S(O)₂—C₁-C₃alkyl, or —N(C₁-3alkyl)₂;
each R¹⁰ is independently hydroxy, halogen, CN, C₁-C₆alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkenyl, —S(O)₂—C₁-C₅alkyl, —S(O)₂—C₂-C₆alkenyl, or —S(O)₂—NH₂;
each R¹¹ is independently hydroxy, CN, alkoxy, —S(O)₂—C₁-C₃alkyl, or cycloalkyl;
each R¹² and R¹³ are independently hydrogen, C₁-C₆alkyl, or C₃-C₆cycloalkyl;
v is 0, 1, 2, 3, or 4;
p is 0, 1, 2, 3, or 4;
q is 0, 1, or 2;
n is 0, 1, or 2; and
m is 0, 1, 2, or 3;
provided that ring A and/or ring B is a 5- or 6-membered heteroaryl comprising 1-4 heteroatoms selected from N, O and S.

4. A compound of claim 1, having Formula (Ia) or (Ib): 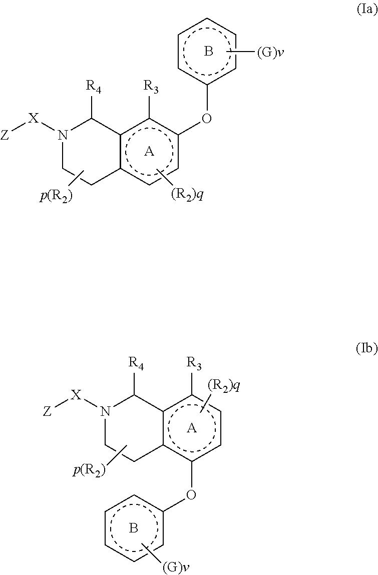
or a pharmaceutically acceptable salt, a tautomer, a stereoisomer, or a deuterated analog thereof, wherein:
ring A is phenyl or a 5- or 6-membered heteroaryl comprising 1-4 heteroatoms selected from N, O and S,
ring B is phenyl or a 5- or 6-membered heteroaryl comprising 1-4 heteroatoms selected from N, O and S,
each G is independently selected from halogen, OH, CN, alkyl optionally substituted with one or more R⁵, and alkoxy optionally substituted with one or more R⁵;
each R² is independently H, halogen, —C(O)O-alkyl, or C₁-C₃alkyl optionally substituted with 1-3 halogens, provided that not more than one R² is —C(O)O-alkyl;
R³ is H; halogen; alkenyl optionally substituted with cycloalkyl or heterocycloalkyl; heterocycloalkyl optionally substituted with —C(O)-alkyl; heterocycloalkenyl optionally substituted with C(O)-alkyl; heterocycloalkylalkyl optionally substituted with C(O)-alkyl; or heteroaryl optionally substituted with haloalkyl, cycloalkyl, or cycloalkylalkyl;
R⁴ is H; alkyl; cycloalkyl optionally substituted with haloalkyl or —C(O)-alkenyl; heterocycloalkyl optionally substituted with —C(O)-alkyl, —C(O)-alkenyl, or —C(O)-cycloalkyl; or heterocycloalkylalkyl optionally substituted with —C(O)-alkyl, —C(O)—CH₂—OH, or heteroaryl;
R⁵ is halogen or OH;
X is —(CH₂)ₘ—S(O)₂—, —(CH₂)ₙ—C(O)—, or —C(O)O—, wherein the right-hand side indicates the point of attachment to Z;
Z is —NR⁶R⁷, C₁-C₆alkyl optionally substituted with 1-4 R⁸, —C(CH₃)-CH₂, —CH₂—CH═C═O, cycloalkyl optionally substituted with 1-4 R⁹, heterocycloalkyl optionally substituted with 1-4 R¹⁰, aryl optionally substituted with 1-4 R¹⁰, or heteroaryl optionally substituted with 1-4 R¹⁰;
R⁶ is hydrogen, C₁-C₆alkyl, cycloalkyl, heterocycloalkyl, aryl, or heteroaryl, wherein each C₁-C₆alkyl, cycloalkyl, heterocycloalkyl, aryl, or heteroaryl is optionally substituted with 1-3 R¹¹;
R⁷ is hydrogen or C₁-C₆alkyl; or
R⁶ and R⁷, together with the nitrogen to which they are attached, form a heterocycloalkyl;
R⁸ is —NR⁶R⁷, CN, hydroxy, alkoxy, haloalkoxy, —S—C₁-C₅alkyl, —S(O)—C₁-C₃alkyl, —S(O)₂—C₁-C₃alkyl, —S(O)₂—C₁-C₃haloalkyl, —S(O)₂—NH₂, —S(O)(NH)—C₁-C₃alkyl, —C(O)—NH₂, —C(O)OH, —C(O)O—C₁-C₃alkyl, —C(O)C₁-C₃haloalkyl, —N(H)—C(O)—NR⁶R⁷, —N(H)—C(O)—C₁-C₅alkyl optionally substituted with —NR¹²R¹³ or CN, —N(H)—C(O)—C₂-C₆alkenyl, —N(H)—C(O)—C₁-C₃haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₃haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰; or
two R⁸, together with the carbon atom to which they are attached, join together to form a cycloalkyl;
each R⁹ is independently CN, —S(O)₂—C₁-C₃alkyl, or —N(C₁-3alkyl)₂;
each R¹⁰ is independently hydroxy, halogen, CN, C₁-C₆alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkenyl, —S(O)₂—C₁-C₃alkyl, —S(O)₂—C₂-C₆alkenyl, or —S(O)₂—NH₂;
R¹¹ is CN;
each R¹² and R¹³ are independently hydrogen, C₁-C₆alkyl, or C₃-C₆cycloalkyl;
v is 0, 1, 2, 3, or 4;
p is 0, 1, 2, 3, or 4;
q is 0, 1, or 2;
n is 0, 1, or 2; and
m is 0, 1, 2, or 3;
provided that ring A and/or ring B is a 5- or 6-membered heteroaryl comprising 1-4 heteroatoms selected from N, O and S.

5. The compound of claim 3, wherein:
ring A is phenyl or a 5- or 6-membered heteroaryl comprising 1-4 heteroatoms selected from N, O and S,
ring B is phenyl or a 5- or 6-membered heteroaryl comprising 1-4 heteroatoms selected from N, O and S,
each G is independently selected from halogen, OH, CN, alkyl optionally substituted with one or more R⁵, and alkoxy optionally substituted with one or more R⁵;
each R² is independently H, halogen, —C(O)O-alkyl, or C₁-C₃alkyl optionally substituted with 1-3 halogens, provided that not more than one R² is —C(O)O-alkyl;
R³ is H; halogen; alkenyl optionally substituted with cycloalkyl or heterocycloalkyl; heterocycloalkyl optionally substituted with —C(O)-alkyl; heterocycloalkenyl optionally substituted with C(O)-alkyl; heterocycloalkylalkyl optionally substituted with C(O)-alkyl; or heteroaryl optionally substituted with haloalkyl, cycloalkyl, or cycloalkylalkyl;
R⁴ is H; alkyl; cycloalkyl optionally substituted with haloalkyl or —C(O)-alkenyl; heterocycloalkyl optionally substituted with —C(O)-alkyl, —C(O)-alkenyl, or —C(O)-cycloalkyl; or heterocycloalkylalkyl optionally substituted with —C(O)-alkyl, —C(O)—CH₂—OH, or heteroaryl;
R⁵ is halogen or OH;
X is —(CH₂)ₘ—S(O)₂—, —(CH₂)ₙ—C(O)—, or —C(O)O—, wherein the right-hand side indicates the point of attachment to Z;
Z is —NR⁶R⁷, C₁-C₆alkyl optionally substituted with 1-4 R⁸, —CH₂—CH═C═O, aryl optionally substituted with 1-4 R¹⁰, or heteroaryl optionally substituted with 1-4 R¹⁰;
R⁶ is hydrogen, C₁-C₆alkyl, cycloalkyl, heterocycloalkyl, aryl, or heteroaryl, wherein each C₁-C₆alkyl, cycloalkyl, heterocycloalkyl, aryl, or heteroaryl is optionally substituted with 1-3 R¹¹;
R⁷ is hydrogen or C₁-C₆alkyl; or
R⁶ and R⁷, together with the nitrogen to which they are attached, form a heterocycloalkyl;
R⁸ is —NR⁶R⁷, CN, hydroxy, alkoxy, haloalkoxy, —S—C₁-C₃alkyl, —S(O)—C₁-C₅alkyl, —S(O)₂—C₁-C₃alkyl, —S(O)₂—C₁-C₃haloalkyl, —S(O)₂—NH₂, —S(O)(NH)—C₁-C₃alkyl, —C(O)—NH₂, —C(O)OH, —C(O)O—C₁-C₃alkyl, —C(O)C₁-C₃haloalkyl, —N(H)—C(O)—NR⁶R⁷, —N(H)—C(O)—C₁-C₅alkyl optionally substituted with —NR¹²R¹³ or CN, —N(H)—C(O)—C₂-C₆alkenyl, —N(H)—C(O)—C₁-C₅haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₃haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰; or
two R⁸, together with the carbon atom to which they are attached, join together to form a cycloalkyl;
each R⁹ is independently CN, —S(O)₂—C₁-C₃alkyl, or —N(C₁-3alkyl)₂;
each R¹⁰ is independently hydroxy, halogen, CN, C₁-C₆alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkenyl, —S(O)₂—C₁-C₃alkyl, —S(O)₂—C₂-C₆alkenyl, or —S(O)₂—NH₂;
R¹¹ is CN;
each R¹² and R¹³ are independently hydrogen, C₁-C₆alkyl, or C₃-C₆cycloalkyl;
v is 0, 1, 2, 3, or 4;
p is 0, 1, 2, 3, or 4;
q is 0, 1, or 2;
n is 0, 1, or 2; and
m is 0, 1, 2, or 3.

6. The compound according to claim 3, wherein:
v is 0, 1, 2, or 3;
each G is independently selected from halogen, CN, and C₁-C₃alkyl optionally substituted with 1-3 R⁵;
each R² is H, halogen, or CH₃;
R⁵ is halogen or OH;
X is —(CH₂)ₘ—S(O)₂— or —(CH₂)ₙ—C(O)—; and
Z is —NR⁶R⁷, C₁-C₆alkyl optionally substituted with 1-3 R⁸, cycloalkyl optionally substituted with 1-3 R⁹, heterocycloalkyl optionally substituted with 1-3 R¹⁰, aryl optionally substituted with 1-3 R¹⁰, or heteroaryl optionally substituted with 1-3 R¹⁰.

7. The compound according to claim 6, wherein:
v is 0, 1, or 2;
each G is independently selected from C₁, F, CN, and C₁-C₃alkyl substituted with 1-3 R⁵;
each R² is H, C₁, F, or CH₃; and
R⁵ is halogen.

8. The compound of claim 7, wherein R⁵ is Cl or F.

9. The compound of claim 3, having one of the following formulae: 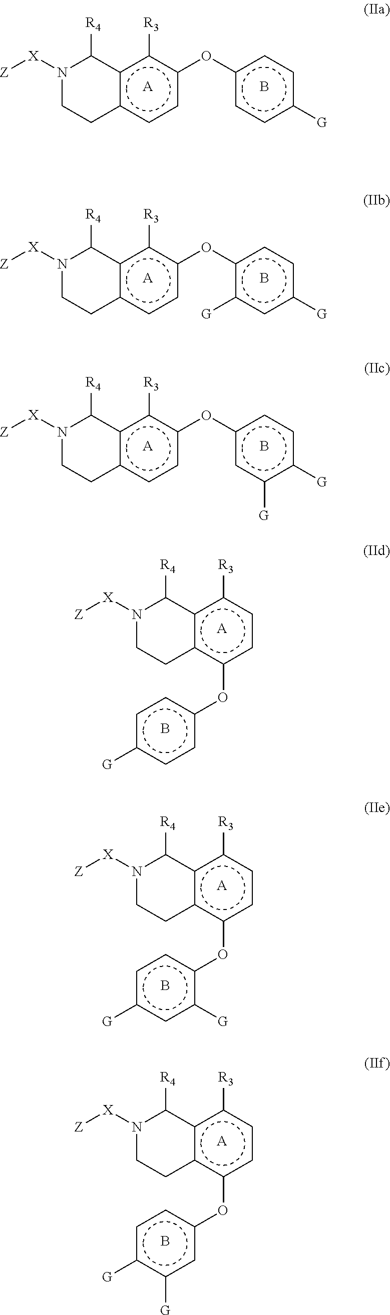
or a pharmaceutically acceptable salt, a tautomer, a stereoisomer, or a deuterated analog of any of formulae (IIa), (IIb), (IIc), (IId), (IIe), or (IIf); wherein:
each G is independently selected from Cl, F, CN, and C₁-C₃alkyl substituted with 1-3 R⁵; and
R⁵ is halogen.

10. The compound according to claim 9, having one of the following formulae: 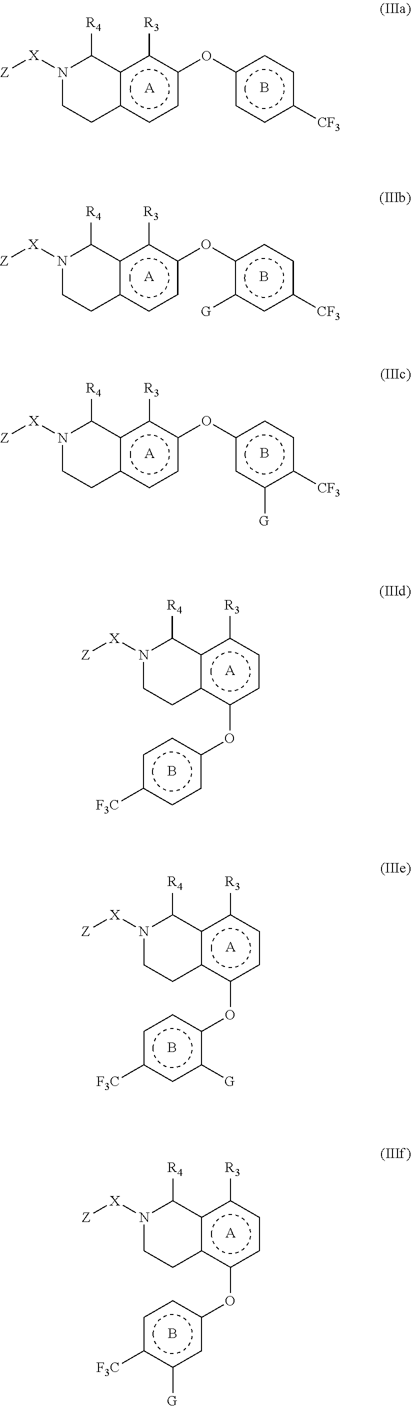
or a pharmaceutically acceptable salt, a tautomer, a stereoisomer, or a deuterated analog of any of formulae (IIIa), (IIIb), (IIIc), (IIId), (IIIe) or (IIIf); wherein:
G is Cl, F, or CN.

11. The compound of claim 9, wherein R³ is H; halogen; C₂-C₄alkenyl optionally substituted with cyclopropyl or heterocycloalkyl; heterocycloalkyl optionally substituted with C(O)—CH₃; heterocycloalkenyl optionally substituted with C(O)—CH₃; heterocycloalkylalkyl optionally substituted with C(O)—CH₃; or 5-6 membered heteroaryl optionally substituted with haloalkyl, cyclopropyl, or cyclopropyl —CH₂—.

12. The compound of claim 9, wherein R⁴ is H; C₁-C₃alkyl; cycloalkyl optionally substituted with haloalkyl or —C(O)-alkenyl; heterocycloalkyl optionally substituted with —C(O)—CH₃, —C(O)—CH—CH₂, or —C(O)-cyclopropyl; heterocycloalkylalkyl optionally substituted with —C(O)—CH₃; —C(O)—CH₂—OH; or 5-6 membered heteroaryl.

13. The compound of claim 3, having one of the following formulae: 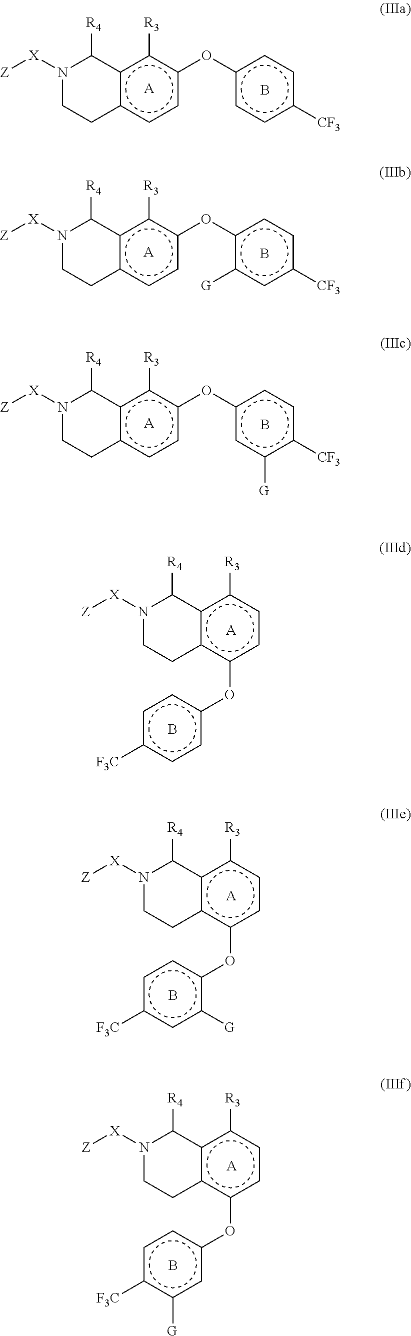
or a pharmaceutically acceptable salt, a tautomer, a stereoisomer, or a deuterated analog of any of formulae (IIIa), (IIIb), (IIIc), (IIId), (IIIe) or (IIIf); wherein:
G is C₁, F, or CN;
R³ is H; halogen; C₂-C₄alkenyl optionally substituted with cyclopropyl or heterocycloalkyl; heterocycloalkyl optionally substituted with C(O)—CH₃; heterocycloalkenyl optionally substituted with C(O)—CH₃; heterocycloalkylalkyl optionally substituted with C(O)—CH₃; or 5-6 membered heteroaryl optionally substituted with haloalkyl, cyclopropyl, or cyclopropyl —CH₂—;
R⁴ is H; C₁-C₃alkyl; cycloalkyl optionally substituted with haloalkyl or —C(O)-alkenyl; heterocycloalkyl optionally substituted with —C(O)—CH₃—C(O)—CH—CH₂, or —C(O)-cyclopropyl; heterocycloalkylalkyl optionally substituted with —C(O)—CH₃; —C(O)—CH₂—OH; or 5-6 membered heteroaryl;
X is —(CH₂)ₙ—C(O)—;
Z is —NR⁶R⁷, C₁-C₆alkyl optionally substituted with 1-4 R⁸, C₃-C₆cycloalkyl optionally substituted with 1-4 R⁹, 5-10 membered heterocycloalkyl optionally substituted with 1-4 R¹⁰, C₆-C₁₂aryl optionally substituted with 1-4 R¹⁰, or 5-10 membered heteroaryl optionally substituted with 1-4 R¹⁰, and
n is 0, 1, or 2.

14. The compound of claim 13, wherein:
Z is —NR⁶R⁷, C₁-C₄alkyl optionally substituted with 1-3 R⁸, C₃-C₆cycloalkyl optionally substituted with 1-3 R⁹, 5-7 membered heterocycloalkyl optionally substituted with 1-3 R¹⁰, C₆-C₁₀aryl optionally substituted with 1-3 R¹⁰, or 5-7 membered heteroaryl optionally substituted with 1-3 R¹⁰, and
n is 0 or 1.

15. The compound of claim 13, wherein:
Z is —NR⁶R⁷, C₂-C₄alkyl optionally substituted with 1-3 R⁸, C₆-C₁₂aryl optionally substituted with 1-3 R¹⁰, or 5-10 membered heteroaryl optionally substituted with 1-3 R¹⁰, and
n is 0 or 1.

16. The compound of claim 13, wherein:
Z is —NR⁶R⁷; and
n is 0 or 1.

17. The compound of claim 13, wherein:
Z is C₂-C₄alkyl optionally substituted with 1-3 R⁸; and
n is 0 or 1.

18. The compound of claim 13, wherein:
Z is C₆-C₁₂aryl optionally substituted with 1-3 R¹⁰; and
n is 0 or 1.

19. The compound of claim 13, wherein:
Z is 5-10 membered heteroaryl optionally substituted with 1-3 R¹⁰, and
n is 0 or 1.

20. The compound of claim 3, wherein R₃ and R₄ are H.

21. A compound of claim 1, having one of the following formulae: 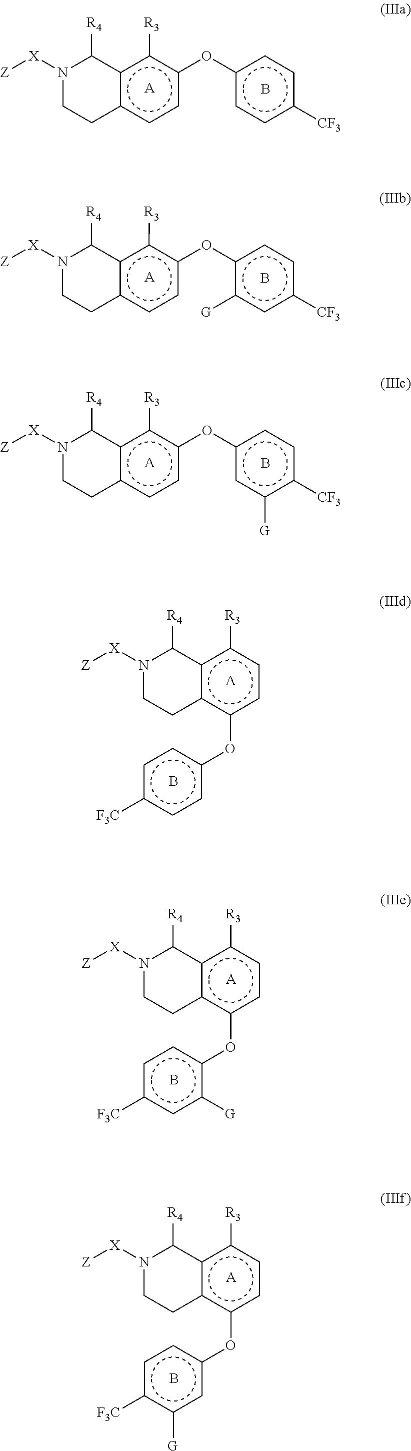 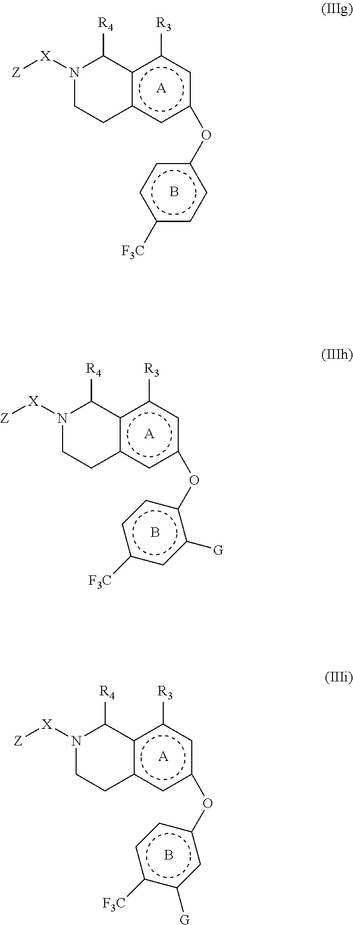
or a pharmaceutically acceptable salt, a tautomer, a stereoisomer, or a deuterated analog of any of formulae (IIIa), (IIIb), (IIIc), (IIId), (IIIe), (IIIf), (IIIg), (IIIh), or (IIIi); wherein:
G is C₁, F, or CN;
R³ is H; halogen; C₂-C₄alkenyl optionally substituted with cyclopropyl or heterocycloalkyl; heterocycloalkyl optionally substituted with C(O)—CH₃; heterocycloalkenyl optionally substituted with C(O)—CH₃; heterocycloalkylalkyl optionally substituted with C(O)—CH₃; or 5-6 membered heteroaryl optionally substituted with haloalkyl, cyclopropyl, or cyclopropyl-CH₂—;
R⁴ is H; C₁-C₅alkyl; cycloalkyl optionally substituted with haloalkyl or —C(O)-alkenyl; heterocycloalkyl optionally substituted with —C(O)—CH₃, —C(O)—CH—CH₂, or —C(O)-cyclopropyl; heterocycloalkylalkyl optionally substituted with —C(O)—CH₃; —C(O)—CH₂—OH; or 5-6 membered heteroaryl;
X is —(CH₂)ₙ—C(O)—;
Z is —NR⁶R⁷, C₂-C₄alkenyl, C₂-C₄alkyl optionally substituted with 1-3 R⁸, C₆-C₁₂aryl optionally substituted with 1-3 R¹⁰, or 5-10 membered heteroaryl optionally substituted with 1-3 R¹⁰;
R⁶ is hydrogen, C₁-C₆alkyl, cycloalkyl, heterocycloalkyl, aryl, or heteroaryl, wherein each C₁-C₆alkyl cycloalkyl, heterocycloalkyl, aryl, or heteroaryl is optionally substituted with 1-3 R¹¹;
R⁷ is hydrogen or C₁-C₆alkyl; or
R⁸ is halogen, —NR⁶R⁷, CN, hydroxy, alkoxy, haloalkyl, haloalkoxy, —S—C₁-C₃alkyl, —S(O)—C₁-C₃alkyl, —S(O)₂—C₁-C₃alkyl, —S(O)₂—C₁-C₃haloalkyl, —S(O)₂—NH₂, —S(O)₂ (NH)—C₁-C₃alkyl, —C(O)—NH₂, —C(O)OH, —C(O)O—C₁-C₃alkyl, —C(O)C₁-C₅haloalkyl, —N(H)—C(O)—NR⁶R⁷, —N(H)—C(O)—C₁-C₃alkyl optionally substituted with —NR¹²R¹³ or CN, —N(H)—C(O)—C₂-Calkenyl, —N(H)—C(O)—C₁-C₅haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₅haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰; or
two R⁸, together with the carbon atom to which they are attached, join together to form a cycloalkyl;
each R¹⁰ is independently hydroxy, halogen, CN, C₁-C₆alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkyl, —NH—C(O)-alkenyl, —NH—S(O)₂—C₁-C₃alkyl, —S(O)₂—C₁-C₃haloalkyl, —S(O)₂—C₁-C₃alkyl, —S(O)₂—C₂-C₆alkenyl, —S(O)₂—NH₂ or —S(O)₂—NH—C₁-C₃alkyl;
R¹¹ is CN;
each R¹² and R¹³ are independently hydrogen, C₁-C₆alkyl, or C₃-C₆cycloalkyl; and
n is 0 or 1;
provided that ring A and/or ring B is a 5- or 6-membered heteroaryl comprising 1-4 heteroatoms selected from N, O and S when —O— is attached to Ring A at the 5 or 7 position.

22. A compound of claim 1, having one of the following formulae: 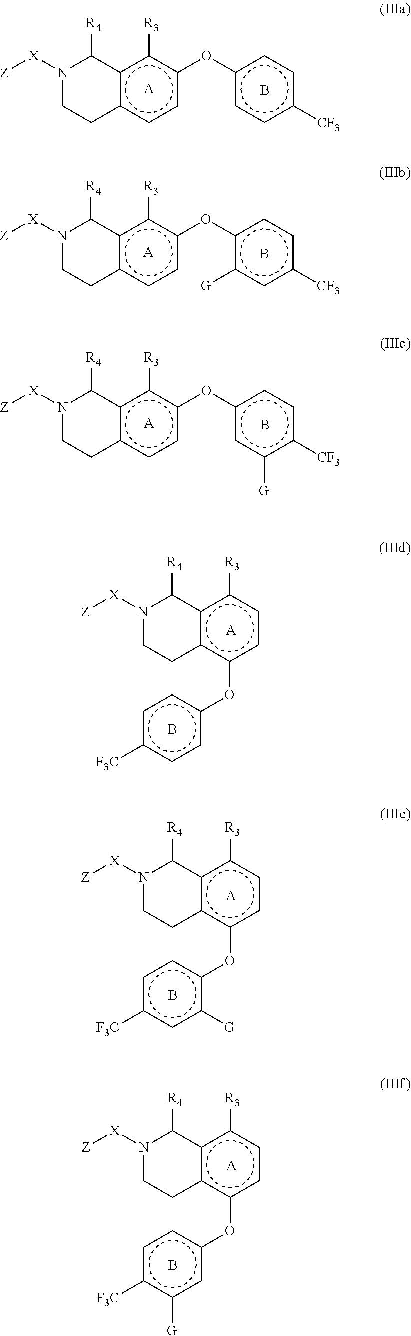
or a pharmaceutically acceptable salt, a tautomer, a stereoisomer, or a deuterated analog of any of formulae (IIIa), (IIIb), (IIIc), (IIId), (IIIe) or (IIIf); wherein:
G is C₁, F, or CN;
R³ is H; halogen; C₂-C₄alkenyl optionally substituted with cyclopropyl or heterocycloalkyl; heterocycloalkyl optionally substituted with C(O)—CH₃; heterocycloalkenyl optionally substituted with C(O)—CH₃; heterocycloalkylalkyl optionally substituted with C(O)—CH₃; or 5-6 membered heteroaryl optionally substituted with haloalkyl, cyclopropyl, or cyclopropyl —CH₂—;
R⁴ is H; C₁-C₃alkyl; cycloalkyl optionally substituted with haloalkyl or —C(O)-alkenyl; heterocycloalkyl optionally substituted with —C(O)—CH₃, —C(O)—CH═CH₂, or —C(O)-cyclopropyl; heterocycloalkylalkyl optionally substituted with —C(O)—CH₃; —C(O)—CH₂—OH; or 5-6 membered heteroaryl;
X is —(CH₂)ₙ—C(O)—;
Z is —NR⁶R⁷, C₂-C₄alkyl optionally substituted with 1-3 R⁸, C₆-C₁₂aryl optionally substituted with 1-3 R¹⁰, or 5-10 membered heteroaryl optionally substituted with 1-3 R¹⁰;
R⁶ is hydrogen, C₁-C₆alkyl, cycloalkyl, heterocycloalkyl, aryl, or heteroaryl, wherein each C₁-C₆alkyl cycloalkyl, heterocycloalkyl, aryl, or heteroaryl is optionally substituted with 1-3 R¹¹;
R⁷ is hydrogen or C₁-C₆alkyl; or
R⁸ is —NR⁶R⁷, CN, hydroxy, alkoxy, haloalkoxy, —S—C₁-C₃alkyl, —S(O)—C₁-C₃alkyl, —S(O)₂—C₁-C₃alkyl, —S(O)₂—C₁-C₅haloalkyl, —S(O)₂—NH₂, —S(O)(NH)—C₁-C₃alkyl, —C(O)—NH₂, —C(O)OH, —C(O)O—C₁-C₃alkyl, —C(O)C₁-C₅haloalkyl, —N(H)—C(O)—NR⁶R⁷, —N(H)—C(O)—C₁-C₅alkyl optionally substituted with —NR¹²R¹³ or CN, —N(H)—C(O)—C₂-C₆alkenyl, —N(H)—C(O)—C₁-C₅haloalkyl, —N(H)—C(O)O—C₁-C₆alkyl, —N(H)—C(NH)—NH₂, —N(H)—S(O)₂—NR¹²R¹³, —N(R⁷)—S(O)₂—C₁-C₃alkyl, —N(R⁷)—S(O)₂-cycloalkyl, —N(H)—S(O)₂—C₁-C₃haloalkyl, —P(O)(OH)₂, —P(O)(C₁-C₆alkyl)₂, cycloalkyl optionally substituted with 1-3 R¹⁰, heterocycloalkyl optionally substituted with 1-3 R¹⁰, or heteroaryl optionally substituted with 1-3 R¹⁰; aryl optionally substituted with 1-3 R¹⁰; or
two R⁸, together with the carbon atom to which they are attached, join together to form a cycloalkyl;
each R¹⁰ is independently hydroxy, halogen, CN, C₁-C₆alkyl, haloalkyl, cycloalkyl, C₂-C₆ alkenyl, heteroaryl, —NH₂, —C(O)-alkenyl, —C(O)—NH₂, —C(O)O-alkyl, —NH—C(O)-alkenyl, —S(O)₂—C₁-C₃alkyl, —S(O)₂—C₂-C₆alkenyl, or —S(O)₂—NH₂;
R¹¹ is CN;
each R¹² and R¹³ are independently hydrogen, C₁-C₆alkyl, or C₃-C₆cycloalkyl; and
n is 0 or 1
provided that ring A and/or ring B is a 5- or 6-membered heteroaryl comprising 1-4 heteroatoms selected from N, O and S.

23. The compound of claim 22, wherein:
Z is —(CH₂)₂—S(O)₂—CH₃ or —CH₂—N(H)—C(O)—NH₂.

24. A formic acid salt according to claim 1.

25. A compound selected from Table 1, or a pharmaceutically acceptable salt thereof.

26. A compound according to claim 1, wherein the compound is a non-covalent inhibitor of TEAD.

27. A pharmaceutical composition comprising a compound of claim 1, and a pharmaceutically acceptable carrier.

28. The pharmaceutical composition of claim 27, further comprising a second pharmaceutical agent.

29. A method for treating a subject with a disease or condition mediated by YAP/TEAD, said method comprising administering to the subject an effective amount of a compound of claim 1, or a pharmaceutically acceptable salt, deuterated analog, a tautomer or a stereoisomer thereof, or a pharmaceutical composition thereof.

30. The method of claim 29, wherein the disease or condition is a cancer, a neurodegenerative disease, a heart related disorder, or a kidney-related disorder.

31. The method of claim 29, wherein the disease or condition is polycystic kidney disease, Alzheimer's disease, arrhythmogenic cardiomyopathy, Holt-Oram syndrome, liver cancer, epithelioid hemangioendothelioma, breast cancer, lung cancer, malignant mesothelioma, pancreatic cancer, kaposi sarcoma, uveal melanoma, renal cell carcinoma, colorectal cancer, multiple myeloma, neurofibromatosis Type 2, glioma, or glioblastoma.

32. The method according to claim 29, further comprising administering one or more additional therapeutic agents.

33. The method according to claim 32, wherein the one or more additional therapeutic agents is one or more of i) an alkylating agent; ii) an antibiotic; iii) an antimetabolite; iv) an immune checkpoint agent; v) a hormone or hormone antagonist; vi) a taxane; vii) a retinoid; viii) an alkaloid; ix) an antiangiogenic agent; x) a topoisomerase inhibitor; xi) a kinase inhibitor; xii) a targeted signal transduction inhibitor; xiii) a biological response modifier; xiv) an IDO inhibitor; xv) a chemotherapeutic agent; xvi) a BRAF inhibitor; xvii) a Mek inhibitor; xviii) c-Kit mutant inhibitor, xix) an EGFR inhibitor, xx) an epigenetic modulator; xxi) other adenosine axis blockade agents; and xxiii) an anti-ErbB2 mAb.
